(19) United States
(12) Patent Application Publication
(10) Pub. No.: US 2026/0354019 A1
(43) Pub. Date: Nov. 5, 2026

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Dongjin JANG, Suwon-si (KR); Giwook KANG, Suwon-si (KR); Eunhye AN, Suwon-si (KR); Jisoo SHIN, Suwon-si (KR); Soonhyung KWON, Suwon-si (KR); Seokwoo KANG, Suwon-si (KR); Hayun SONG, Suwon-si (KR)

(21) Appl. No.: 19/642,910

(22) Filed: Apr. 9, 2026

(30) Foreign Application Priority Data
Apr. 11, 2025 (KR) 10-2025-0047618

(51) Int. Cl. H10K 85/60; C07B 59/00; C07D 403/14; C09K 11/02; H10K 50/12; H10K 85/40; H10K 101/00

(52) U.S. Cl. CPC H10K 85/6572; C07B 59/002; C07D 403/14; C09K 11/02; H10K 85/40; H10K 85/654; H10K 50/121; H10K 2101/27

(57) ABSTRACT

A compound, a composition including the compound, an organic optoelectronic device including the compound or composition, and a display device including the organic optoelectronic device, the compound being represented by a combination of Chemical Formula 1 and Chemical Formula 2:

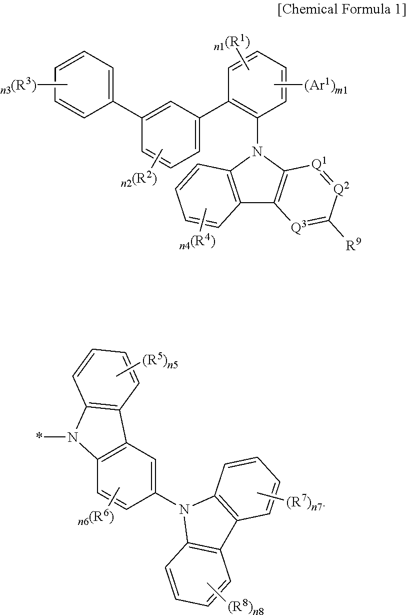

### CROSS-REFERENCE TO RELATED APPLICATION

This application claims priority to and the benefit of Korean Patent Application No. 10-2025-0047618 filed with the Korean Intellectual Property Office on Apr. 11, 2025, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., an organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode may be a device that converts electrical energy into light, and the performance of the organic light emitting diode may be influenced by an organic material between electrodes.

### SUMMARY

Embodiments are directed to a compound for an organic optoelectronic device represented by a combination of Chemical Formula 1 and Chemical Formula 2:

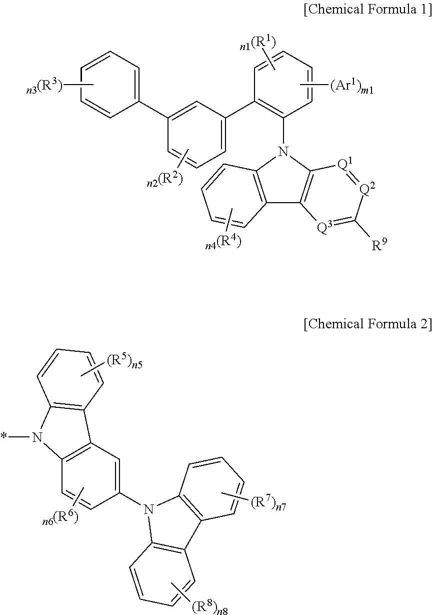

wherein, in Chemical Formula 1 and Chemical Formula 2, Q¹, Q² and Q³ are each independently C—R^{a} or a linking carbon linked at * of Chemical Formula 2, provided that one of Q¹, Q² and Q³ is a linking carbon linked at * of Chemical Formula 2, R^{a} and R¹ to R⁹ are each independently hydrogen, deuterium, a substituted or unsubstituted C1 to C10 alkyl group, or a substituted or unsubstituted C6 to C12 aryl group, Ar¹ is a substituted or unsubstituted phenyl group or a substituted or unsubstituted biphenyl group, n1 and n6 are each independently an integer of 1 to 3, n2, n4, n5, n7, and n8 are each independently an integer of 1 to 4, n3 is an integer of 1 to 5, m1 is 0 or 1, and when n1 to n8 is 2 or more, each R¹ to R⁸ is the same or different from each other R¹ to R⁸, respectively.

The embodiments may be realized by providing a composition for an organic optoelectronic device, including a first compound and a second compound, wherein the first compound is the compound for an organic optoelectronic device according to some embodiments, and the second compound is represented by Chemical Formula 3:

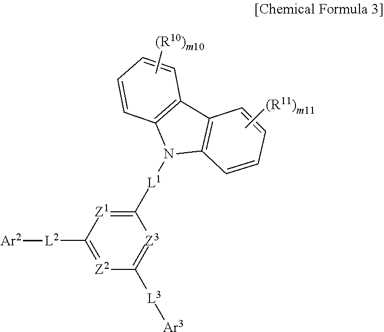

in Chemical Formula 3, Z¹ to Z³ are each independently N or CR^{b}, provided that at least two of Z¹ to Z³ are N, L¹ to L³ are each independently a single bond, a substituted or unsubstituted C6 to C20 arylene group, or a substituted or unsubstituted divalent C2 to C20 heterocyclic group, Ar² and Ar³ are each independently a substituted or unsubstituted C6 to C30 aryl group, a substituted or unsubstituted C2 to C20 heterocyclic group, or a substituted or unsubstituted silyl group, R^{b}, R¹⁰, and R¹¹ are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, each R¹⁰ and R¹¹ are separately present or two adjacent ones are linked to form a ring, and m10 and m11 are each independently an integer of 1 to 4.

The embodiments may be realized by providing an organic optoelectronic device including an anode and a cathode facing each other, and an organic layer between the anode and the cathode, wherein the organic layer includes the compound for an organic optoelectronic device according to an embodiment.

The embodiments may be realized by providing an organic optoelectronic device including an anode and a cathode facing each other, and an organic layer between the anode and the cathode, wherein the organic layer includes the composition for an organic optoelectronic device according to an embodiment.

The embodiments may be realized by providing a display device including the organic optoelectronic device according to an embodiment.

### BRIEF DESCRIPTION OF THE DRAWINGS

Features will become apparent to those of skill in the art by describing in detail exemplary embodiments with reference to the attached drawings in which:

FIG. 1 is a cross-sectional view showing an example of an organic light emitting diode, which is an example of an organic optoelectronic device according to some example embodiments, and

FIG. 2 is a cross-sectional view showing another example of an organic light emitting diode, which is an example of an organic optoelectronic device according to some example embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present embodiments, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present embodiments, the “substituted” refers to replacement of at least one hydrogen of a substituent or a compound by deuterium, a C1 to C20 alkyl group, a C3 to C20 cycloalkyl group, a C6 to C30 aryl group, or a cyano group. In specific example of the present embodiments, the “substituted” refers to replacement of at least one hydrogen of a substituent or a compound by deuterium, a C1 to C5 alkyl group, a C3 to C10 cycloalkyl group, a C6 to C18 aryl group, or a cyano group. In specific example of the present embodiments, the “substituted” refers to replacement of at least one hydrogen of a substituent or a compound by deuterium, a cyano group, a methyl group, an ethyl group, a propyl group, a butyl group, a tert-butyl group, a cyclopropyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

As an example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to some example embodiments is described.

The compound for an organic optoelectronic device according to some example embodiments may be, e.g., represented by a combination of Chemical Formula 1 and Chemical Formula 2.

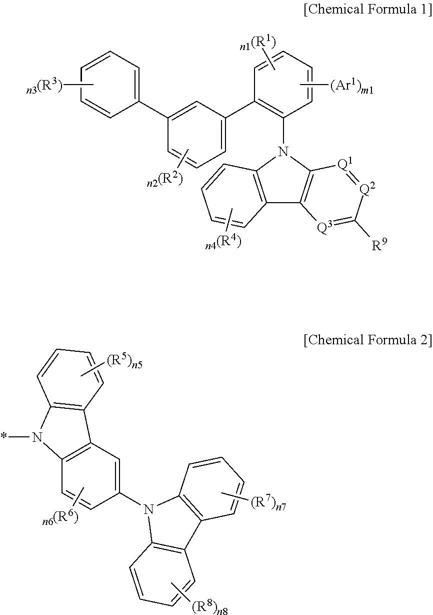

In Chemical Formula 1 and Chemical Formula 2, Q¹, Q² and Q³ may each independently be, e.g., C—R^{a} or a linking carbon linked at * of Chemical Formula 2.

In an implementation, one of Q¹, Q² and Q³ is linked to Chemical Formula 2.

R^{a} and R¹ to R⁹ may each independently be or include, e.g., hydrogen. deuterium, a substituted or unsubstituted C1 to C10 alkyl group, or a substituted or unsubstituted C6 to C12 aryl group.

Ar¹ may be, e.g., a substituted or unsubstituted phenyl group or a substituted or unsubstituted biphenyl group.

n1 and n6 may each independently be, e.g., an integer of 1 to 3.

n2, n4, n5, n7, and n8 may each independently be, e.g., an integer of 1 to 4.

n3 may be, e.g., an integer of 1 to 5.

m1 may be, e.g., an integer of 0 or 1.

In an implementation, n1 may be, e.g., 2 or 3, and each R¹ may be the same or different each other.

In an implementation, n2 may be, e.g., 2, 3, or 4, and each R² may be the same or different each other.

In an implementation, n3 may be, e.g., 2, 3, 4, or 5, and each R³ may be the same or different each other.

In an implementation, n4 may be, e.g., 2, 3, or 4, and each R⁴ may be the same or different each other.

In an implementation, n5 may be, e.g., 2, 3, or 4, and each R⁵ may be the same or different each other.

In an implementation, n6 may be, e.g., 2 or 3, and each R⁶ may be the same or different each other.

In an implementation, n7 may be, e.g., 2, 3, or 4, and each R⁷ may be the same or different each other.

In an implementation, n8 may be, e.g., 2, 3, or 4, and each R⁸ may be the same or different each other.

The compound represented by the combination of Chemical Formula 1 and Chemical Formula 2 has a structure in which three carbazoles are linked, and thus the hole characteristics may be further strengthened and thus the low-driving characteristics may be exhibited.

In particular, when 9-carbazole is linked at the 1st, 2nd, or 4th position of carbazole, and 9-carbazole is additionally linked at 3rd position with respect to the 9-carbazole, life-span characteristics may be further improved.

In an implementation, additional life-span enhancement may be achieved by introducing a LUMO phore (LUMO part in the molecule) in which the terphenyl group linked to the carbazole is linked in the ortho-meta direction.

In an implementation, Chemical Formula 1 may be, e.g., represented by one of Chemical Formula 1-I to Chemical Formula 1-III depending on the linking position of 9-carbazole.

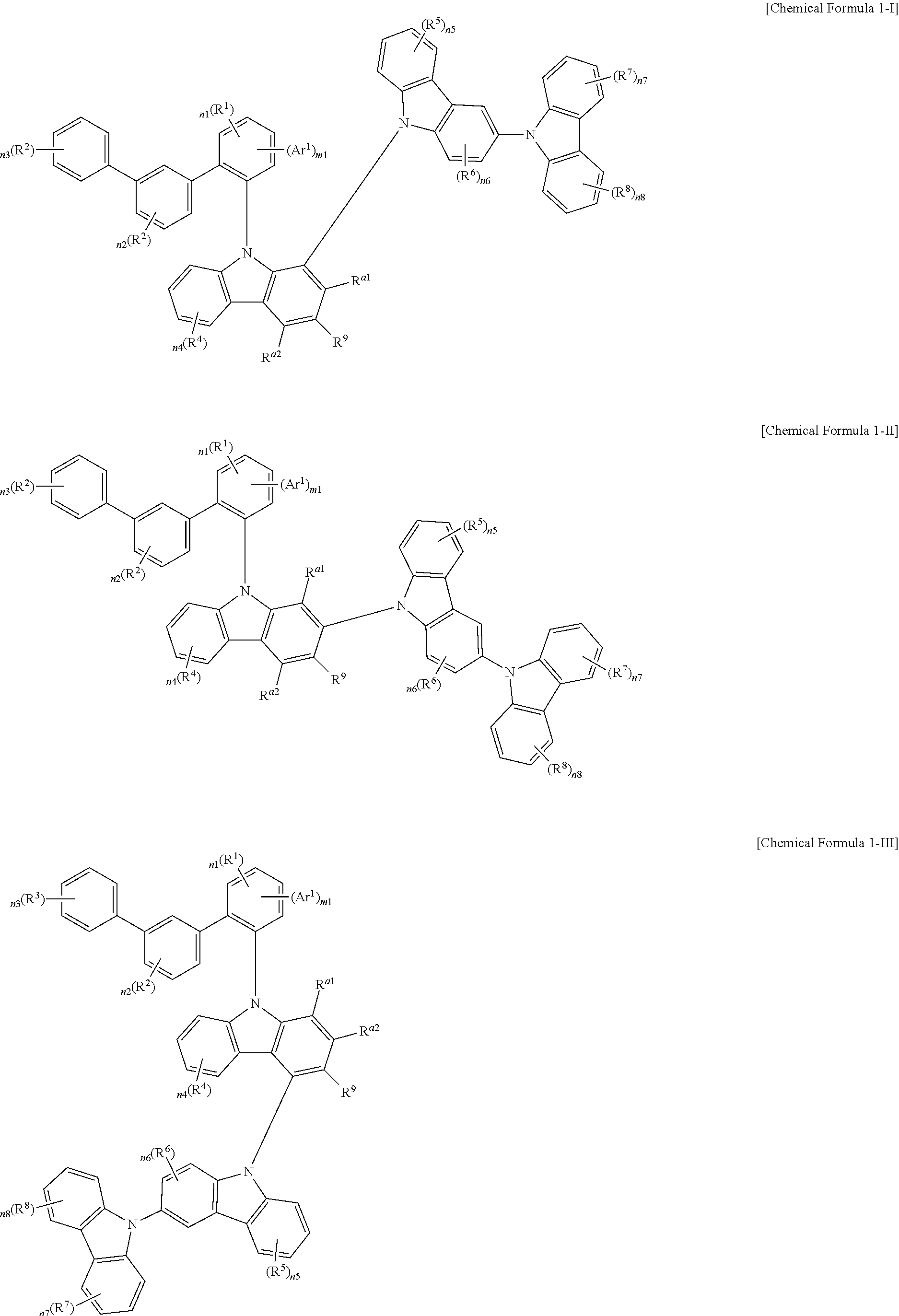

In Chemical Formula 1-I to Chemical Formula 1-III, R¹ to R⁹, Ar¹, n1 to n8 and m1 may be defined the same as those described above.

R^{a1} and R^{a2} may each be defined the same as R^{a} described above.

In an implementation, Chemical Formula 1 may be represented by, e.g., one of the Chemical Formula 1-1 to Chemical Formula 1-4 depending on the substitution position of Ar¹.

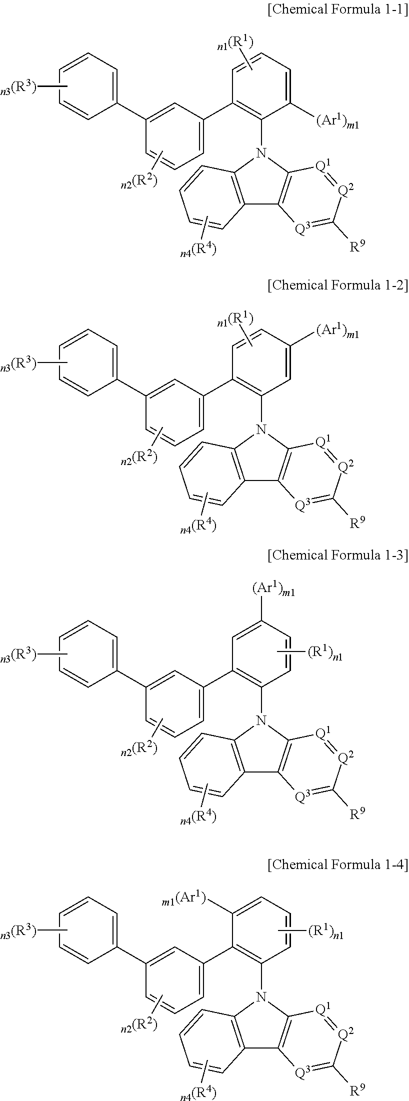

In Chemical Formula 1-1 to Chemical Formula 1-4, Q¹, Q², Q³, R¹ to R⁴, R⁹, Ar¹, n1 to n4, and m1 may be defined the same as those described above.

In an implementation, Chemical Formula 1 may be, e.g., represented by Chemical Formula 1-1 or Chemical Formula 1-2.

In an implementation, Chemical Formula 1 may be, e.g., represented by Chemical Formula 1-1.

In some example embodiments, the combination of Chemical Formula 1 and Chemical Formula 2 may be represented by, e.g., one of Chemical Formula 1-1A, Chemical Formula 1-1B, and Chemical Formula 1-1C.

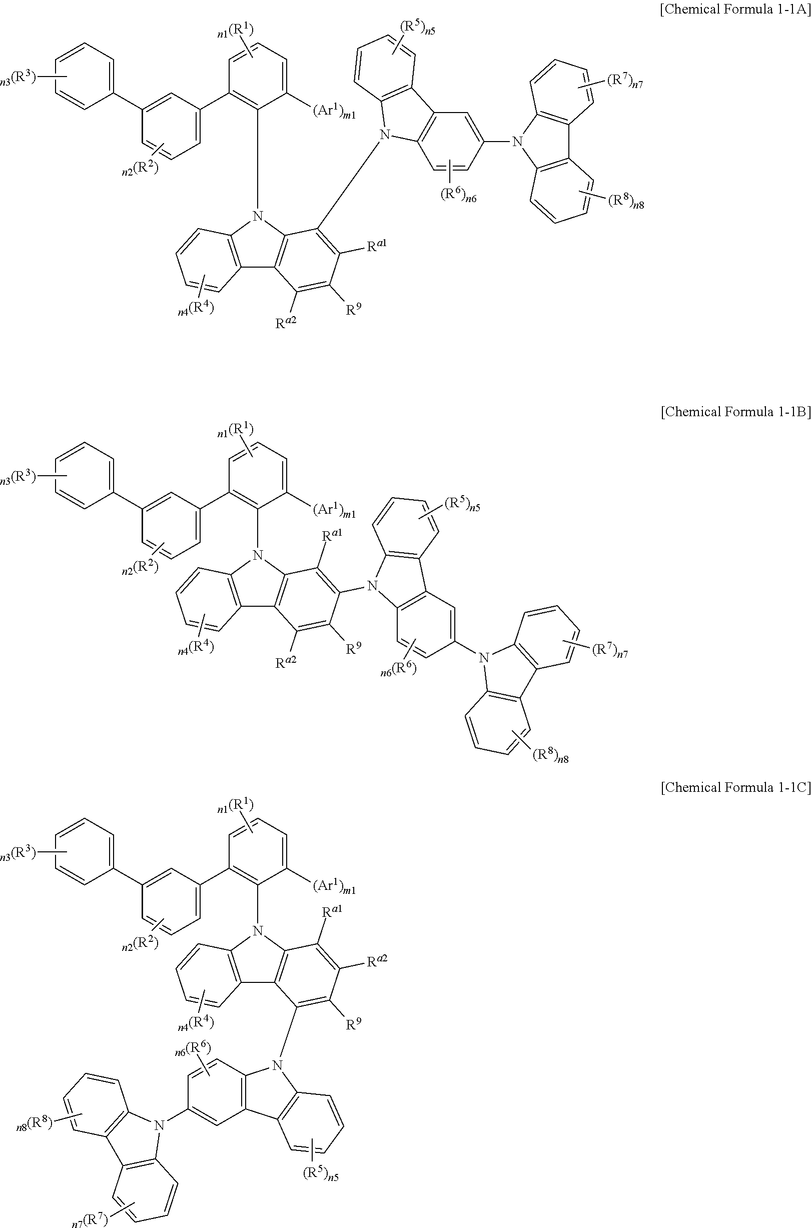

In Chemical Formula 1-1A, Chemical Formula 1-1B, and Chemical Formula 1-1C, R¹ to R⁹, R^{a1}, R^{a2}, Ar¹, n1 to n8, and m1 may be defined the same as those described above.

When Ar¹ is linked in an ortho position to 9-carbazole, the structure may become more advantageous in terms of T1 energy, and the efficiency and life-span of the device may be improved.

In a specific embodiment, the combination of Chemical Formula 1 and Chemical Formula 2 may be represented by Chemical Formula 1-1B.

In an implementation, R^{a} and R¹ to R⁹ may each independently be hydrogen, deuterium, a substituted or unsubstituted C1 to C6 alkyl group, or a substituted or unsubstituted phenyl group.

In an implementation, R^{a} and R¹ to R⁹ may each independently be, e.g., hydrogen, deuterium, or a substituted or unsubstituted phenyl group.

Meanwhile, the compound for an organic optoelectronic device may be substituted with at least one deuterium. The number of substituted deuterium atoms may be from 1 to the maximum number of hydrogen atoms in the chemical formula, e.g., from 1 to 40 or from 1 to 30.

Additional life-span improvements may be achieved through deuterium substitution.

In an implementation, the aforementioned compound for an organic optoelectronic device represented by the combination of Chemical Formula 1 and Chemical Formula 2 may be, e.g., a compound of Group 1.

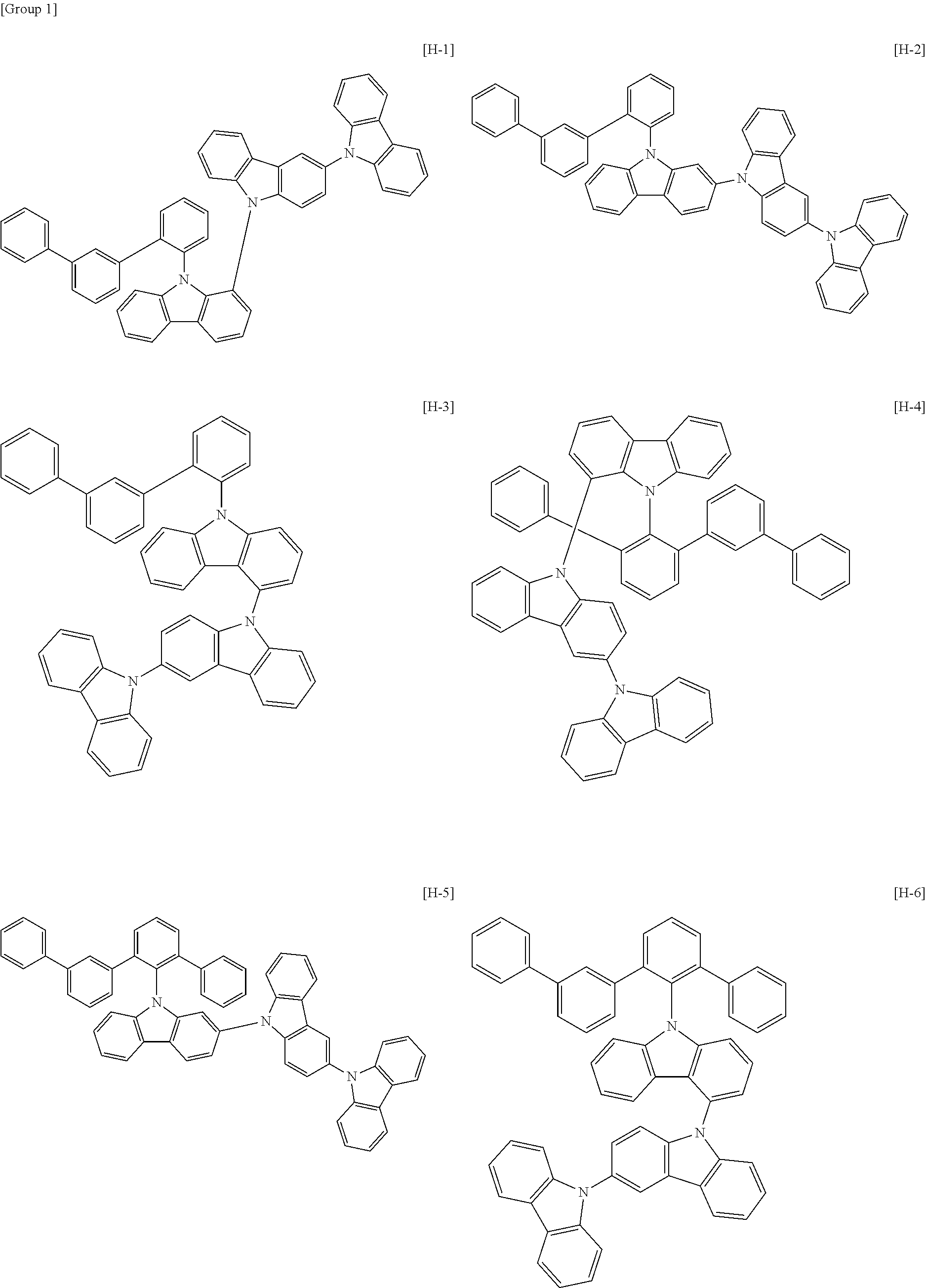

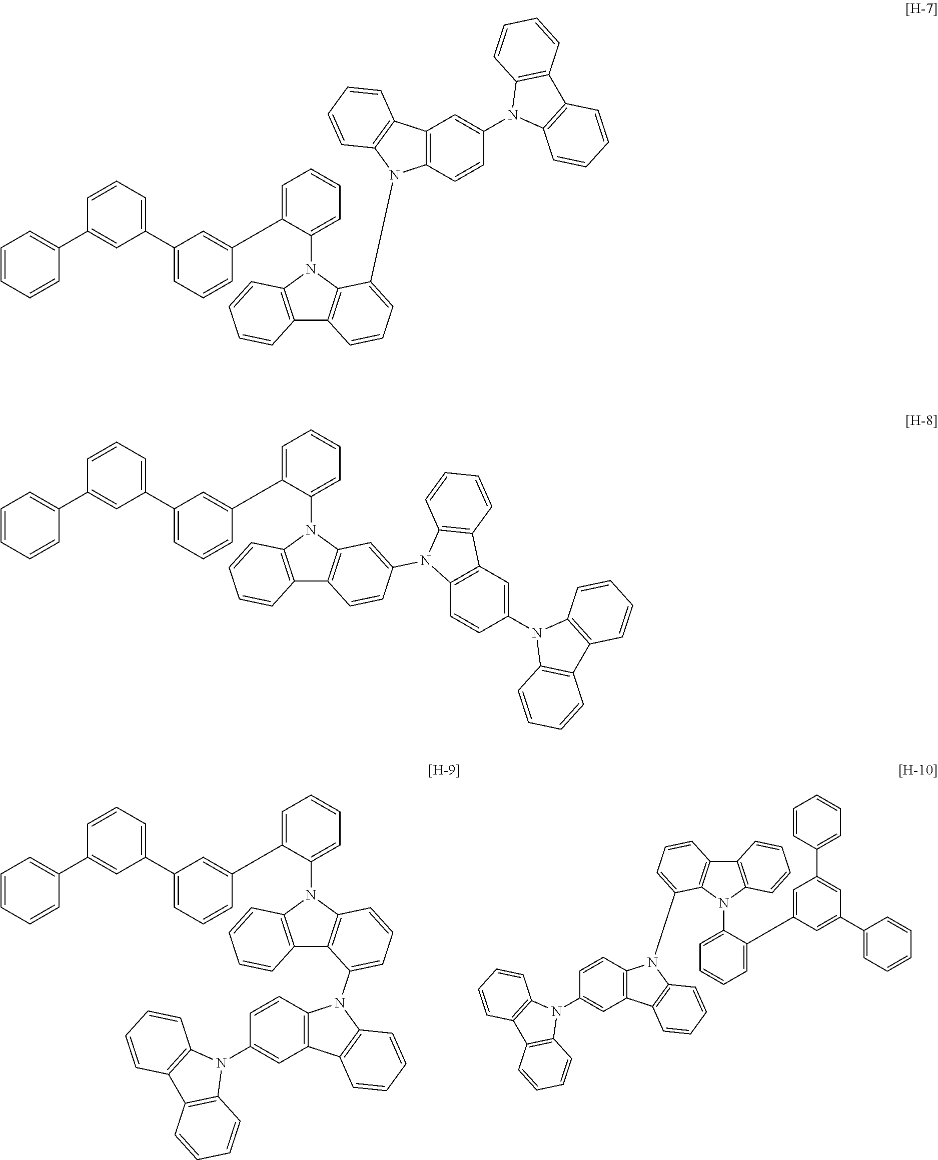 [SMILES: c1ccc(-c2cccc(-c3cccc(-c4ccccc4-n4c5ccccc5c5cccc(-n6c7ccccc7c7cc(-n8c9ccccc9c9ccccc98)ccc76)c54)c3)c2)cc1 ; c1ccc(-c2cccc(-c3cccc(-c4ccccc4-n4c5ccccc5c5ccc(-n6c7ccccc7c7cc(-n8c9ccccc9c9ccccc98)ccc76)cc54)c3)c2)cc1 ; c1ccc(-c2cccc(-c3cccc(-c4ccccc4-n4c5ccccc5c5c(-n6c7ccccc7c7cc(-n8c9ccccc9c9ccccc98)ccc76)cccc54)c3)c2)cc1 ; c1ccc(-c2cc(-c3ccccc3)cc(-c3ccccc3-n3c4ccccc4c4cccc(-n5c6ccccc6c6cc(-n7c8ccccc8c8ccccc87)ccc65)c43)c2)cc1]

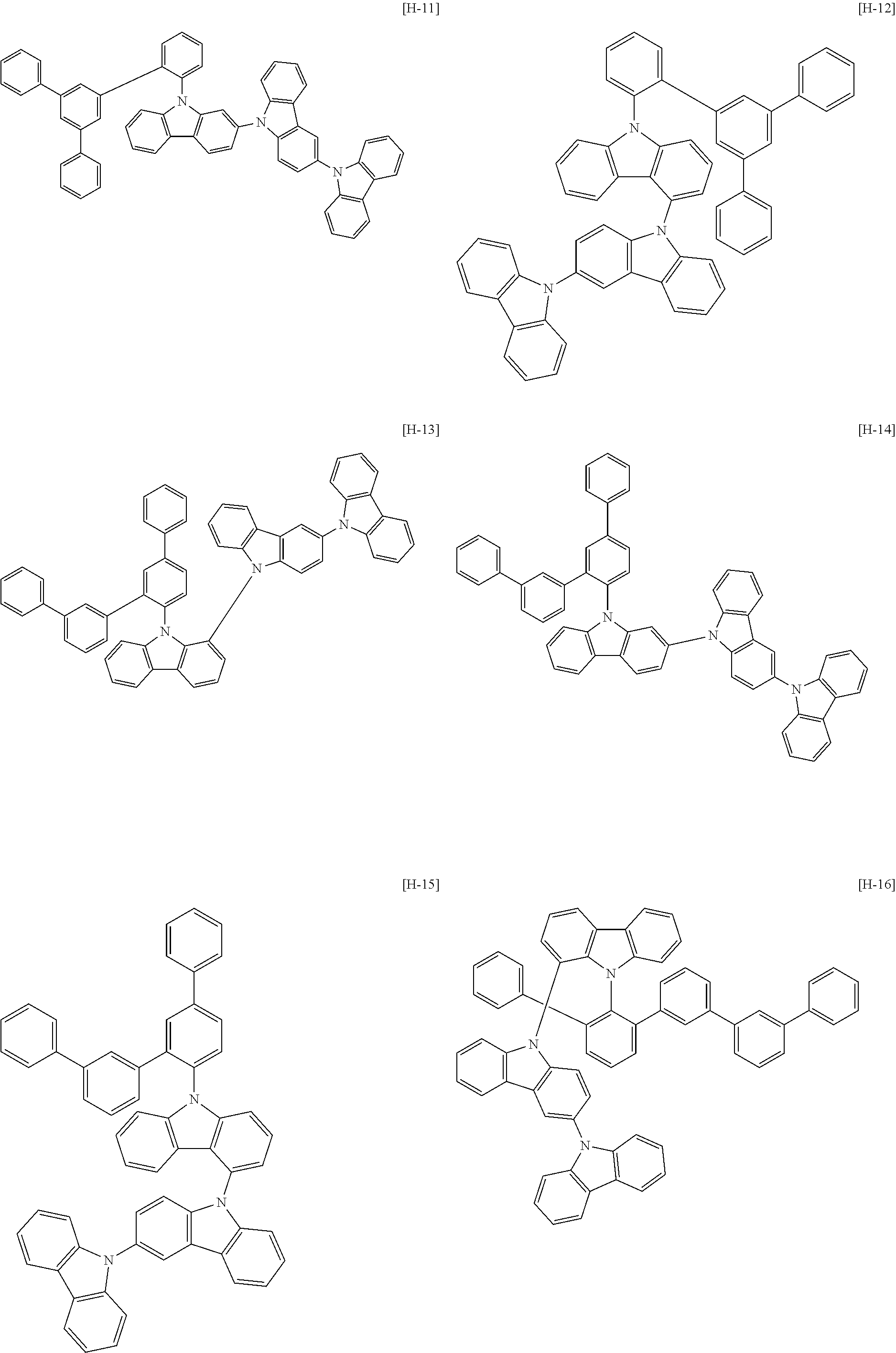

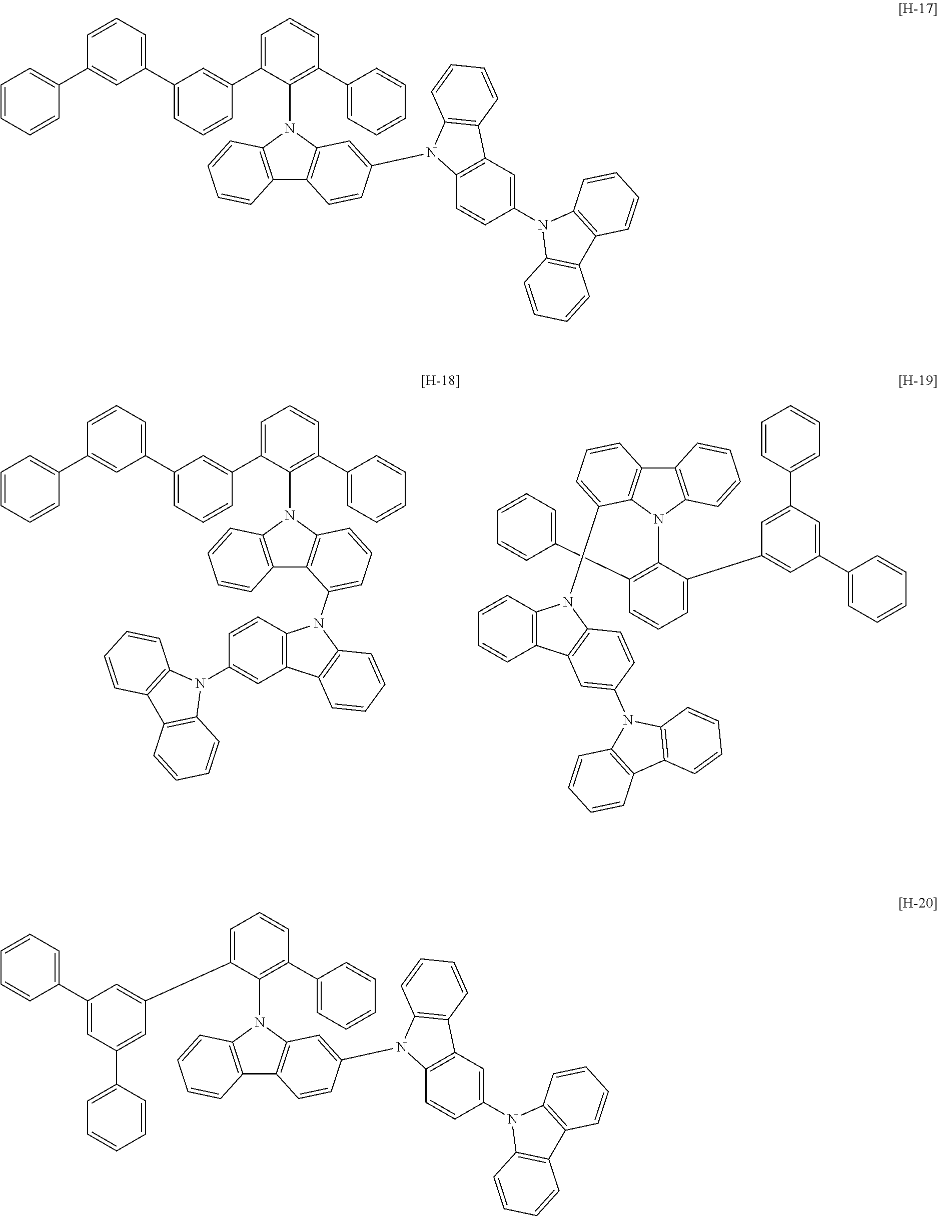 [SMILES: c1ccc(-c2cccc(-c3cccc(-c4cccc(-c5ccccc5)c4-n4c5ccccc5c5ccc(-n6c7ccccc7c7cc(-n8c9ccccc9c9ccccc98)ccc76)cc54)c3)c2)cc1 ; c1ccc(-c2cccc(-c3cccc(-c4cccc(-c5ccccc5)c4-n4c5ccccc5c5c(-n6c7ccccc7c7cc(-n8c9ccccc9c9ccccc98)ccc76)cccc54)c3)c2)cc1 ; c1ccc(-c2cc(-c3ccccc3)cc(-c3cccc(-c4ccccc4)c3-n3c4ccccc4c4cccc(-n5c6ccccc6c6cc(-n7c8ccccc8c8ccccc87)ccc65)c43)c2)cc1 ; c1ccc(-c2cc(-c3ccccc3)cc(-c3cccc(-c4ccccc4)c3-n3c4ccccc4c4ccc(-n5c6ccccc6c6cc(-n7c8ccccc8c8ccccc87)ccc65)cc43)c2)cc1]

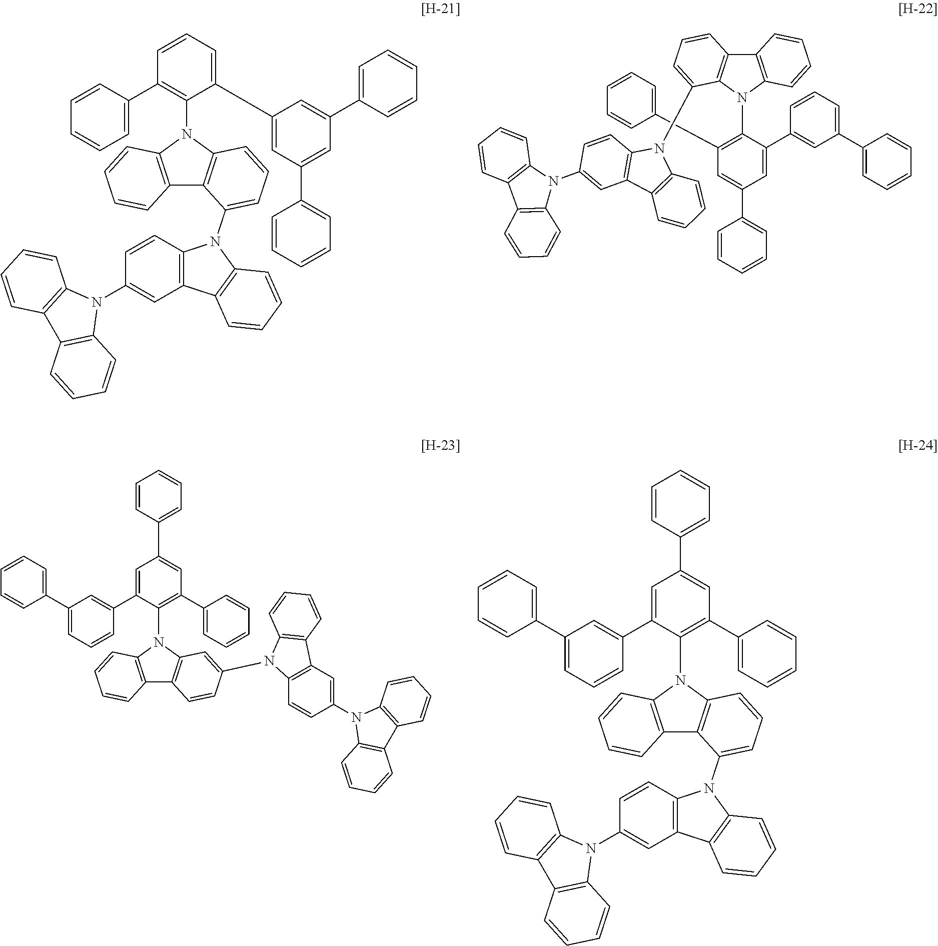 [SMILES: c1ccc(-c2cc(-c3ccccc3)cc(-c3cccc(-c4ccccc4)c3-n3c4ccccc4c4c(-n5c6ccccc6c6cc(-n7c8ccccc8c8ccccc87)ccc65)cccc43)c2)cc1 ; c1ccc(-c2cccc(-c3cc(-c4ccccc4)cc(-c4ccccc4)c3-n3c4ccccc4c4cccc(-n5c6ccccc6c6cc(-n7c8ccccc8c8ccccc87)ccc65)c43)c2)cc1 ; c1ccc(-c2cccc(-c3cc(-c4ccccc4)cc(-c4ccccc4)c3-n3c4ccccc4c4ccc(-n5c6ccccc6c6cc(-n7c8ccccc8c8ccccc87)ccc65)cc43)c2)cc1 ; c1ccc(-c2cccc(-c3cc(-c4ccccc4)cc(-c4ccccc4)c3-n3c4ccccc4c4c(-n5c6ccccc6c6cc(-n7c8ccccc8c8ccccc87)ccc65)cccc43)c2)cc1]

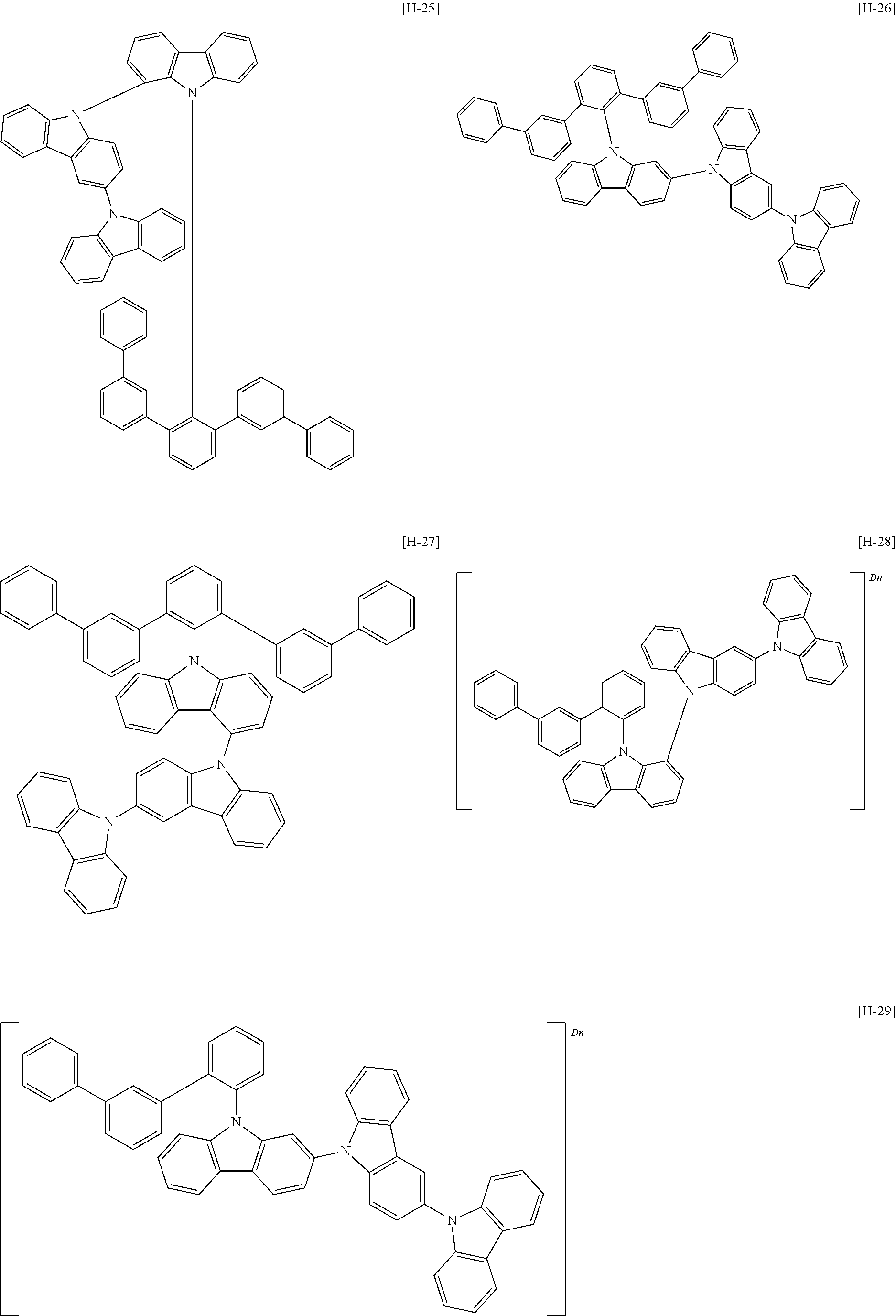

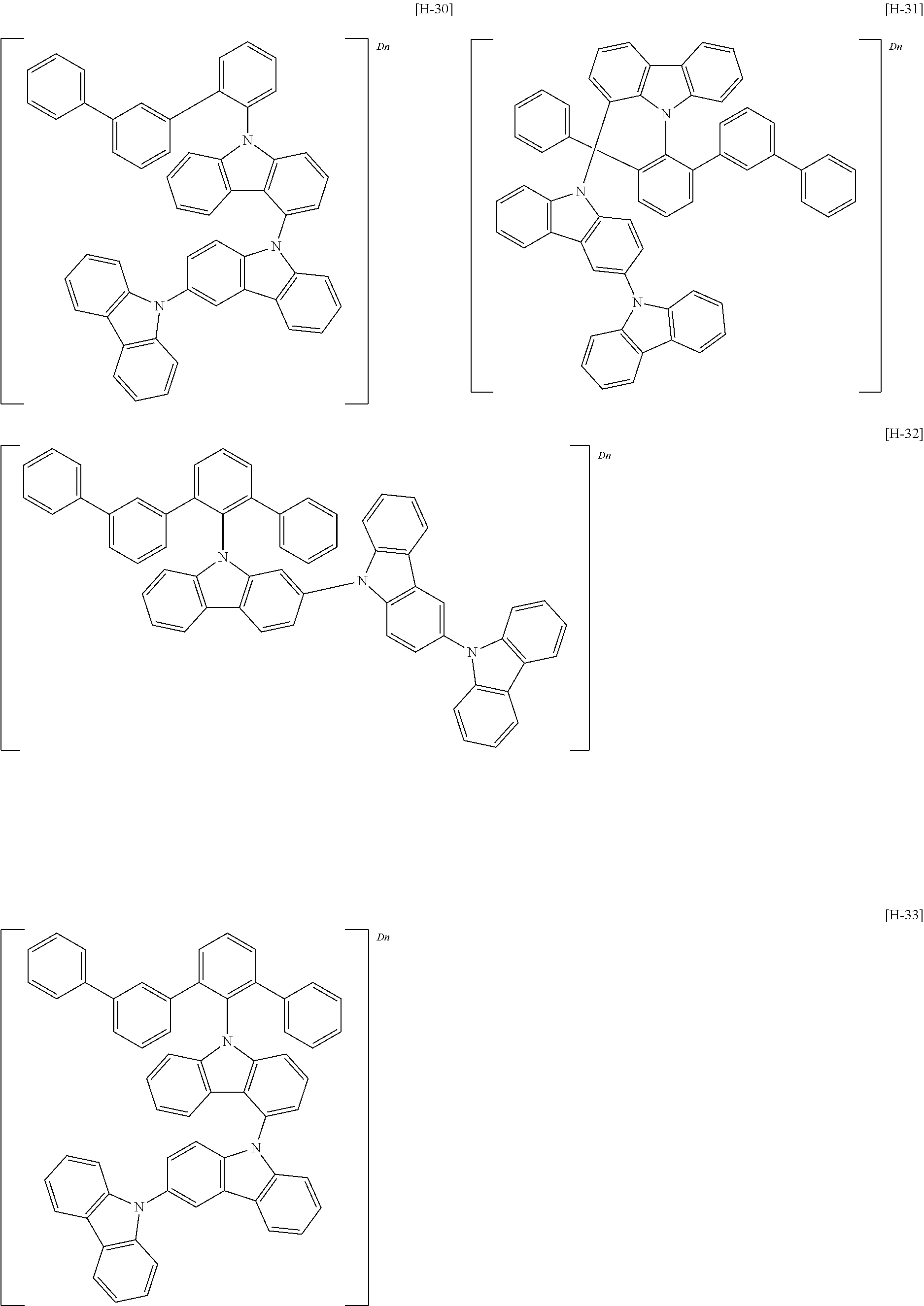

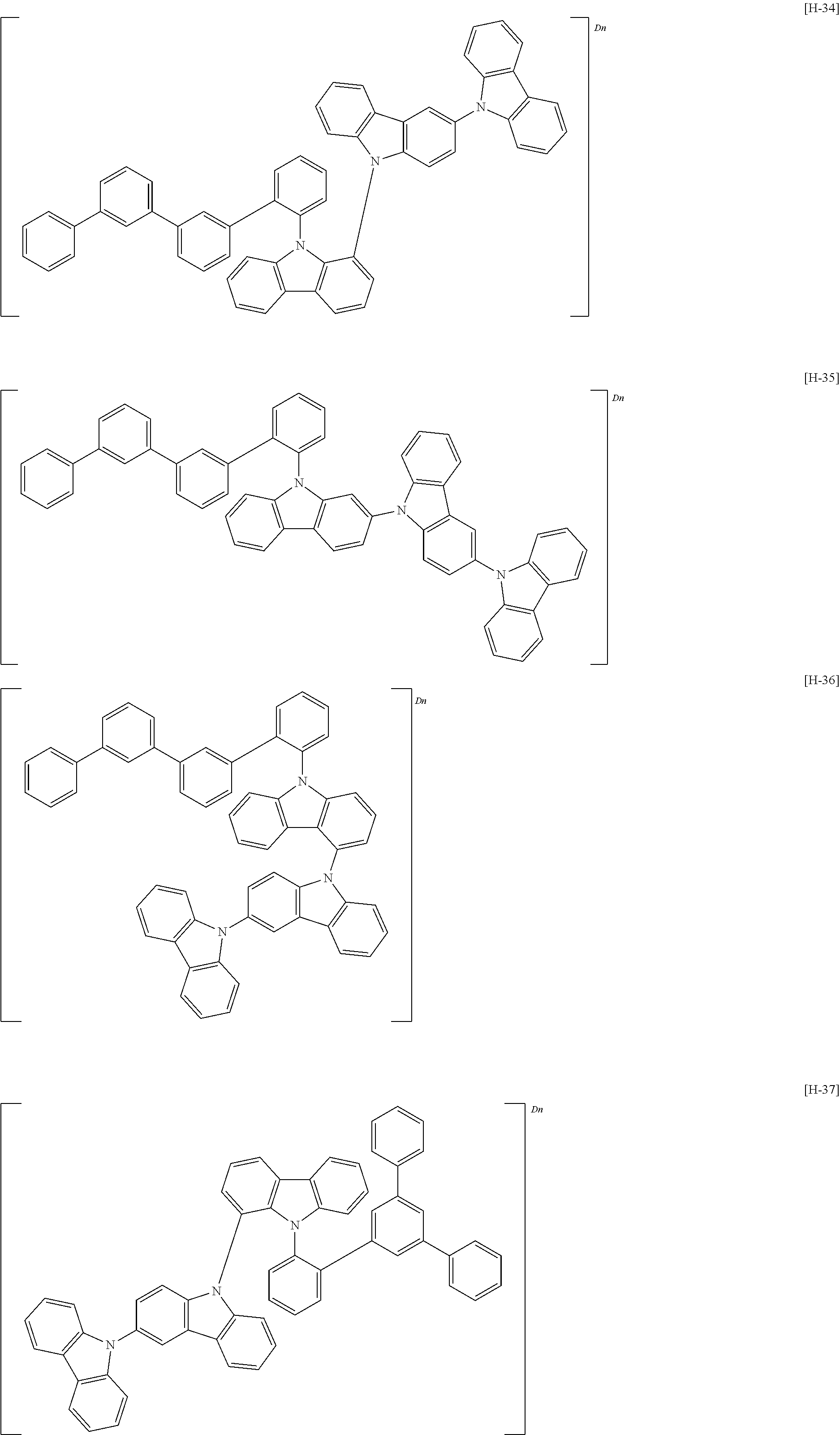

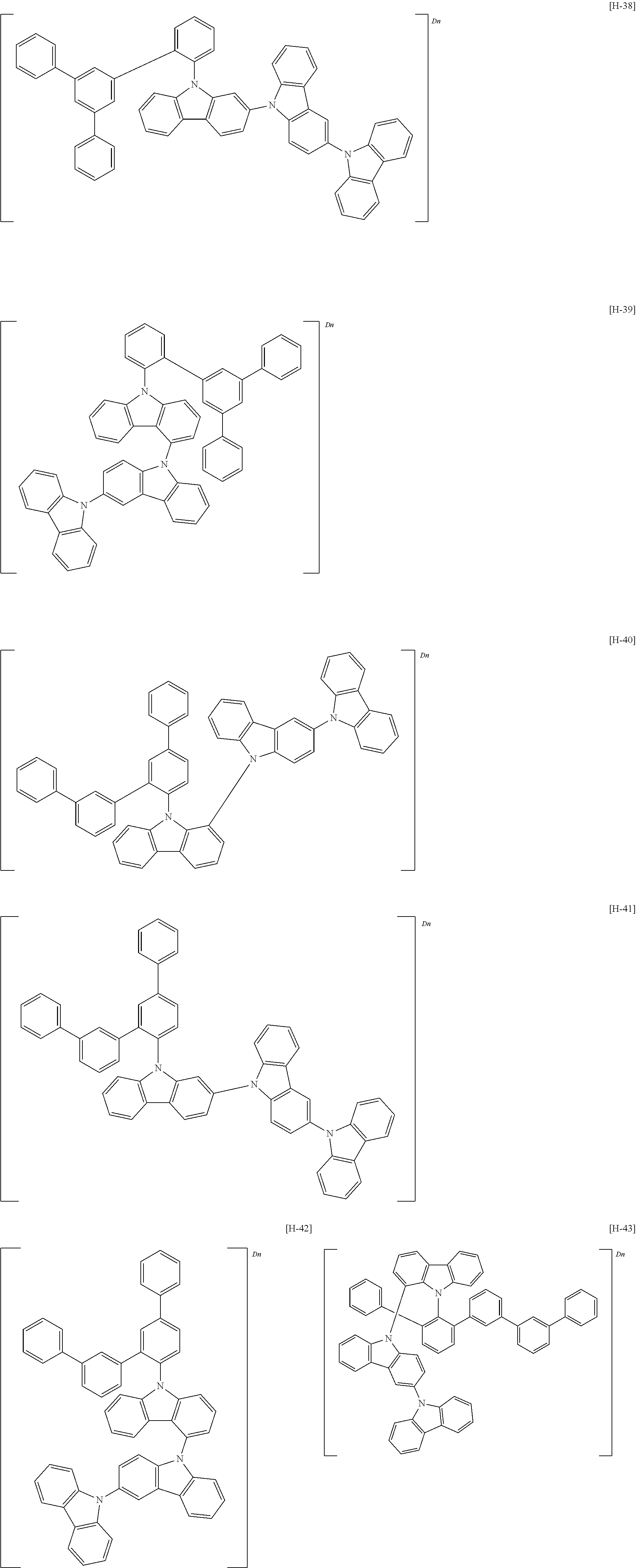

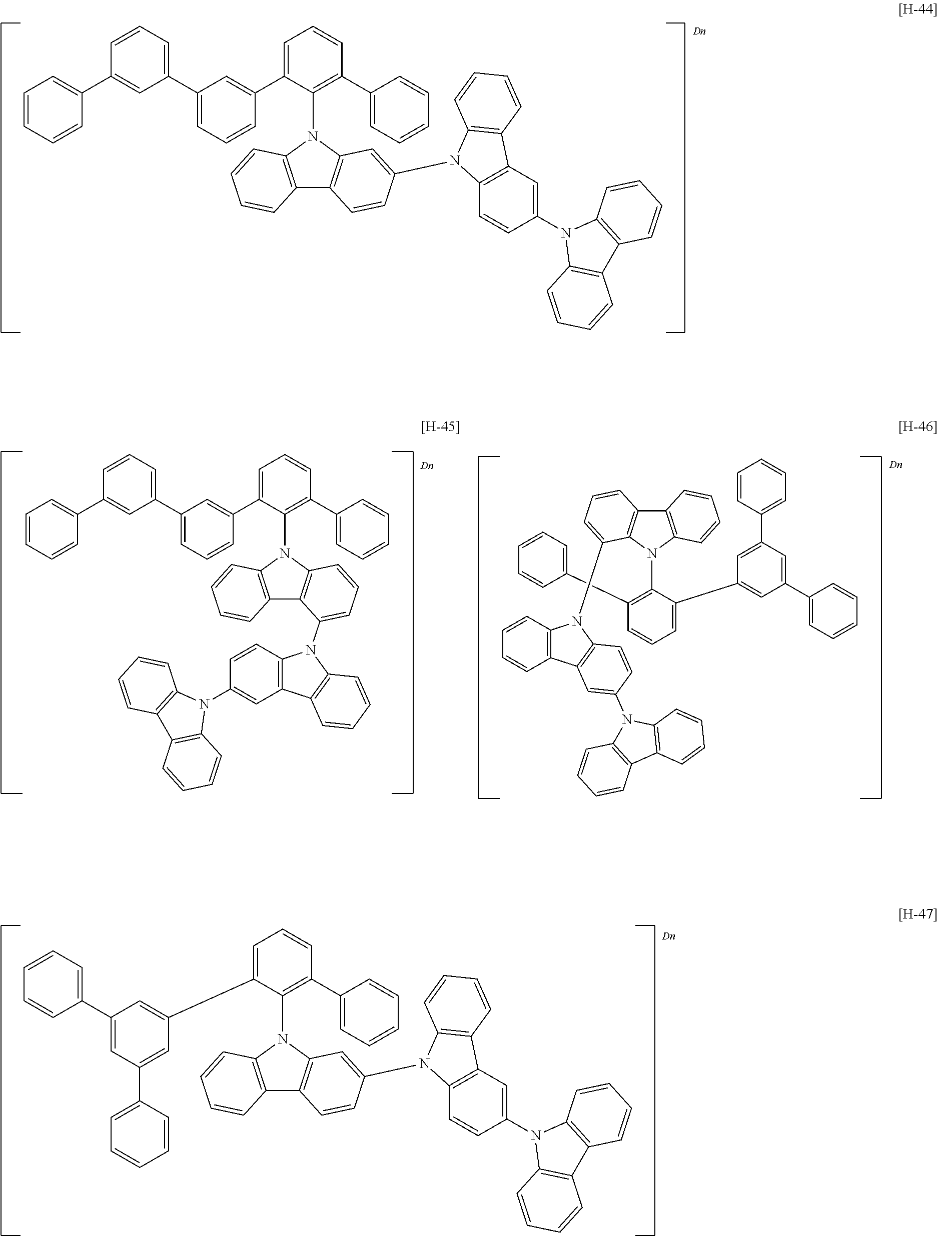

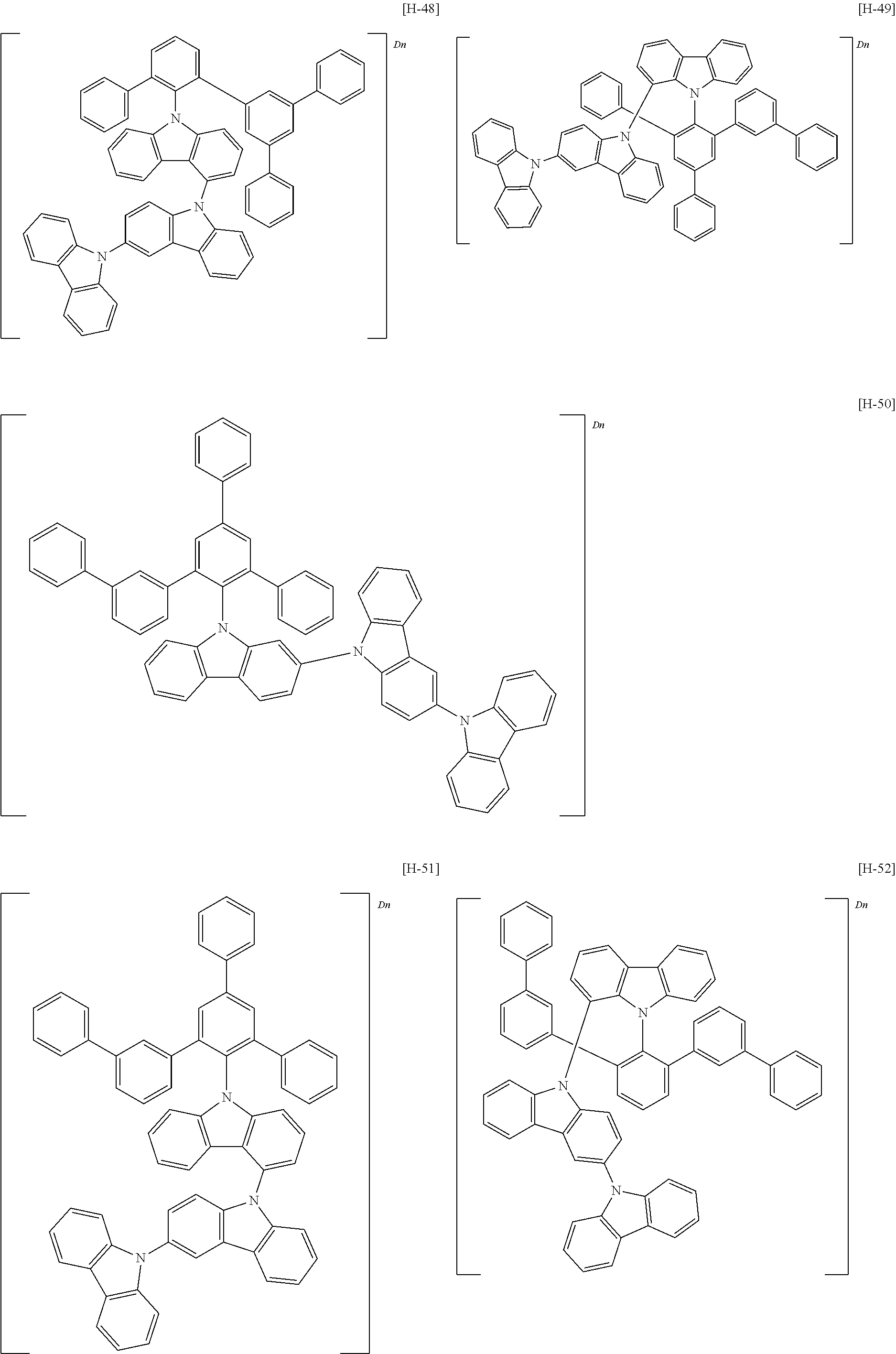

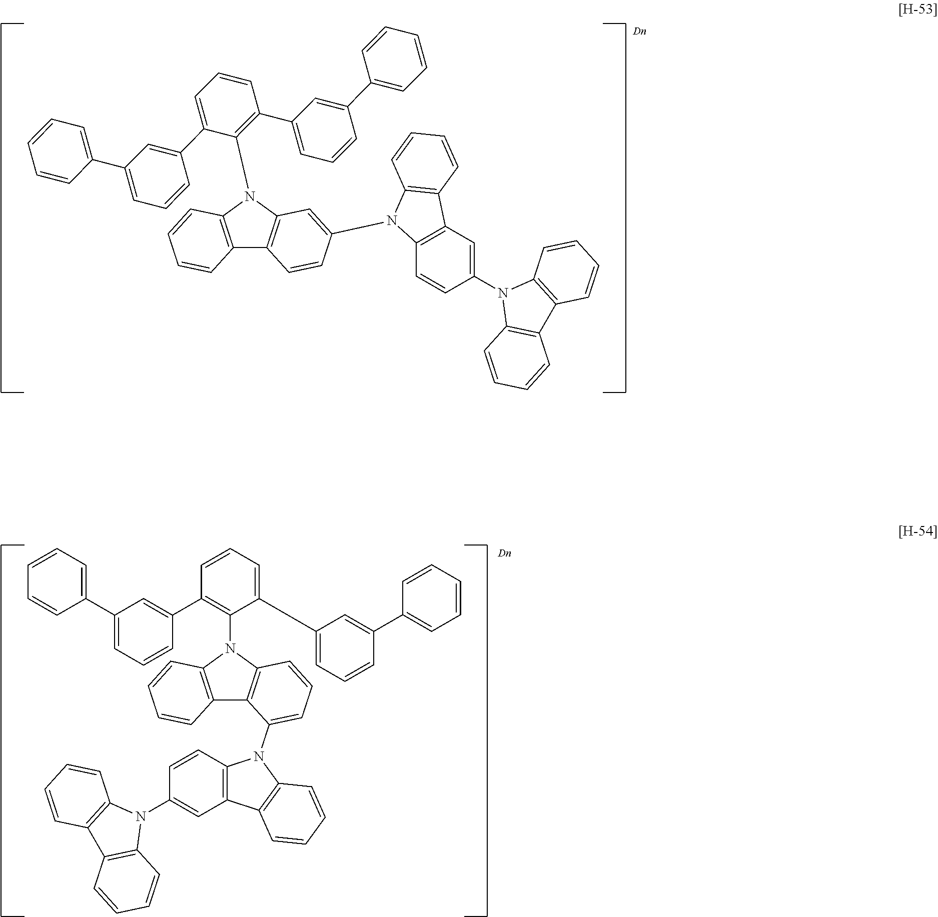

Dn indicates the number of hydrogen atoms replaced by deuterium, and n is an integer between 1 and the maximum number of possible replacements.

In an implementation, the compound for an organic optoelectronic device may be substituted with deuterium and may be, e.g., a compound of Group 1-1.

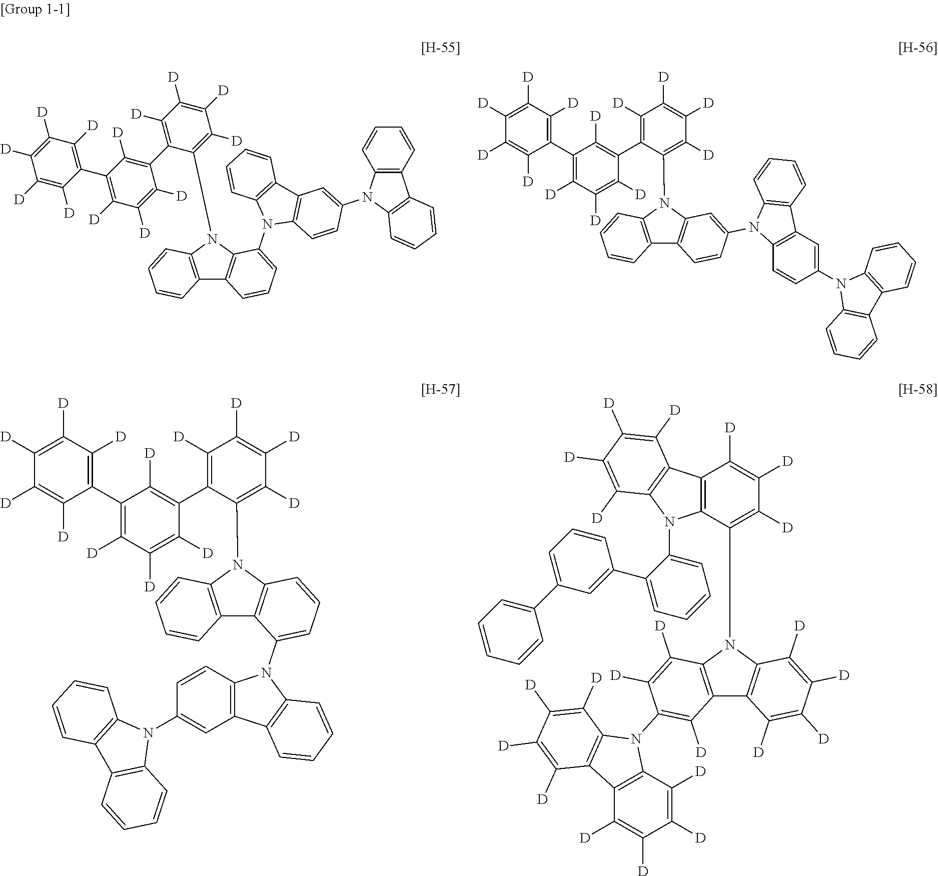

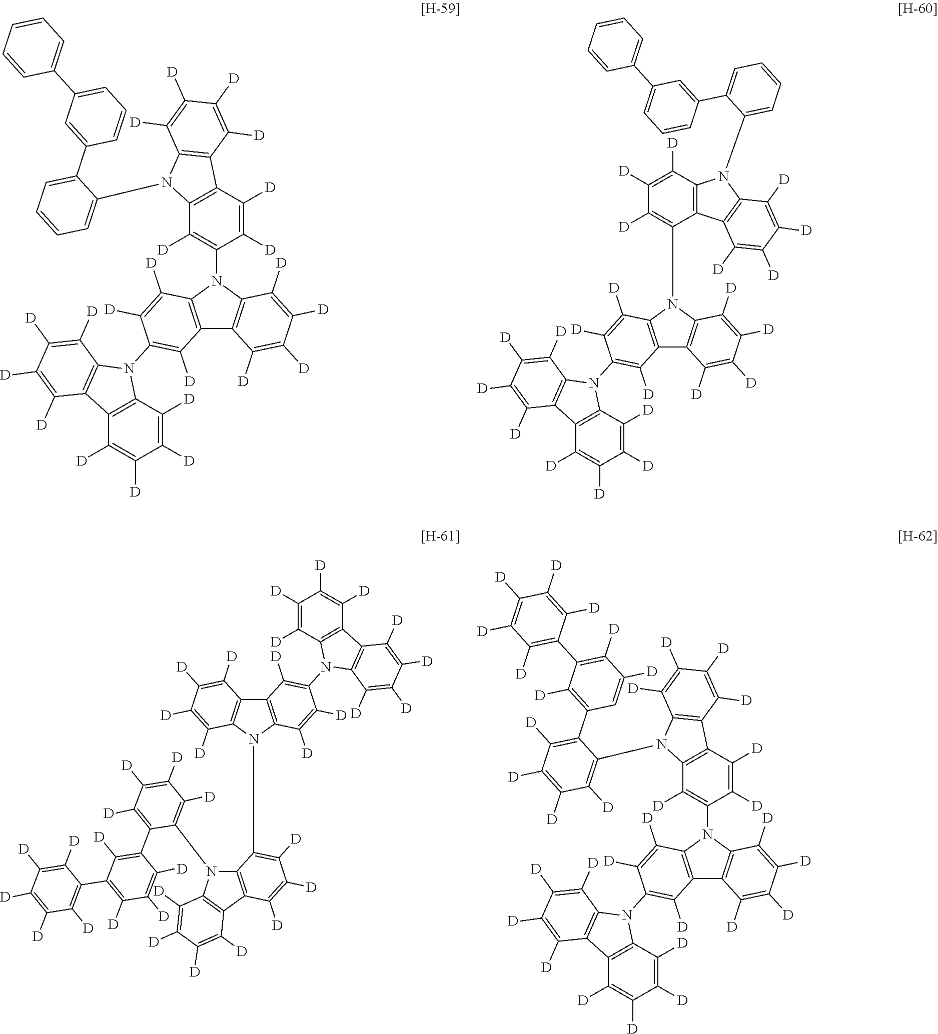

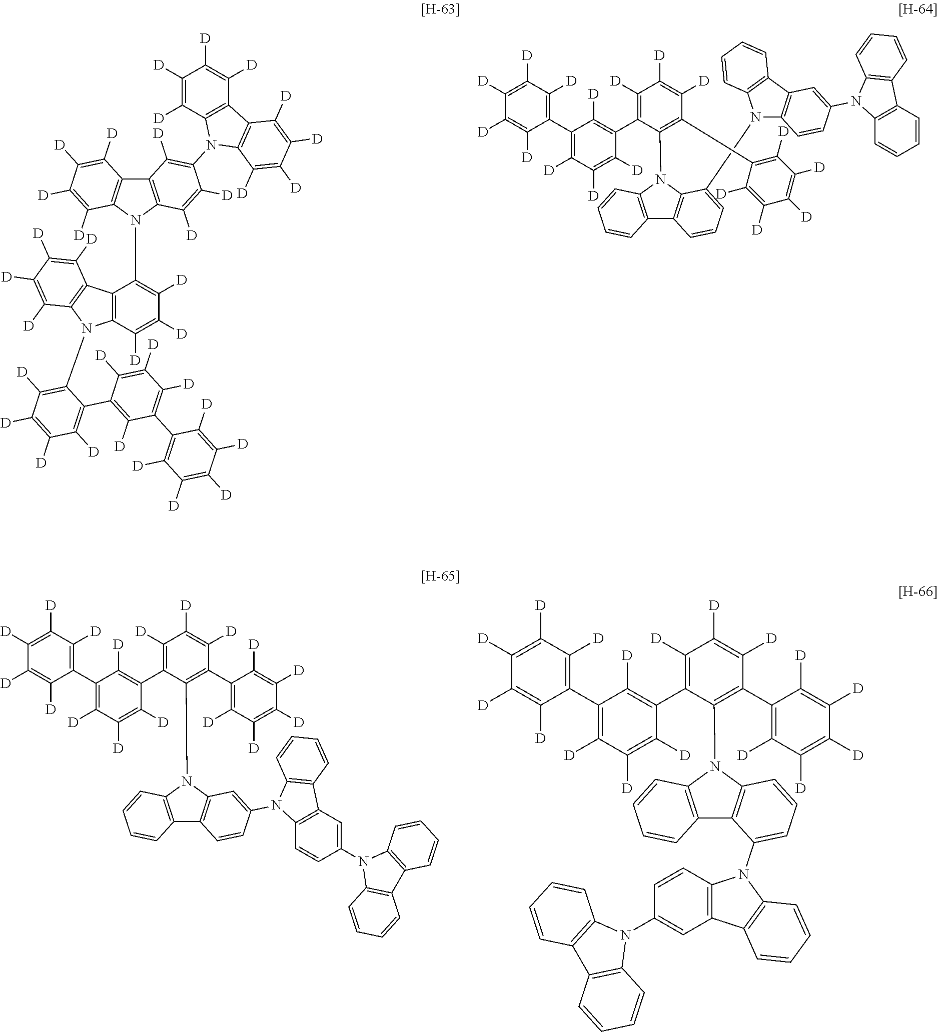

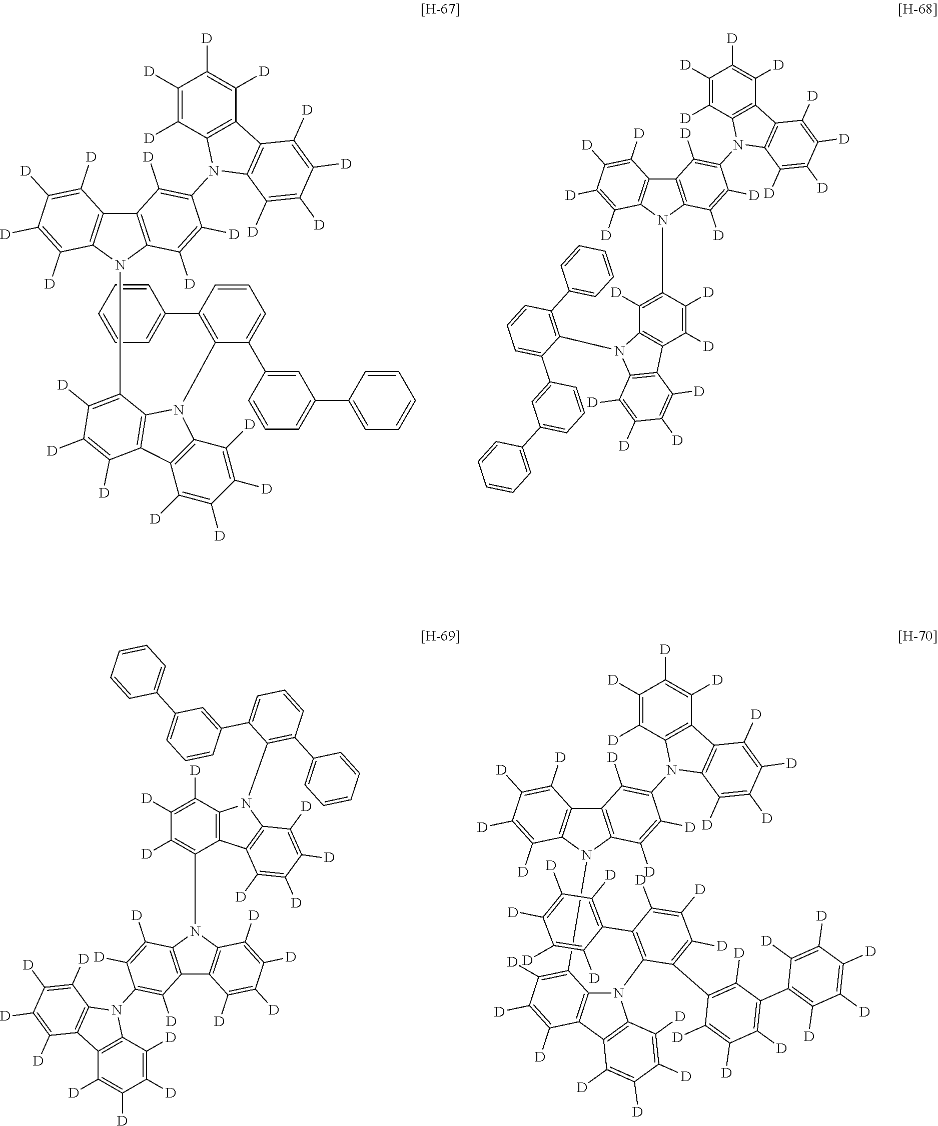

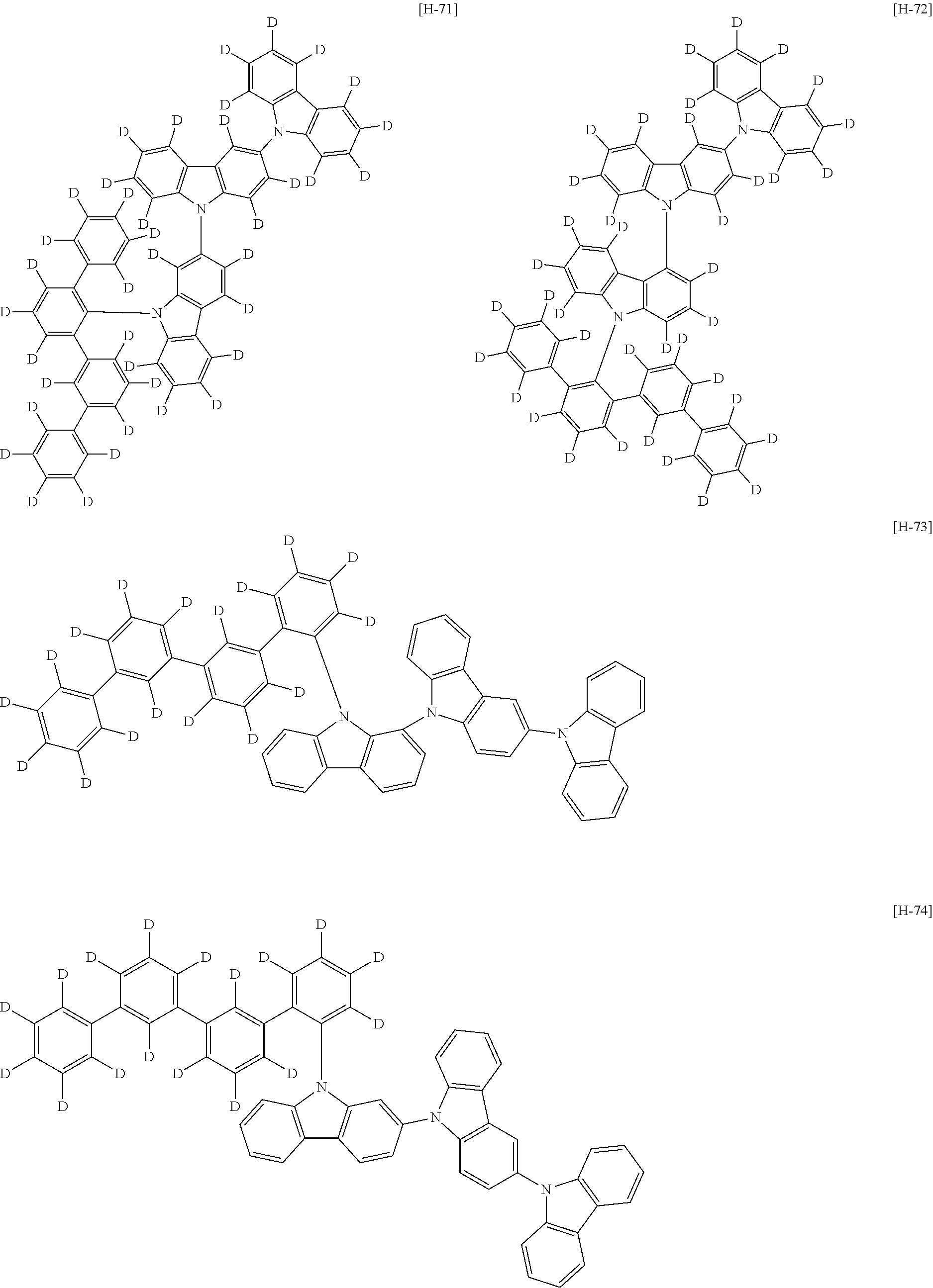

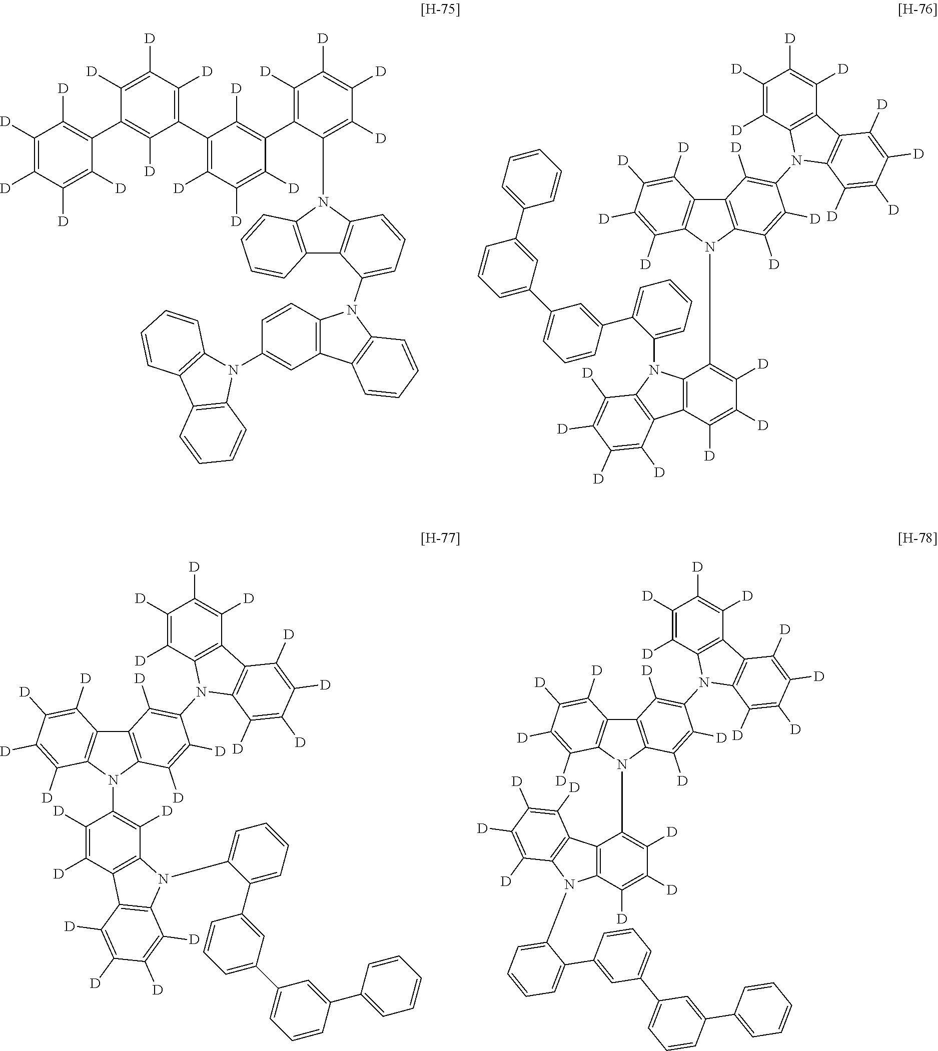

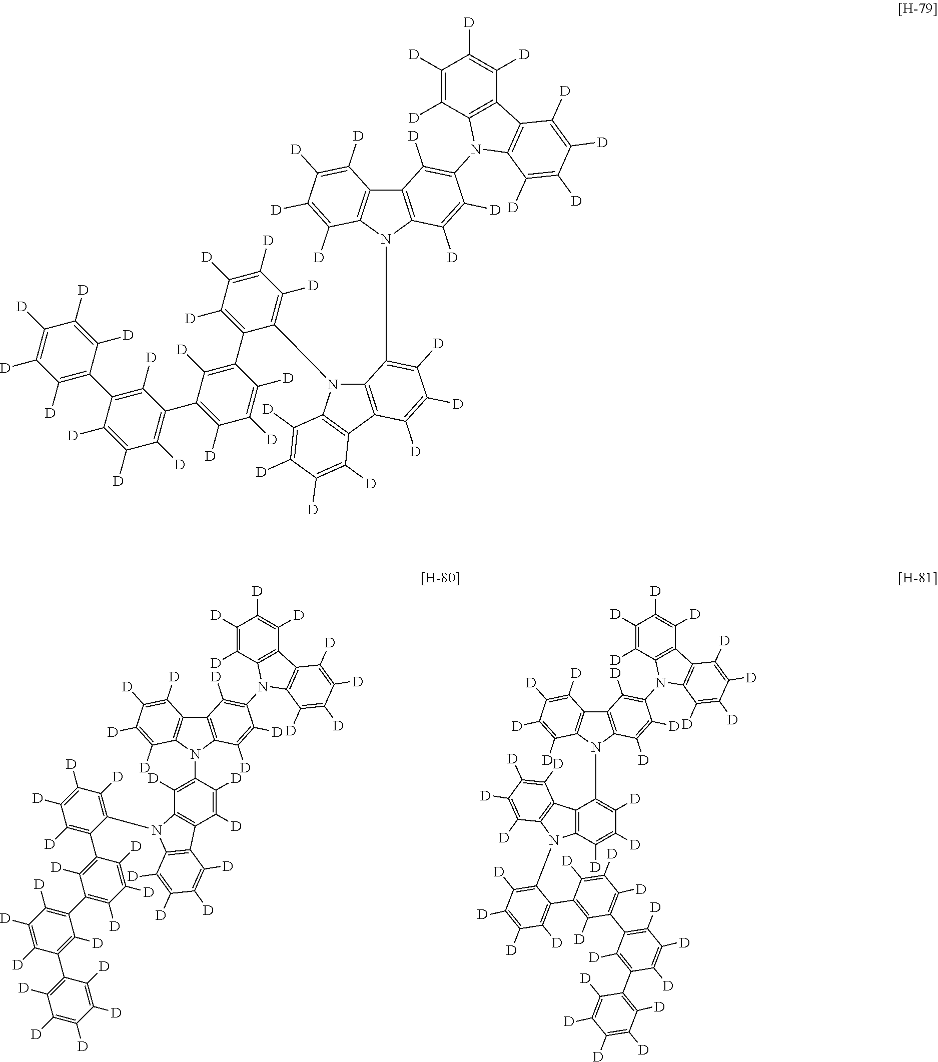

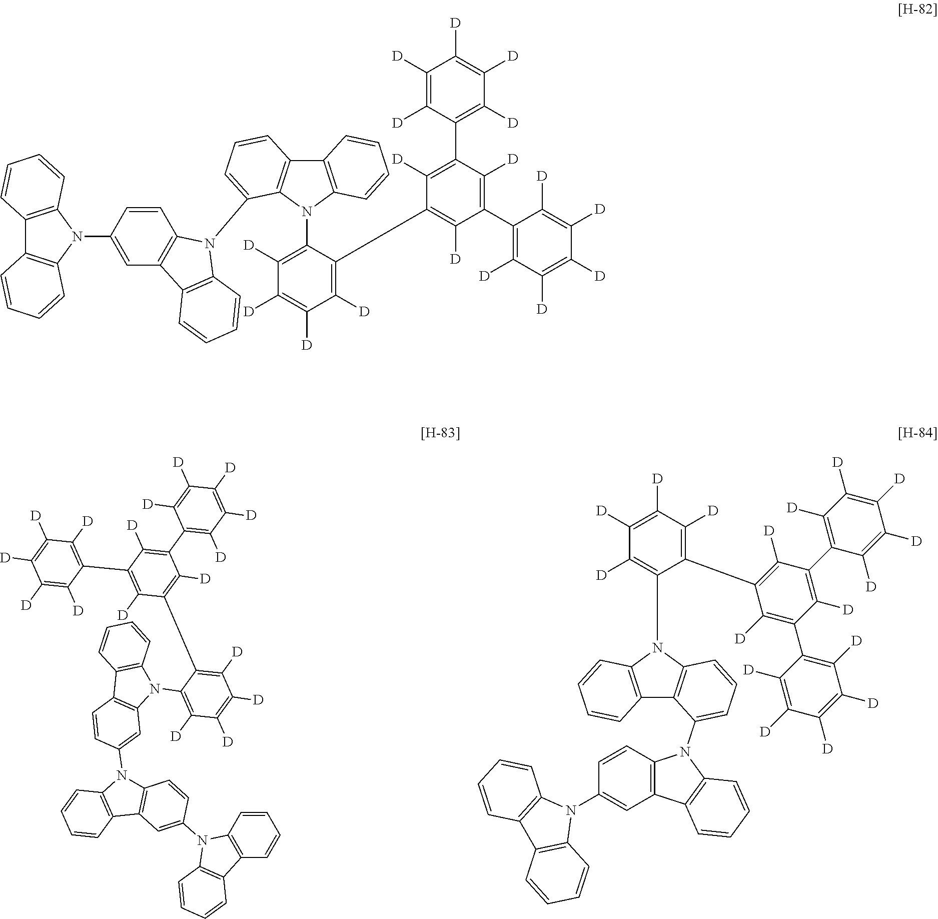

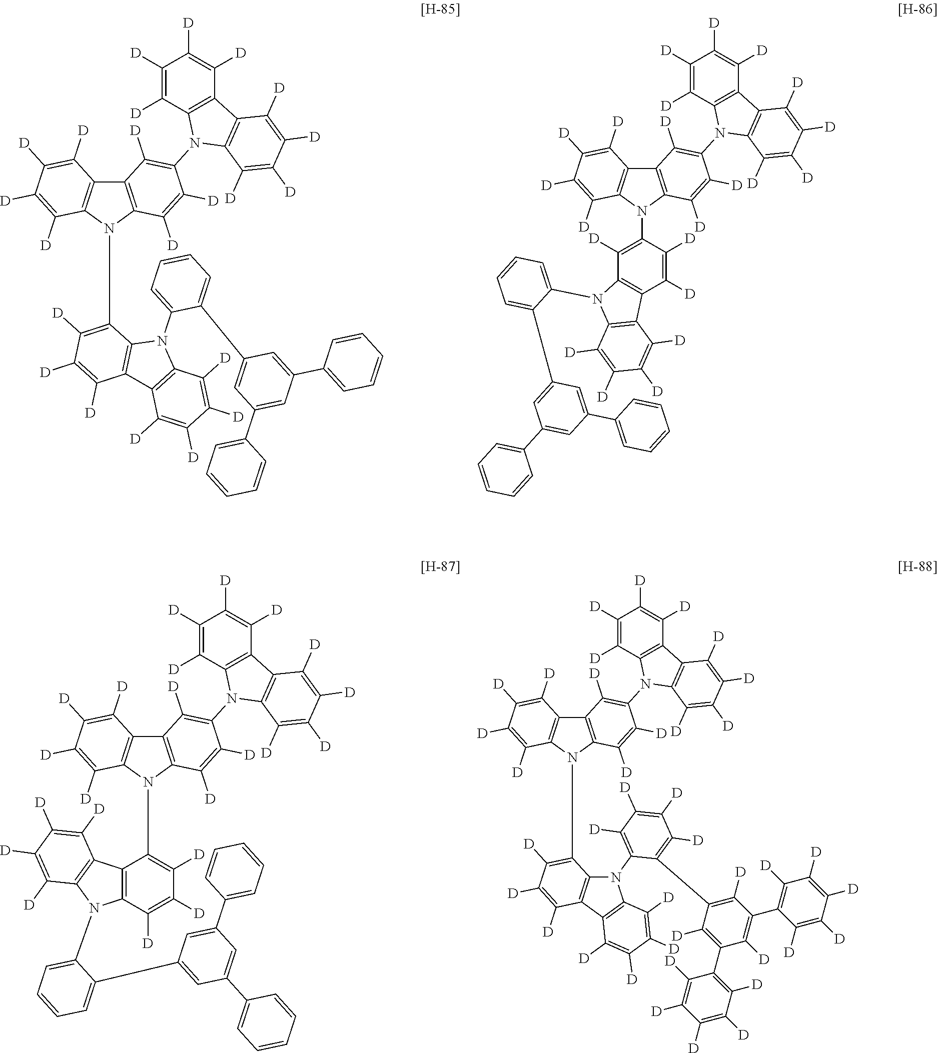

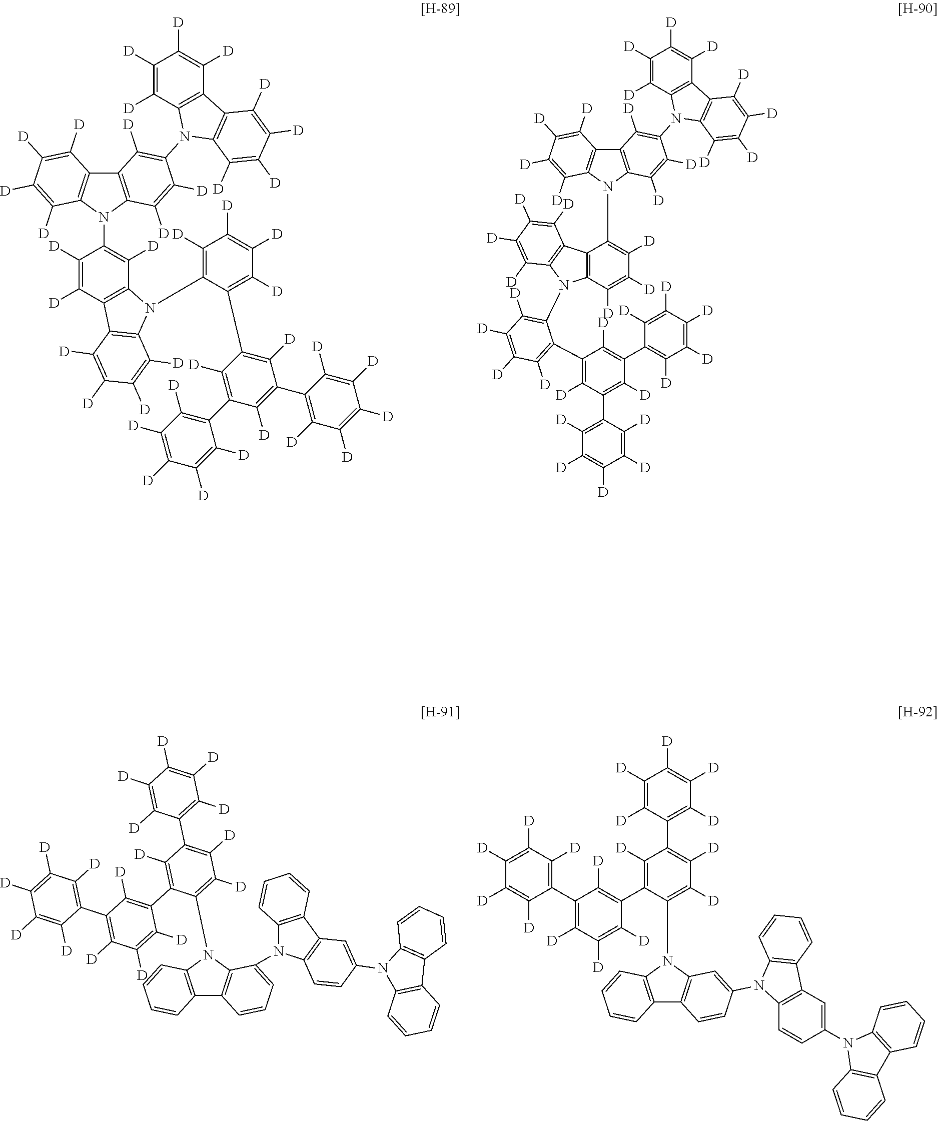

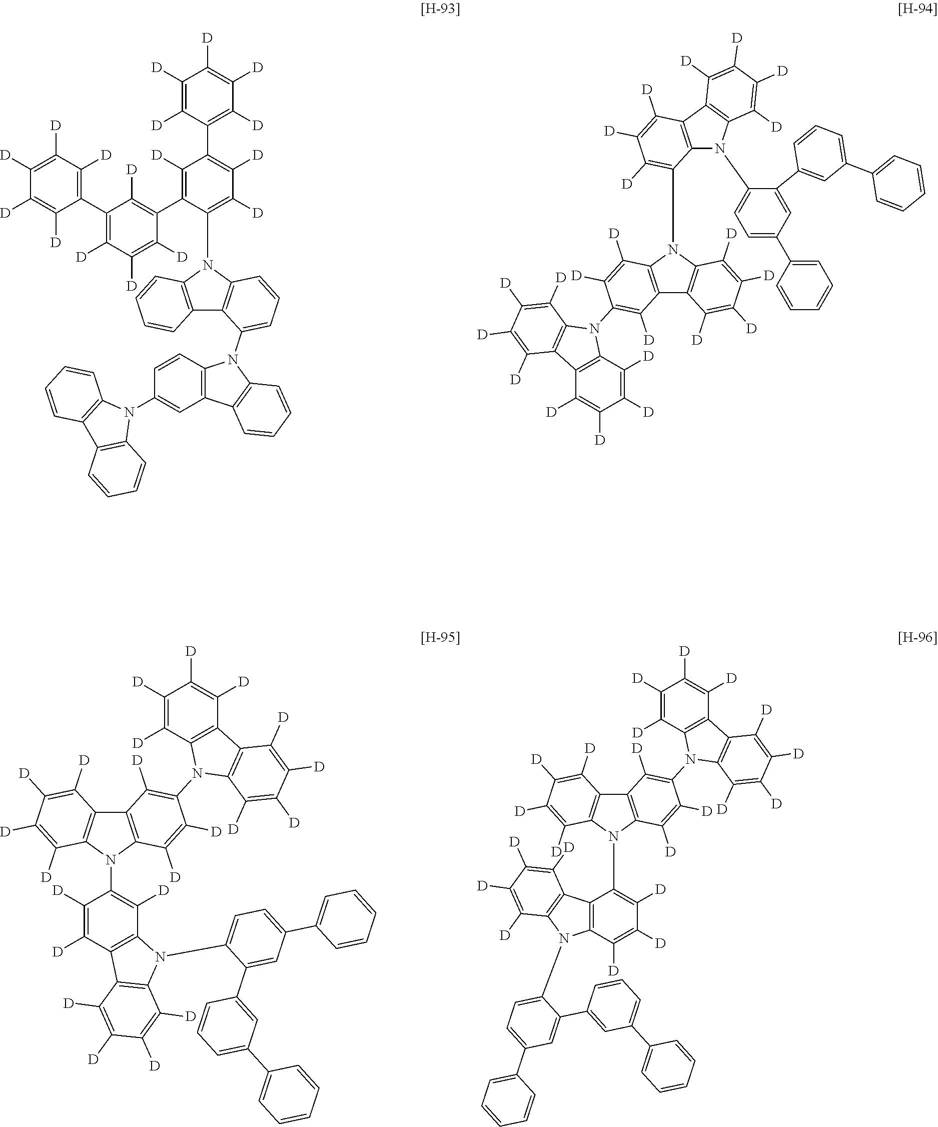

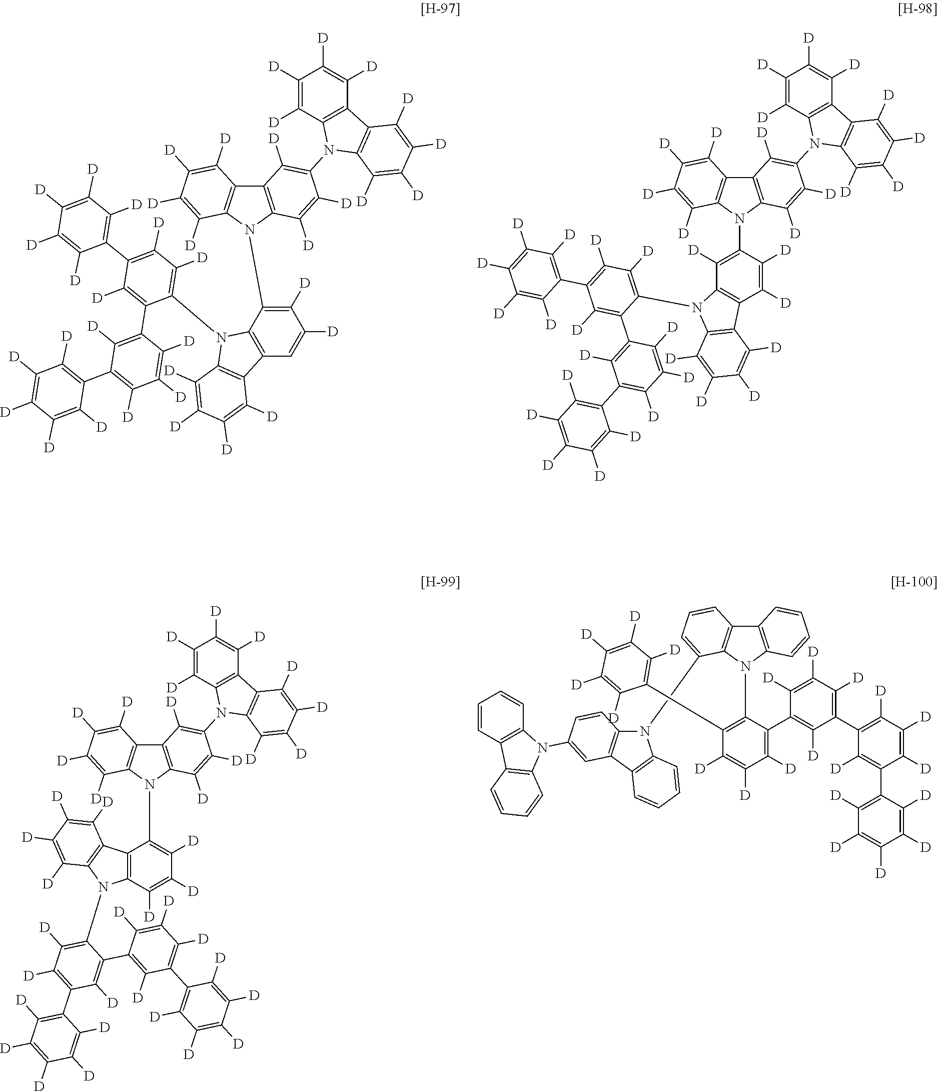

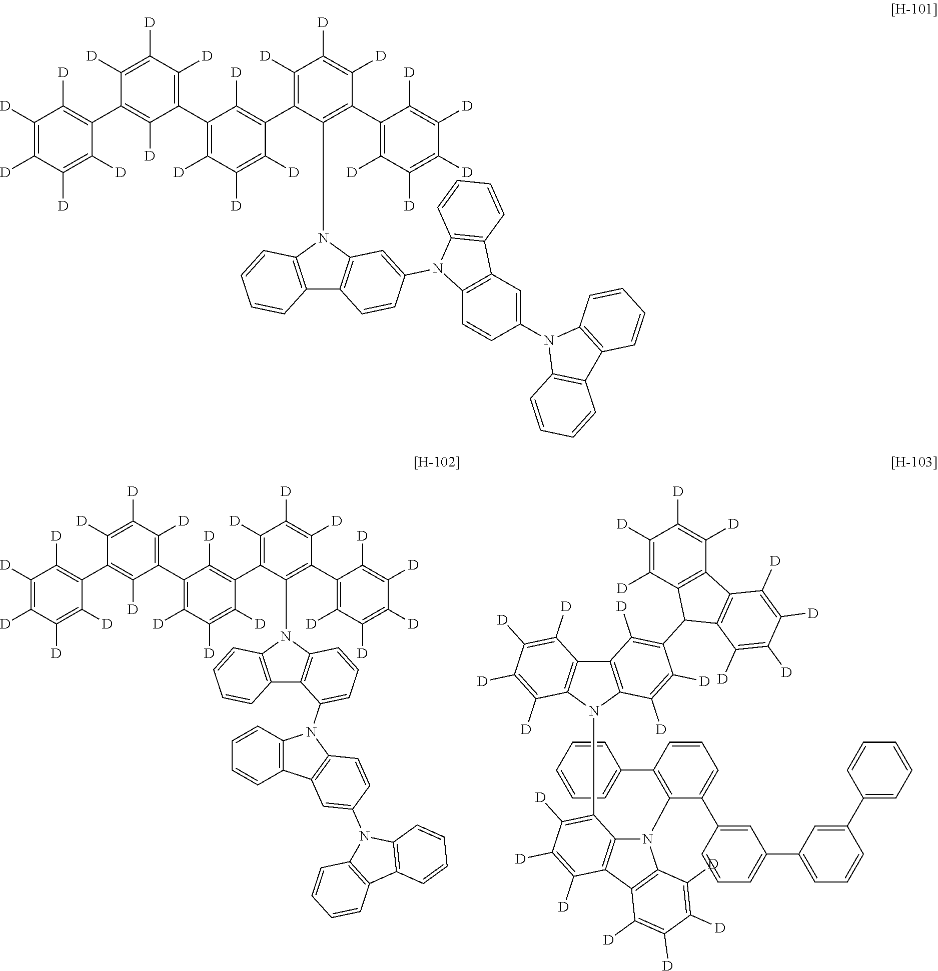

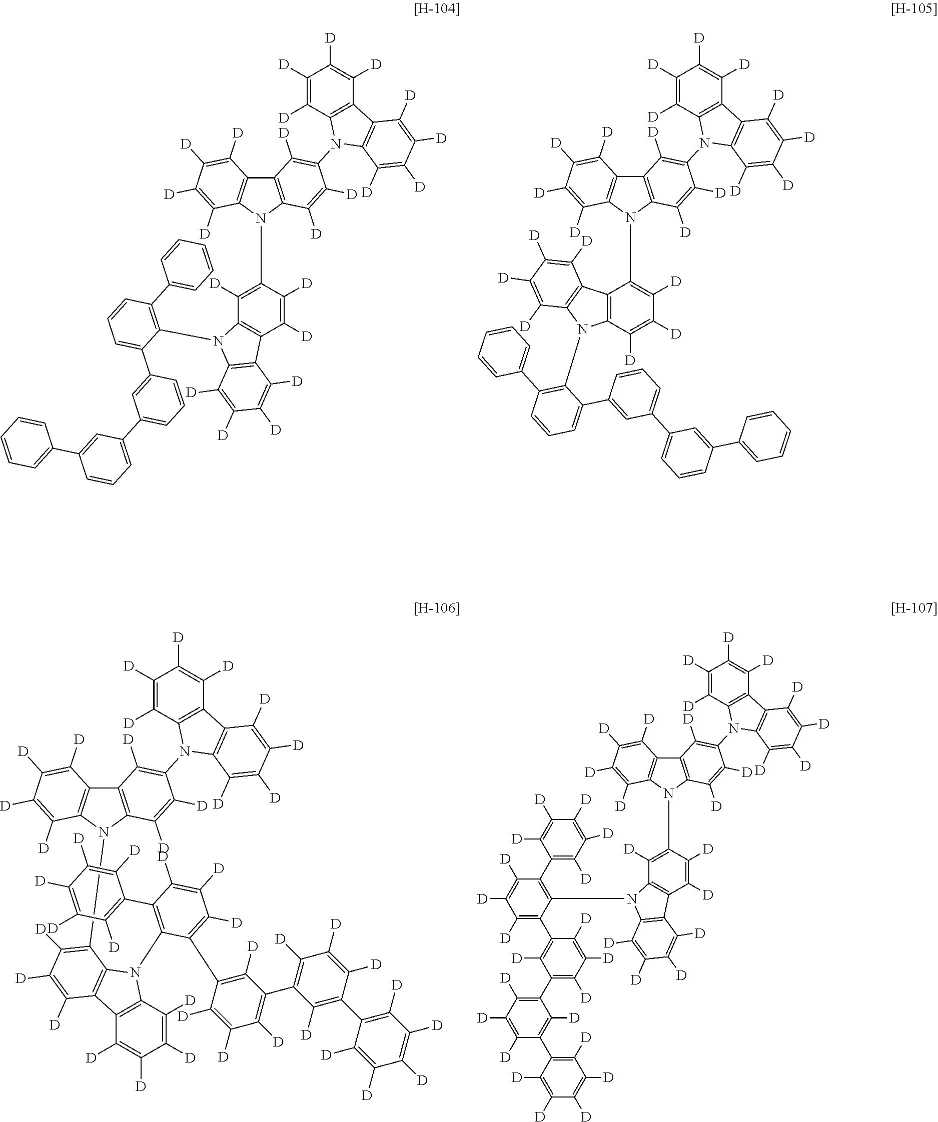

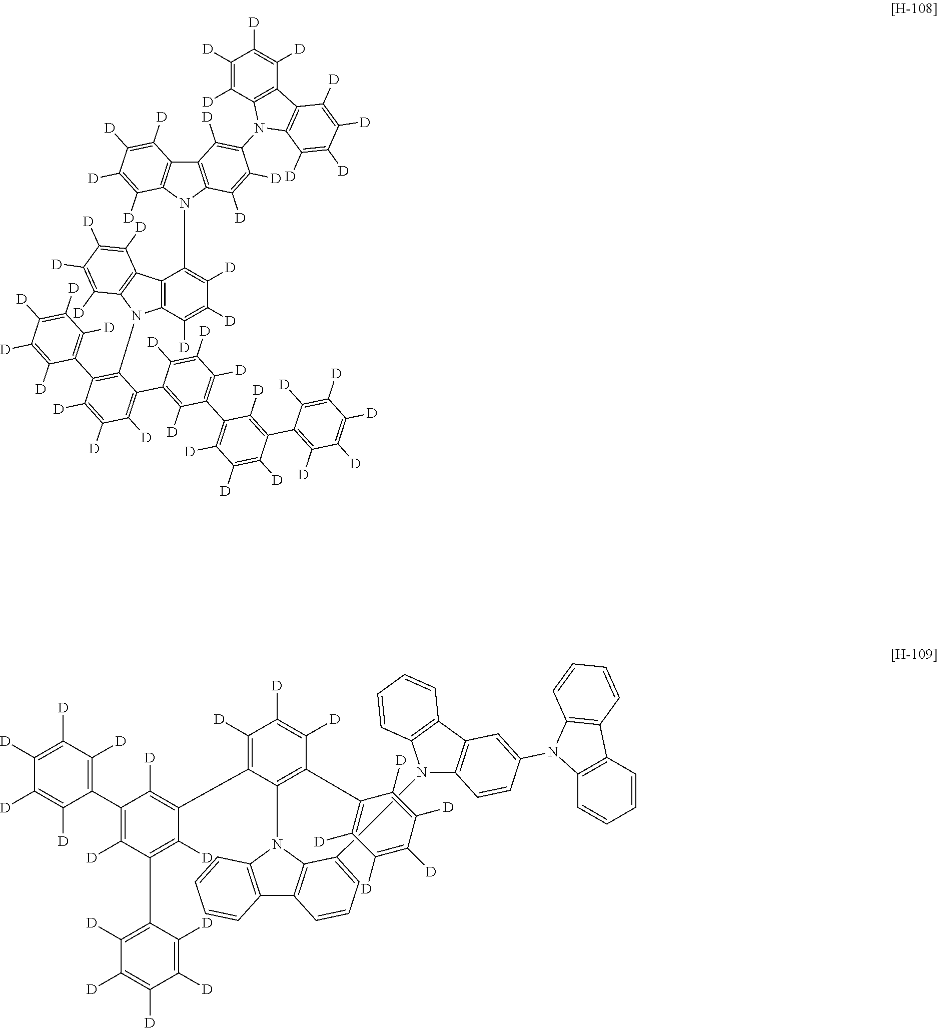

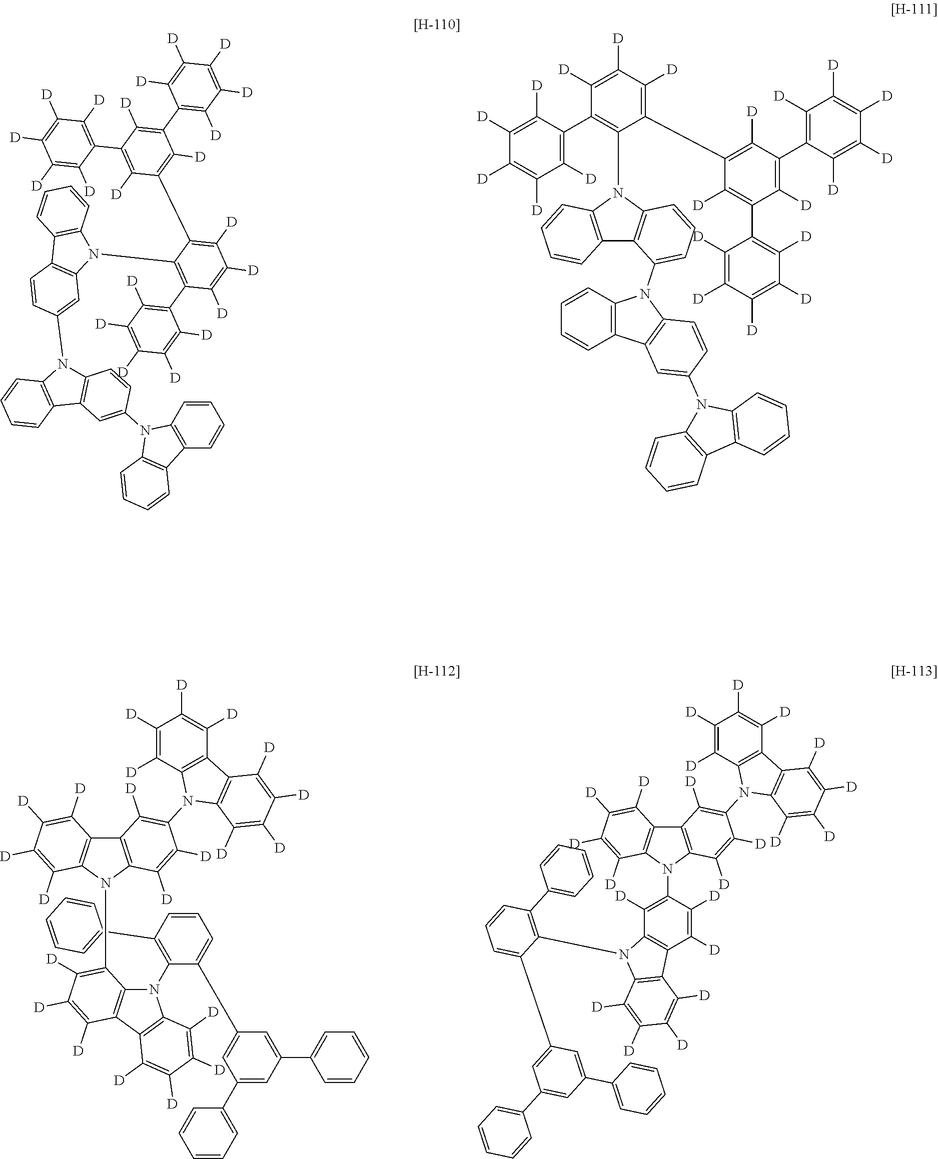

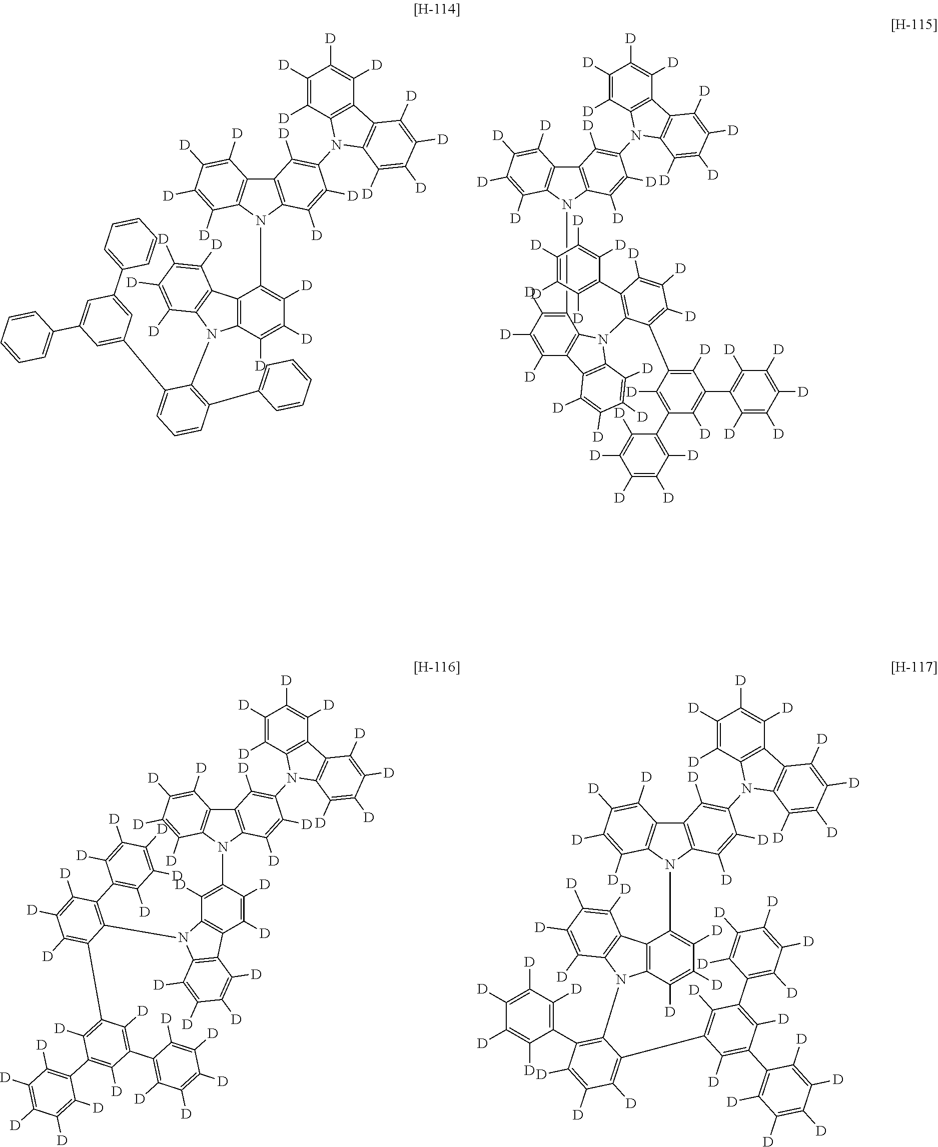

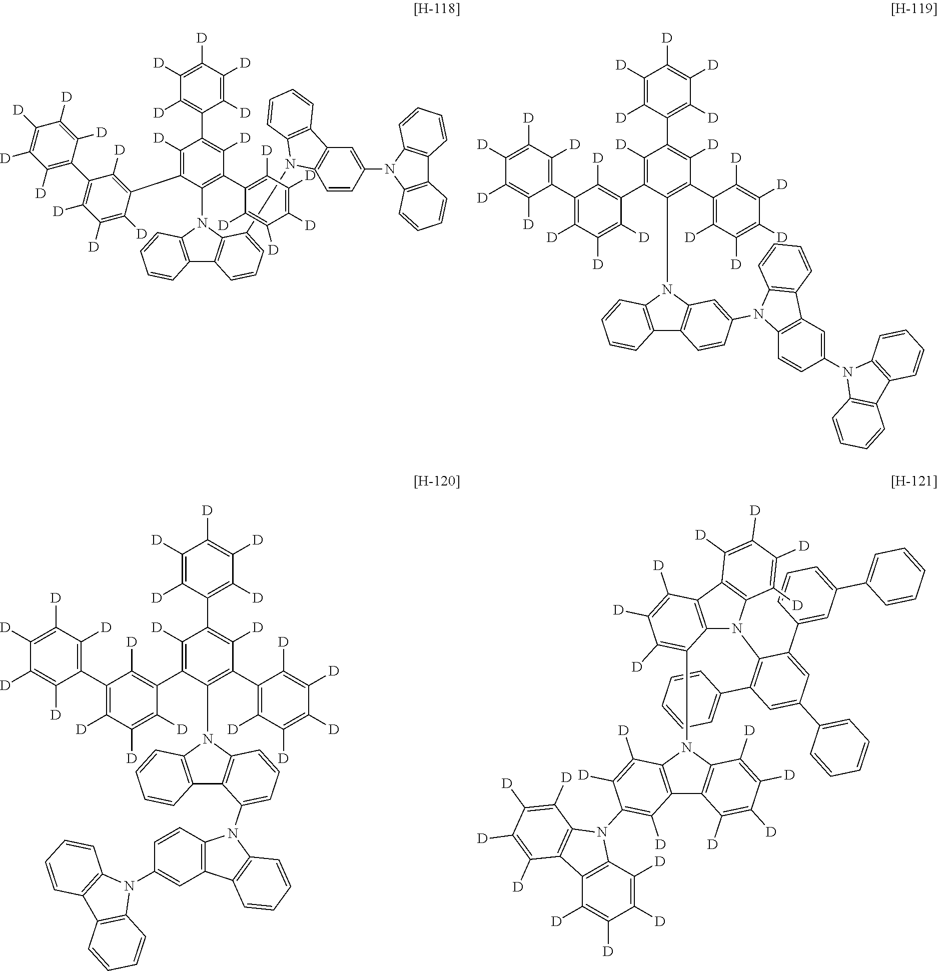

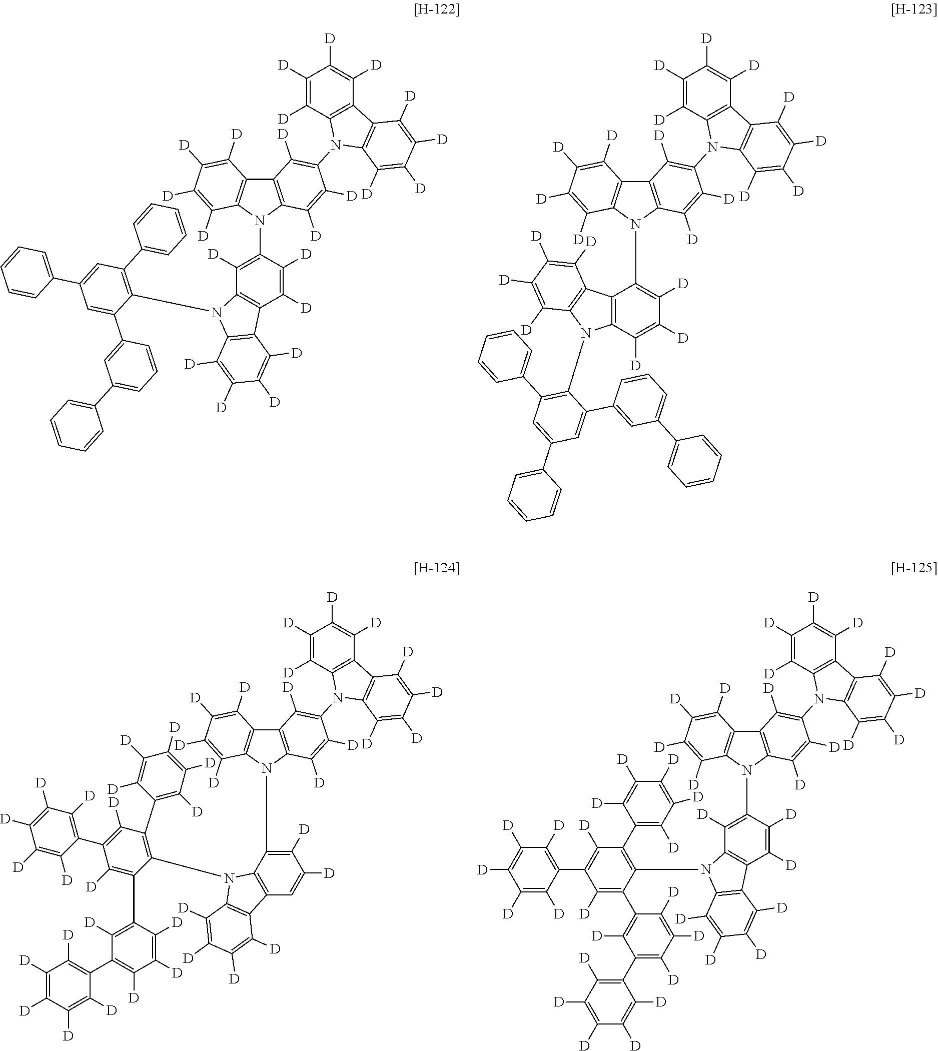

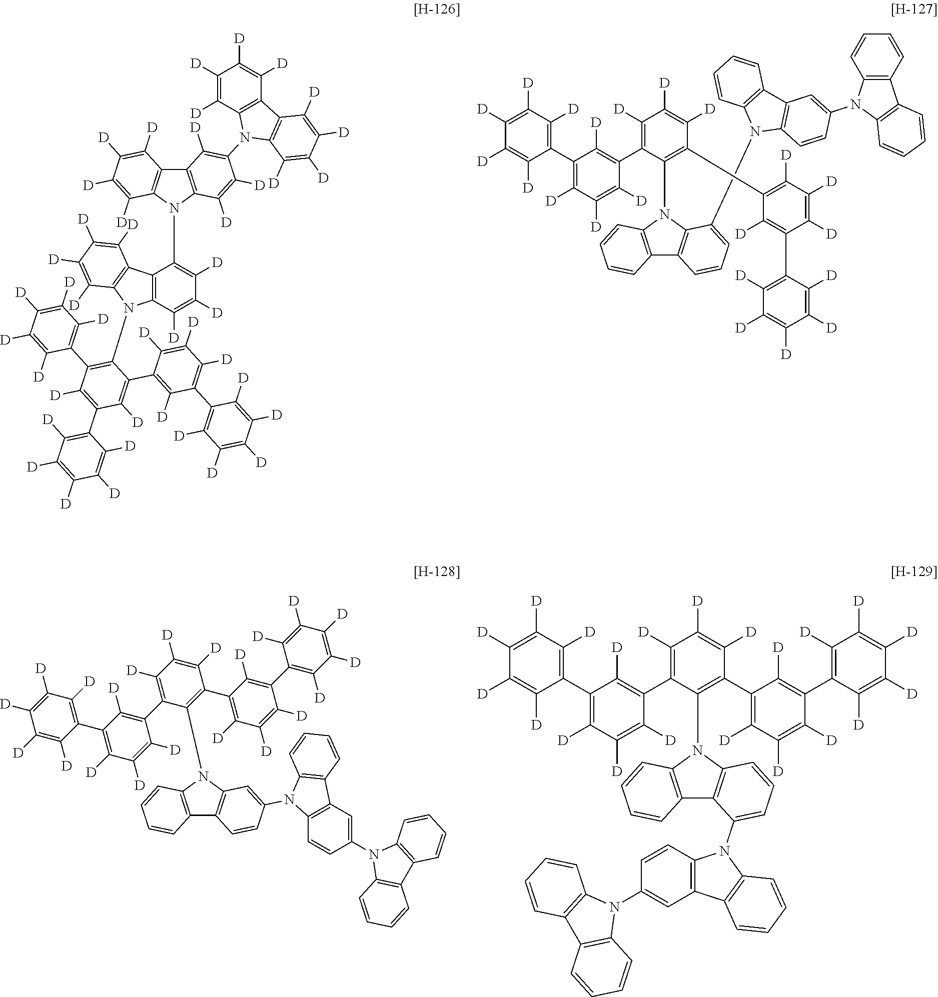

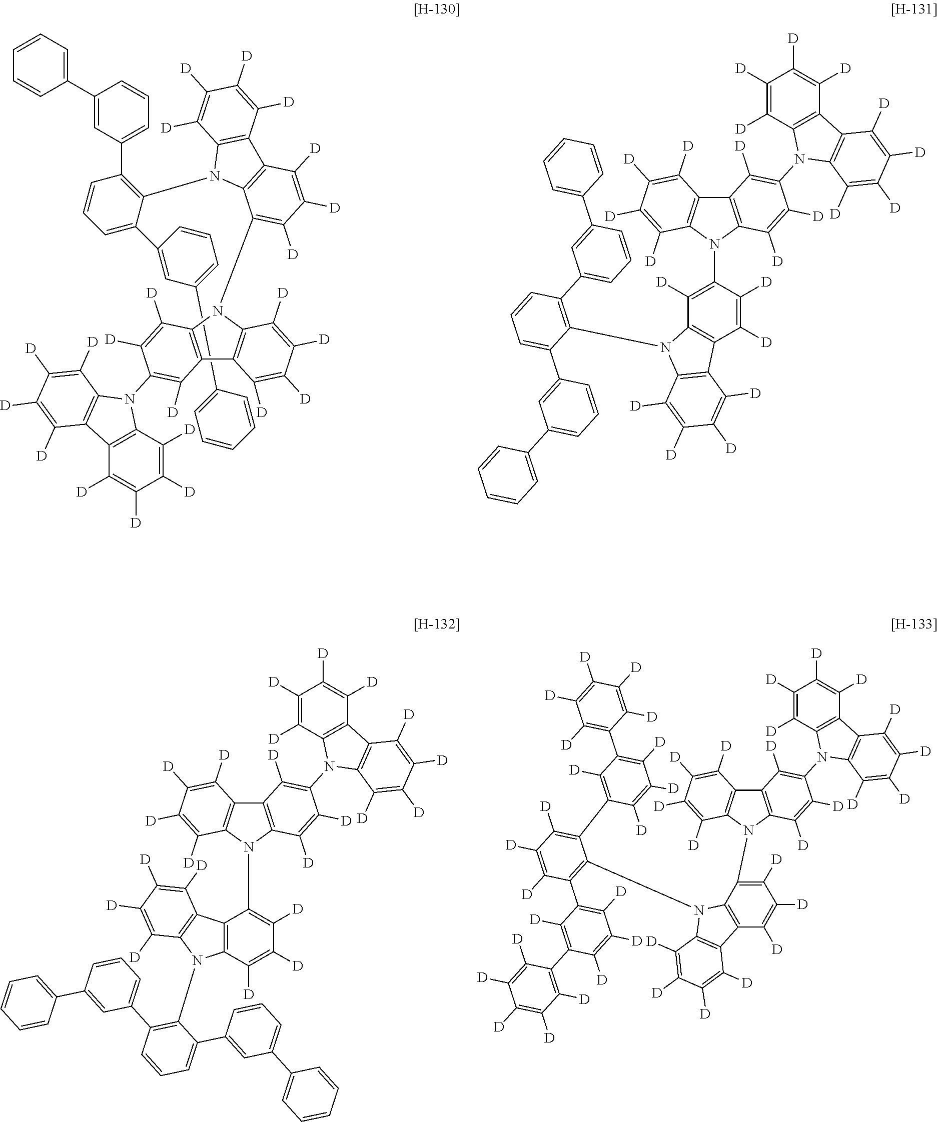

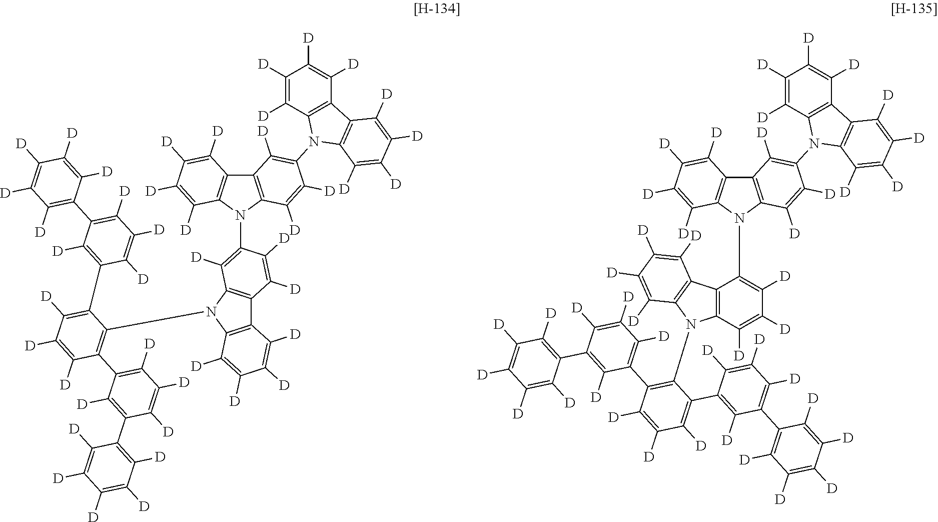

The aforementioned compound for an organic optoelectronic device may be used as a composition together with other compounds to exhibit a synergistic effect in improving the performance of organic optoelectronic devices such as organic light emitting diodes.

A composition for an organic optoelectronic device according to some example embodiments may include, e.g., the aforementioned compound (the first compound) and a second compound different from the first compound.

The second compound may have a structure in which one or more substituted or unsubstituted carbazolyl groups may be, e.g., directly or indirectly bonded to a nitrogen-containing ring, thereby helping effectively control the HOMO energy level and the LUMO energy level and helping secure stability for the movement of electrons (negative polaron). Accordingly, the composition for an organic optoelectronic device may help increase charge mobility and lower the driving voltage in the organic optoelectronic device while exhibiting improved life-span characteristics.

In an implementation, the second compound may be represented by Chemical Formula 3.

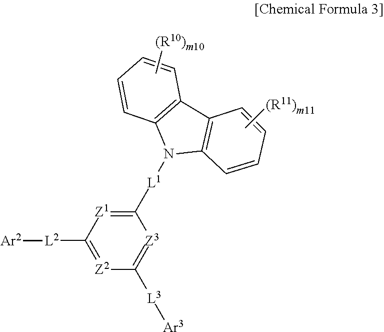

In Chemical Formula 3, Z¹ to Z³ may each independently be, e.g., N or CR^{b}.

In an implementation, at least two of Z¹ to Z³ are N.

L¹ to L³ may each independently be, e.g., a single bond, a substituted or unsubstituted C6 to C20 arylene group, or a substituted or unsubstituted divalent C2 to C20 heterocyclic group.

Ar² and Ar³ may each independently be, e.g., a substituted or unsubstituted C6 to C30 aryl group, a substituted or unsubstituted C2 to C20 heterocyclic group, or a substituted or unsubstituted silyl group.

R^{b}, R¹⁰, and R¹¹ may each independently be, e.g., hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen.

Each R¹⁰ and R¹¹ may be, e.g., separately present or two adjacent ones may be linked to form a ring.

m10 and m11 may each independently be, e.g., an integer of 1 to 4.

In an implementation, two of Z¹ to Z³ may be, e.g., N and the remaining one may be, e.g., CR^{b}.

In an implementation, Z¹ to Z³ may each be, e.g., N.

In an implementation, at least one of Ar² and Ar³ may be, e.g., a substituted or unsubstituted carbazolyl group.

In an implementation, at least one of Ar² and Ar³ may be, e.g., a substituted or unsubstituted silyl group.

In an implementation, either of Ar² and Ar³ may be a substituted or unsubstituted carbazolyl group, as represented in Chemical Formula 3a.

In an implementation, either of Ar² and Ar³ may be a substituted or unsubstituted silyl group, as represented in Chemical Formula 3b.

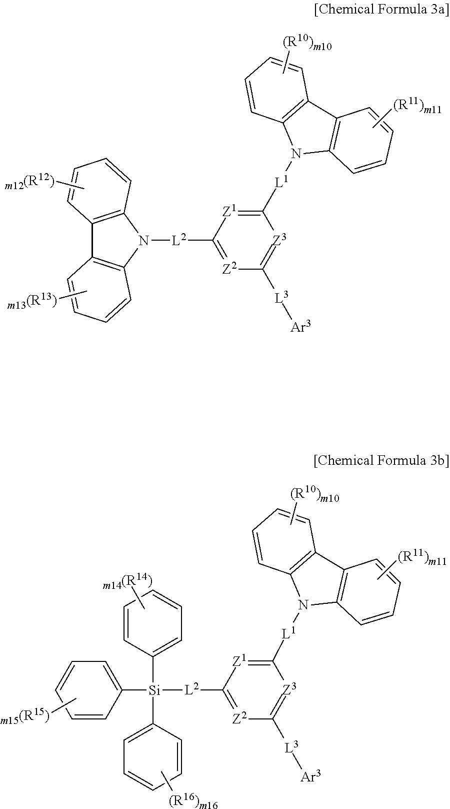

In Chemical Formula 3a and Chemical Formula 3b, Z¹ to Z³, L¹ to L³, m10, m11, R¹⁰ and R¹¹ may be defined the same as those of Chemical Formula 3.

R¹² to R¹⁶ may each independently be, e.g., hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen.

m12 and m13 may each independently be, e.g., an integer of 1 to 4.

m14 to m16 may each independently be, e.g., an integer of 1 to 5.

Each R¹² to R¹⁶ may be separately present or two adjacent ones may be linked to form a ring.

In an implementation, L¹ may be, e.g., a single bond.

In an implementation, L² may be, e.g., a single bond, a substituted or unsubstituted phenylene group, a substituted or unsubstituted biphenylene group, a substituted or unsubstituted terphenylene group, a substituted or unsubstituted naphthylene group, a substituted or unsubstituted triphenylenylene group, a substituted or unsubstituted dibenzofuranylene group, a substituted or unsubstituted dibenzothiophenylene group, or a substituted or unsubstituted carbazolylene group.

In an implementation, L³ may be, e.g., a single bond or a substituted or unsubstituted phenylene group.

In an implementation, Ar² and Ar³ may each independently be, e.g., a substituted or unsubstituted phenyl group, a substituted or unsubstituted biphenyl group, a substituted or unsubstituted terphenyl group, a substituted or unsubstituted dibenzofuranyl group, a substituted or unsubstituted dibenzothiophenyl group, a substituted or unsubstituted carbazolyl group, or a substituted or unsubstituted triphenylsilyl group.

In an implementation, R¹² to R¹⁶ and R^{b} may each independently be, e.g., hydrogen, deuterium, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, a substituted or unsubstituted silyl group, or a cyano group.

In an implementation, at least one of the first compound and the second compound may be substituted with one or more deuterium atoms. Here, the number of deuterium substituted in each compound may be, e.g., from 1 to the maximum number of hydrogens in the chemical formula, e.g., from 1 to 40 or from 1 to 30.

In an implementation, the second compound represented by Chemical Formula 3 may include, e.g., a compound of Group 2.

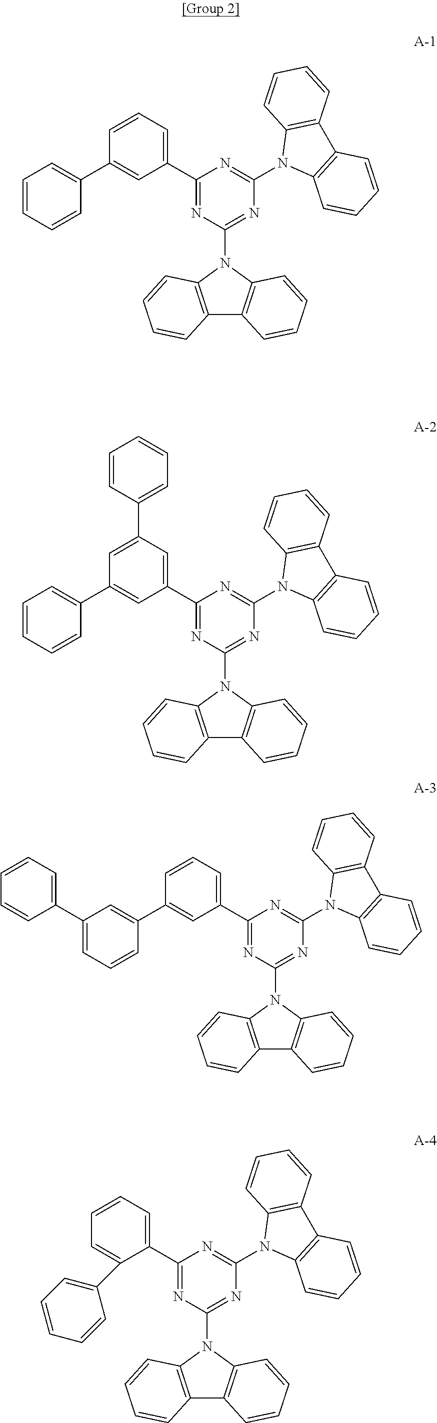 [SMILES: c1ccc(-c2cccc(-c3nc(-n4c5ccccc5c5ccccc54)nc(-n4c5ccccc5c5ccccc54)n3)c2)cc1 ; c1ccc(-c2cc(-c3ccccc3)cc(-c3nc(-n4c5ccccc5c5ccccc54)nc(-n4c5ccccc5c5ccccc54)n3)c2)cc1 ; c1ccc(-c2cccc(-c3cccc(-c4nc(-n5c6ccccc6c6ccccc65)nc(-n5c6ccccc6c6ccccc65)n4)c3)c2)cc1 ; c1ccc(-c2ccccc2-c2nc(-n3c4ccccc4c4ccccc43)nc(-n3c4ccccc4c4ccccc43)n2)cc1]

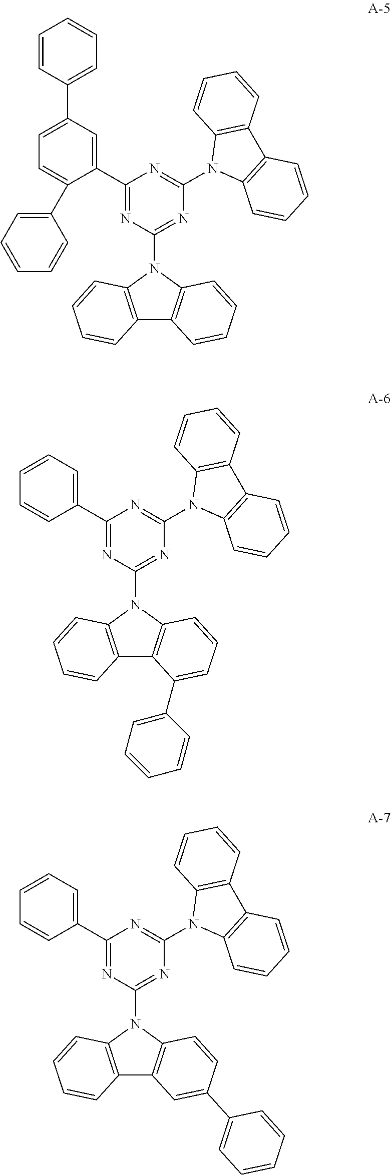 [SMILES: c1ccc(-c2ccc(-c3ccccc3)c(-c3nc(-n4c5ccccc5c5ccccc54)nc(-n4c5ccccc5c5ccccc54)n3)c2)cc1 ; c1ccc(-c2nc(-n3c4ccccc4c4ccccc43)nc(-n3c4ccccc4c4c(-c5ccccc5)cccc43)n2)cc1 ; c1ccc(-c2ccc3c(c2)c2ccccc2n3-c2nc(-c3ccccc3)nc(-n3c4ccccc4c4ccccc43)n2)cc1]

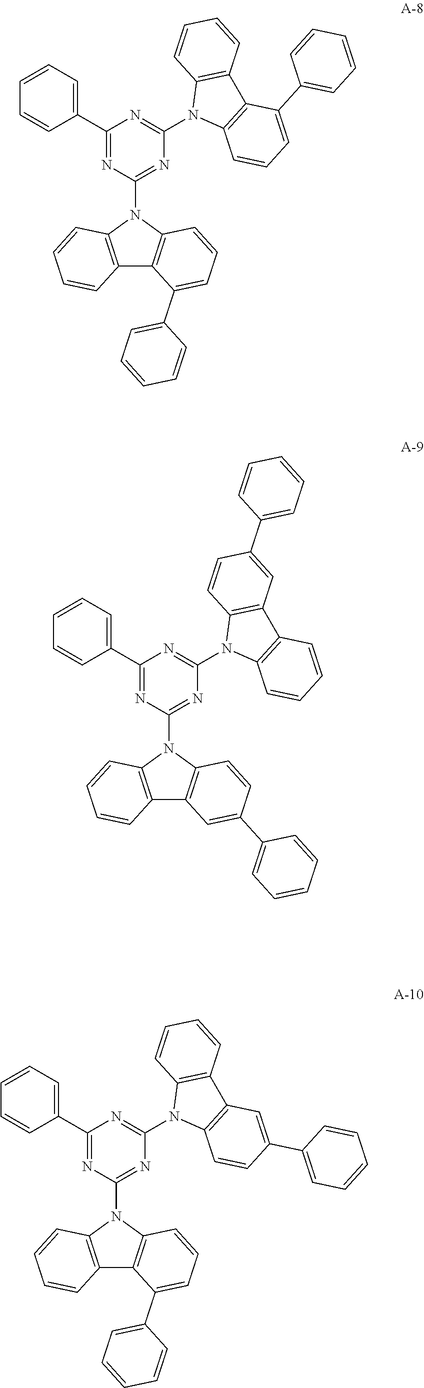 [SMILES: c1ccc(-c2nc(-n3c4ccccc4c4c(-c5ccccc5)cccc43)nc(-n3c4ccccc4c4c(-c5ccccc5)cccc43)n2)cc1 ; c1ccc(-c2ccc3c(c2)c2ccccc2n3-c2nc(-c3ccccc3)nc(-n3c4ccccc4c4cc(-c5ccccc5)ccc43)n2)cc1 ; c1ccc(-c2ccc3c(c2)c2ccccc2n3-c2nc(-c3ccccc3)nc(-n3c4ccccc4c4c(-c5ccccc5)cccc43)n2)cc1]

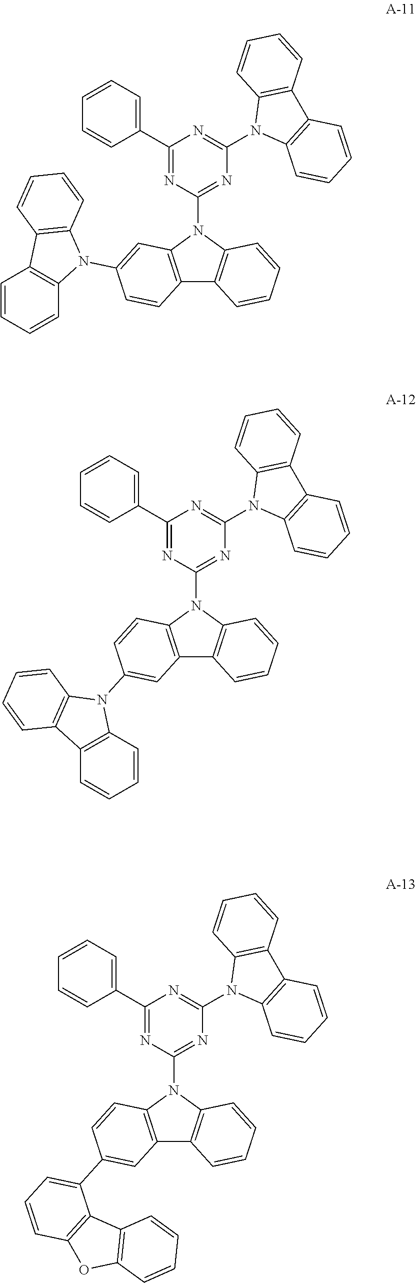 [SMILES: c1ccc(-c2nc(-n3c4ccccc4c4ccccc43)nc(-n3c4ccccc4c4ccc(-n5c6ccccc6c6ccccc65)cc43)n2)cc1 ; c1ccc(-c2nc(-n3c4ccccc4c4ccccc43)nc(-n3c4ccccc4c4cc(-n5c6ccccc6c6ccccc65)ccc43)n2)cc1 ; c1ccc(-c2nc(-n3c4ccccc4c4ccccc43)nc(-n3c4ccccc4c4cc(-c5cccc6oc7ccccc7c56)ccc43)n2)cc1]

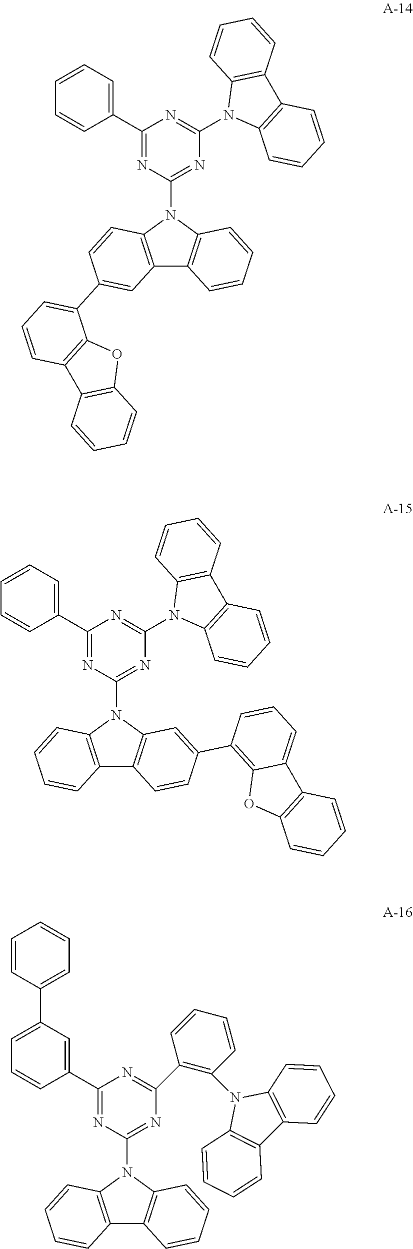 [SMILES: c1ccc(-c2nc(-n3c4ccccc4c4ccccc43)nc(-n3c4ccccc4c4cc(-c5cccc6c5oc5ccccc56)ccc43)n2)cc1 ; c1ccc(-c2nc(-n3c4ccccc4c4ccccc43)nc(-n3c4ccccc4c4ccc(-c5cccc6c5oc5ccccc56)cc43)n2)cc1 ; c1ccc(-c2cccc(-c3nc(-c4ccccc4-n4c5ccccc5c5ccccc54)nc(-n4c5ccccc5c5ccccc54)n3)c2)cc1]

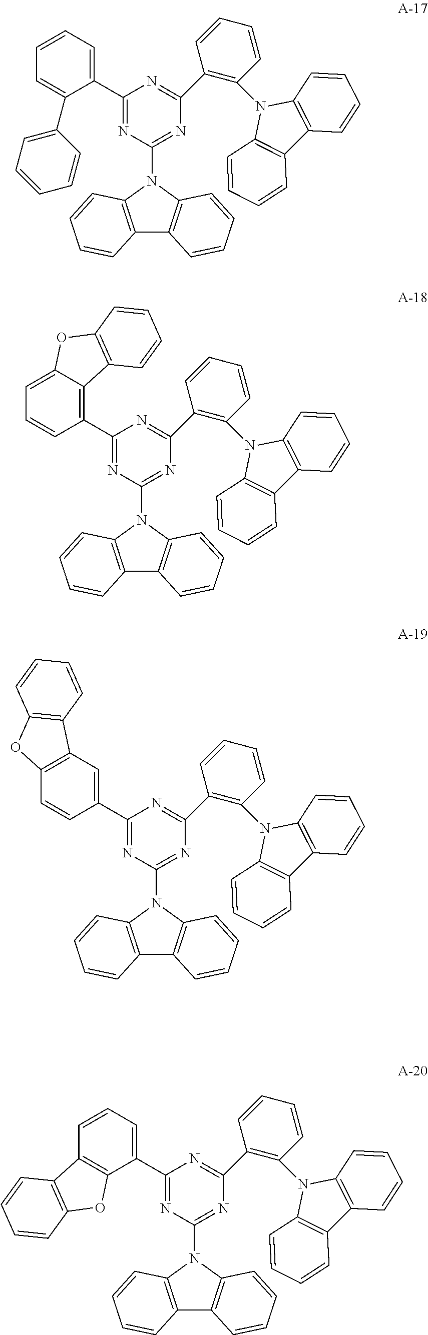 [SMILES: c1ccc(-c2ccccc2-c2nc(-c3ccccc3-n3c4ccccc4c4ccccc43)nc(-n3c4ccccc4c4ccccc43)n2)cc1 ; c1ccc(-n2c3ccccc3c3ccccc32)c(-c2nc(-c3cccc4oc5ccccc5c34)nc(-n3c4ccccc4c4ccccc43)n2)c1 ; c1ccc(-n2c3ccccc3c3ccccc32)c(-c2nc(-c3ccc4oc5ccccc5c4c3)nc(-n3c4ccccc4c4ccccc43)n2)c1 ; c1ccc(-n2c3ccccc3c3ccccc32)c(-c2nc(-c3cccc4c3oc3ccccc34)nc(-n3c4ccccc4c4ccccc43)n2)c1]

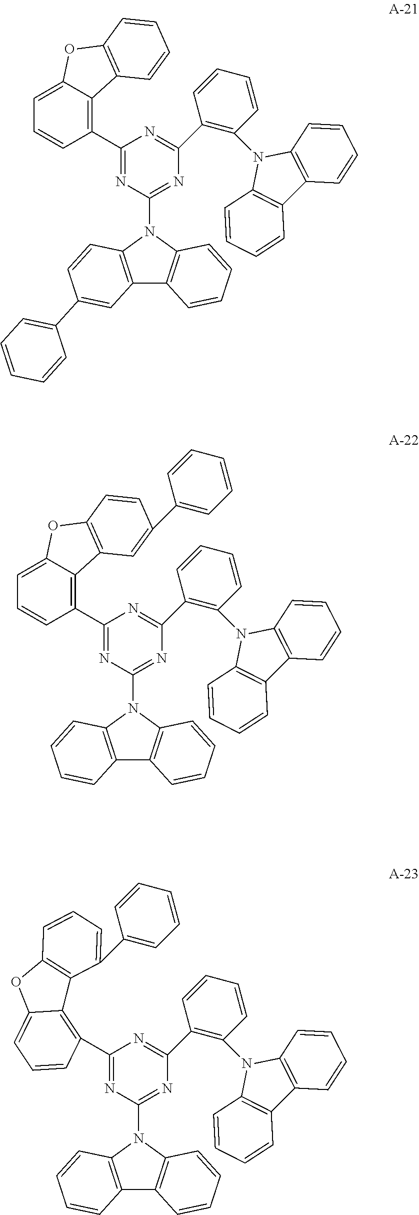 [SMILES: c1ccc(-c2ccc3c(c2)c2ccccc2n3-c2nc(-c3ccccc3-n3c4ccccc4c4ccccc43)nc(-c3cccc4oc5ccccc5c34)n2)cc1 ; c1ccc(-c2ccc3oc4cccc(-c5nc(-c6ccccc6-n6c7ccccc7c7ccccc76)nc(-n6c7ccccc7c7ccccc76)n5)c4c3c2)cc1 ; c1ccc(-c2cccc3oc4cccc(-c5nc(-c6ccccc6-n6c7ccccc7c7ccccc76)nc(-n6c7ccccc7c7ccccc76)n5)c4c23)cc1]

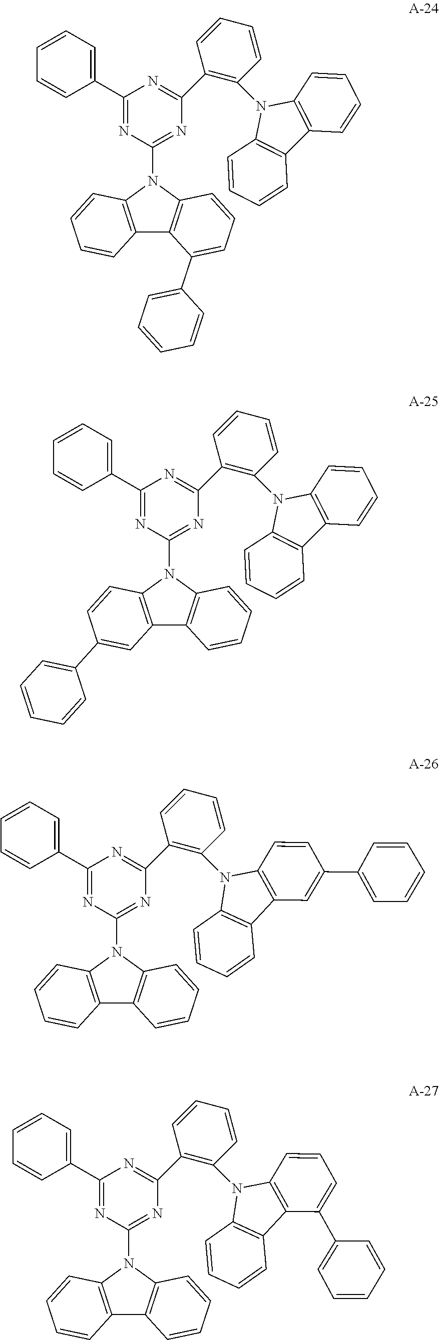 [SMILES: c1ccc(-c2nc(-c3ccccc3-n3c4ccccc4c4ccccc43)nc(-n3c4ccccc4c4c(-c5ccccc5)cccc43)n2)cc1 ; c1ccc(-c2ccc3c(c2)c2ccccc2n3-c2nc(-c3ccccc3)nc(-c3ccccc3-n3c4ccccc4c4ccccc43)n2)cc1 ; c1ccc(-c2ccc3c(c2)c2ccccc2n3-c2ccccc2-c2nc(-c3ccccc3)nc(-n3c4ccccc4c4ccccc43)n2)cc1 ; c1ccc(-c2nc(-c3ccccc3-n3c4ccccc4c4c(-c5ccccc5)cccc43)nc(-n3c4ccccc4c4ccccc43)n2)cc1]

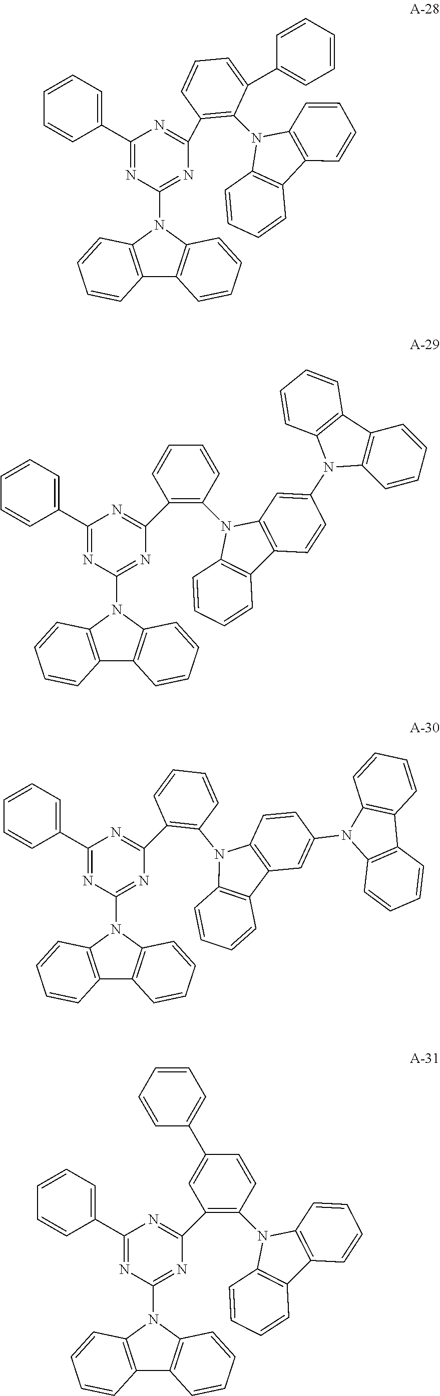 [SMILES: c1ccc(-c2nc(-c3cccc(-c4ccccc4)c3-n3c4ccccc4c4ccccc43)nc(-n3c4ccccc4c4ccccc43)n2)cc1 ; c1ccc(-c2nc(-c3ccccc3-n3c4ccccc4c4ccc(-n5c6ccccc6c6ccccc65)cc43)nc(-n3c4ccccc4c4ccccc43)n2)cc1 ; c1ccc(-c2nc(-c3ccccc3-n3c4ccccc4c4cc(-n5c6ccccc6c6ccccc65)ccc43)nc(-n3c4ccccc4c4ccccc43)n2)cc1 ; c1ccc(-c2ccc(-n3c4ccccc4c4ccccc43)c(-c3nc(-c4ccccc4)nc(-n4c5ccccc5c5ccccc54)n3)c2)cc1]

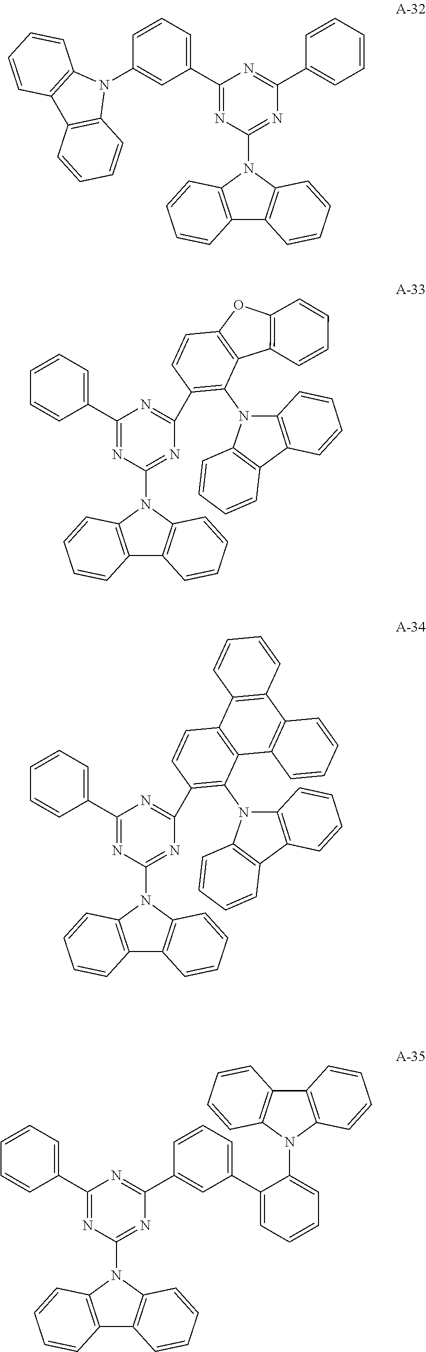 [SMILES: c1ccc(-c2nc(-c3cccc(-n4c5ccccc5c5ccccc54)c3)nc(-n3c4ccccc4c4ccccc43)n2)cc1 ; c1ccc(-c2nc(-c3ccc4oc5ccccc5c4c3-n3c4ccccc4c4ccccc43)nc(-n3c4ccccc4c4ccccc43)n2)cc1 ; c1ccc(-c2nc(-c3ccc4c5ccccc5c5ccccc5c4c3-n3c4ccccc4c4ccccc43)nc(-n3c4ccccc4c4ccccc43)n2)cc1 ; c1ccc(-c2nc(-c3cccc(-c4ccccc4-n4c5ccccc5c5ccccc54)c3)nc(-n3c4ccccc4c4ccccc43)n2)cc1]

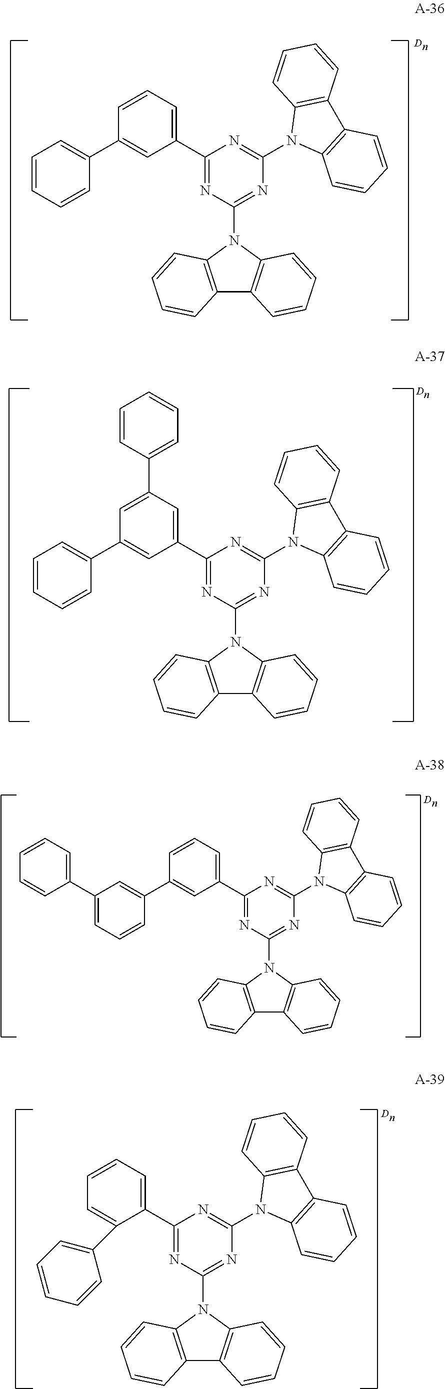

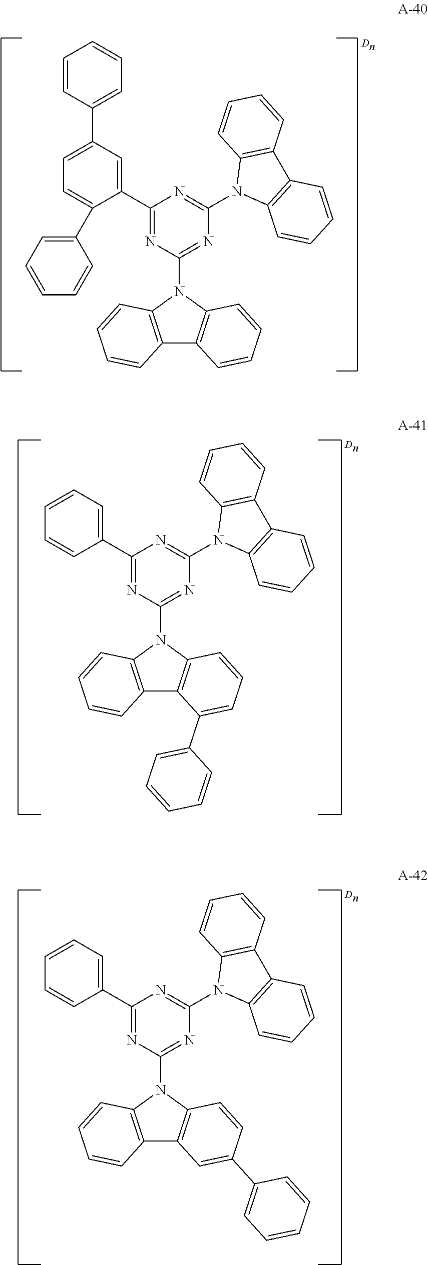

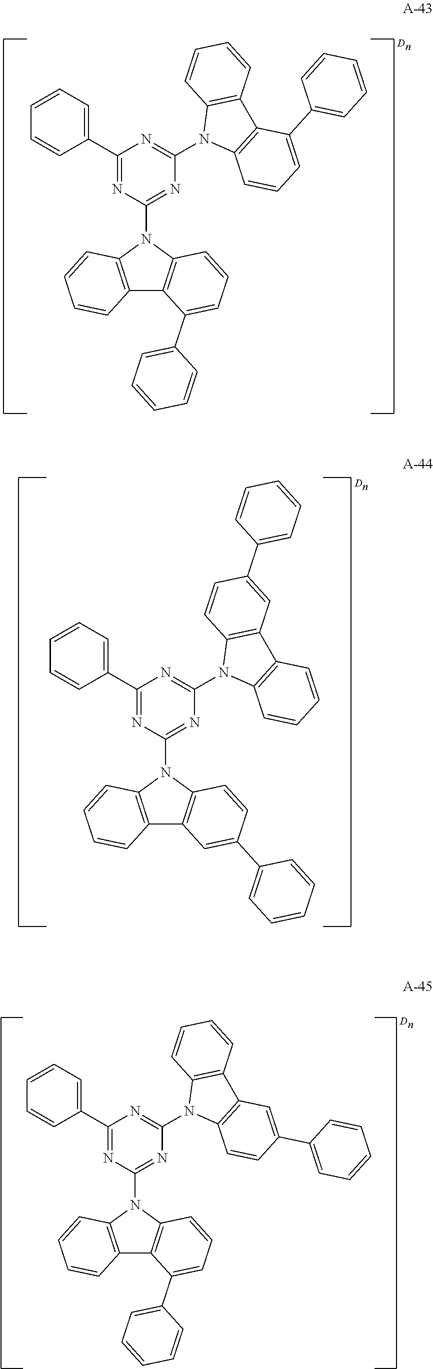

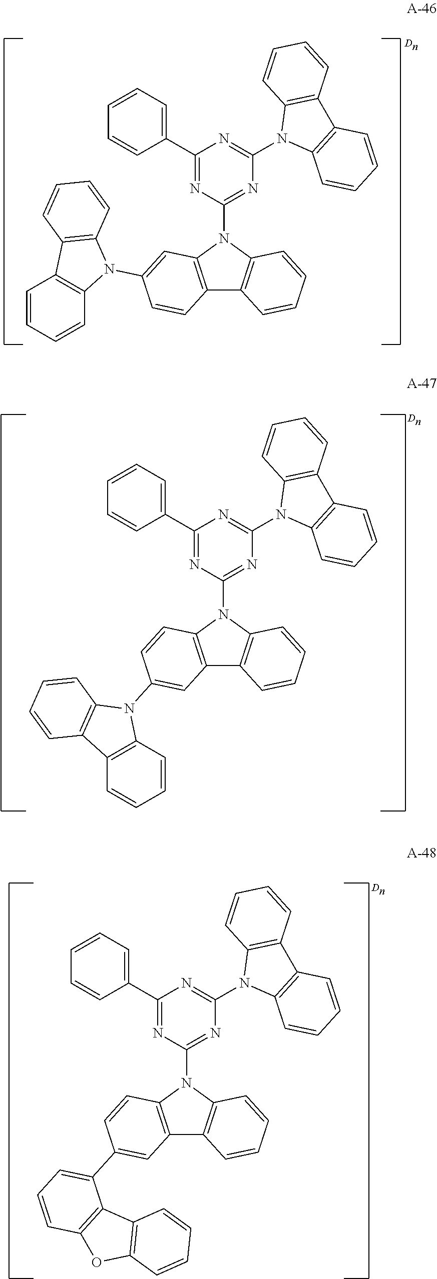

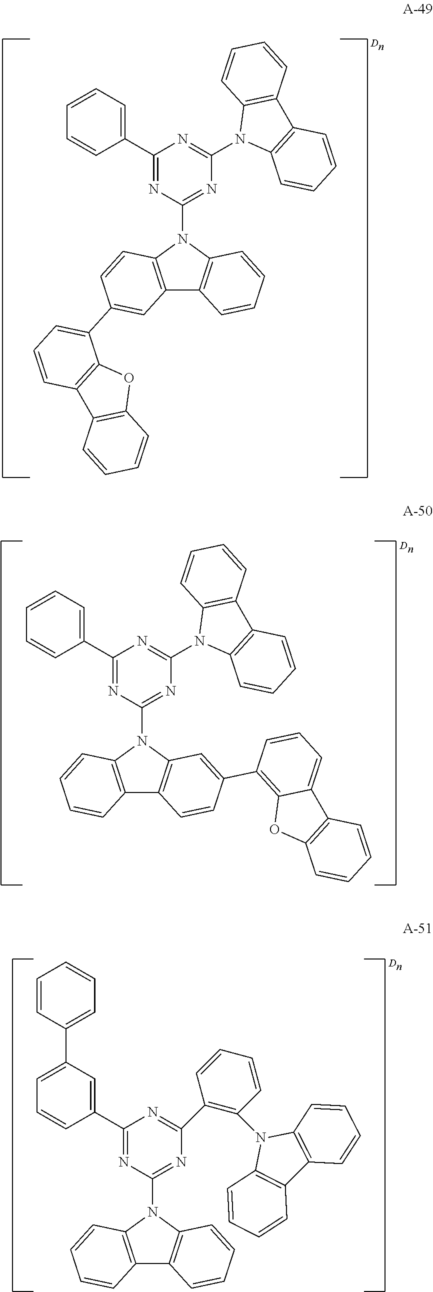

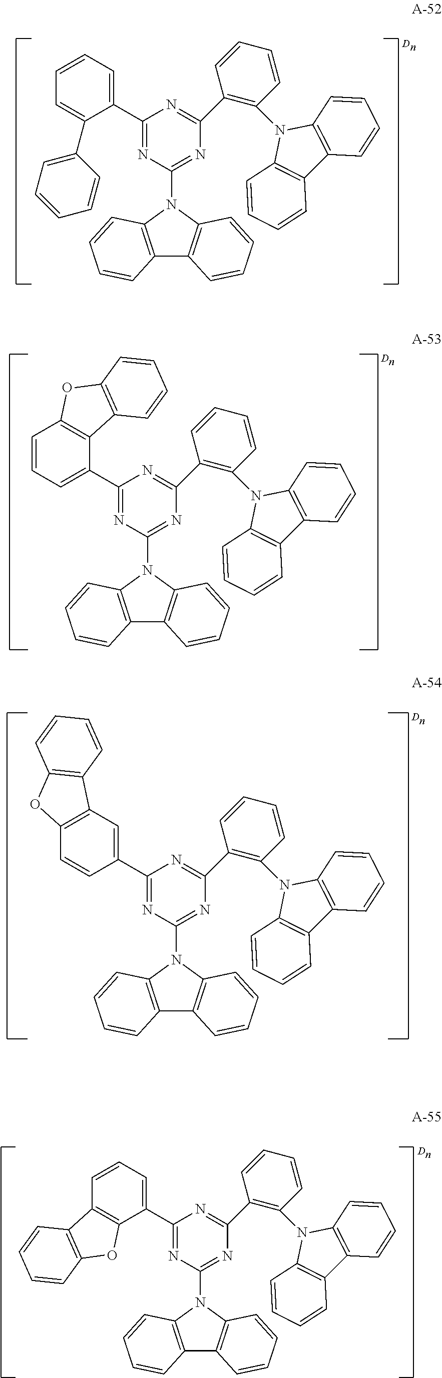

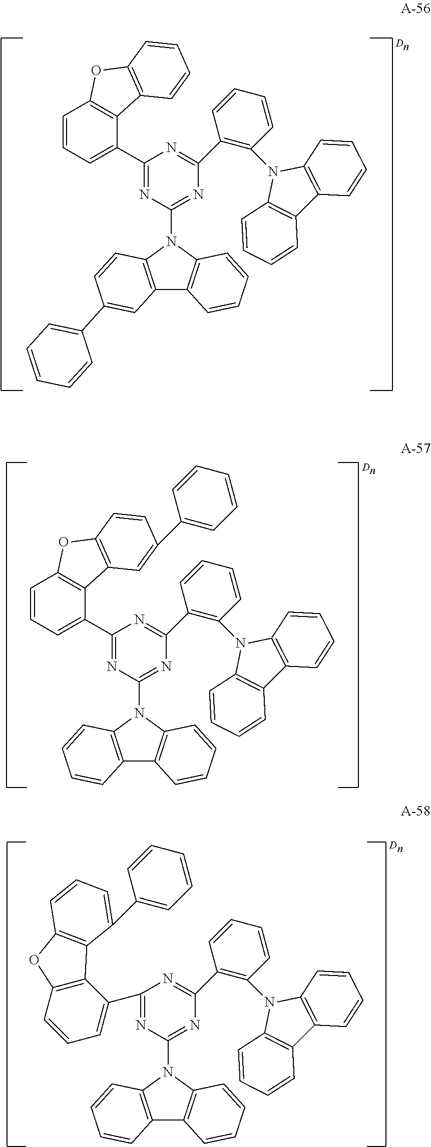

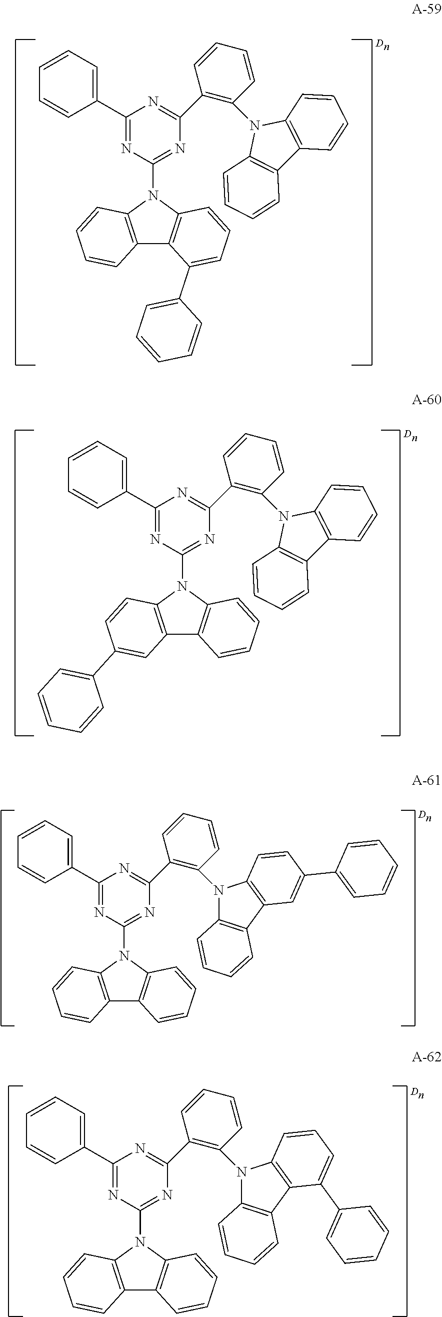

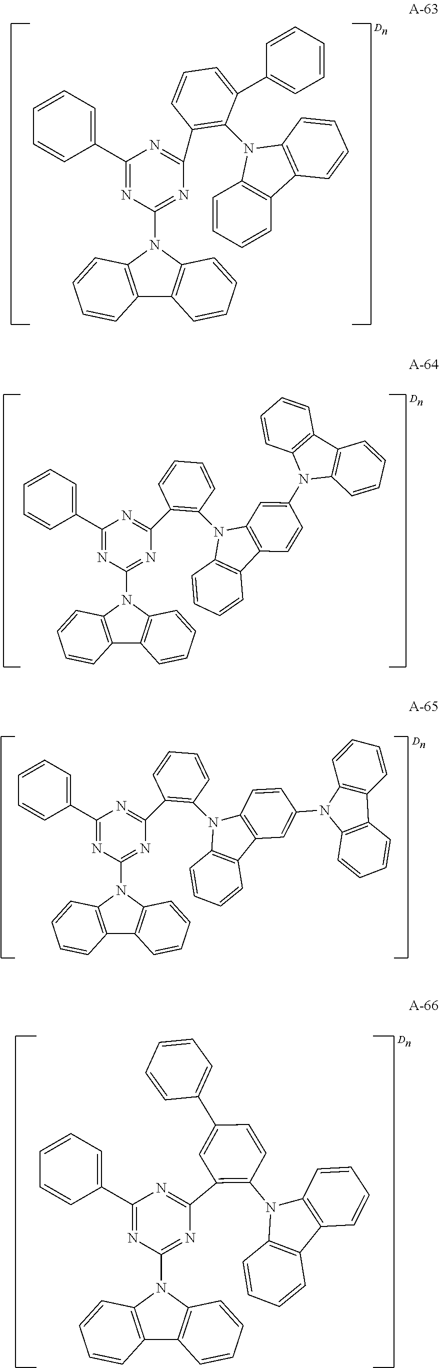

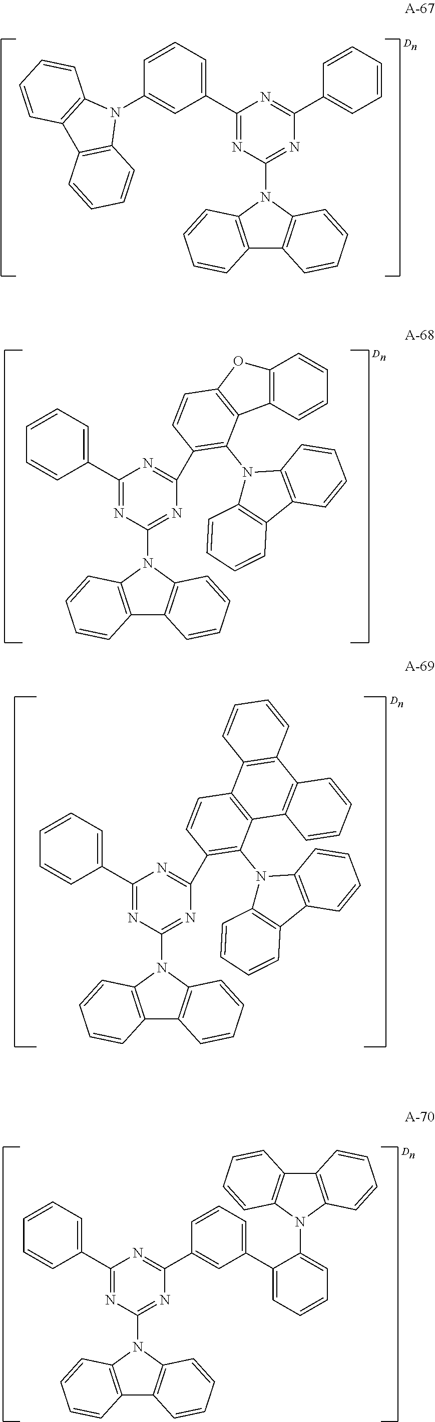

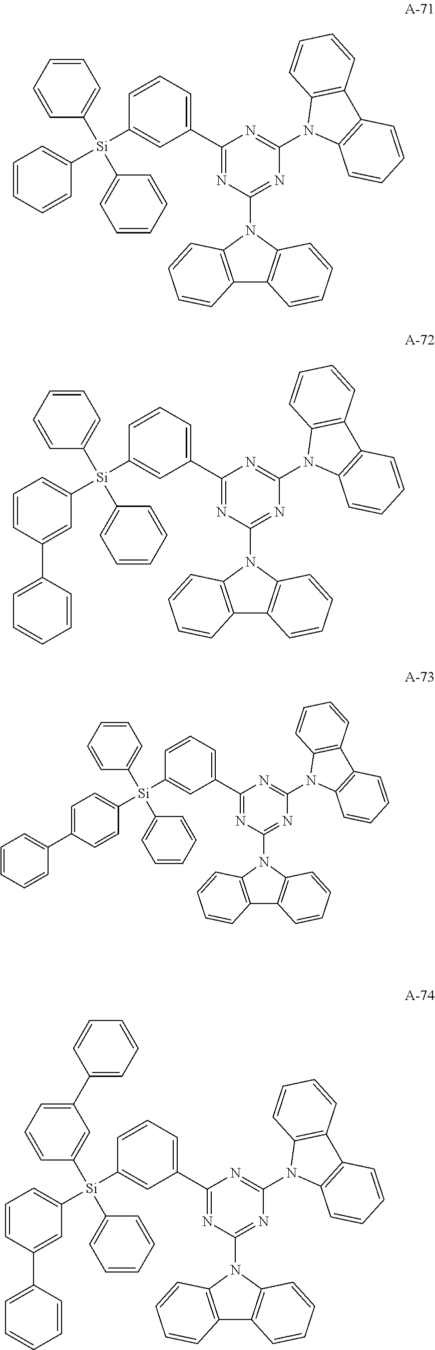 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc(-c2cccc([Si](c3ccccc3)(c3ccccc3)c3cccc(-c4nc(-n5c6ccccc6c6ccccc65)nc(-n5c6ccccc6c6ccccc65)n4)c3)c2)cc1 ; c1ccc(-c2ccc([Si](c3ccccc3)(c3ccccc3)c3cccc(-c4nc(-n5c6ccccc6c6ccccc65)nc(-n5c6ccccc6c6ccccc65)n4)c3)cc2)cc1 ; c1ccc(-c2cccc([Si](c3ccccc3)(c3cccc(-c4ccccc4)c3)c3cccc(-c4nc(-n5c6ccccc6c6ccccc65)nc(-n5c6ccccc6c6ccccc65)n4)c3)c2)cc1]

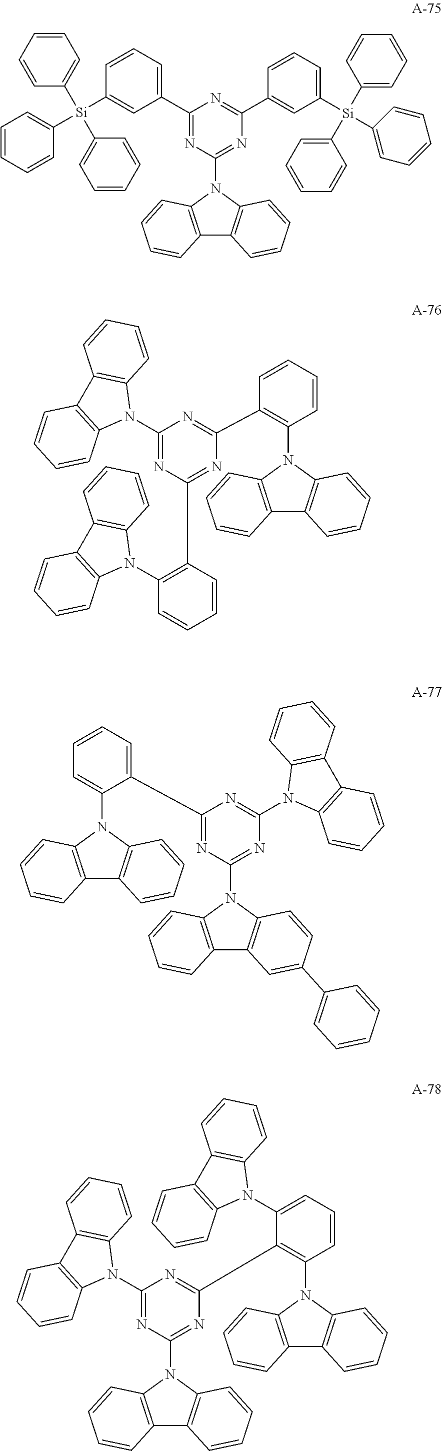 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc(-n2c3ccccc3c3ccccc32)c(-c2nc(-c3ccccc3-n3c4ccccc4c4ccccc43)nc(-n3c4ccccc4c4ccccc43)n2)c1 ; c1ccc(-c2ccc3c(c2)c2ccccc2n3-c2nc(-c3ccccc3-n3c4ccccc4c4ccccc43)nc(-n3c4ccccc4c4ccccc43)n2)cc1 ; c1cc(-n2c3ccccc3c3ccccc32)c(-c2nc(-n3c4ccccc4c4ccccc43)nc(-n3c4ccccc4c4ccccc43)n2)c(-n2c3ccccc3c3ccccc32)c1]

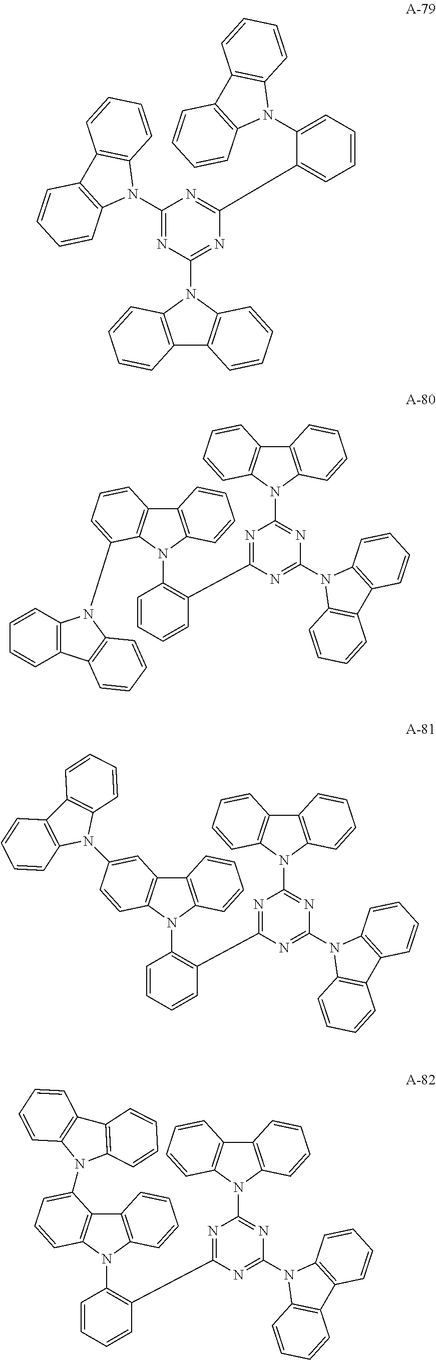 [SMILES: c1ccc(-n2c3ccccc3c3ccccc32)c(-c2nc(-n3c4ccccc4c4ccccc43)nc(-n3c4ccccc4c4ccccc43)n2)c1 ; c1ccc(-n2c3ccccc3c3cccc(-n4c5ccccc5c5ccccc54)c32)c(-c2nc(-n3c4ccccc4c4ccccc43)nc(-n3c4ccccc4c4ccccc43)n2)c1 ; c1ccc(-n2c3ccccc3c3cc(-n4c5ccccc5c5ccccc54)ccc32)c(-c2nc(-n3c4ccccc4c4ccccc43)nc(-n3c4ccccc4c4ccccc43)n2)c1 ; c1ccc(-n2c3ccccc3c3c(-n4c5ccccc5c5ccccc54)cccc32)c(-c2nc(-n3c4ccccc4c4ccccc43)nc(-n3c4ccccc4c4ccccc43)n2)c1]

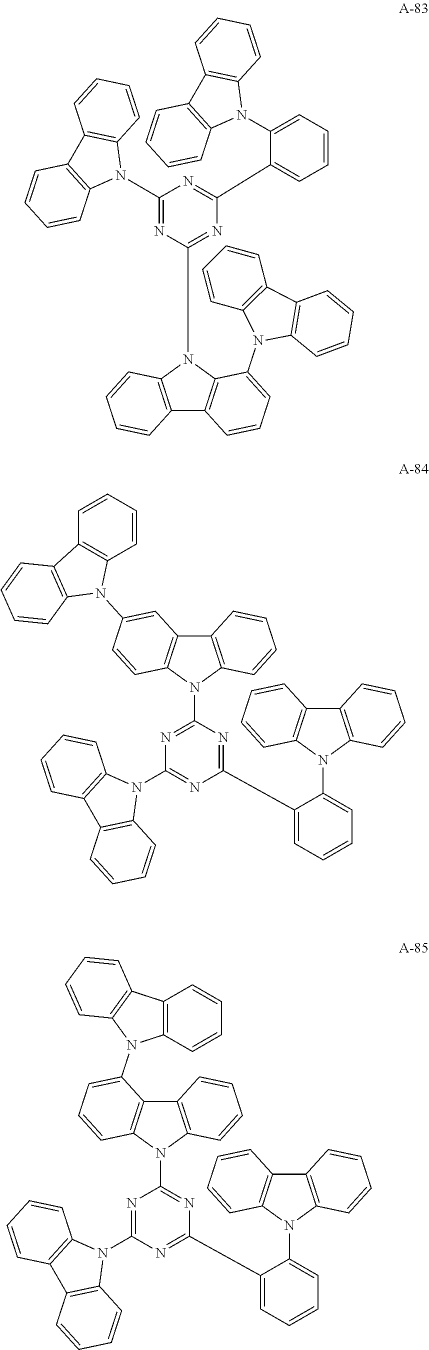 [SMILES: c1ccc(-n2c3ccccc3c3ccccc32)c(-c2nc(-n3c4ccccc4c4ccccc43)nc(-n3c4ccccc4c4cccc(-n5c6ccccc6c6ccccc65)c43)n2)c1 ; c1ccc(-n2c3ccccc3c3ccccc32)c(-c2nc(-n3c4ccccc4c4ccccc43)nc(-n3c4ccccc4c4cc(-n5c6ccccc6c6ccccc65)ccc43)n2)c1 ; c1ccc(-n2c3ccccc3c3ccccc32)c(-c2nc(-n3c4ccccc4c4ccccc43)nc(-n3c4ccccc4c4c(-n5c6ccccc6c6ccccc65)cccc43)n2)c1]

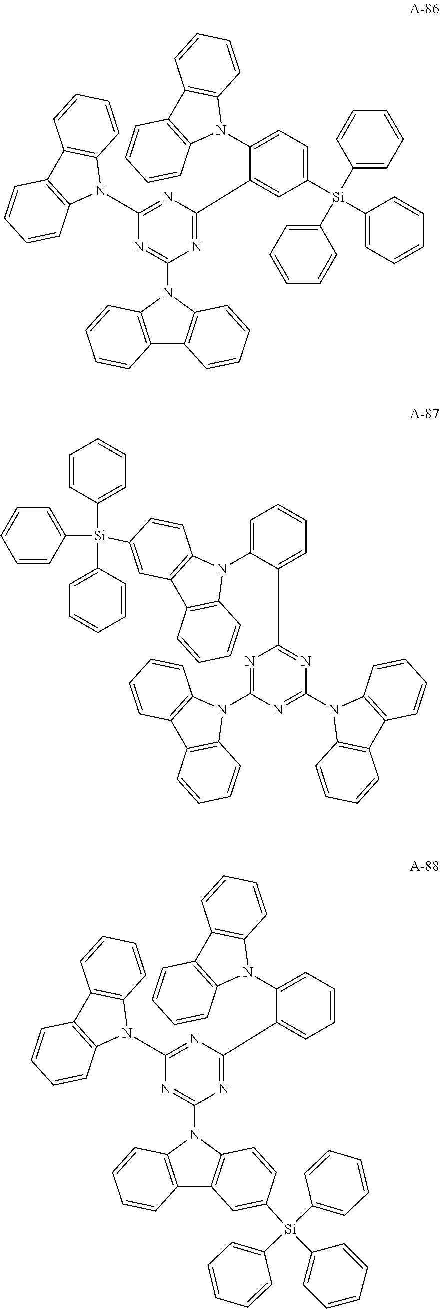 [SMILES: c1ccc([Si](c2ccccc2)(c2ccccc2)c2ccc(-n3c4ccccc4c4ccccc43)c(-c3nc(-n4c5ccccc5c5ccccc54)nc(-n4c5ccccc5c5ccccc54)n3)c2)cc1 ; c1ccc([Si](c2ccccc2)(c2ccccc2)c2ccc3c(c2)c2ccccc2n3-c2ccccc2-c2nc(-n3c4ccccc4c4ccccc43)nc(-n3c4ccccc4c4ccccc43)n2)cc1 ; c1ccc([Si](c2ccccc2)(c2ccccc2)c2ccc3c(c2)c2ccccc2n3-c2nc(-c3ccccc3-n3c4ccccc4c4ccccc43)nc(-n3c4ccccc4c4ccccc43)n2)cc1]

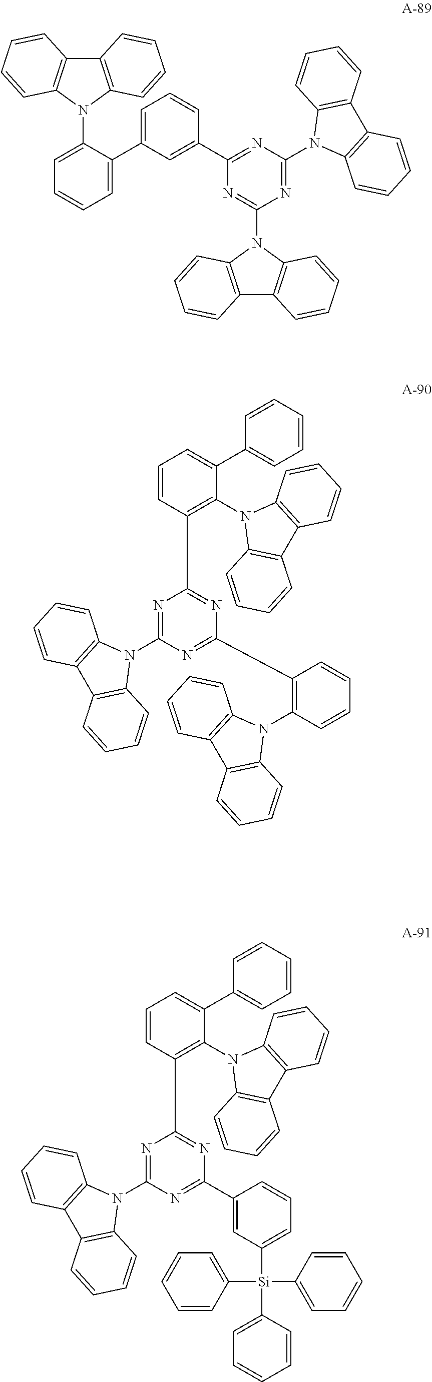 [SMILES: c1cc(-c2nc(-n3c4ccccc4c4ccccc43)nc(-n3c4ccccc4c4ccccc43)n2)cc(-c2ccccc2-n2c3ccccc3c3ccccc32)c1 ; c1ccc(-c2cccc(-c3nc(-c4ccccc4-n4c5ccccc5c5ccccc54)nc(-n4c5ccccc5c5ccccc54)n3)c2-n2c3ccccc3c3ccccc32)cc1 ; c1ccc(-c2cccc(-c3nc(-c4cccc([Si](c5ccccc5)(c5ccccc5)c5ccccc5)c4)nc(-n4c5ccccc5c5ccccc54)n3)c2-n2c3ccccc3c3ccccc32)cc1]

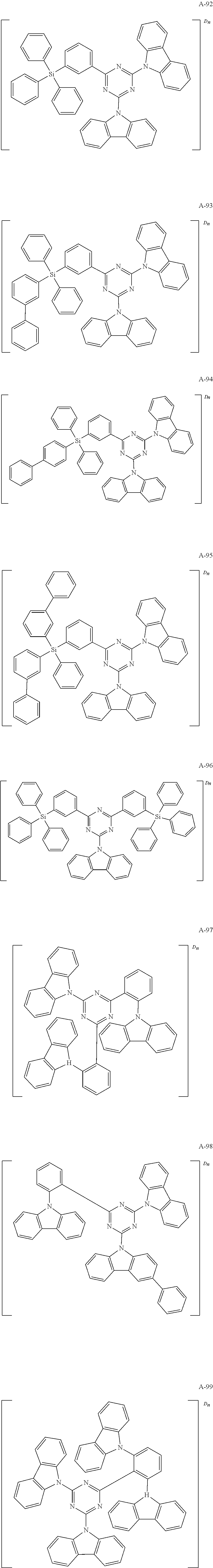

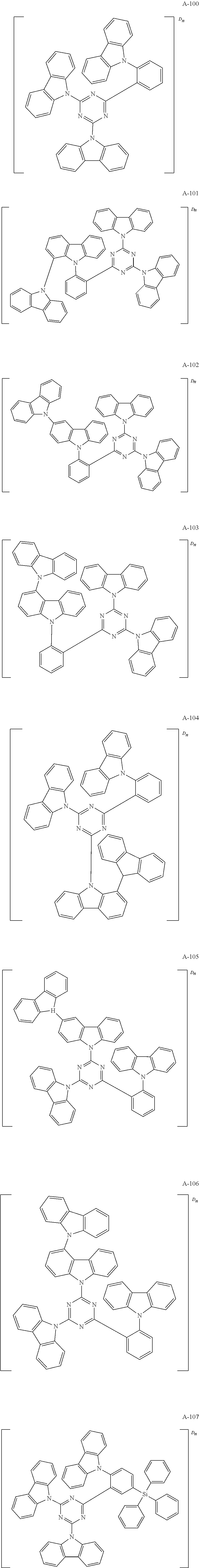

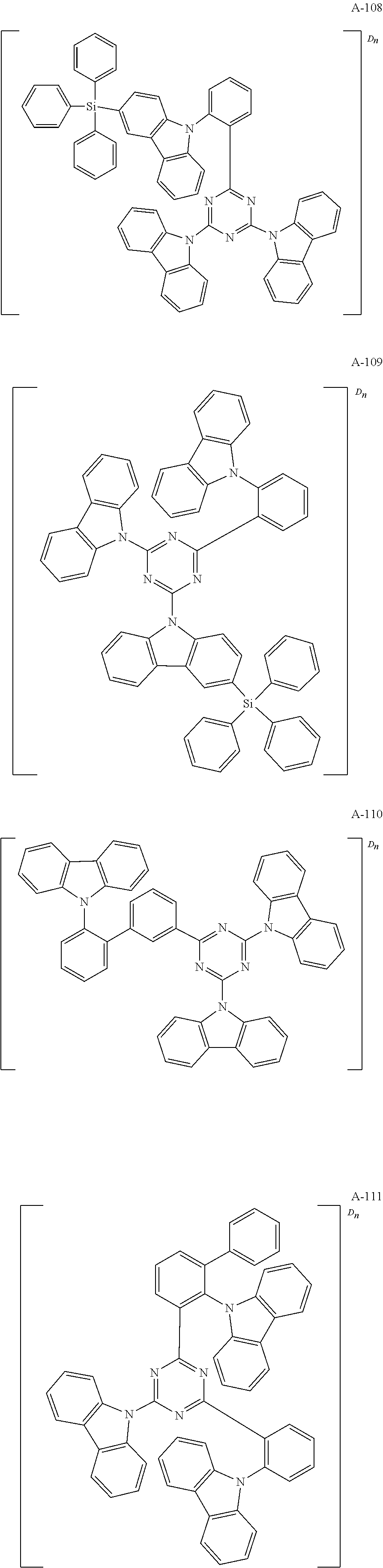

Dn indicates the number of hydrogen atoms replaced by deuterium, and n is an integer between 1 and the maximum number of possible replacements.

By including the first compound and the second compound together, good interface characteristics may be exhibited, and by finely controlling the mobility of holes and electrons, the balance of electrons and holes in the active layer (e.g., the light emitting layer, 130) of the organic optoelectronic device may be increased, thereby helping increase luminescence efficiency, while at the same time helping improve life-span by reducing the unbound charge due to the imbalance in the mobility of electrons and holes.

In an implementation, the first compound and the second compound may be included as a host, and an organic optoelectronic device 100 including a light emitting layer 130 to which the first compound and the second compound are applied may be finely controlled so that holes and electrons injected from the anode 110 and the cathode 120, respectively, have appropriate mobility within the light emitting layer 130, thereby strongly inducing exciton generation within the light emitting layer and helping increase luminous efficiency within the light emitting layer.

The composition for an organic optoelectronic device may include the first compound and the second compound in various ratios. In an implementation, the composition for an organic optoelectronic device may include the first compound and the second compound in a weight ratio of, e.g., 10:90 to 90:10, and within the above range, may include the first compound and the second compound in a weight ratio of, e.g., about 20:80 to about 80:20, about 30:70 to about 70:30, about 40:60 to about 60:40, or about 50:50.

In an implementation, the first compound may be included in an amount greater than or equal to that of the second compound, e.g., the first compound may be included in an amount of about 50 wt % to about 90 wt %, based on the total weight of the first compound and the second compound.

In an implementation, the first compound may be included in an amount less than or equal to that of the second compound, e.g., the first compound may be included in an amount of about 10 wt % to about 50 wt %, based on the total weight of the first compound and the second compound.

The composition for an organic optoelectronic device may further include a luminescent dopant in addition to the first compound and the second compound. The luminescent dopant may be a material that causes luminescence when mixed in a small amount in the composition for an organic optoelectronic device and generally a material such as a metal complex that emits luminescence by multiple excitation that excites to a triplet state or higher may be used. The luminescent dopant may be, e.g., an inorganic, organic, or organic/inorganic compound, and may be included in one or more types.

The luminescent dopant may be, e.g., a phosphorescent dopant, a delayed fluorescent dopant, a fluorescent dopant, or a combination thereof. In an implementation, using a phosphorescent dopant or a delayed fluorescent dopant as a phosphorescent sensitizer or an assist dopant may help effectively transfer energy received from the host to the fluorescent dopant.

The phosphorescent dopant may be an organometallic compound, and the energy transfer to the fluorescent dopant may be increased, so that the excitons formed in the light emitting layer rapidly emit light within the light emitting layer, thereby helping reduce the deterioration of the light emitting diode.

The phosphorescent dopant may be an organometallic compound, and may be e.g., an organometallic compound including iridium (Ir), platinum (Pt), osmium (Os), titanium (Ti), zirconium (Zr), hafnium (Hf), europium (Eu), terbium (Tb), thulium (Tm), rhodium (Rh), or a combination thereof, and may be an organometallic compound including an organic ligand including a nitrogen-containing ring. The nitrogen-containing ring may be, e.g., a substituted or unsubstituted pyridine, a substituted or unsubstituted pyrimidine, a substituted or unsubstituted triazine, a substituted or unsubstituted carbazole, a substituted or unsubstituted imidazole, a substituted or unsubstituted benzimidazole, or a combination thereof.

The phosphorescent dopant may be, e.g., one of Compounds P1 to P32 and P41 to P52.

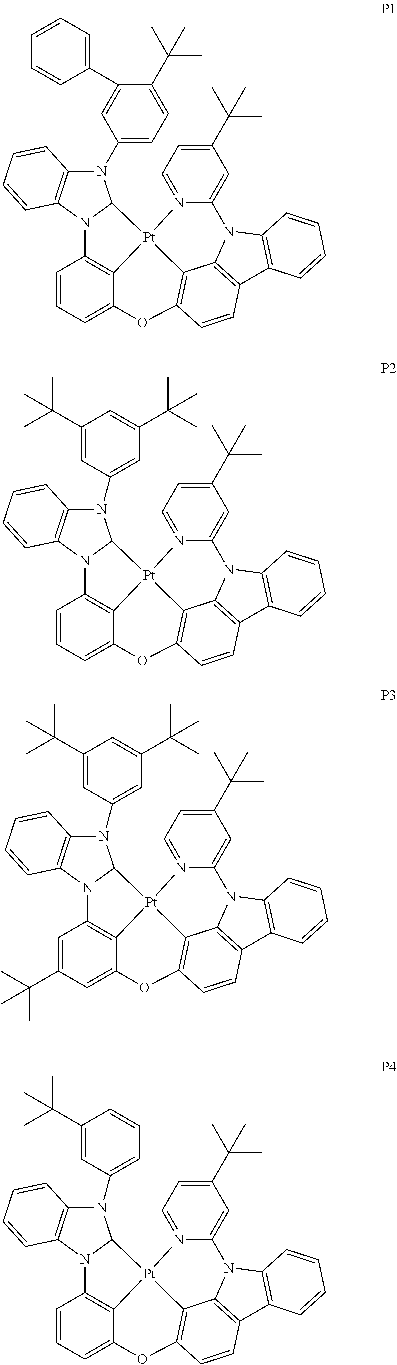

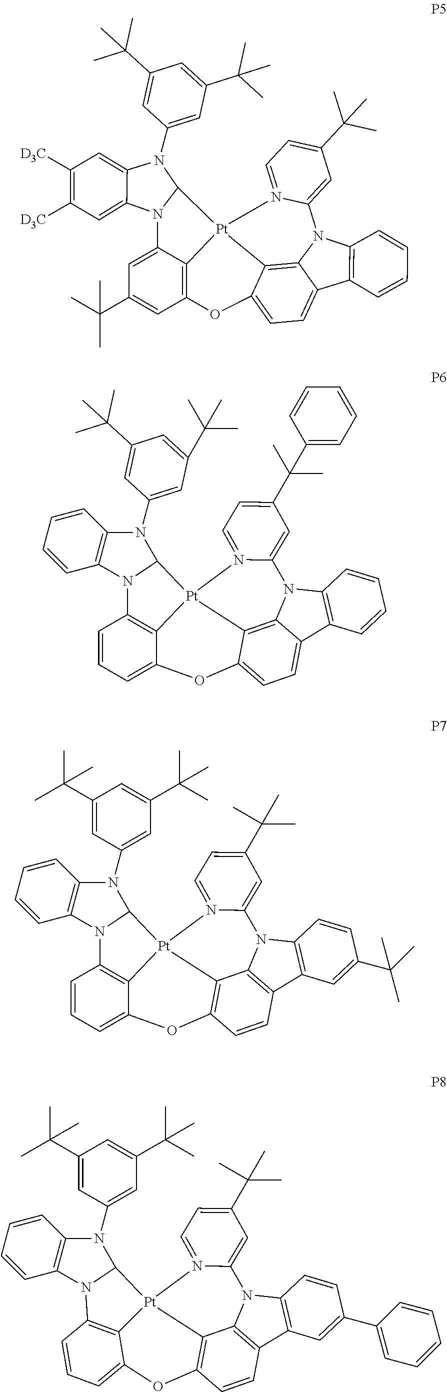

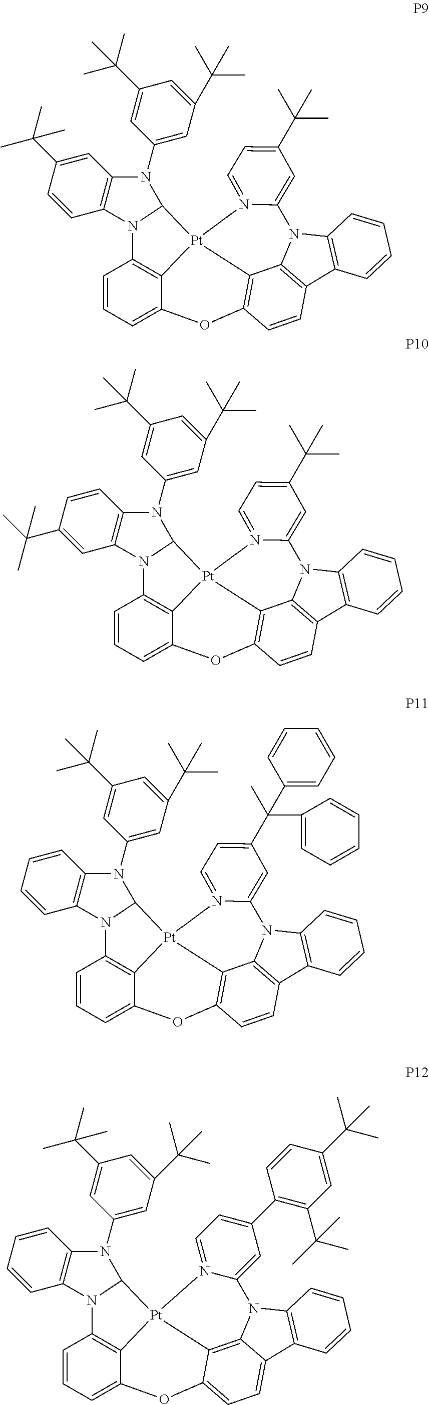

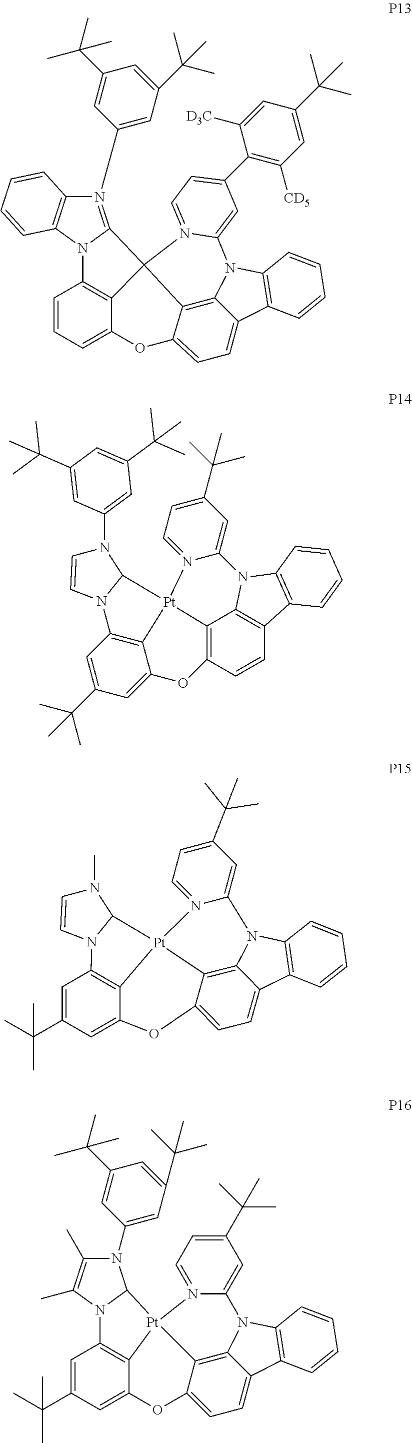

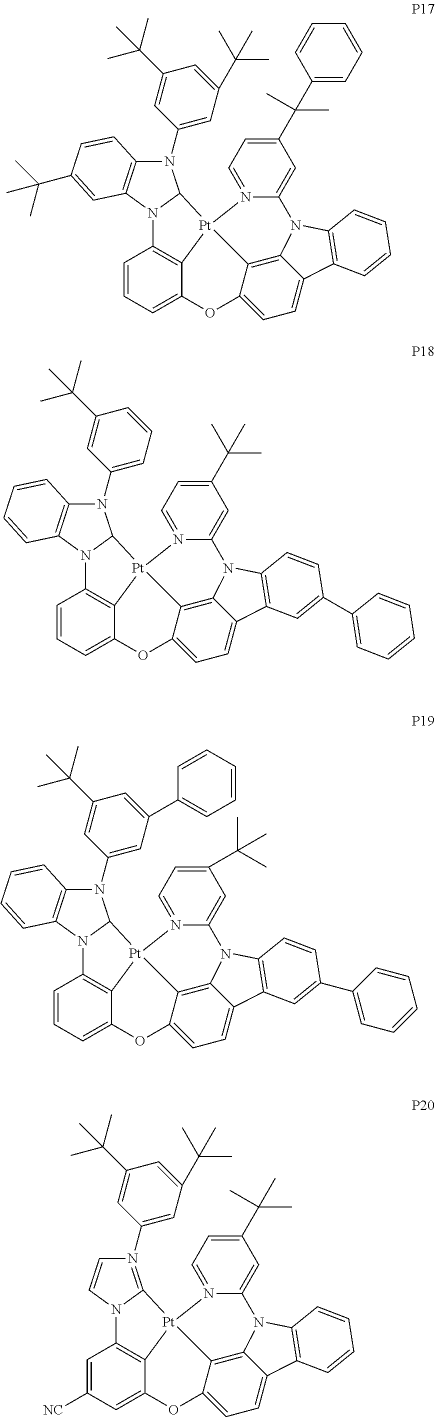

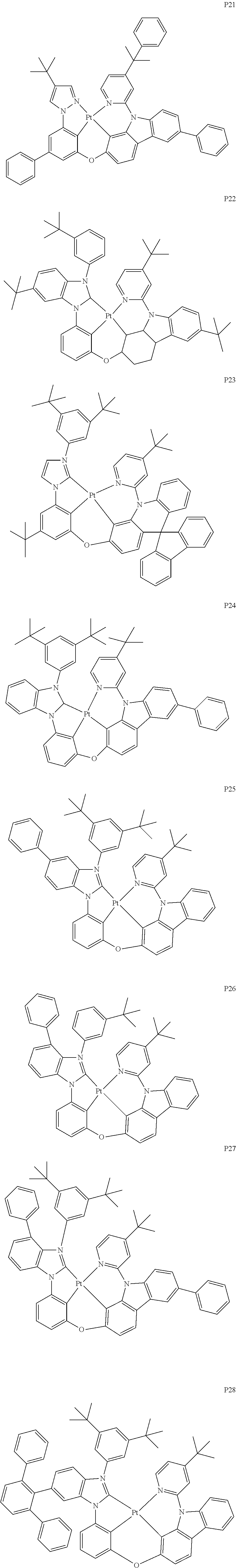

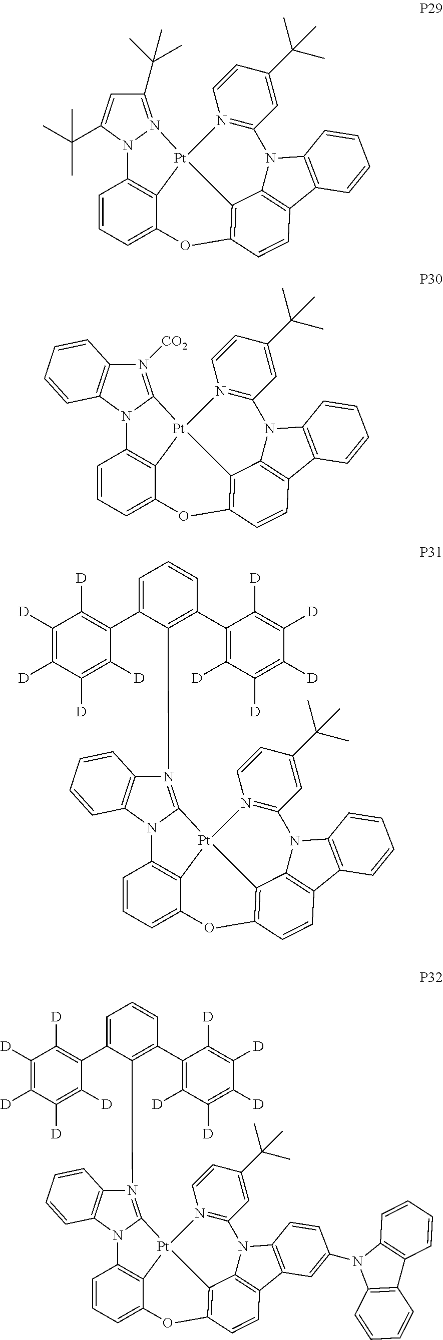

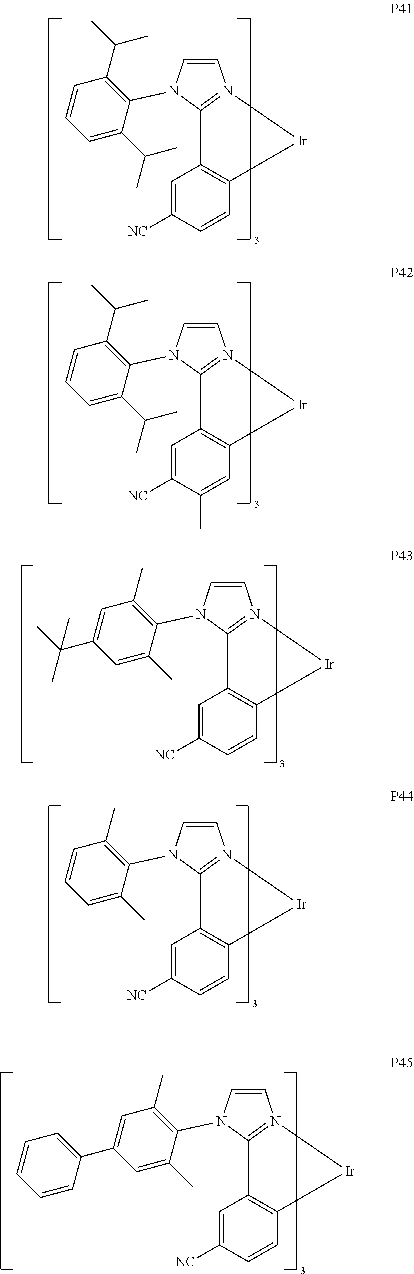

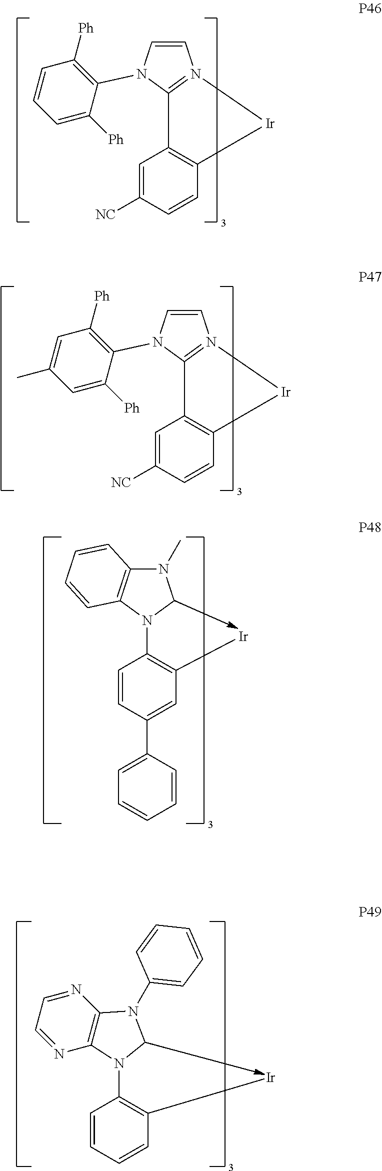

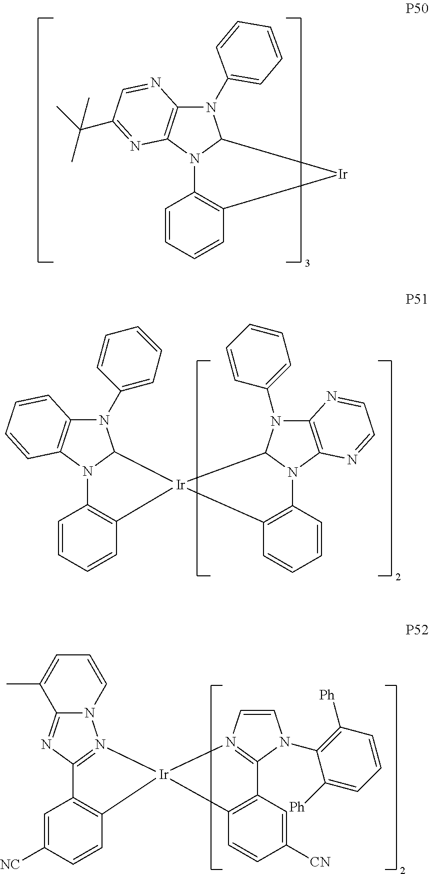

The delayed fluorescent dopant may be a metal atom-free compound with a ΔEST of less than or equal to 0.2 eV, and because the up-conversion process by reverse intersystem crossing (RISC) may be advantageous, the efficiency of a light emitting diode including it may be improved.

The delayed fluorescent dopant may be, e.g., one of Compounds T1 to T12.

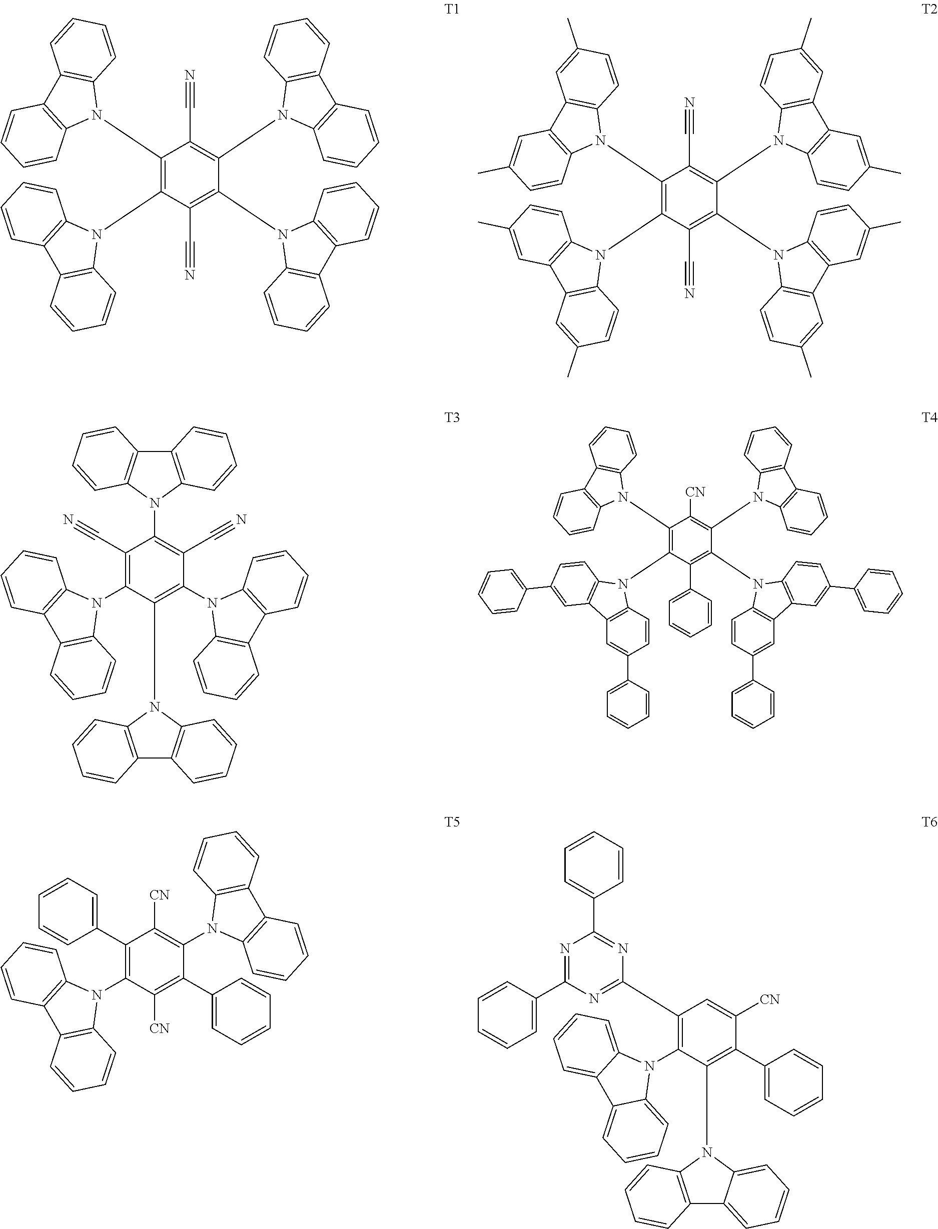

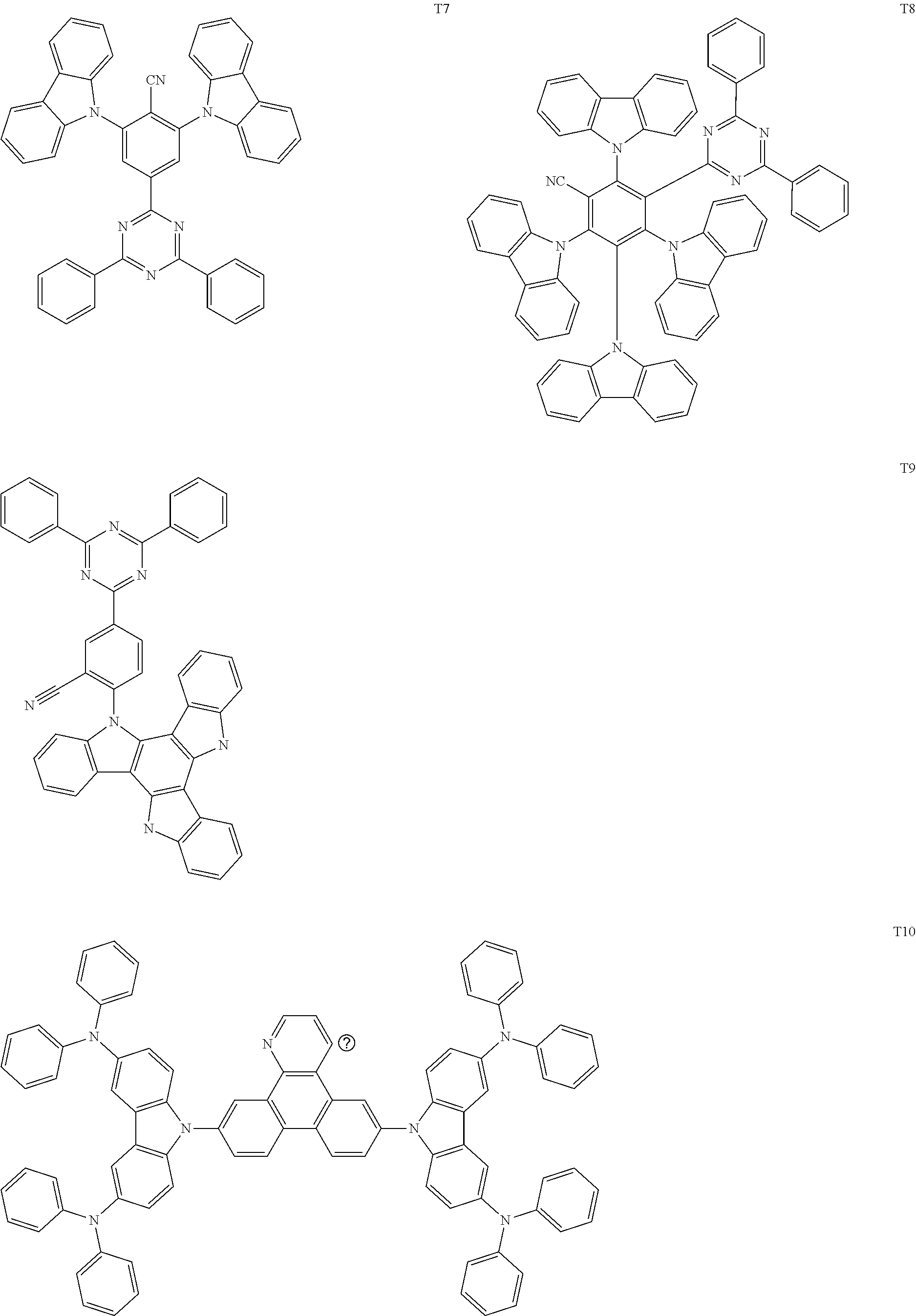 [SMILES: N#Cc1c(-n2c3ccccc3c3ccccc32)cc(-c2nc(-c3ccccc3)nc(-c3ccccc3)n2)cc1-n1c2ccccc2c2ccccc21 ; N#Cc1c(-n2c3ccccc3c3ccccc32)c(-c2nc(-c3ccccc3)nc(-c3ccccc3)n2)c(-n2c3ccccc3c3ccccc32)c(-n2c3ccccc3c3ccccc32)c1-n1c2ccccc2c2ccccc21 ; N#Cc1cc(-c2nc(-c3ccccc3)nc(-c3ccccc3)n2)ccc1-n1c2ccccc2c2c3[nH]c4ccccc4c3c3[nH]c4ccccc4c3c21 ; c1ccc(N(c2ccccc2)c2ccc3c(c2)c2cc(N(c4ccccc4)c4ccccc4)ccc2n3-c2ccc3c(c2)c2cccnc2c2cc(-n4c5ccc(N(c6ccccc6)c6ccccc6)cc5c5cc(N(c6ccccc6)c6ccccc6)ccc54)ccc32)cc1]

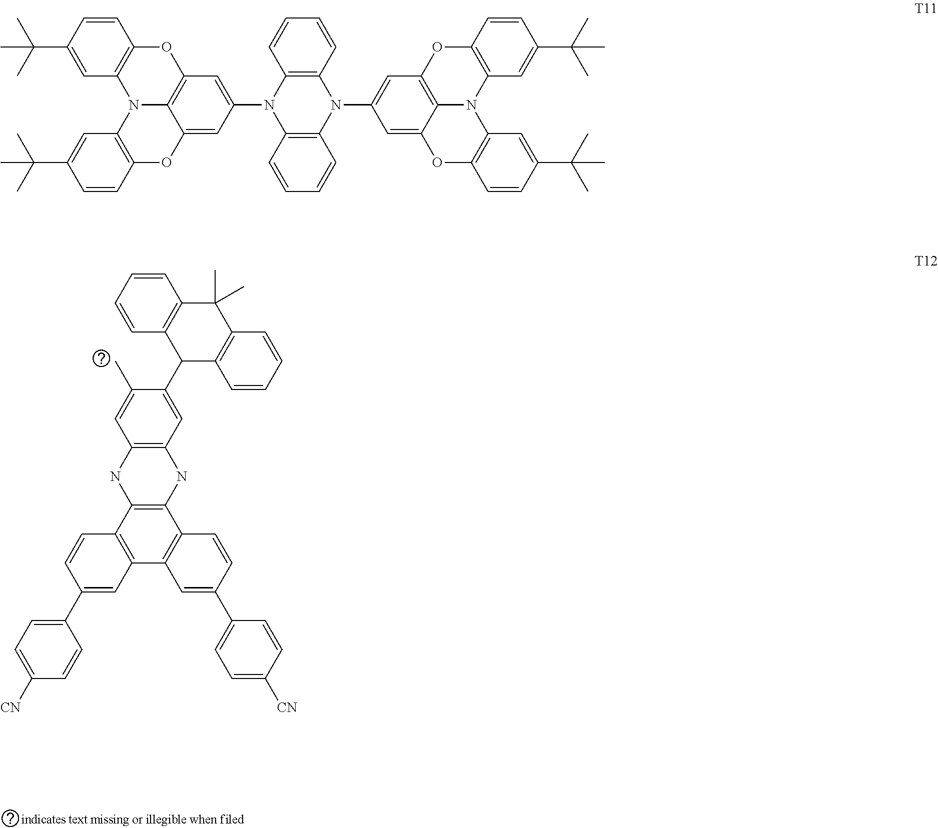

The fluorescent dopant may be, e.g., a polycyclic compound, and may improve luminescence efficiency and life-span characteristics of the light emitting diode by receiving energy transfer within the light emitting layer due to high absorbance.

The fluorescent dopant may be a condensed polycyclic compound containing, e.g., boron (B), nitrogen (N) or a combination thereof, and may be, e.g., one of Compounds D1 to D38.

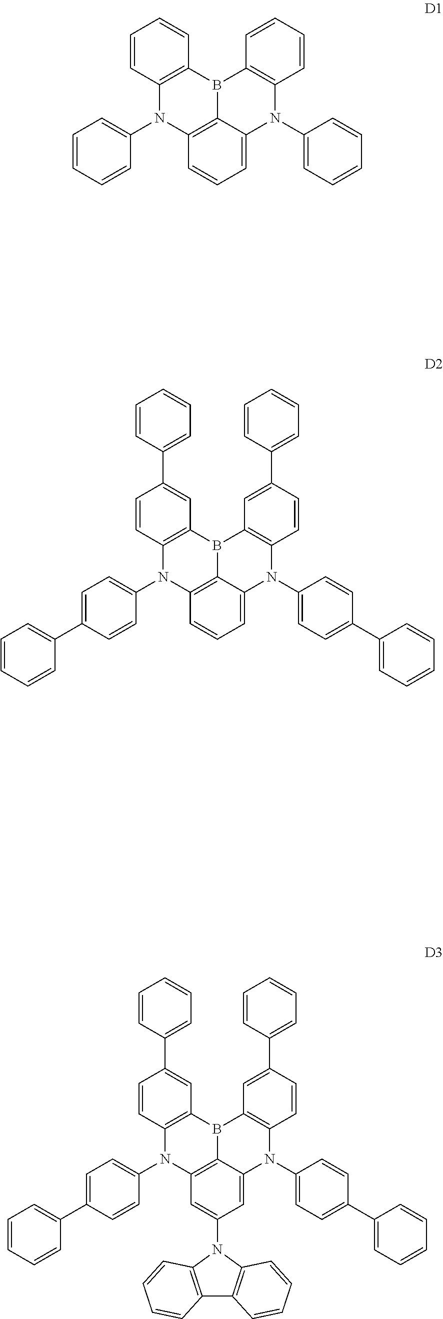 [SMILES: c1ccc(N2c3ccccc3B3c4ccccc4N(c4ccccc4)c4cccc2c43)cc1 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1]

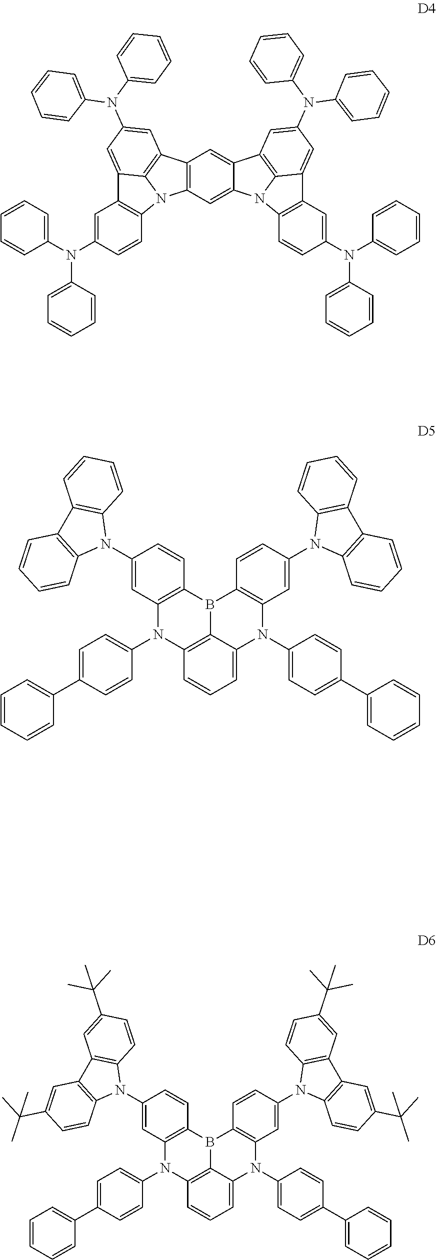 [SMILES: c1ccc(N(c2ccccc2)c2ccc3c(c2)c2cc(N(c4ccccc4)c4ccccc4)cc4c5cc6c7cc(N(c8ccccc8)c8ccccc8)cc8c9cc(N(c%10ccccc%10)c%10ccccc%10)ccc9n(c6cc5n3c24)c87)cc1 ; c1ccc(-c2ccc(N3c4cc(-n5c6ccccc6c6ccccc65)ccc4B4c5ccc(-n6c7ccccc7c7ccccc76)cc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ccc(-n2c4ccc(C(C)(C)C)cc4c4cc(C(C)(C)C)ccc42)cc1N3c1ccc(-c2ccccc2)cc1]

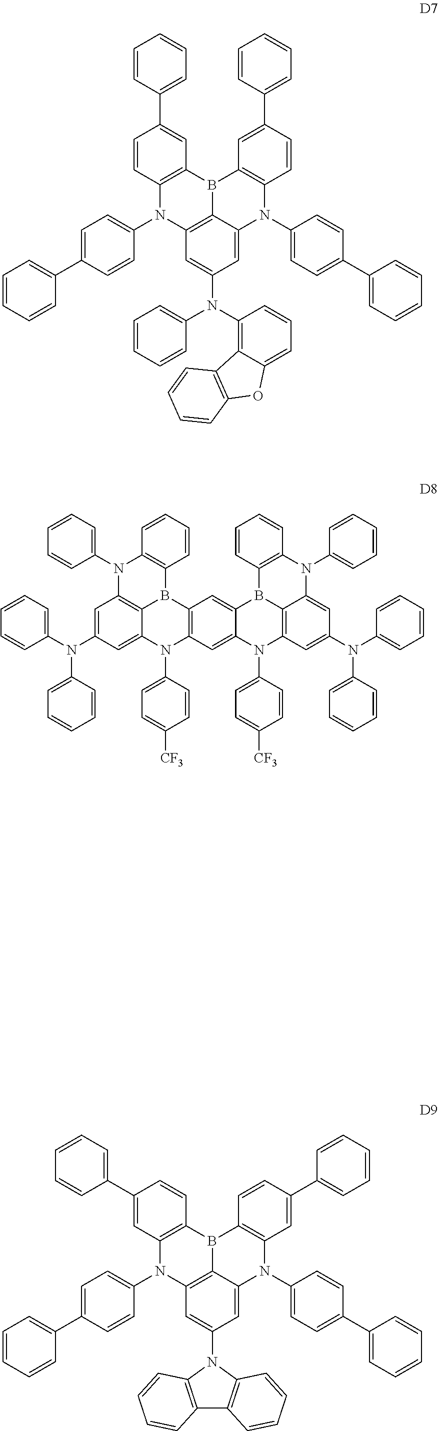 [SMILES: c1ccc(-c2ccc(N3c4ccc(-c5ccccc5)cc4B4c5cc(-c6ccccc6)ccc5N(c5ccc(-c6ccccc6)cc5)c5cc(N(c6ccccc6)c6cccc7oc8ccccc8c67)cc3c54)cc2)cc1 ; FC(F)(F)c1ccc(N2c3cc4c(cc3B3c5ccccc5N(c5ccccc5)c5cc(N(c6ccccc6)c6ccccc6)cc2c53)B2c3ccccc3N(c3ccccc3)c3cc(N(c5ccccc5)c5ccccc5)cc(c32)N4c2ccc(C(F)(F)F)cc2)cc1 ; c1ccc(-c2ccc(N3c4cc(-c5ccccc5)ccc4B4c5ccc(-c6ccccc6)cc5N(c5ccc(-c6ccccc6)cc5)c5cc(-n6c7ccccc7c7ccccc76)cc3c54)cc2)cc1]

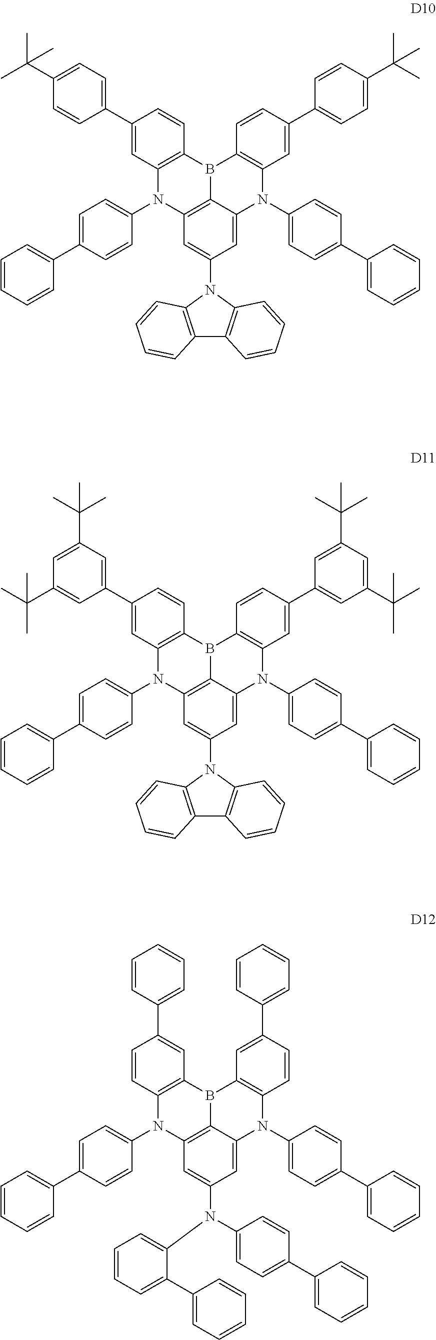 [SMILES: CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1 ; CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1 ; c1ccc(-c2ccc(N(c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)c3ccccc3-c3ccccc3)cc2)cc1]

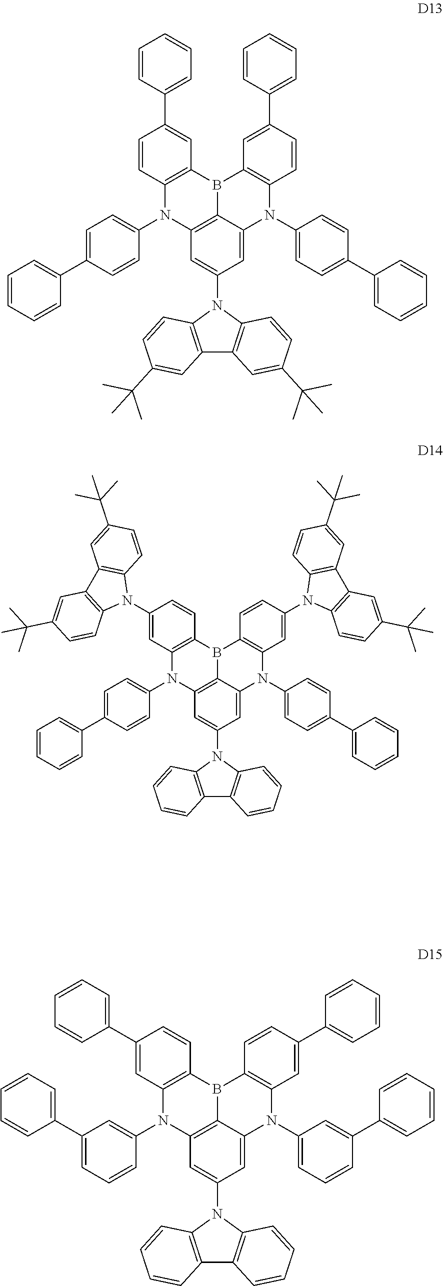 [SMILES: CC(C)(C)c1ccc2c(c1)c1cc(C(C)(C)C)ccc1n2-c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccccc2)cc1 ; c1ccc(-c2cccc(N3c4cc(-c5ccccc5)ccc4B4c5ccc(-c6ccccc6)cc5N(c5cccc(-c6ccccc6)c5)c5cc(-n6c7ccccc7c7ccccc76)cc3c54)c2)cc1]

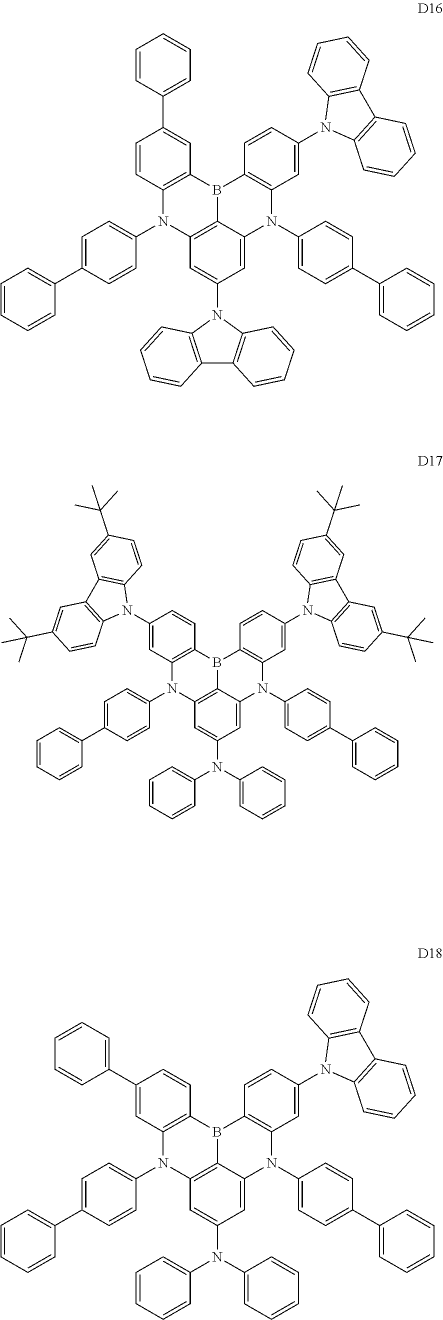 [SMILES: c1ccc(-c2ccc(N3c4ccc(-c5ccccc5)cc4B4c5ccc(-n6c7ccccc7c7ccccc76)cc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1ccc(-n2c4ccc(C(C)(C)C)cc4c4cc(C(C)(C)C)ccc42)cc1N3c1ccc(-c2ccccc2)cc1 ; c1ccc(-c2ccc(N3c4cc(-c5ccccc5)ccc4B4c5ccc(-n6c7ccccc7c7ccccc76)cc5N(c5ccc(-c6ccccc6)cc5)c5cc(N(c6ccccc6)c6ccccc6)cc3c54)cc2)cc1]

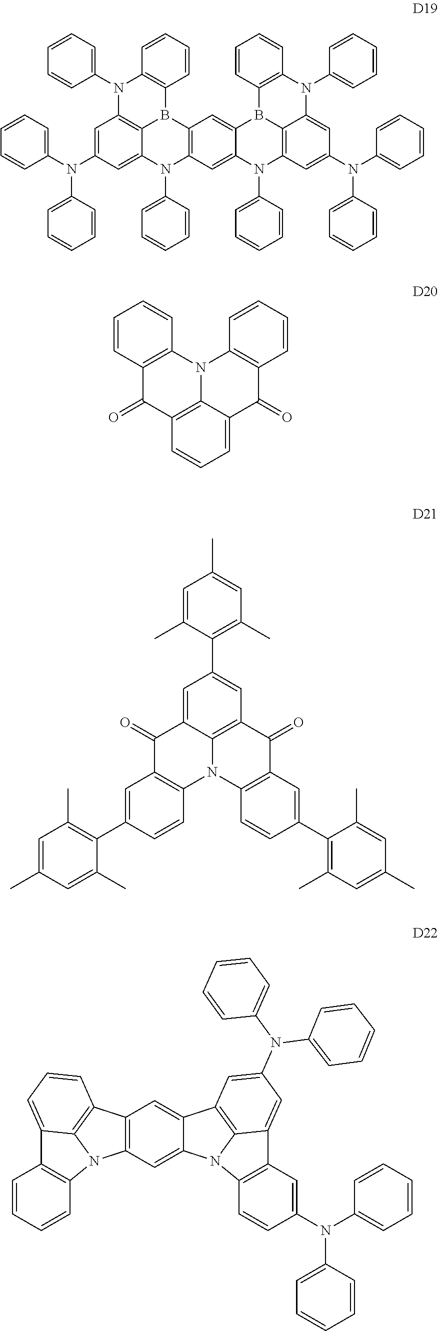

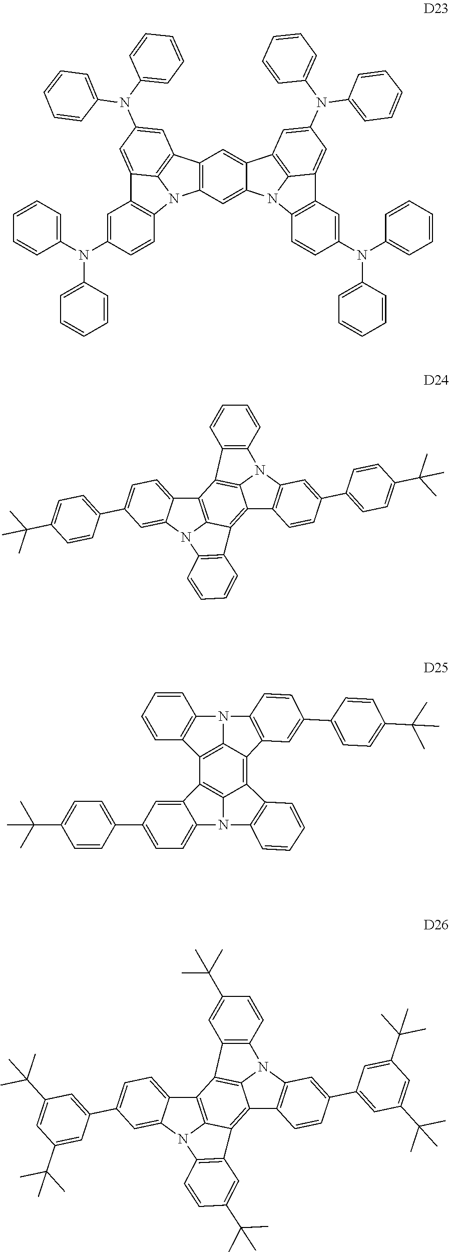

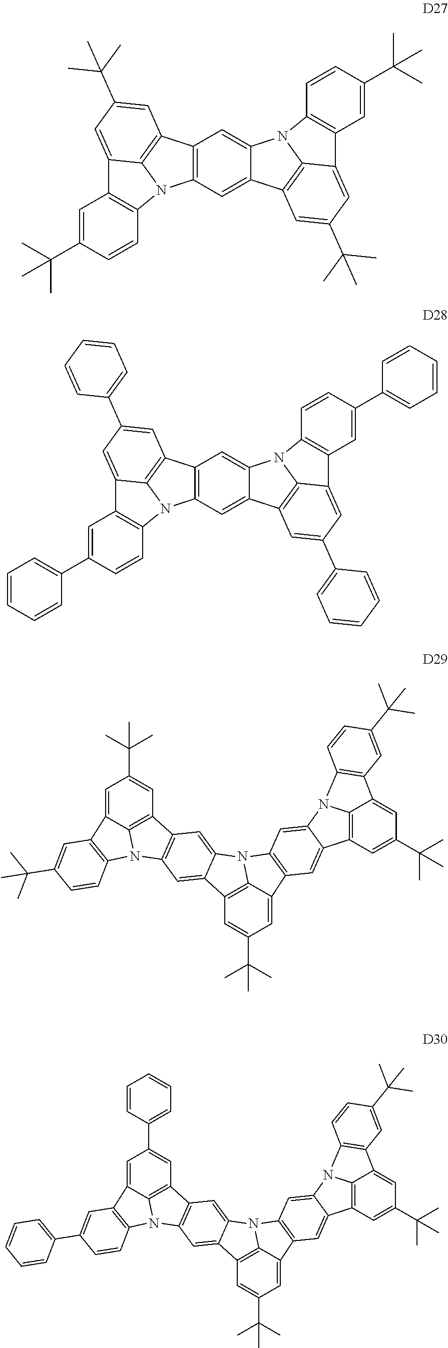

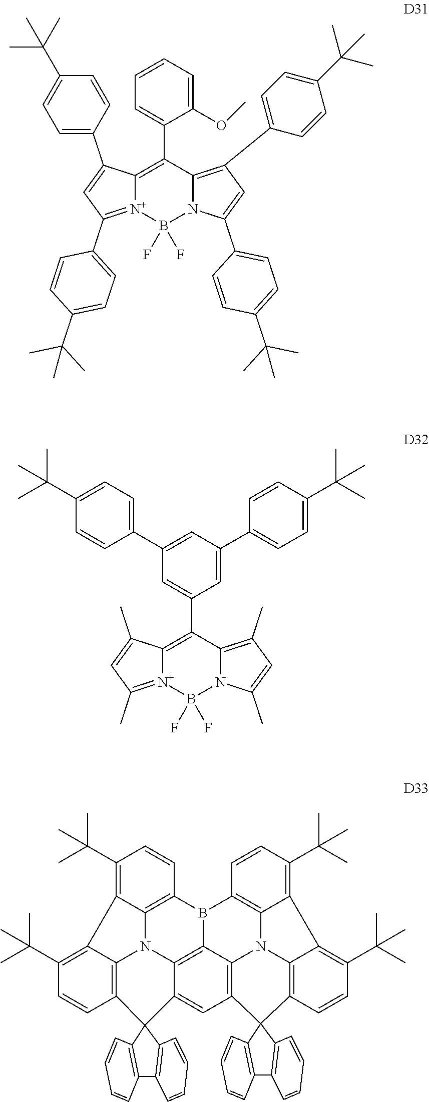

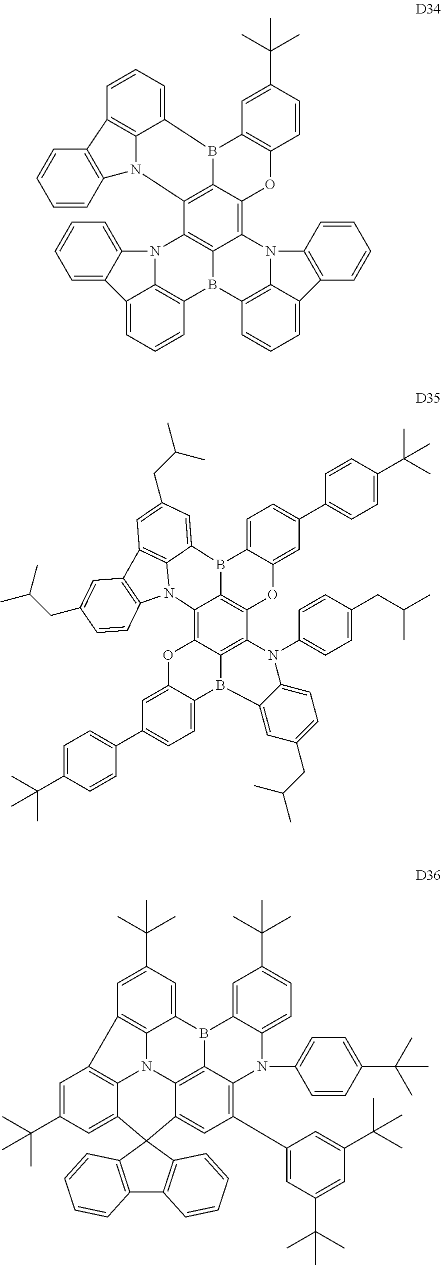

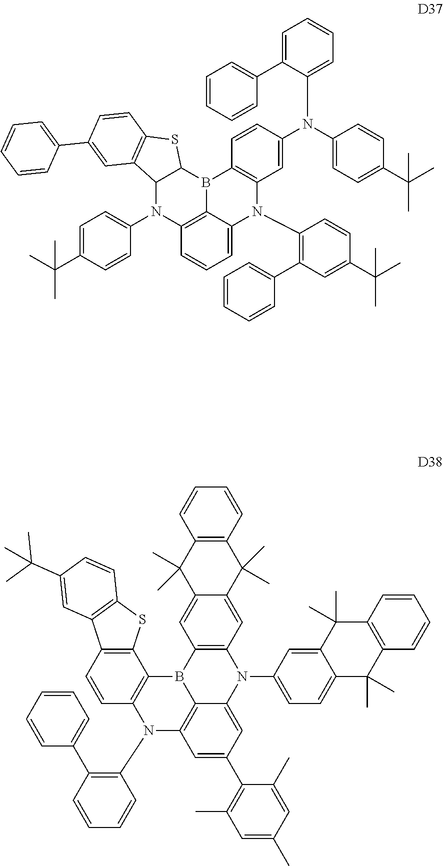

The phosphorescent dopant, the delayed fluorescent dopant, or the fluorescent dopant may be included in an amount of, e.g., less than or equal to about 20 wt %, respectively, and within the above range, may be included in an amount of, e.g., about 0.1 to about 20 wt %, about 0.1 to about 15 wt %, about 0.1 to about 10 wt %, about 0.1 to about 7 wt %, about 0.1 to about 5 wt %, about 0.1 to about 4 wt %, about 1 to about 20 wt %, about 1 to about 15 wt %, about 1 to about 10 wt %, about 1 to about 7 wt %, about 1 to about 5 wt % or about 1 to about 4 wt %, respectively, based on the total amount of the composition for an organic optoelectronic device.

The composition for an organic optoelectronic device may additionally include an additive, and the additive may be, e.g., an organic material, an inorganic material, an organic/inorganic material, or a combination thereof.

Hereinafter, an organic optoelectronic device using the aforementioned compound for an organic optoelectronic device or composition for an organic optoelectronic device will be described.

The organic optoelectronic device is not particularly limited as long as it is a device that may convert electrical energy and optical energy into each other, and examples thereof may include an organic light emitting diode, an organic photoelectric device, organic solar cell, or an organic photo-conductor drum.

The organic optoelectronic device may include, e.g., an anode and a cathode facing each other, and an organic layer between the anode and the cathode, and the organic layer may include the aforementioned compound or composition. The organic layer may include, e.g., an active layer, such as a light emitting layer or a light absorbing layer, and the aforementioned compound or composition may be included in the active layer. The organic layer may include, e.g., an auxiliary layer between the anode and the active layer or between the cathode and the active layer, and the aforementioned compound or composition may be included in the auxiliary layer.

Here, an organic light emitting diode, which is an example of an organic optoelectronic device, is described as an example, but it may be equally applied to other organic optoelectronic devices.

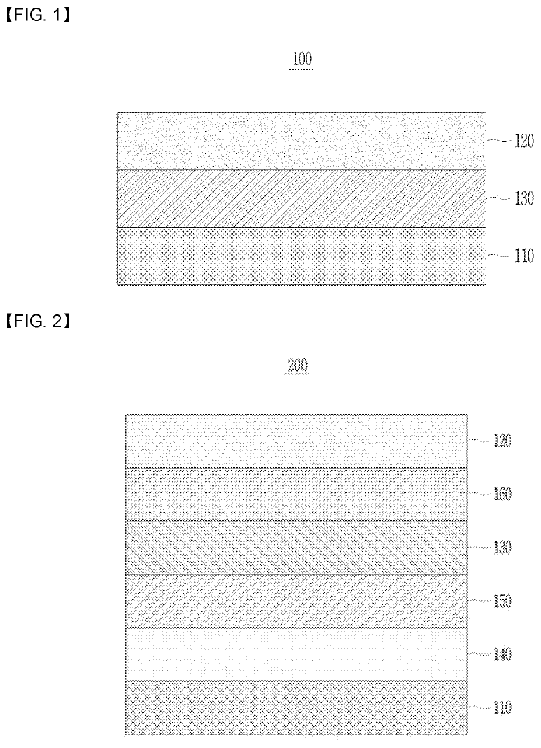

FIG. 1 is a cross-sectional view showing an example of an organic light emitting diode, which is an example of an organic optoelectronic device according to some example embodiments.

Referring to FIG. 1, an organic optoelectronic device 100 according to some example embodiments may include, e.g., an anode 110, a cathode 120 facing each other, and a light emitting layer 130 between the anode 110 and the cathode 120.

The anode 110 may be made of a conductor with a high work function to facilitate hole injection and may be, e.g., made of a metal, a metal oxide, or a conductive polymer. The anode 110 may be, e.g., made of a metal such as nickel, platinum, vanadium, chromium, copper, zinc, gold, or an alloy thereof, a metal oxide such as zinc oxide, indium oxide, indium tin oxide (ITO), or indium zinc oxide (IZO); a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 120 may be, e.g., made of a conductor with a low work function to help facilitate electron injection, e.g., and may be made of a metal, a metal oxide, or a conductive polymer. The cathode 120 may be made of a metal such as magnesium, calcium, sodium, potassium, titanium, indium, yttrium, lithium, gadolinium, aluminum, silver, tin, lead, cesium, barium, or an alloy thereof; or a multilayer structure material such as LiF/Al, LiO₂/Al, LiF/Ca, or BaF₂/Ca.

The light emitting layer 130 may include the aforementioned compound for an organic optoelectronic device or composition for an organic optoelectronic device. The light emitting layer 130 may further include another organic compound as a mixed host. The light emitting layer 130 may further include the aforementioned luminescent dopant and may include, e.g., a fluorescent dopant, a phosphorescent photosensitizer, or a combination thereof as described above.

In an implementation, the light emitting layer 130 may emit light in the blue emission spectrum by combining the aforementioned compound for an organic optoelectronic device or composition for an organic optoelectronic device and a luminescent dopant. The peak wavelength of the blue emission spectrum may be, e.g., between about 410 nm and 480 nm, and within that range may be, e.g., between about 420 nm and 470 nm or between about 430 nm and 470 nm. In an implementation, at least one of the first compound and the aforementioned second compound of the composition for an organic optoelectronic device may have a higher triplet energy level, thereby helping facilitate exciton transfer to a blue luminescent dopant, thereby helping enable implementation of a high-efficiency, long life-span organic optoelectronic device.

The aforementioned compound for an organic optoelectronic device may increase the stability of the material during device operation and improve the life-span and driving voltage by helping transfer the LUMO phore (LUMO part in the molecule) to an additionally connected phenyl group.

In an implementation, the aforementioned composition for an organic optoelectronic device may help further improve life-span by helping further increase the balance of electrons and holes within the light emitting layer 130 and helping further increase luminous efficiency, while reducing the unbound charge due to the imbalance in the mobility of electrons and holes, by including the first compound, which may be a bipolar compound having relatively strong hole transport characteristics, and the second compound, which may be a bipolar compound having relatively strong electron transport characteristics.

In an implementation, an organic optoelectronic device 100 including a light emitting layer 130 in which an organic optoelectronic device composition may be applied as a mixed host may help enhance luminous efficiency in the light emitting layer 130 by helping finely control holes and electrons injected from the anode 110 and cathode 120 respectively to have appropriate mobility within the light emitting layer 130, thereby strongly inducing exciton generation within the light emitting layer 130.

In an implementation, a difference in mobility of holes and electrons injected from the anode 110 and cathode 120 respectively within the light emitting layer 130 may help reduce or prevent the generation of excitons at inappropriate locations, such as the interface between the light emitting layer 130 and adjacent layers, or the accumulation of unbound charges at the interface between the light emitting layer 130 and adjacent layers. Accordingly, the roll-off phenomenon in which the luminous efficiency of the organic optoelectronic device 100 rapidly decreases due to non-luminescent excitons or non-bound charges may be reduced or prevented, and thus the life-span of the organic optoelectronic device 100 may ultimately be improved.

The organic optoelectronic device 100 may be manufactured by forming an anode 110 or a cathode 120 on a substrate, forming a light emitting layer 130 using dry film forming methods such as vacuum evaporation, sputtering, plasma plating, and ion plating, and forming a cathode 120 or an anode 110 thereon.

FIG. 2 is a cross-sectional view showing another example of an organic light emitting diode, which is an example of an organic optoelectronic device according to some example embodiments.

Referring to FIG. 2, the organic optoelectronic device 200 according to the present embodiments may include, e.g., an anode 110, a cathode 120, and a light emitting layer 130, similar to the aforementioned embodiments. However, unlike the aforementioned embodiments, the organic optoelectronic device 200 according to the embodiment illustrated in FIG. 2 further includes, e.g., a hole transport layer 140, a hole transport auxiliary layer 150, and an electron transport layer 160.

The hole transport layer 140 may be, e.g., between the anode 110 and the light emitting layer 130, and the hole transport auxiliary layer 150 may be, e.g., between the light emitting layer 130 and the hole transport layer 140. The electron transport layer 160 may be, e.g., located between the cathode 120 and the light emitting layer 130.

The hole transport layer 140 may help facilitate hole transfer from the anode 110 to the light emitting layer 130, and may include, e.g., an amine compound. In an implementation, the amine compound may have at least one aryl group or heteroaryl group with hole characteristics. In an implementation, the amine compound may be represented by Chemical Formula 6A or 6B.

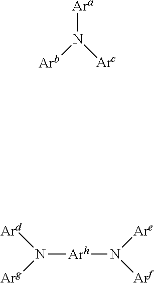

In Chemical Formula 6A or 6B, Ar^{a} to Ar^{g} may each independently be, e.g., hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof.

At least one of Ar^{a} to Ar^{c} and at least one of Ar^{d} to Ar^{g} may be, e.g., a substituted or unsubstituted C6 to C30 aryl group, a substituted or unsubstituted C2 to C30 heteroaryl group, or a combination thereof.

Ar^{h} may be, e.g., a single bond, a substituted or unsubstituted C1 to C20 alkylene group, a substituted or unsubstituted C6 to C30 arylene group, a substituted or unsubstituted C2 to C30 heteroarylene group, or a combination thereof.

The hole transport auxiliary layer 150 may form an interface with the light emitting layer 130 by being between the hole transport layer 140 and the light emitting layer 130 and in contact with the light emitting layer 130. The hole transport auxiliary layer 150 may help further reduce or prevent exciton generation at inappropriate locations, such as the interface between the aforementioned light emitting layer 130 and adjacent layers, or accumulation of unbound charges at the interface between the light emitting layer 130 and adjacent layers. Accordingly, the roll-off phenomenon in which the luminous efficiency of the organic optoelectronic device 100 rapidly decreases due to non-luminescent excitons or unbound charges may be further reduced or prevented, and thus the life-span of the organic optoelectronic device 100 may be further improved.

The electron transport layer 160 may help further increase electron injection or electron mobility and block holes between the cathode 120 and the light emitting layer 130.

The electron transport layer 160 may include, e.g., one or more compounds of Group 3.

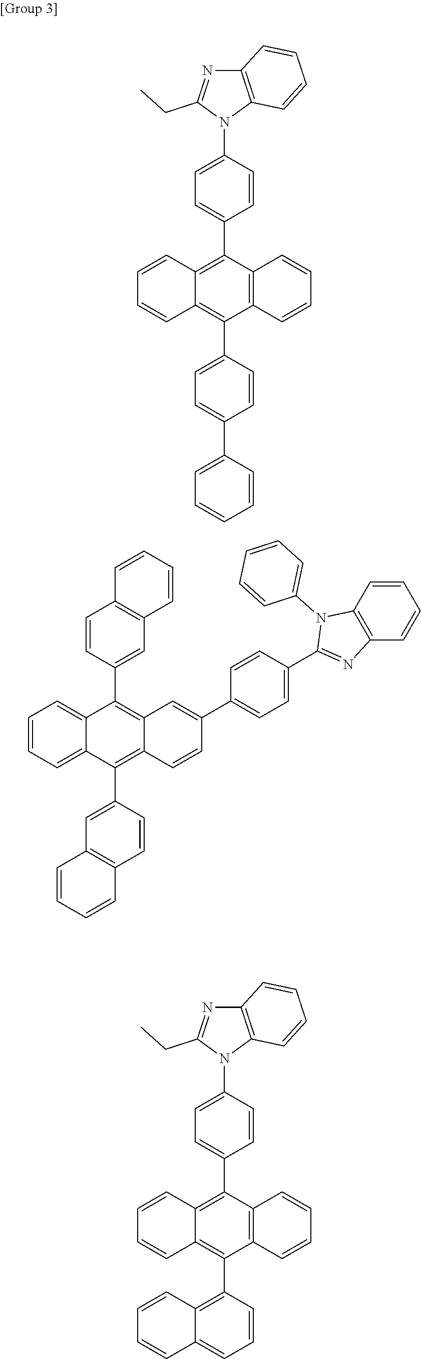

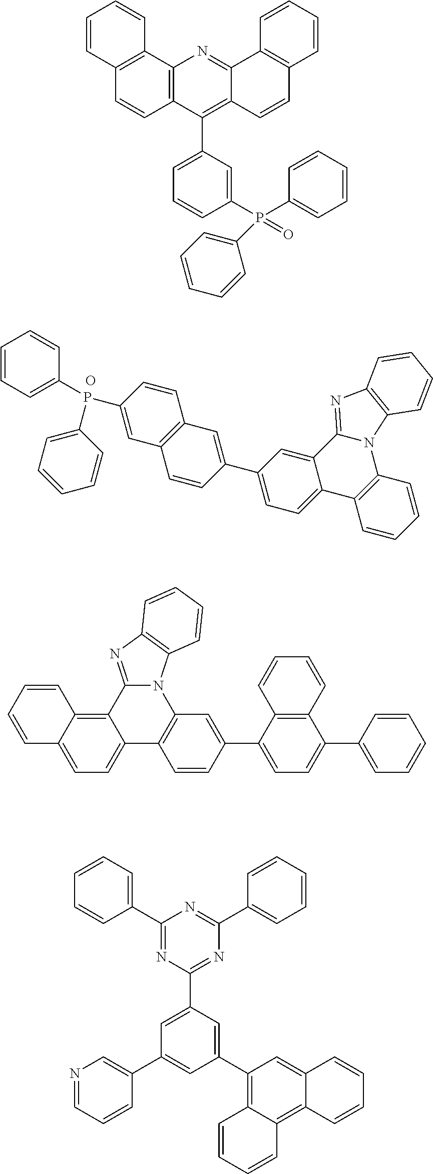

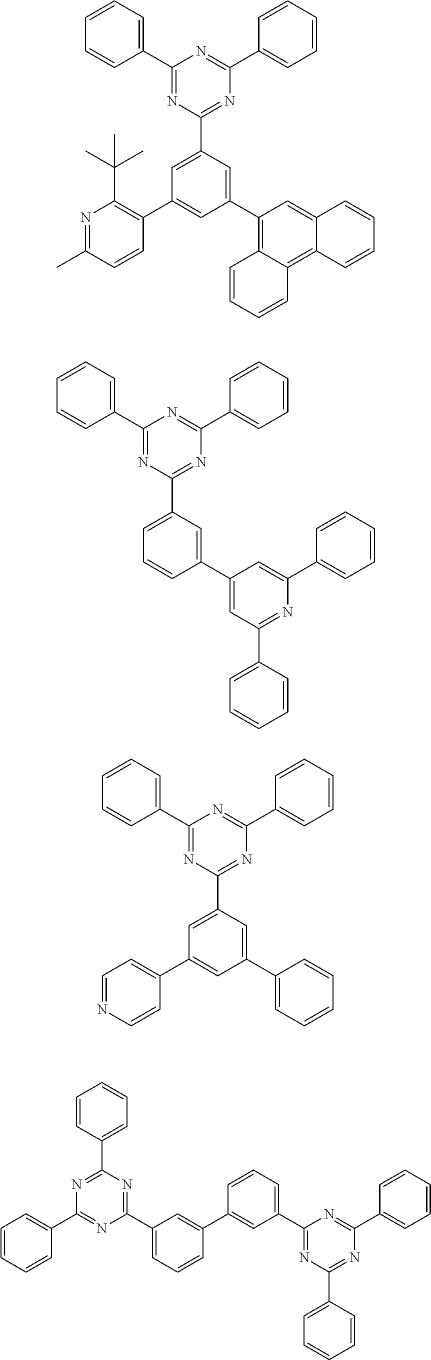

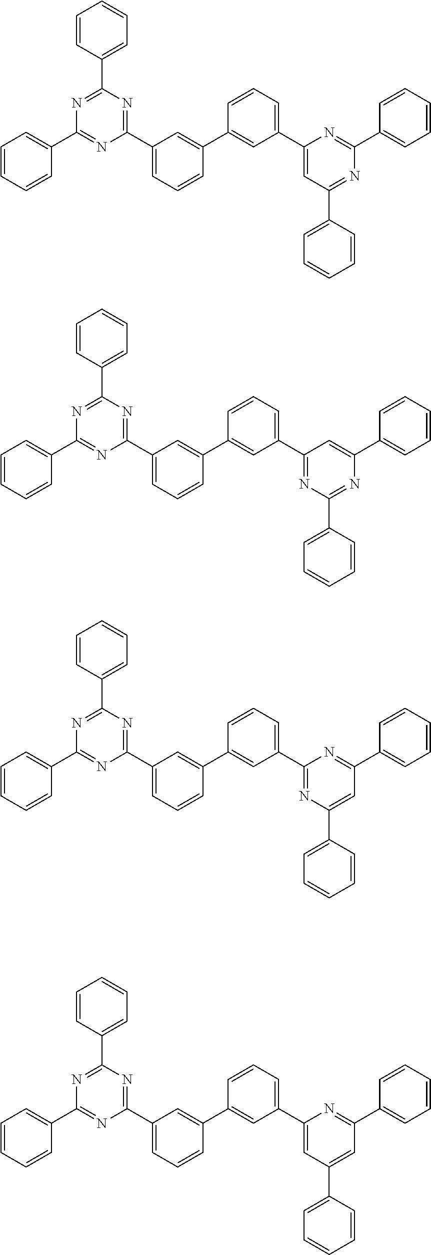 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1]

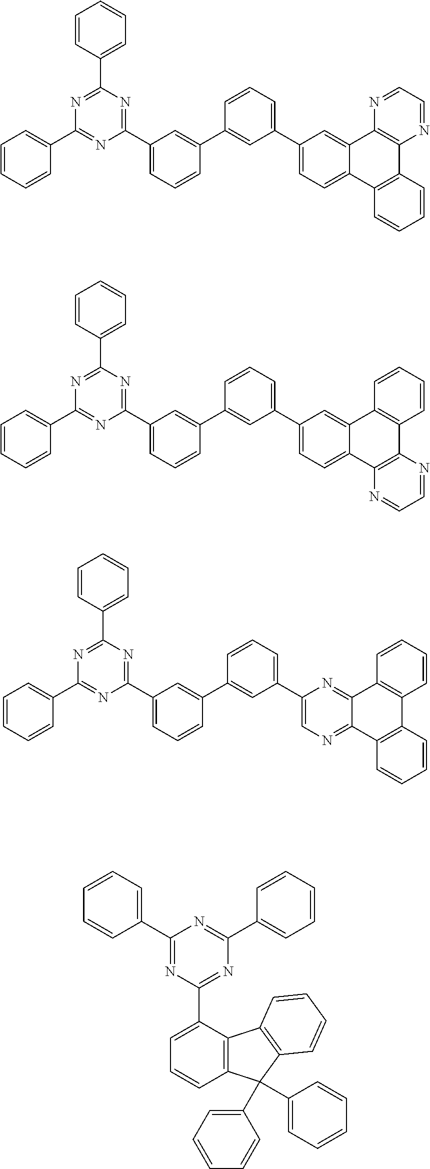 [SMILES: c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1]

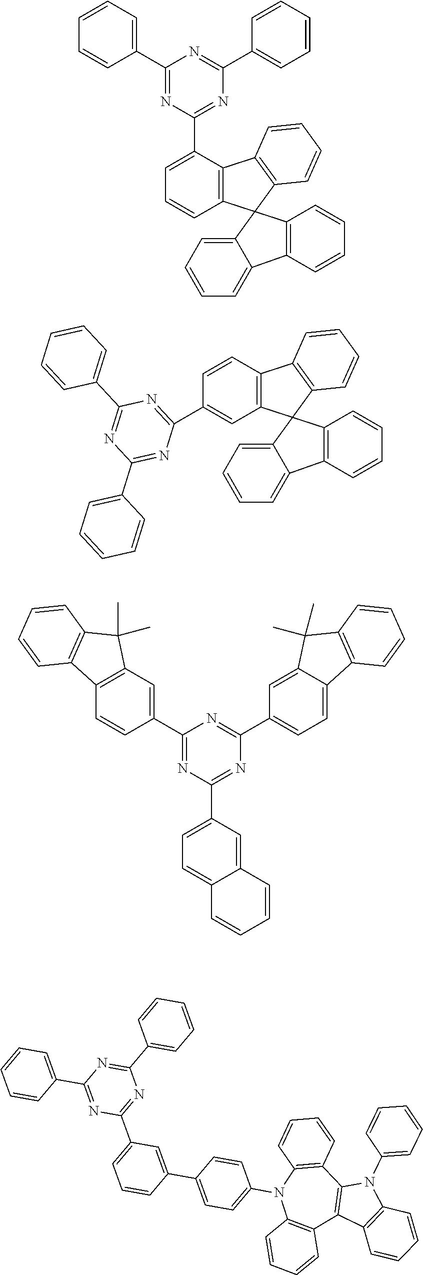

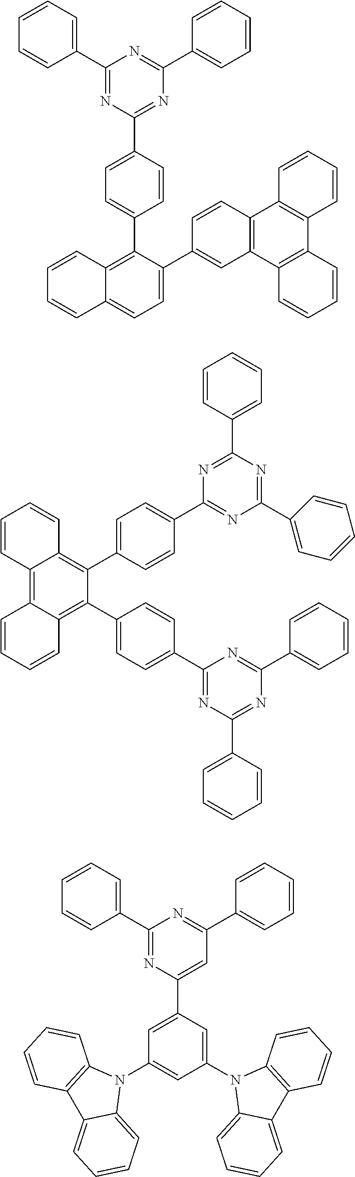 [SMILES: c1ccc(-c2nc(-c3ccccc3)nc(-c3ccc(-c4c(-c5ccc6c7ccccc7c7ccccc7c6c5)ccc5ccccc45)cc3)n2)cc1 ; c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2cc(-c3cc(-n4c5ccccc5c5ccccc54)cc(-n4c5ccccc5c5ccccc54)c3)nc(-c3ccccc3)n2)cc1]

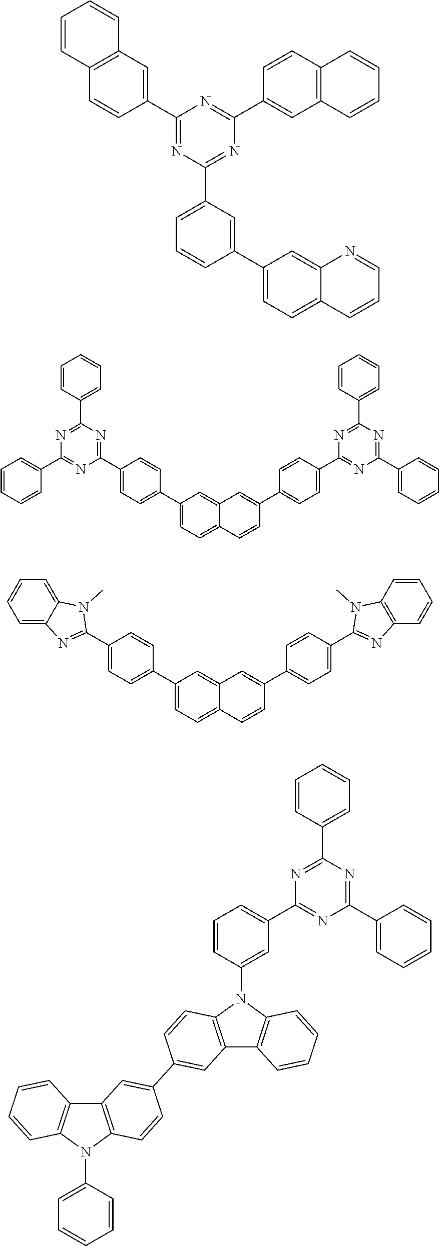 [SMILES: c1cc(-c2ccc3cccnc3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

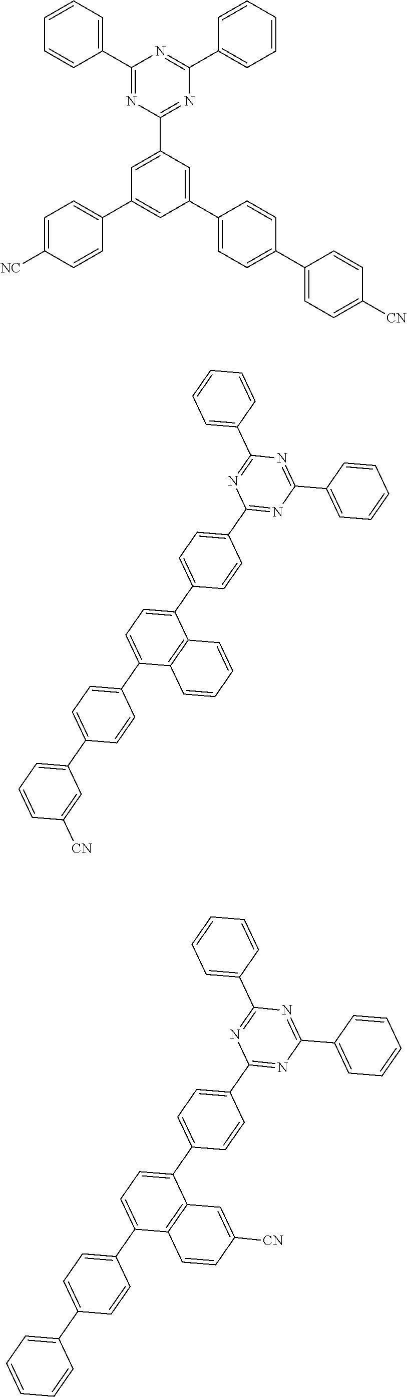 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

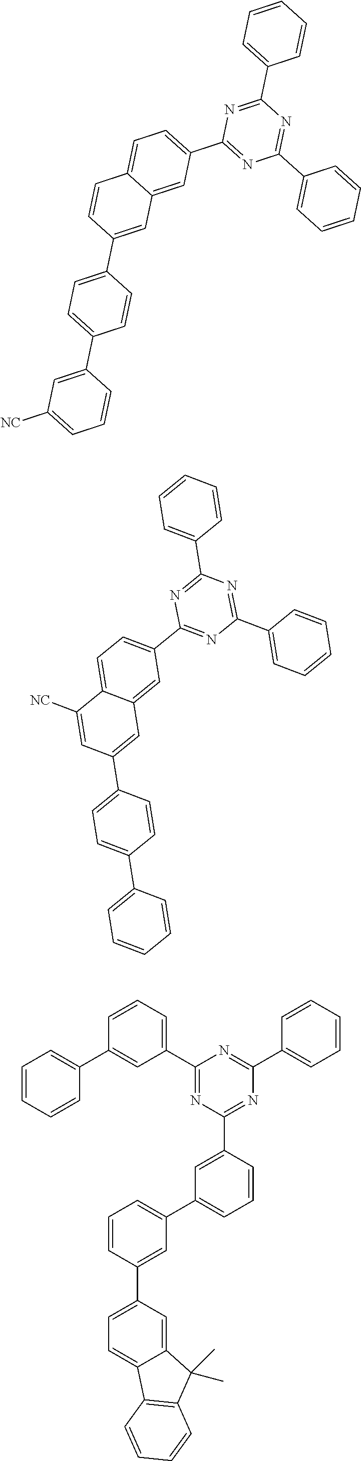

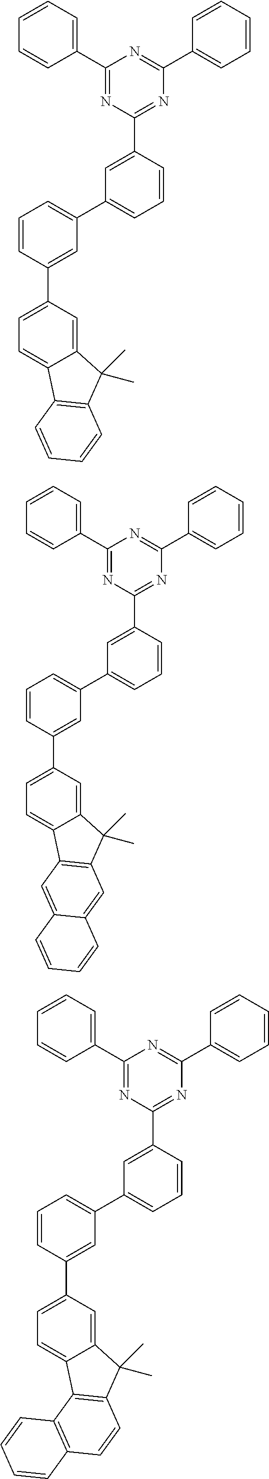

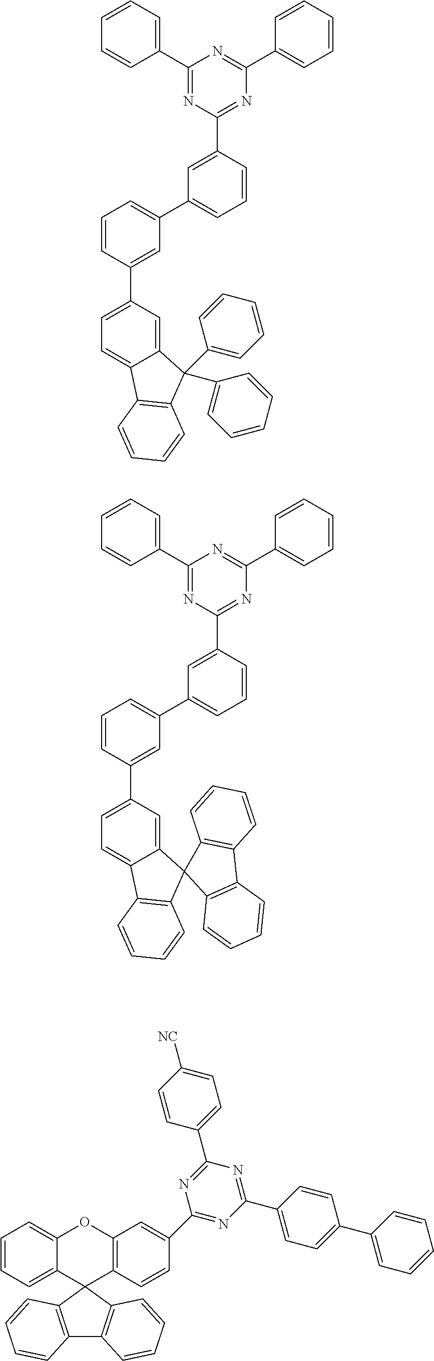

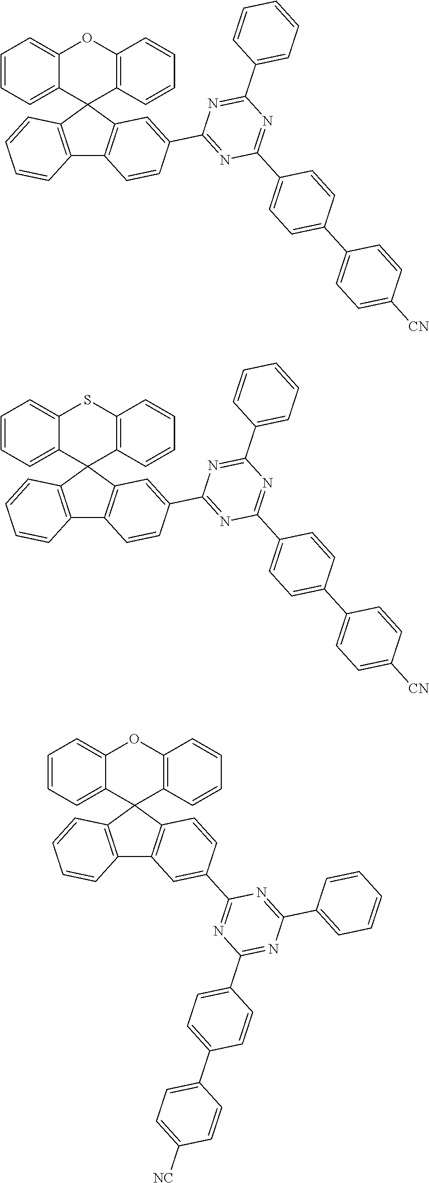 [SMILES: N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1]

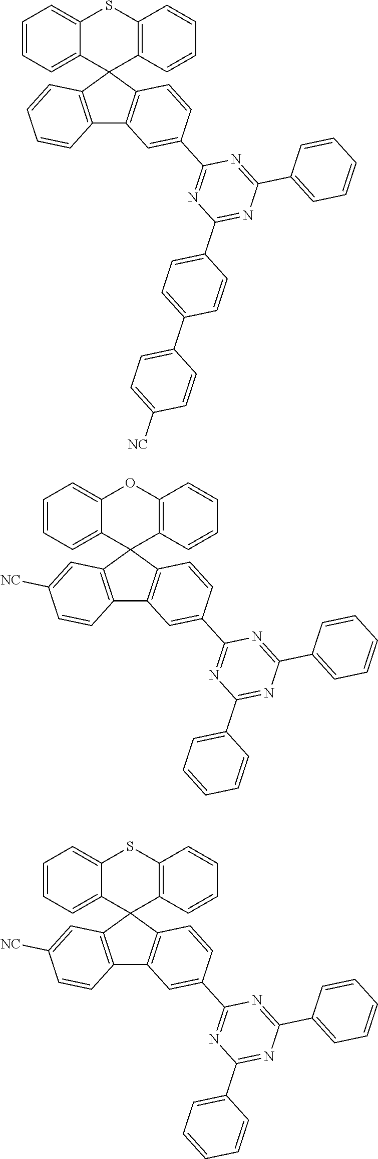

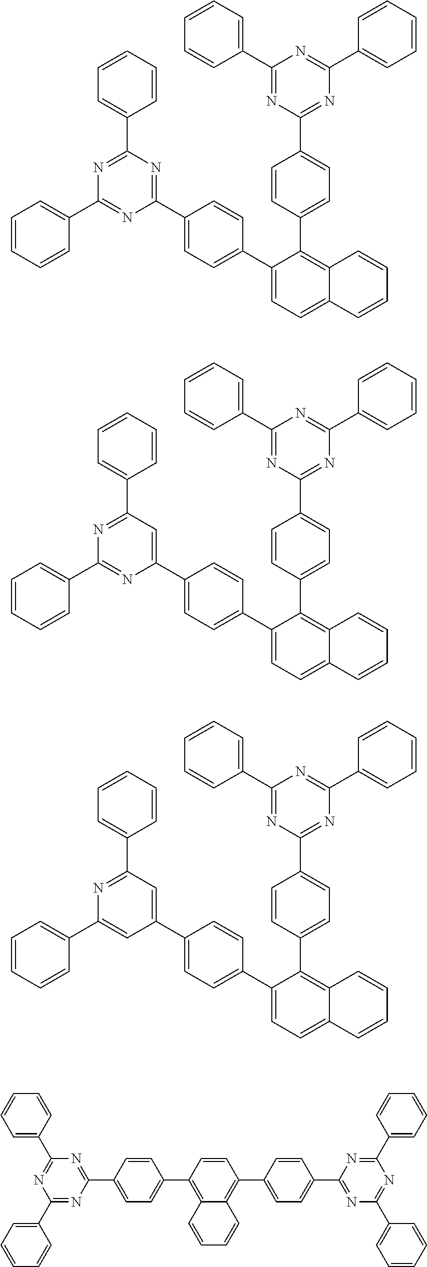 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1]

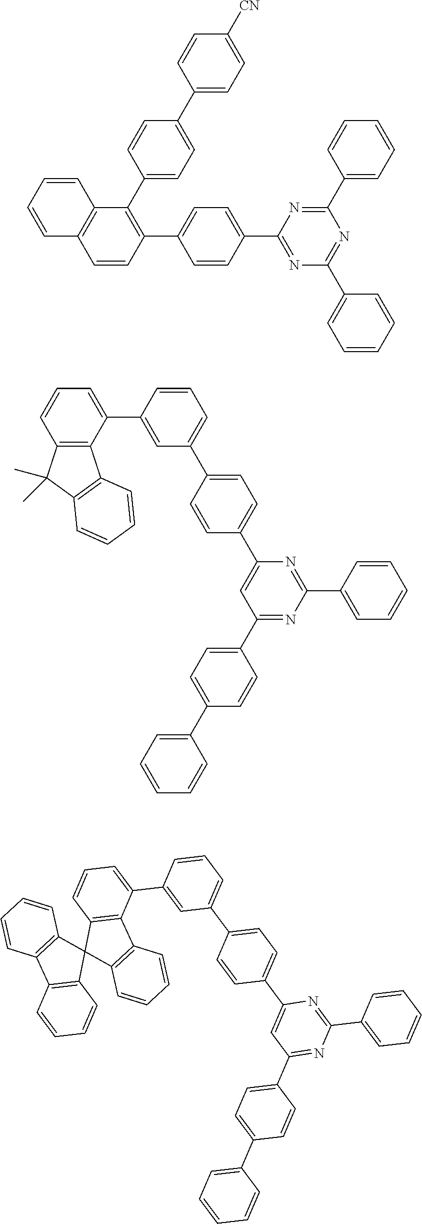 [SMILES: N#Cc1ccc(-c2ccc(-c3c(-c4ccc(-c5nc(-c6ccccc6)nc(-c6ccccc6)n5)cc4)ccc4ccccc34)cc2)cc1 ; CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1]

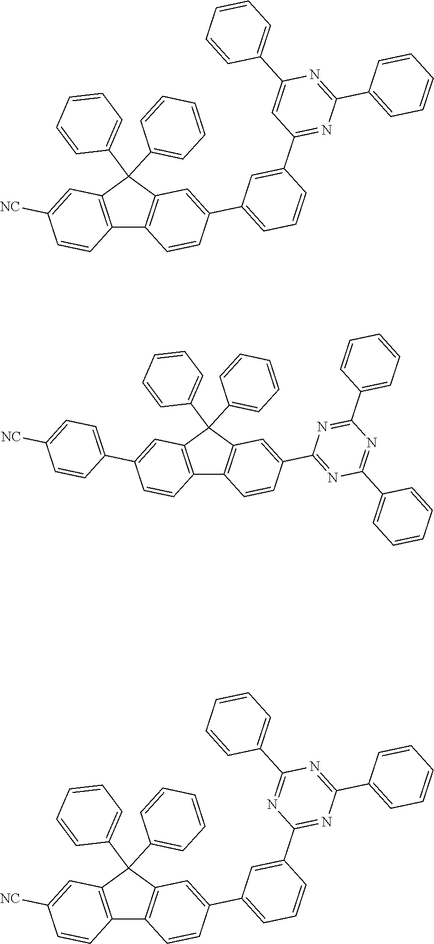 [SMILES: N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

The organic optoelectronic device, including the aforementioned organic light emitting diodes, can be applied to display devices.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry or P&H tech as far as there is no particular comment or were synthesized by known methods.

### Preparation of Compound for Organic Optoelectronic Device

As a more specific example of the compound of the present embodiments, the compound presented was synthesized through the following steps.

### Synthesis of First Compound

### Synthesis Example 1: Synthesis of Compound H-2

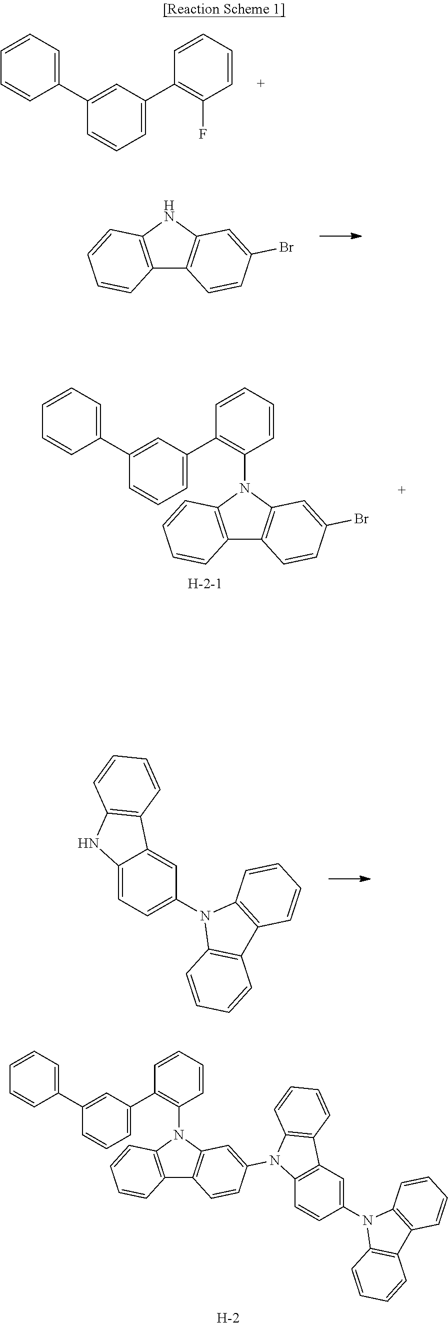

### First Step: Synthesis of Compound H-2-1

2-fluoro-1,1′:3′,1″-terphenyl (15 g, 60.4 mmol), 2-bromo-9H-carbazole (14.9 g, 60.4 mmol), and cesium carbonate (49.2 g, 151.0 mmol) were added to 302 ml of N-methyl-2-pyrrolidone and then, stirred at 180° C. for 18 hours. After cooling to ambient temperature, water was added thereto to produce a solid, which was filtered under reduced pressure and purified through column chromatography to obtain 17.5 g (yield: 61.0%) of Intermediate H-2-1.

### Second Step: Synthesis of Compound H-2

17.5 g (36.9 mmol) of Intermediate H-2-1, 12.3 g (36.9 mmol) of 9H-3,9′-bicarbazole, 1.7 g (1.8 mmol) of tris(dibenzylideneacetone)dipalladium (0) (Pd₂(dba)₃), 4.3 g (44.3 mmol) of sodium tert-butoxide (NaOtBu), and 1.1 g (5.5 mmol) of tri(tert-butyl)phosphine (P(tBu)₃) were added to 184 mL of toluene and then, stirred under reflux for 12 hours. When a reaction was completed, the resultant was purified through column chromatography (dichloromethane:n-hexane) to obtain 21.7 g (29.9 mmol, 81.0%) of Compound H-2.

### Synthesis Example 2: Synthesis of Compound H-62

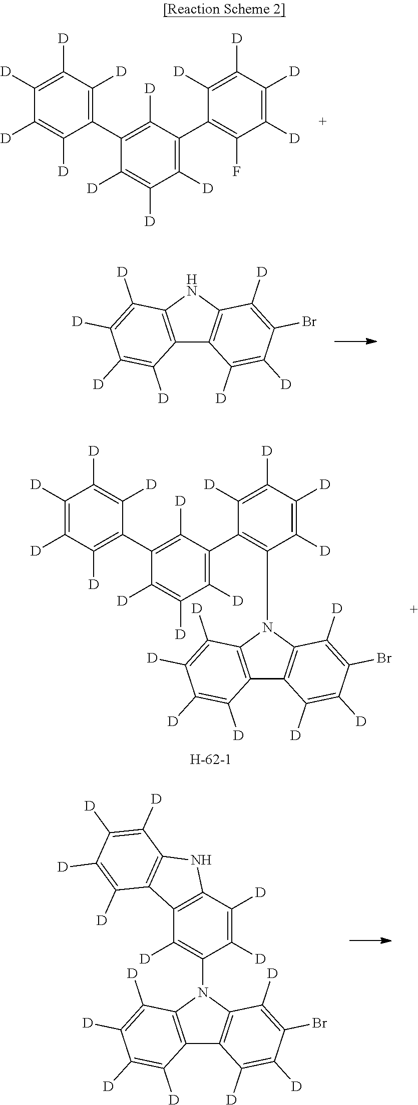 [SMILES: [2H]c1c([2H])c([2H])c(-c2c([2H])c([2H])c([2H])c(-c3c([2H])c([2H])c([2H])c([2H])c3F)c2[2H])c([2H])c1[2H] ; [2H]c1c([2H])c([2H])c2c([nH]c3c([2H])c(Br)c([2H])c([2H])c32)c1[2H] ; [2H]c1c([2H])c([2H])c(-c2c([2H])c([2H])c([2H])c(-c3c([2H])c([2H])c([2H])c([2H])c3-n3c4c([2H])c([2H])c([2H])c([2H])c4c4c([2H])c([2H])c(Br)c([2H])c43)c2[2H])c([2H])c1[2H] ; [2H]c1c([2H])c([2H])c2c([nH]c3c([2H])c([2H])c(-n4c5c([2H])c([2H])c([2H])c([2H])c5c5c([2H])c([2H])c(Br)c([2H])c54)c([2H])c32)c1[2H]]

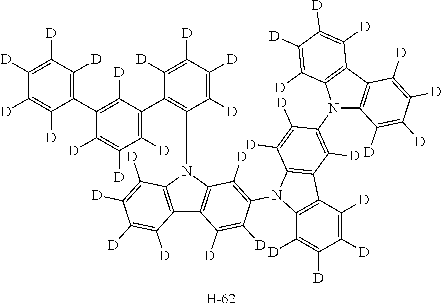

### First Step: Synthesis of Intermediate H-62-1

2-fluoro-1,1′:3′,1″-terphenyl-2′,2″,3,3″,4,4′,4″,5,5′,5″,6,6′,6″-d13 (12 g, 45.9 mmol), 2-bromo-9H-carbazole-1,3,4,5,6,7,8-d7 (11.6 g, 45.9 mmol), and cesium carbonate (37.4 g, 114.8 mmol) were added to 230 ml of N-methyl-2-pyrrolidone and then, stirred at 180° C. for 18 hours. After cooling to ambient temperature, water was added thereto to produce a solid, which was filtered under reduced pressure and purified through column chromatography to obtain 15.2 g (yield: 67.0%) of Intermediate H-62-1.

### Second Step: Synthesis of Compound H-62

15.2 g (30.7 mmol) of Intermediate H-62-1, 10.7 g (30.7 mmol) of 9H-3,9′-bicarbazole-1,1′,2,2′,3′,4,4′,5,5′,6,6′,7,7′,8,8′-d15, tris(dibenzylideneacetone)dipalladium (0) Pd₂(dba)₃) (1.4 g, 1.5 mmol), sodium tert-butoxide (NaOtBu) (3.5 g, 36.9 mmol), and tri(tert-butyl)phosphine (P(tBu)₃) (0.9 g, 4.6 mmol) were added to 154 mL of toluene and then, stirred under reflux for 12 hours. When a reaction was completed, the resultant was purified through column chromatography (dichloromethane:n-hexane) to obtain 18.5 g (24.3 mmol, 79.0%) of Compound H-62.

### Synthesis Example 3: Synthesis of Compound H-5

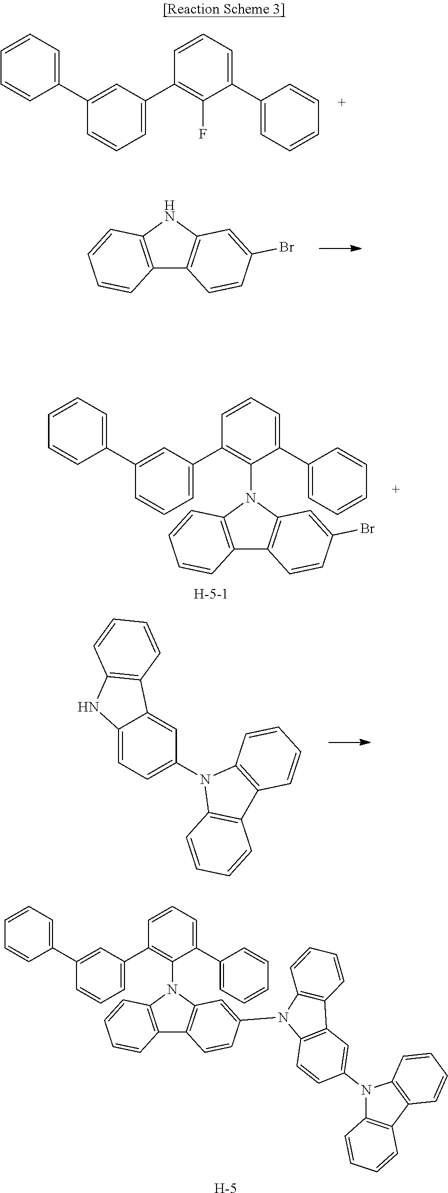

### First Step: Synthesis of Intermediate H-5-1

2′-fluoro-1,1′:3′,1″:3″,1′″-quaterphenyl (10 g, 30.8 mmol), 2-bromo-9H-carbazole (7.6 g, 30.8 mmol), and 25.1 g (77.1 mmol) of cesium carbonate were added to 154 ml of N-methyl-2-pyrrolidone and then, stirred at 180° C. for 18 hours. After cooling to ambient temperature, water was added thereto to produce a solid, which was filtered under reduced pressure and purified through column chromatography to obtain 9.8 g (yield: 58.0%) of Intermediate H-5-1.

### Second Step: Synthesis of Compound H-5

9.8 g (17.8 mmol) of Intermediate H-5-1, 5.9 g (17.8 mmol) of 9H-3,9′-bicarbazole, 0.8 g (0.9 mmol) of tris(dibenzylideneacetone)dipalladium (0) (Pd₂(dba)₃), sodium tert-butoxide (NaOtBu) (2.1 g, 21.4 mmol), and tri(tert-butyl)phosphine (P(tBu)₃) (0.5 g, 2.7 mmol) were added to 89 mL of toluene and then, stirred under reflux for 12 hours. When a reaction was completed, the resultant was purified through column chromatography (dichloromethane:n-hexane) to obtain 10.3 g (12.8 mmol, 72.0%) of Compound H-5.

### Synthesis Example 4: Synthesis of Compound H-71

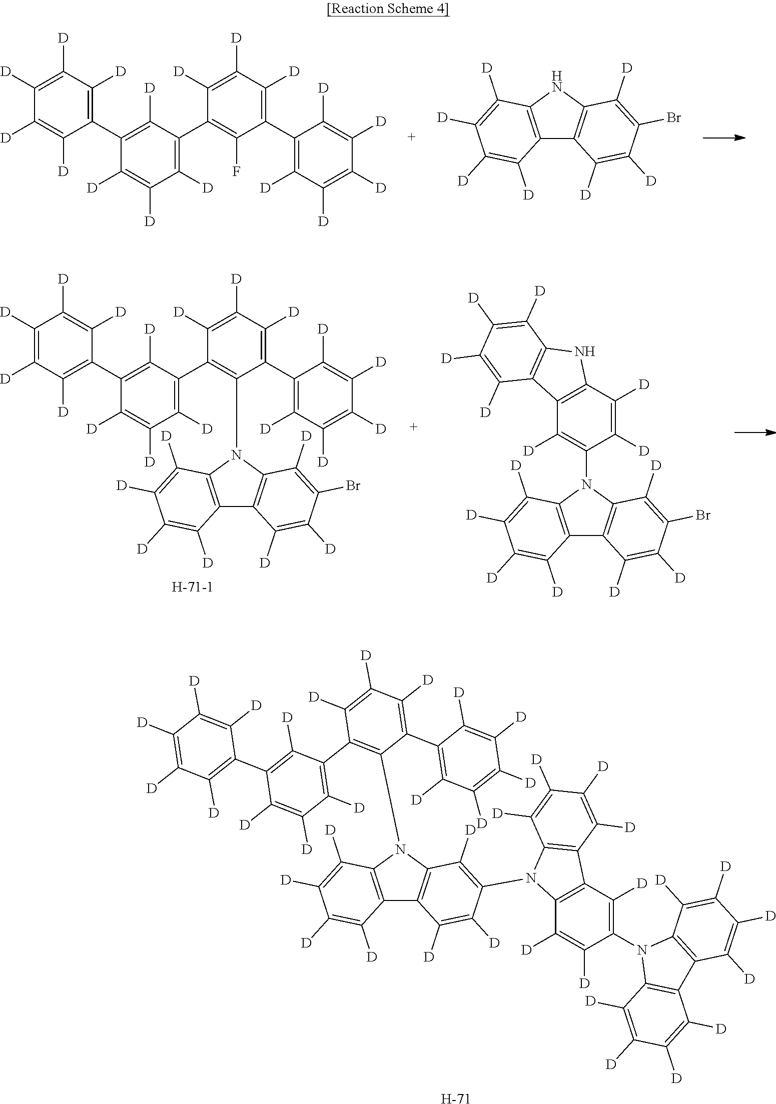

### First Step: Synthesis of Intermediate H-71-1

2′-fluoro-1,1′:3′,1″:3″,1′″-quaterphenyl-2,2″,2′″,3,3′″,4,4′,4″,4′″,5,5′,5,5′″,6,6′,6″,6′″-d7 (12 g, 35.1 mmol), 2-bromo-9H-carbazole-1,3,4,5,6,7,8-d7 (8.9 g, 35.1 mmol), and cesium carbonate (28.6 g, 87.8 mmol) were added to 176 ml of N-methyl-2-pyrrolidone and then, stirred at 180° C. for 18 hours. After cooling to ambient temperature, water was added thereto to produce a solid, which was filtered under reduced pressure and purified through column chromatography to obtain 12.3 g (yield: 61.0%) of Intermediate H-71-1.

### Second Step: Synthesis of Compound H-71

12.3 g (21.4 mmol) of Intermediate H-71-1, 7.4 g (21.4 mmol) of 9H-3,9′-bicarbazole-1,1′,2,2′,3′,4,4′,5,5′,6,6′,7,7′,8,8′-d15, tris(dibenzylideneacetone)dipalladium (0) (Pd₂(dba)₃) (1.0 g, 1.1 mmol), sodium tert-butoxide (NaOtBu) (2.5 g, 25.7 mmol), and tri(tert-butyl)phosphine (P(tBu)₃) (0.6 g, 3.2 mmol) were added to 107 mL of toluene and then, stirred under reflux for 12 hours. When a reaction was completed, the resultant was purified through column chromatography (dichloromethane:n-hexane) to obtain 13.9 g (16.5 mmol, 77.0%) of Compound H-71.

### Synthesis Example 5: Synthesis of Compound A-100 (Dn=28)

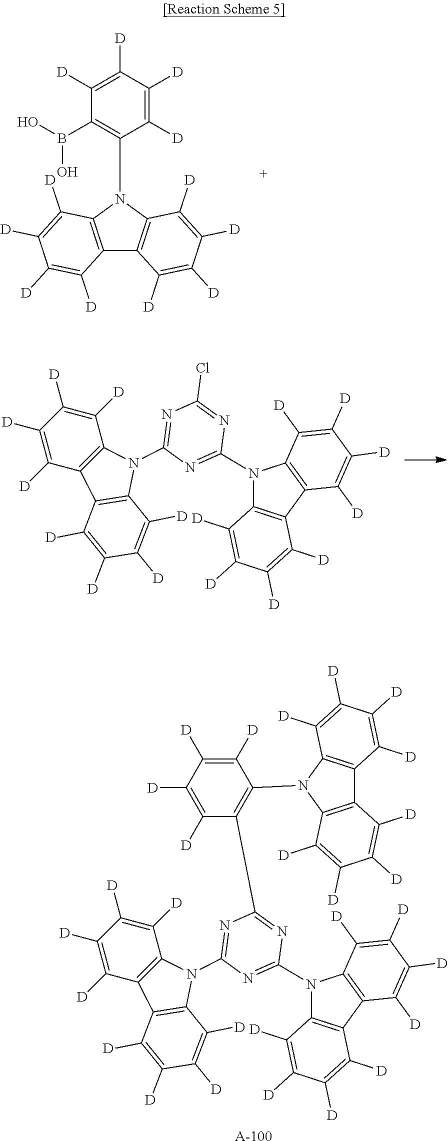

(2-(9H-carbazol-9-yl-d8)phenyl-3,4,5,6-d4)boronic acid (10 g, 33.4 mmol), 9,9′-(6-chloro-1,3,5-triazine-2,4-diyl)bis(9H-carbazole-1,2,3,4,5,6,7,8-d8) (15.4 g, 33.4 mmol), and Pd(PPh₃)₄ (1.9 g, 1.7 mmol), and potassium carbonate (K₂CO₃) (13.9 g, 100.3 mmol) were added to 111 mL of THF and 56 mL of DIW and then, stirred under reflux for 12 hours. When a reaction was completed, the resultant was purified through column chromatography (dichloromethane:n-hexane) to obtain 20.7 g (yield: 91.0%) of Compound A-100 (Dn=28).

### Synthesis Example 6: Synthesis of Compound A-92 (Dn=35)

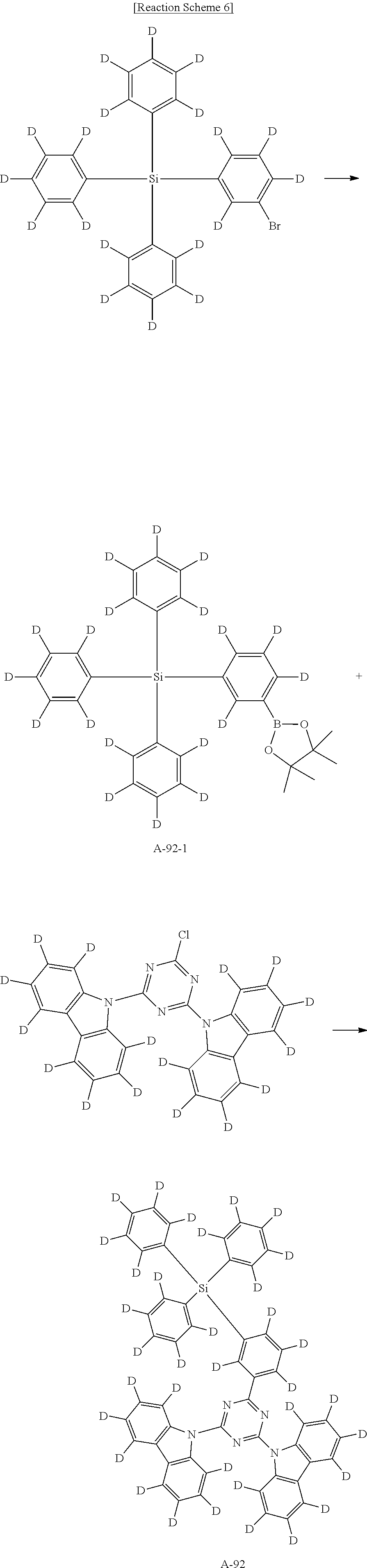

### First Step: Synthesis of Intermediate A-92-1

(3-bromophenyl-2,4,5,6-d4)tris(phenyl-d5)silane (15.0 g, 34.5 mmol), 4,4,4′,4′,5,5,5′,5′-octamethyl-2,2′-bi(1,3,2-dioxaborolane)(B₂Pin₂) (11.4 g, 44.9 mmol), [1,1′-bis(diphenylphosphino)ferrocene]dichloropalladium(II) (Pd(dppf)Cl₂) (1.4 g, 1.7 mmol), and potassium acetate (KOAc) (10.2 g, 103.6 mmol) were added to 173 mL of xylene and then, stirred under reflux for 12 hours. When a reaction was completed, the resultant was purified through column chromatography (dichloromethane:n-hexane) to obtain 10.5 g (yield: 63.0%) of Intermediate A-92-1.

### Second Step: Synthesis of Compound A-92 (Dn=35)

Intermediate A-92-1 (10.5 g, 21.8 mmol), 9,9′-(6-chloro-1,3,5-triazine-2,4-diyl)bis(9H-carbazole-1,2,3,4,5,6,7,8-d8) (10.1 g, 21.8 mmol), Pd(PPh₃)₄ (1.3 g, 1.1 mmol), and potassium carbonate (K₂CO₃) (9.0 g, 65.4 mmol) were added to 73 mL of THF and 36 mL of DIW and then, stirred under reflux for 12 hours. When a reaction was completed, the resultant was purified through column chromatography (dichloromethane:n-hexane) to obtain 15.0 g (yield: 88.0%) of Compound A-92 (Dn=35).

### Comparative Synthesis Example 1: Synthesis of Compound R-1

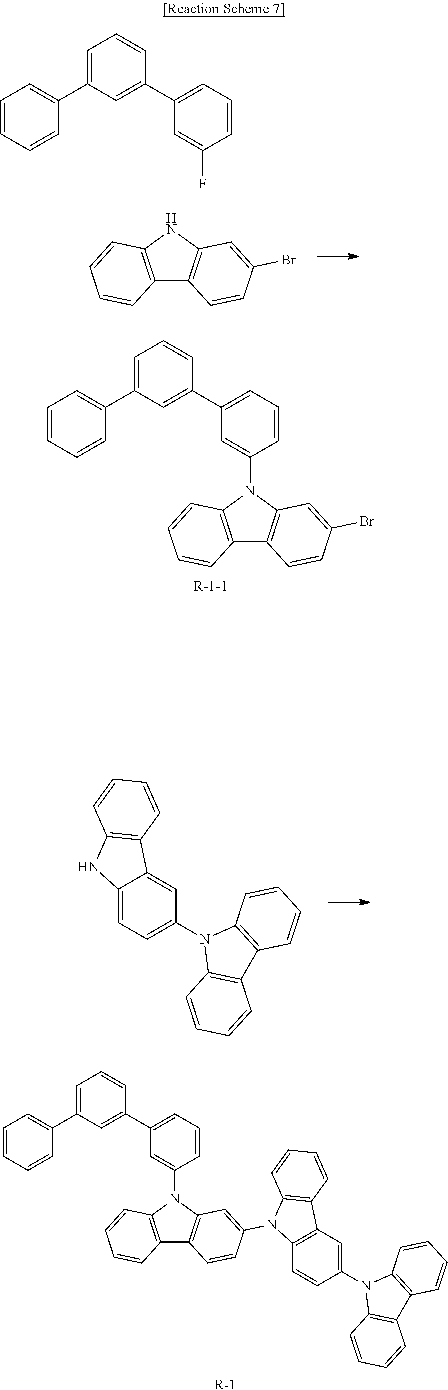 [SMILES: Fc1cccc(-c2cccc(-c3ccccc3)c2)c1 ; Brc1ccc2c(c1)[nH]c1ccccc12 ; Brc1ccc2c3ccccc3n(-c3cccc(-c4cccc(-c5ccccc5)c4)c3)c2c1 ; c1ccc2c(c1)[nH]c1ccc(-n3c4ccccc4c4ccccc43)cc12 ; c1ccc(-c2cccc(-c3cccc(-n4c5ccccc5c5ccc(-n6c7ccccc7c7cc(-n8c9ccccc9c9ccccc98)ccc76)cc54)c3)c2)cc1 ; "R-1"]

### First Step: Synthesis of Intermediate R-1-1

3-fluoro-1,1′:3′,1″-terphenyl (15 g, 60.4 mmol), 2-bromo-9H-carbazole (14.9 g, 60.4 mmol), and cesium carbonate (49.2 g, 151.0 mmol) were added to 302 ml of N-methyl-2-pyrrolidone and then, stirred at 180° C. for 18 hours. After cooling to ambient temperature, water was added thereto to produce a solid, which was filtered under a reduced pressure and purified through column chromatography to obtain 21.5 g (yield: 75.0%) of Intermediate R-1-1.

### Second Step: Synthesis of Compound R-1

21.5 g (45.3 mmol) of Intermediate R-1-1, 15.1 g (45.3 mmol) of 9H-3,9′-bicarbazole, tris(dibenzylideneacetone)dipalladium (0) (Pd₂(dba)₃) (2.1 g, 2.3 mmol), sodium tert-butoxide (NaOtBu) (5.2 g, 54.4 mmol), and tri(tert-butyl)phosphine (P(tBu)₃) (1.4 g, 6.8 mmol) were added to 227 mL of toluene and then, stirred under reflux for 12 hours. When a reaction was completed, the resultant was purified through column chromatography (dichloromethane:n-hexane) to obtain 25.7 g (35.3 mmol, 78.0%) of Compound R-1.

### Comparative Synthesis Example 2: Synthesis of Compound R-2

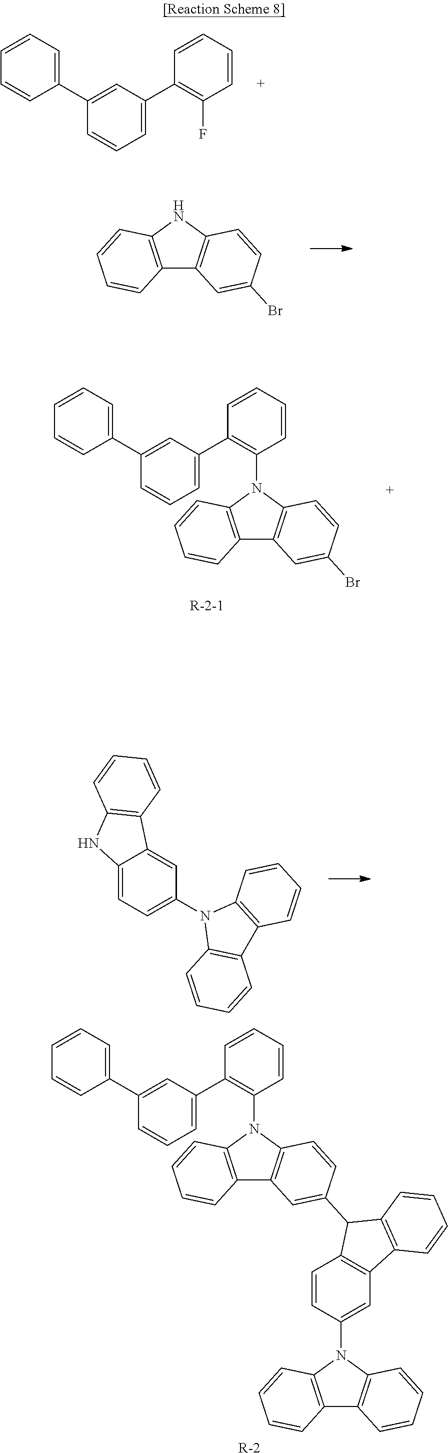

### First Step: Synthesis of Intermediate R-2-1

2-fluoro-1,1′:3′,1″-terphenyl (15 g, 60.4 mmol), 3-bromo-9H-carbazole (14.9 g, 60.4 mmol), and cesium carbonate 49.2 g (151.0 mmol) were added to 302 ml of N-methyl-2-pyrrolidone and then, stirred at 180° C. for 18 hours. After cooling to ambient temperature, water was added thereto to produce a solid, which was filtered under reduced pressure and purified through column chromatography to obtain 18.3 g (yield: 64.0%) of Intermediate R-2-1.

### Second Step: Synthesis of Compound R-2

18.3 g (38.6 mmol) of Intermediate R-2-1, 12.8 g (38.6 mmol) of 9H-3,9′-bicarbazole, and tris(dibenzylideneacetone)dipalladium (0) (Pd₂(dba)₃) (1.8 g, 1.9 mmol), sodium tert-butoxide (NaOtBu) (4.4 g, 46.3 mmol), and tri(tert-butyl)phosphine (P(tBu)₃) (1.2 g, 5.5 mmol) were added to 193 mL of toluene and then, stirred under reflux for 12 hours. When a reaction was completed, the resultant was purified through column chromatography (dichloromethane:n-hexane) to obtain 23.5 g (32.4 mmol, 84.0%) of Compound R-2.

### Comparative Synthesis Example 3: Synthesis of Compound R-3

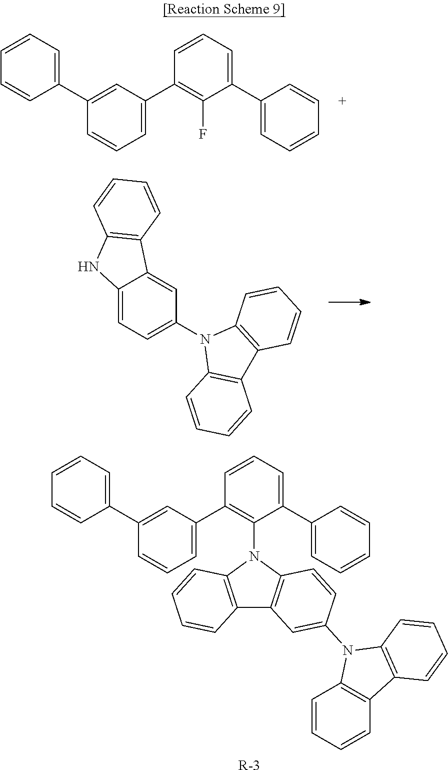

### First Step: Synthesis of Compound R-3

2′-fluoro-1,1′:3′,1″:3″,1′″-quaterphenyl (11 g, 33.9 mmol), 9H-3,9′-bicarbazole (11.3 g, 33.9 mmol), and cesium carbonate (27.6 g, 84.8 mmol) were added to 170 ml of N-methyl-2-pyrrolidone and then, stirred at 180° C. for 18 hours. After cooling to ambient temperature, water was added thereto to produce a solid, which was filtered under reduced pressure and purified through column chromatography to obtain 13.4 g (21.0 mmol, 62.0%) of Compound R-3.

### Comparative Synthesis Example 4: Synthesis of Compound R-4

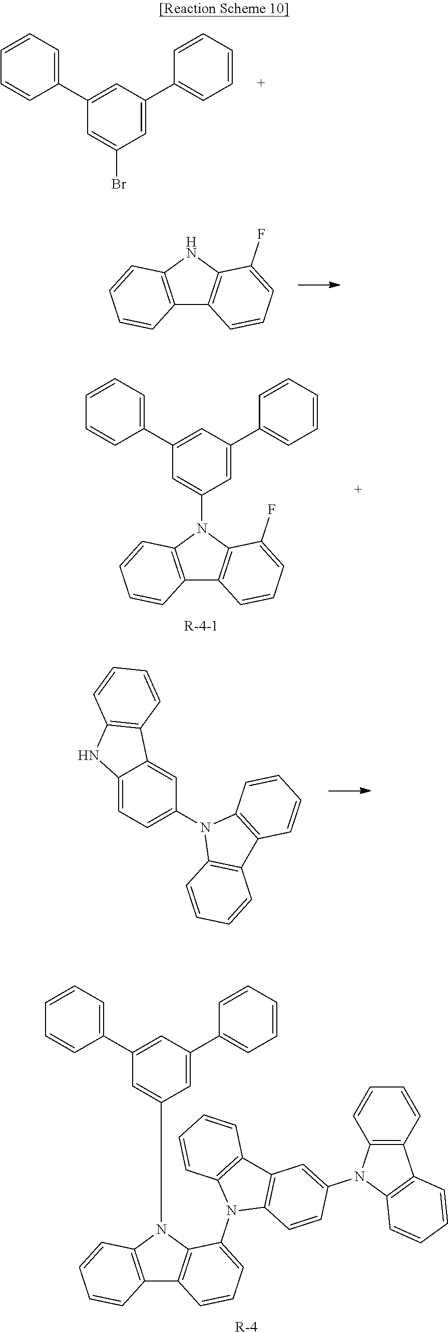

### First Step: Synthesis of Intermediate R-4-1

5′-bromo-1,1′:3′,1″-terphenyl (13.0 g, 42.0 mmol), 1-fluoro-9H-carbazole (7.8 g, 42.0 mmol), tris(dibenzylideneacetone)dipalladium (0) (Pd₂(dba)₃) (1.9 g, 2.1 mmol), sodium tert-butoxide (NaOtBu) (4.8 g, 50.5 mmol), and tri(tert-butyl)phosphine (P(tBu)₃) (1.3 g, 6.3 mmol) were added to 210 mL of toluene and then, stirred under reflux for 12 hours. When a reaction was completed, the resultant was purified through column chromatography (dichloromethane:n-hexane) to obtain 12.2 g (yield: 70.0%) of Intermediate R-4-1.

### Second Step: Synthesis of Compound R-4

12.2 g (29.5 mmol) of Intermediate R-4-1, 9.8 g (29.5 mmol) of 9H-3,9′-bicarbazole, and 24.0 g (73.8 mmol) of cesium carbonate were added to 148 ml of N-methyl-2-pyrrolidone and then, stirred at 180° C. for 18 hours. After cooling to ambient temperature, water was added thereto to produce a solid, which was filtered under reduced pressure and purified through column chromatography to obtain 8.8 g (12.1 mmol, 41.0%) of Compound R-4.

### Comparative Synthesis Example 5: Synthesis of Compound R-5

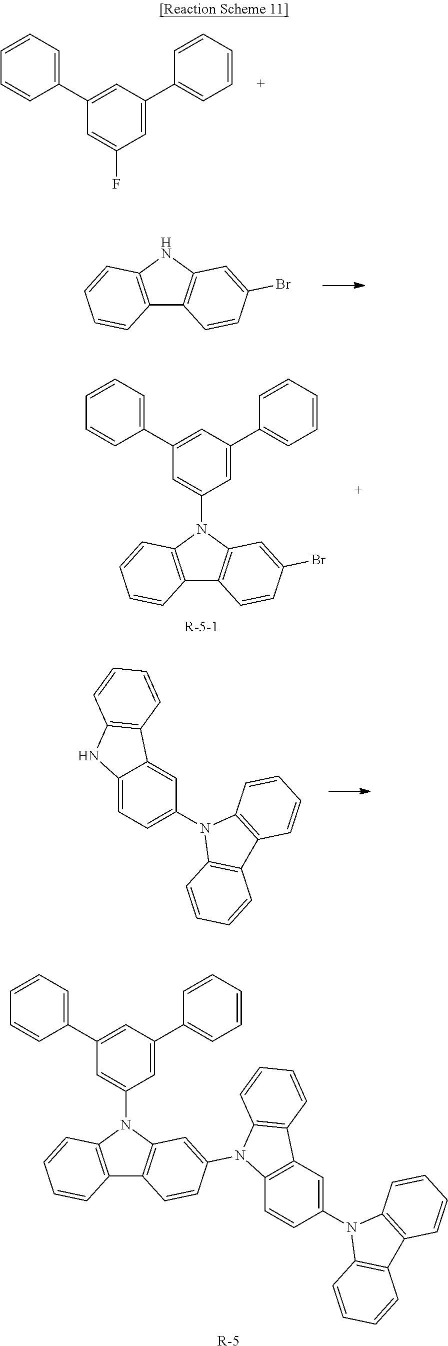 [SMILES: Fc1cc(-c2ccccc2)cc(-c2ccccc2)c1 ; Brc1ccc2c(c1)[nH]c1ccccc12 ; Brc1ccc2c3ccccc3n(-c3cc(-c4ccccc4)cc(-c4ccccc4)c3)c2c1 ; c1ccc2c(c1)[nH]c1ccc(-n3c4ccccc4c4ccccc43)cc12 ; c1ccc(-c2cc(-c3ccccc3)cc(-n3c4ccccc4c4ccc(-n5c6ccccc6c6cc(-n7c8ccccc8c8ccccc87)ccc65)cc43)c2)cc1 ; "R-5"]

### First Step: Synthesis of Intermediate R-5-1

5′-fluoro-1,1′:3′,1″-terphenyl (15 g, 60.4 mmol), 2-bromo-9H-carbazole (14.9 g, 60.4 mmol), and cesium carbonate 49.2 g (151.0 mmol) were added to 302 ml of N-methyl-2-pyrrolidone and then, stirred at 180° C. for 18 hours. After cooling to ambient temperature, water was added thereto to produce a solid, which was filtered under reduced pressure and purified through column chromatography to obtain 22.1 g (yield: 77.0%) of Intermediate R-5-1.

### Second Step: Synthesis of Compound R-5

22.1 g (46.6 mmol) of Intermediate R-5-1, 15.5 g (46.6 mmol) of 9H-3,9′-bicarbazole, tris(dibenzylideneacetone)dipalladium (0) (Pd₂(dba)₃) (2.1 g, 2.3 mmol), sodium tert-butoxide (NaOtBu) (5.4 g, 55.9 mmol), and tri(tert-butyl)phosphine (P(tBu)₃) (1.4 g, 7.0 mmol) were added to 233 mL of toluene and then, stirred under reflux for 12 hours. When a reaction was completed, the resultant was purified through column chromatography (dichloromethane:n-hexane) to obtain 25.7 g (35.4 mmol, 76.0%) of Compound R-5.

### Comparative Synthesis Example 6: Synthesis of Compound R-6

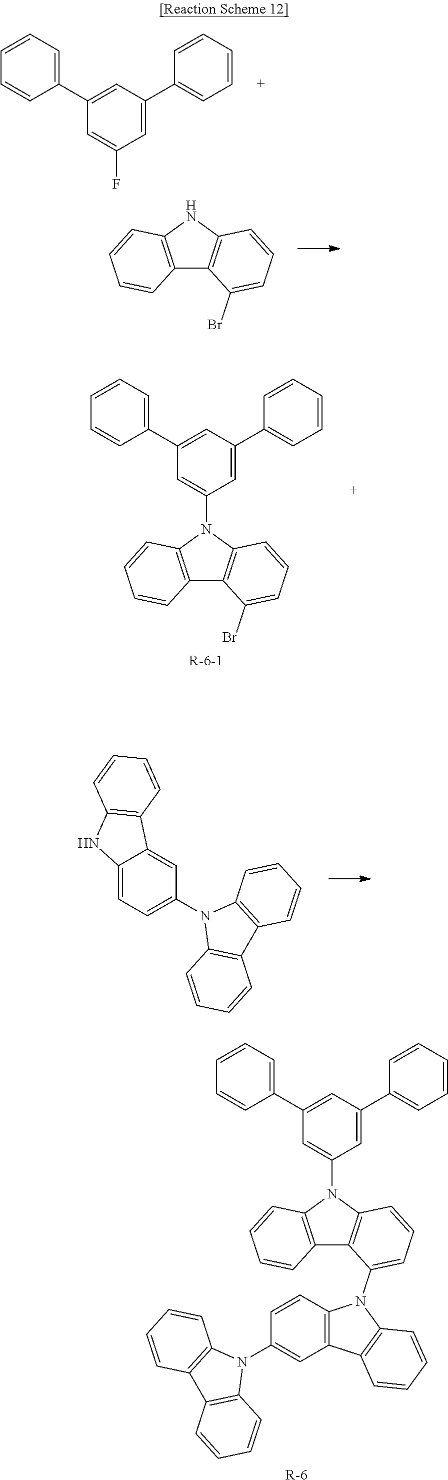

### First Step: Synthesis of Intermediate R-6-1

5′-fluoro-1,1′:3′,1″-terphenyl (15 g, 60.4 mmol), 4-bromo-9H-carbazole (14.9 g, 60.4 mmol), and cesium carbonate (49.2 g, 151.0 mmol) were added to 302 ml of N-methyl-2-pyrrolidone and then, stirred at 180° C. for 18 hours. After cooling to ambient temperature, water was added thereto to produce a solid, which was filtered under reduced pressure and purified through column chromatography to obtain 20.3 g (yield: 71.0%) of Intermediate R-6-1.

### Second Step: Synthesis of Compound R-6

20.3 g (42.8 mmol) of Intermediate R-6-1, 14.2 g (42.8 mmol) of 9H-3,9′-bicarbazole, tris(dibenzylideneacetone)dipalladium (0) (Pd₂(dba)₃) (2.0 g, 2.1 mmol), sodium tert-butoxide (NaOtBu) (4.9 g, 51.3 mmol), and tri(tert-butyl)phosphine (P(tBu)₃) (1.3 g, 6.4 mmol) were added to 214 mL of xylene and then, stirred under reflux for 12 hours. When a reaction was completed, the resultant was purified through column chromatography (dichloromethane:n-hexane) to obtain 18.0 g (24.8 mmol, 58.0%) of Compound R-6.

### Manufacture of Organic Light Emitting Diode I

### Example 1

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. The prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 600 Å to form a hole transport layer. mCP(1,3-bis(carbazol-9-yl)benzene) was deposited to a thickness of 100 Å on the hole transport layer to form a hole transport auxiliary layer. Compounds H-2 (host), obtained in Synthesis Example 1, and P31 (dopant) were vacuum-deposited on the hole transport auxiliary layer to form a 400 Å-thick light emitting layer. Subsequently, BCP(2,9-dimethyl-4,7-diphenyl-1,10-phenanthroline) was deposited on the light emitting layer to a thickness of 50 Å to form an electron transport auxiliary layer, and Compound B and LiQ were simultaneously vacuum deposited at a weight ratio of 1:1 to form a 300 Å-thick electron transport layer. An organic light emitting diode was manufactured by sequentially vacuum depositing 10 Å of LiQ and 1,200 Å of Al on the electron transport layer to form a cathode.

### Compound A: N-(9,9-diphenyl-9H-fluoren-2-yl)-N,9-diphenyl-9H-carbazol-2-amine

### Compound B: 8-{4-[bis(naphthalene-2-yl)-1,3,5-triazin-2-yl]phenyl}quinoline

### Example 2 and Comparative Examples 1 to 6

Each organic light emitting diode of Example 2 and Comparative Examples 1-6 was manufactured in the same manner as in Example 1, except that Compound H-2 was changed to compounds described in Table 1 as the host of the light emitting layer.

### Manufacture of Organic Light Emitting Diode II

### Example 3

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. The prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 600 Å to form a hole transport layer. mCP was deposited to a thickness of 100 Å on the hole transport layer to form a hole transport auxiliary layer. On the hole transport auxiliary layer, Compound H-2 obtained in Synthesis Example 1 and Compound A-100 (Dn=28) obtained in Synthesis Example 5 were used as hosts simultaneously, and 13 wt % of P31 was doped as a phosphorescent sensitizer and 1.5 wt % of D3 was doped as a fluorescent dopant to form a a 400 Å-thick light emitting layer by vacuum deposition. Here, Compound H-2 and Compound A-100 (Dn=28) were used in a weight ratio of 60:40. Subsequently, BCP was deposited on the light emitting layer to a thickness of 50 Å to form an electron transport auxiliary layer, and Compound B and LiQ were simultaneously vacuum deposited in a weight ratio of 1:1 to form a 300 Å-thick electron transport layer. An organic light emitting diode was manufactured by sequentially vacuum depositing 10 Å of LiQ and 1,200 Å of Al on the electron transport layer to form a cathode.

The organic light emitting diode was manufactured with the structure of ITO/Compound A (3% NDP-9 doping, 100 Å)/Compound A (600 Å)/mCP (100 Å)/EML [host (Compound H-2: Compound A-100 (Dn=28)): P31: D3=85.5 wt %: 13 wt %: 1.5 wt %](400 Å)/BCP (50 Å)/Compound B:LiQ (300 Å)/LiQ (10 Å)/Al (1,200 Å).

### Example 4 to 10 and Comparative Examples 7 to 12

Each organic light emitting diode of Examples 4 to 10 and Comparative Examples 7 to 12 was manufactured in the same manner as in Example 1, except that composition according to Example 3 was changed to the compositions in Table 2 as the host of the light emitting layer.

### Evaluation

The driving voltage and life-span characteristics of the organic light emitting diodes according to each of Examples and Comparative Examples were evaluated.

The specific measurement methods are as follows, and the results are in Tables 1 and 2.

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

The luminance and the current density measured from (1) and (2) above and a voltage were used to calculate the luminous efficiency (cd/A) at the same current density (10 mA/cm²).

(4) Measurement of Life-span

Time that the luminous efficiency (cd/A) of the organic light emitting diodes decreased to 95%, while maintaining the luminance (cd/m²) at 2000 cd/m², was measured.

The relative values calculated based on the life-span measurement values according to Comparative Example 1 are listed in Table 1.

The relative values calculated based on the life-span measurement values according to Comparative Example 7 are listed in Table 2.

TABLE 1

| No. | Host | Life-span (%) |
|---|---|---|
| Example 1 | Compound H-2 | 156% |
| Example 2 | Compound H-5 | 145% |
| Comparative | Compound R-1 | 100% |
| Example 1 | | |
| Comparative | Compound R-2 | 105% |
| Example 2 | | |
| Comparative | Compound R-3 | 106% |
| Example 3 | | |
| Comparative | Compound R-4 | 82% |
| Example 4 | | |
| Comparative | Compound R-5 | 98% |
| Example 5 | | |
| Comparative | Compound R-6 | 86% |
| Example 6 | | |

TABLE 2

| | Host | | Life- |
|---|---|---|---|
| | First | Second | span |
| No. | compound | compound | (%) |
| Example 3 | Compound H-2 | A-100 (Dn = 28) | 196% |
| Example 4 | Compound H-62 | A-100 (Dn = 28) | 232% |
| Example 5 | Compound H-5 | A-100 (Dn = 28) | 178% |
| Example 6 | Compound H-71 | A-100 (Dn = 28) | 202% |
| Example 7 | Compound H-2 | A-92 (Dn = 35) | 182% |
| Example 8 | Compound H-62 | A-92 (Dn = 35) | 213% |
| Example 9 | Compound H-5 | A-92 (Dn = 35) | 162% |
| Example 10 | Compound H-71 | A-92 (Dn = 35) | 192% |
| Comparative Example 7 | Compound R-1 | A-92 (Dn = 35) | 100% |
| Comparative Example 8 | Compound R-2 | A-92 (Dn = 35) | 112% |
| Comparative Example 9 | Compound R-3 | A-92 (Dn = 35) | 112% |
| Comparative Example 10 | Compound R-4 | A-92 (Dn = 35) | 70% |
| Comparative Example 11 | Compound R-5 | A-92 (Dn = 35) | 95% |
| Comparative Example 12 | Compound R-6 | A-92 (Dn = 35) | 82% |

Referring to Tables 1 and 2, the organic light emitting diodes according to the Examples exhibited improved luminous efficiency and life-span characteristics compared to the organic light emitting diodes according to the Comparative Examples.

Some example embodiments may provide a compound for an organic optoelectronic device capable of implementing high efficiency and long life-span characteristics.

Some example embodiments may provide a composition for an organic optoelectronic device including the compound for an organic optoelectronic device.

Some example embodiments may provide an organic optoelectronic device including the compound or the composition.

Some example embodiments may provide a display device including the organic optoelectronic device.

An organic optoelectronic device with high efficiency and long life-span characteristics may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by a combination of Chemical Formula 1 and Chemical Formula 2: 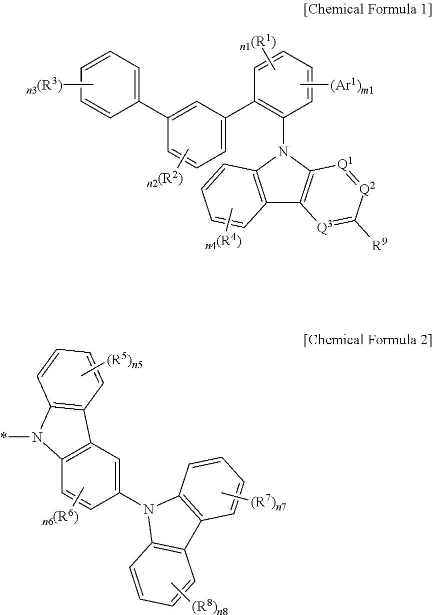
wherein, in Chemical Formula 1 and Chemical Formula 2,
Q¹, Q² and Q³ are each independently C—R^{a} or a linking carbon linked at * of Chemical Formula 2, provided that one of Q¹, Q² and Q³ is a linking carbon linked at * of Chemical Formula 2,
R^{a} and R¹ to R⁹ are each independently hydrogen, deuterium, a substituted or unsubstituted C1 to C10 alkyl group, or a substituted or unsubstituted C6 to C12 aryl group,
Ar¹ is a substituted or unsubstituted phenyl group or a substituted or unsubstituted biphenyl group,
n1 and n6 are each independently an integer of 1 to 3,
n2, n4, n5, n7, and n8 are each independently an integer of 1 to 4,
n3 is an integer of 1 to 5,
m1 is 0 or 1, and
when n1 is 2 or 3, each R¹ is the same or different each other,
when n2 is 2, 3, or 4, each R² is the same or different each other,
when n3 is 2, 3, 4, or 5, each R³ is the same or different each other,
when n4 is 2, 3, or 4, each R⁴ is the same or different each other,
when n5 is 2, 3, or 4, each R⁵ is the same or different each other,
when n6 is 2 or 3, each R⁶ is the same or different each other,
when n7 is 2, 3, or 4, each R⁷ is the same or different each other, and
when n8 is 2, 3, or 4, each R⁸ is the same or different each other.

2. The compound for an organic optoelectronic device as claimed in claim 1, wherein:
the combination of Chemical Formula 1 and Chemical Formula 2 is represented by one of Chemical Formula 1-I to Chemical Formula 1-III: 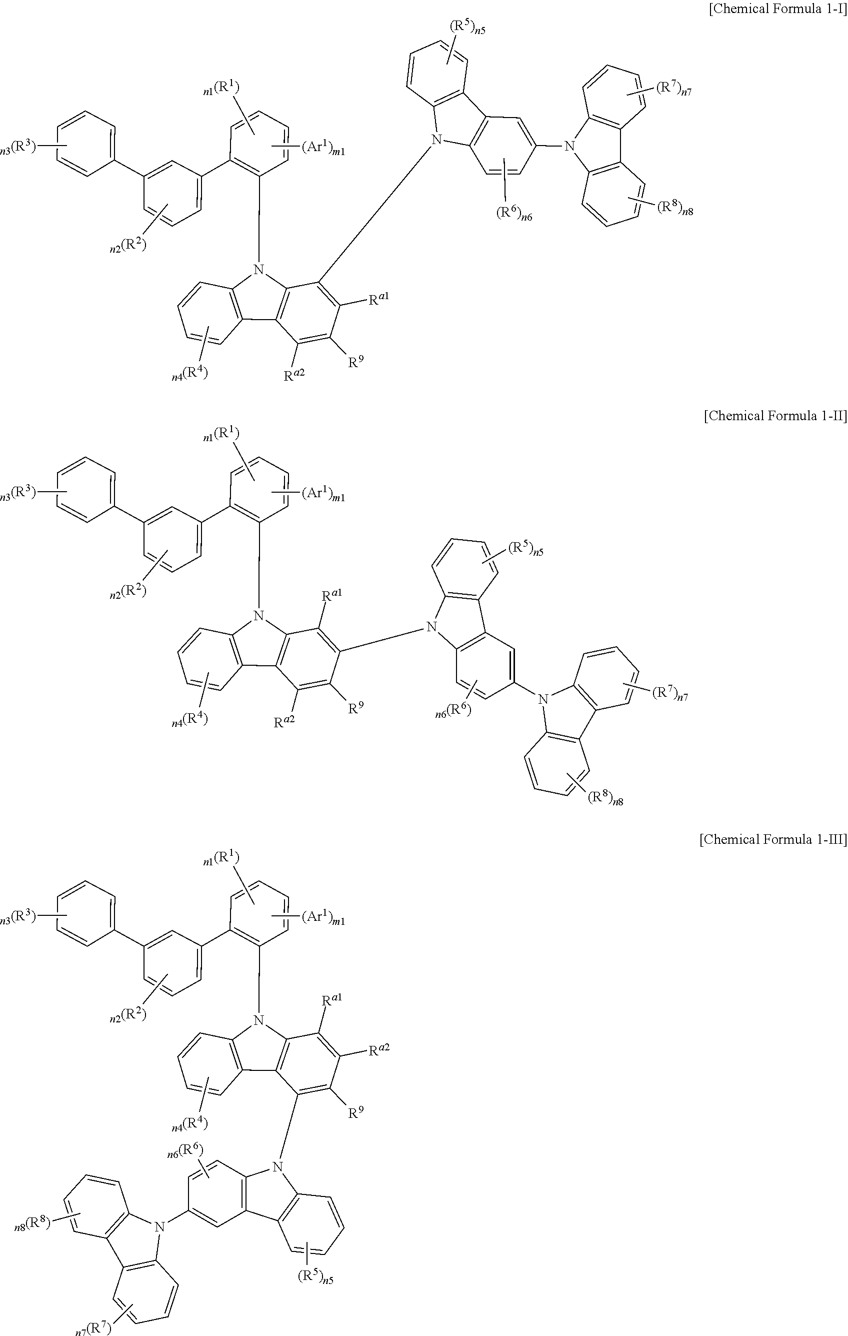
in Chemical Formula 1-I to Chemical Formula 1-III,
R¹ to R⁹, Ar¹, n1 to n8, and m1 are defined the same as those of Chemical Formula 1 and Chemical Formula 2, and
R^{a1} and R^{a2} are each independently defined the same as R^{a} in Chemical Formula 1.

3. The compound for an organic optoelectronic device as claimed in claim 1, wherein:
Chemical Formula 1 is represented by one of Chemical Formula 1-1 to Chemical Formula 1-4: 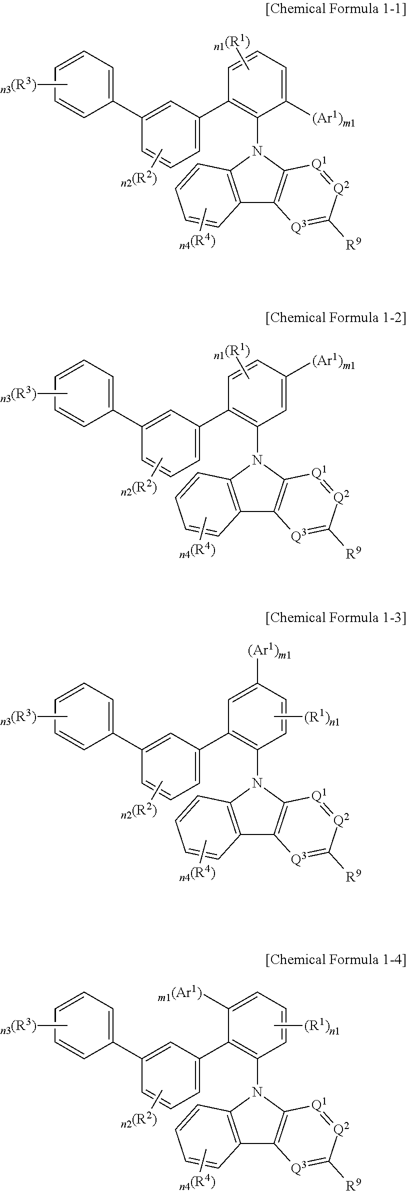
in Chemical Formula 1-1 to Chemical Formula 1-4, Q¹, Q², Q³, R¹ to R⁴, R⁹, Ar¹, n1 to n4, and m1 are defined the same as those of Chemical Formula 1.

4. The compound for an organic optoelectronic device as claimed in claim 3, wherein:
the combination of Chemical Formula 1 and Chemical Formula 2 is represented by one of Chemical Formula 1-1A, Chemical Formula 1-1B, or Chemical Formula 1-1C: 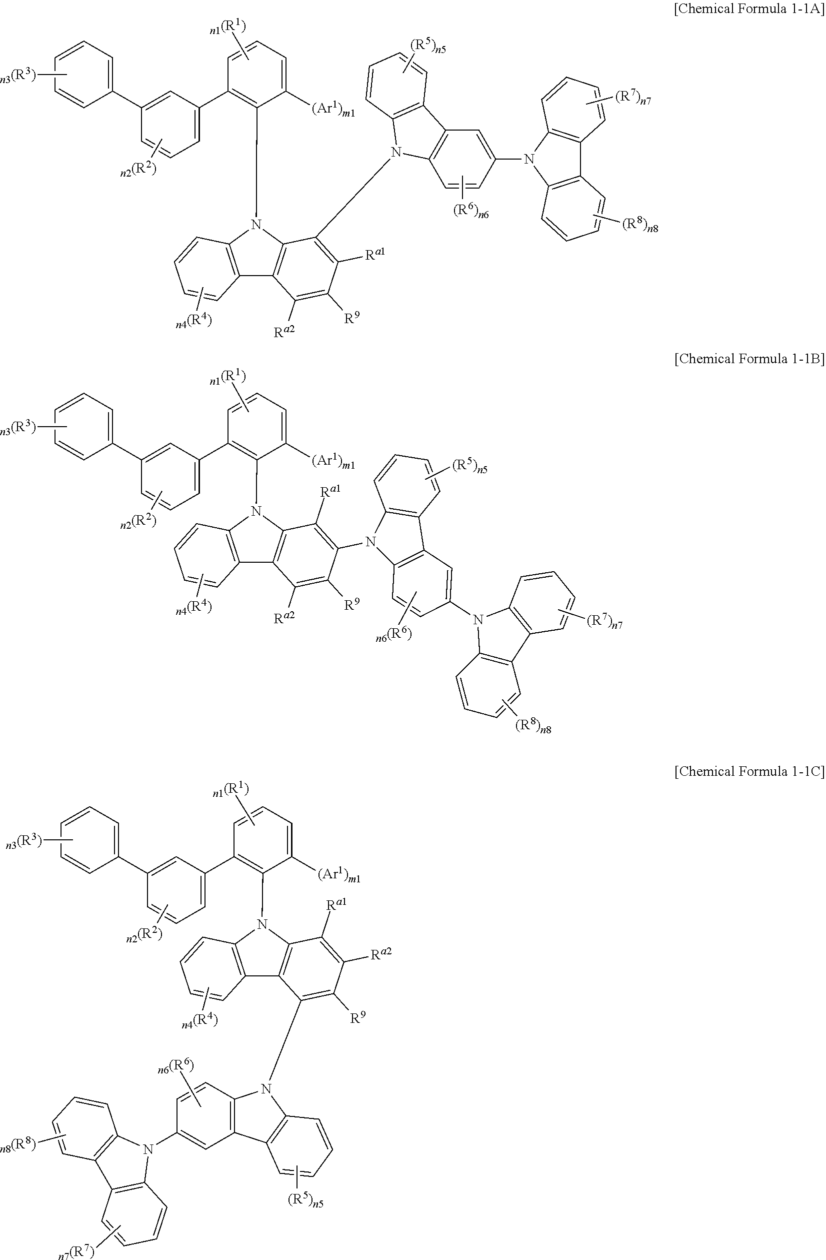
in Chemical Formula 1-1A, Chemical Formula 1-1B, and Chemical Formula 1-1C,
R¹ to R⁴, R⁹, Ar¹, n1 to n4, and m1 are defined the same as those of Chemical Formula 1-1,
R⁵ to R¹, R^{a1}, and R^{a2} are each independently hydrogen, deuterium, a substituted or unsubstituted C1 to C10 alkyl group, or a substituted or unsubstituted C6 to C12 aryl group,
n5, n7, and n8 are each independently an integer of 1 to 4, and
n6 is an integer of 1 to 3.

5. The compound for an organic optoelectronic device as claimed in claim 4, wherein the combination of Chemical Formula 1 and Chemical Formula 2 is represented by Chemical Formula 1-1B.

6. The compound for an organic optoelectronic device as claimed in claim 1, wherein:
the compound is a compound of Group 1: 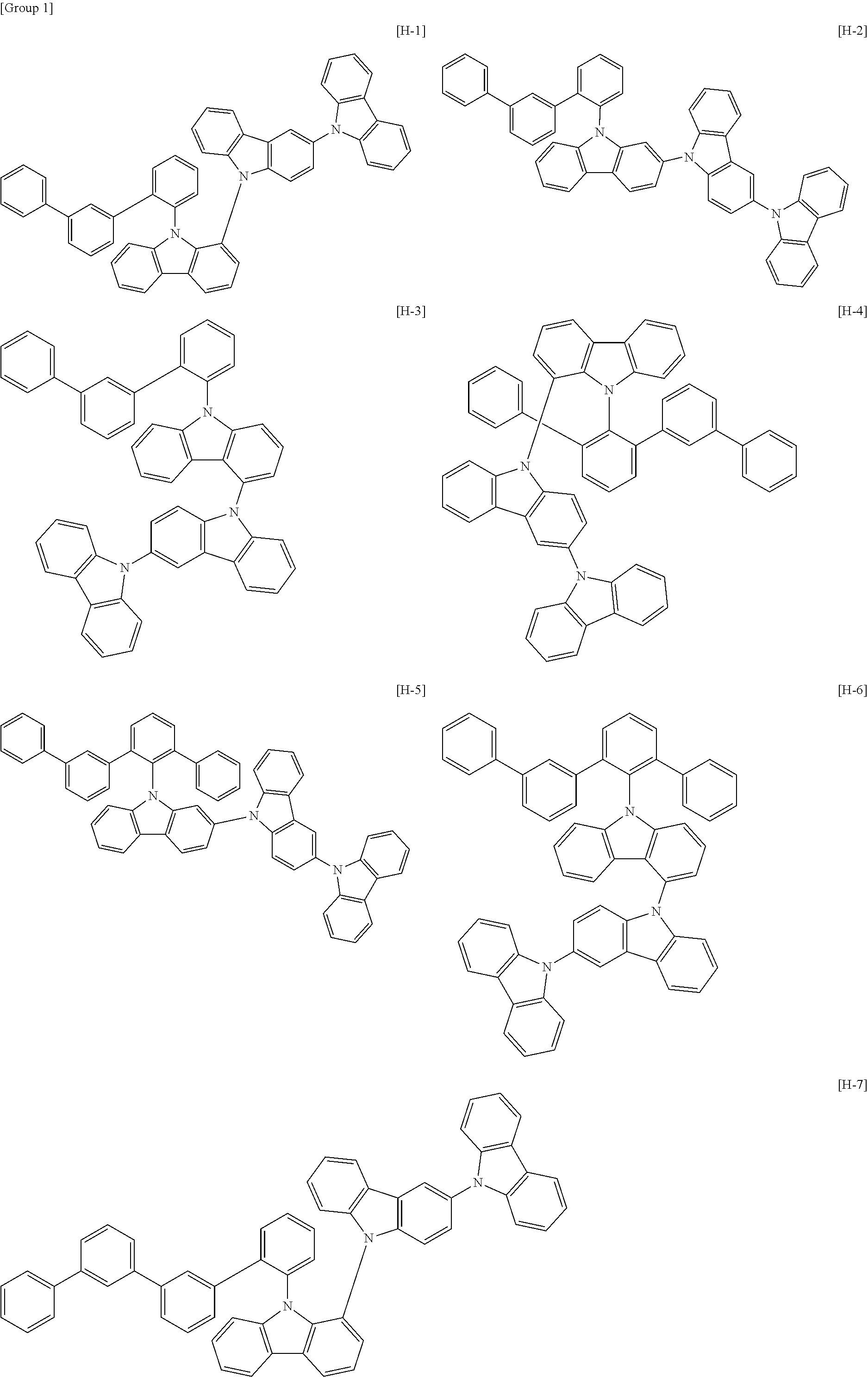 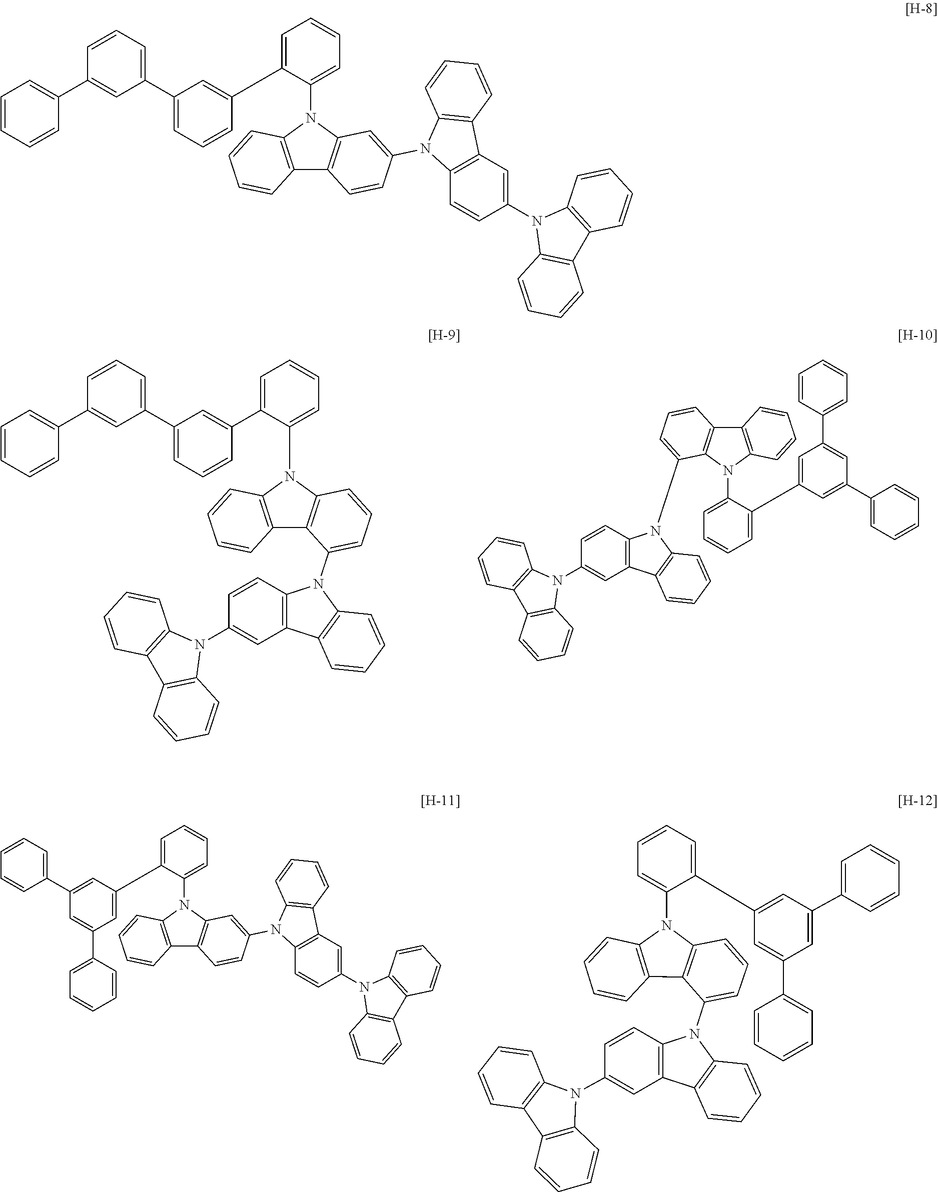 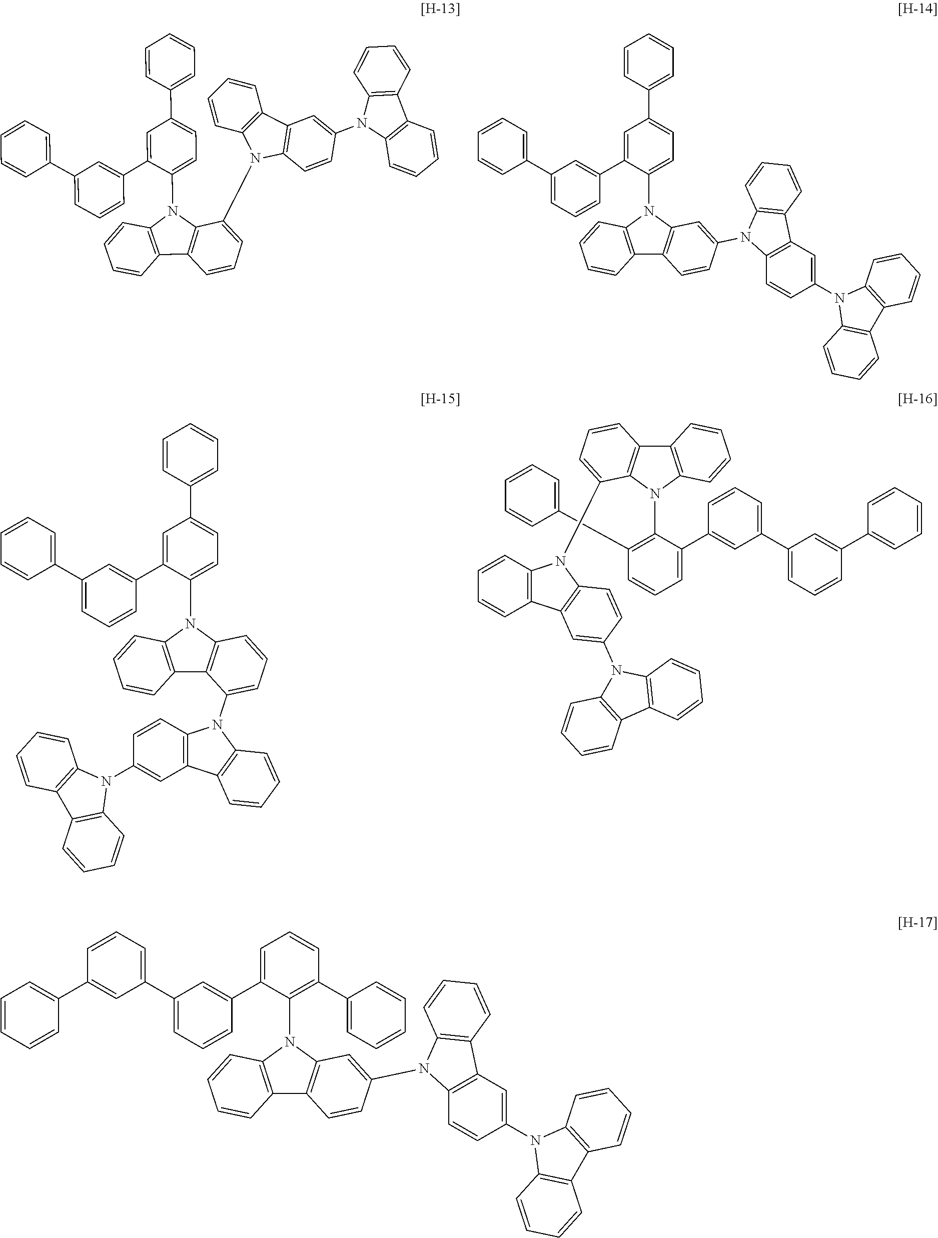 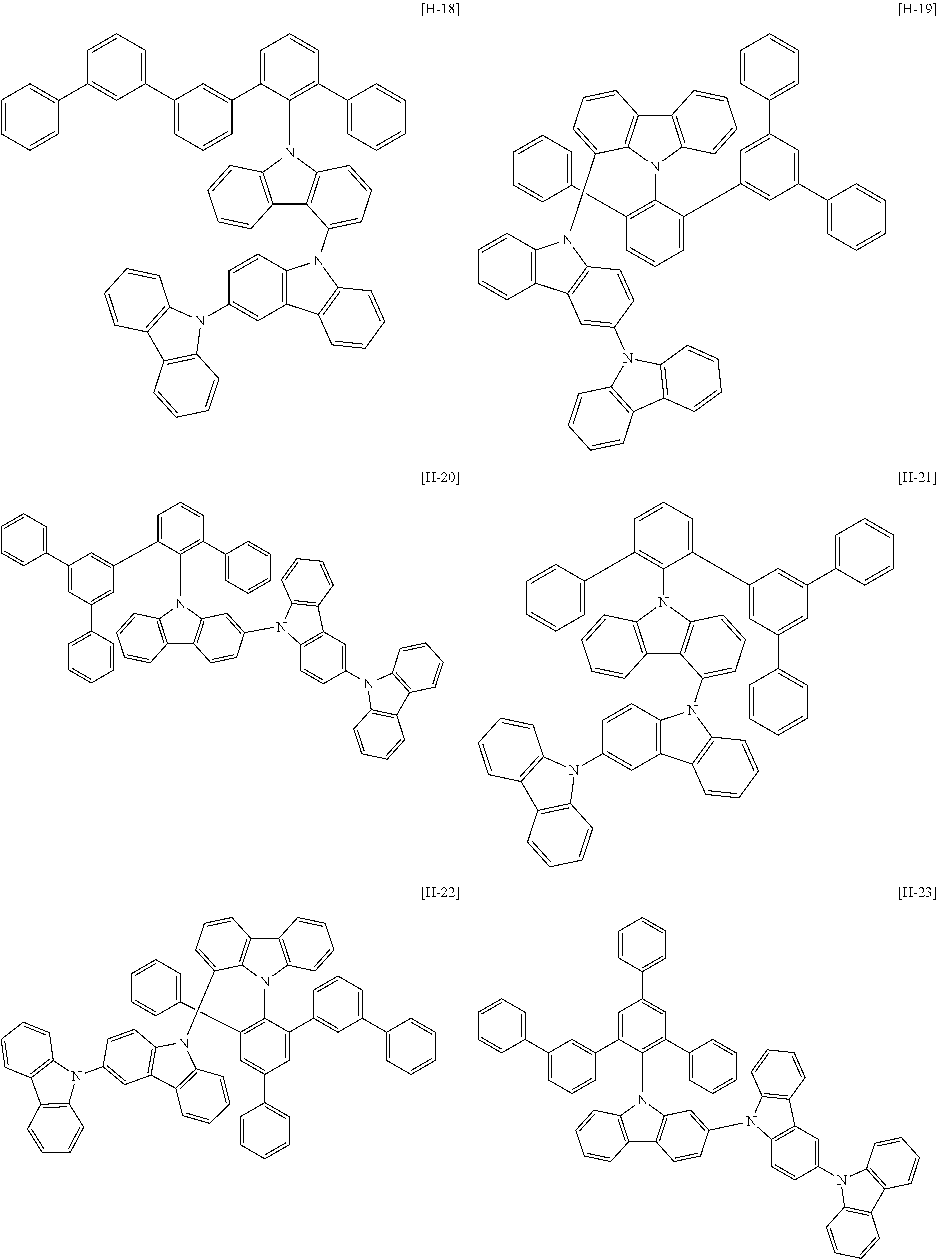 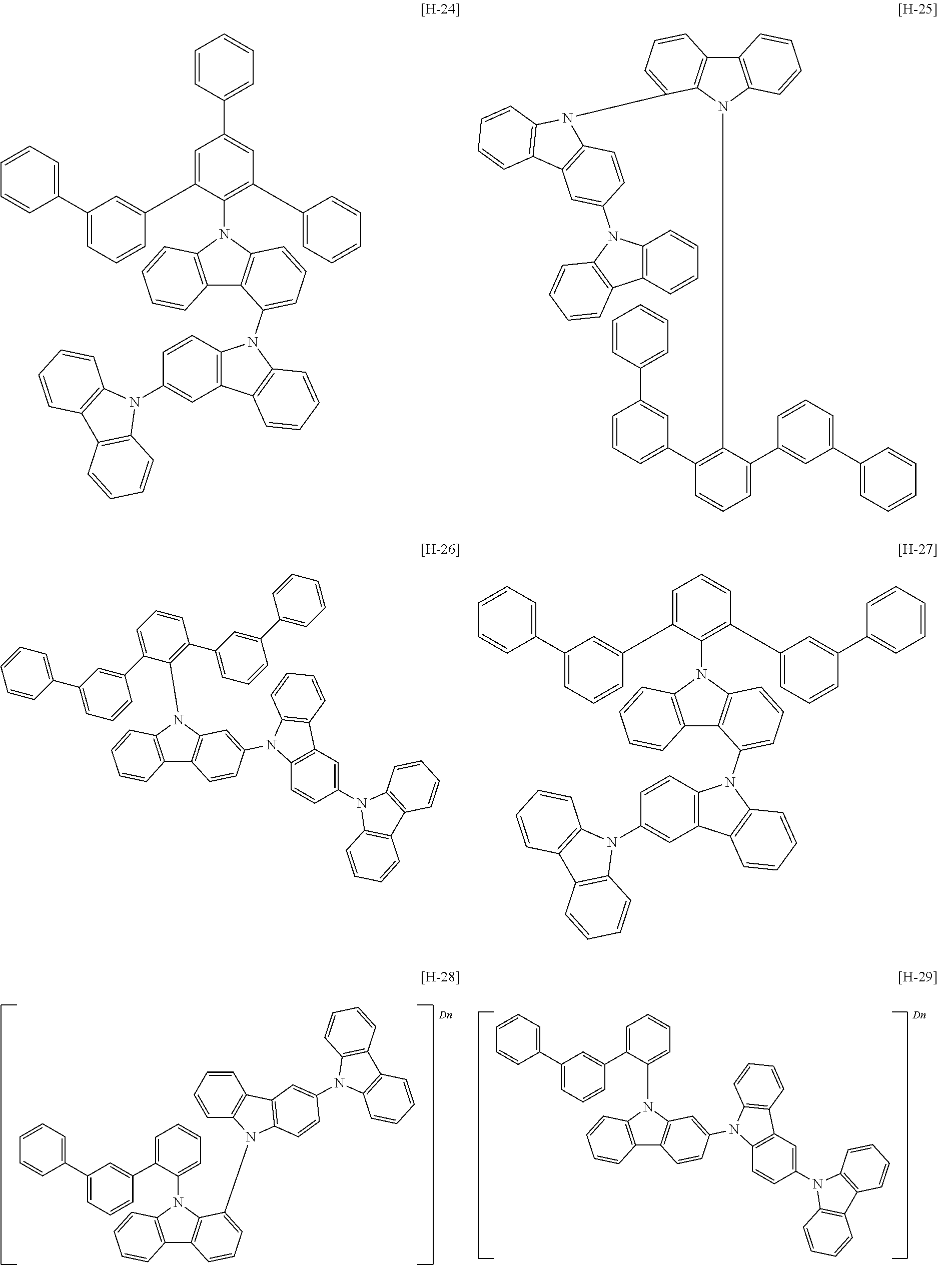 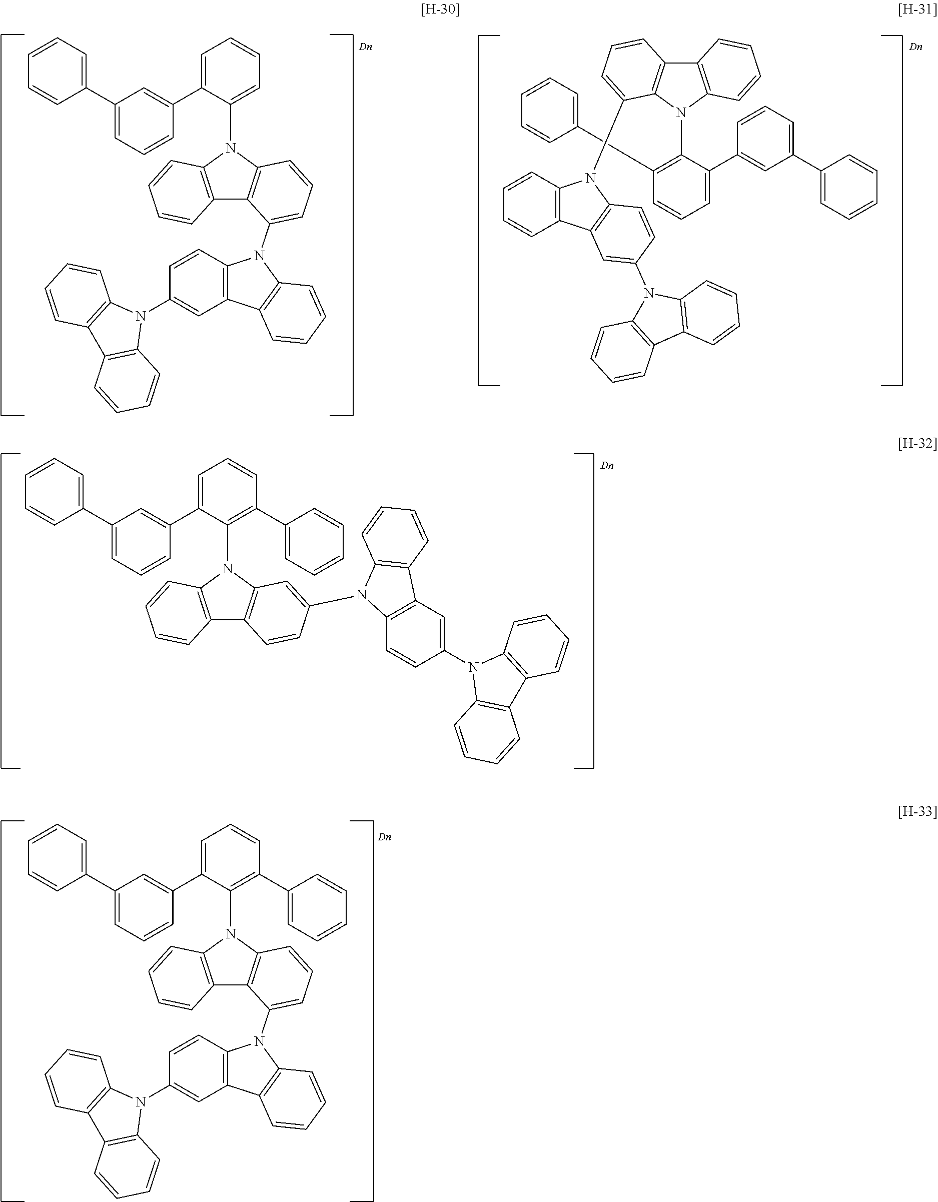 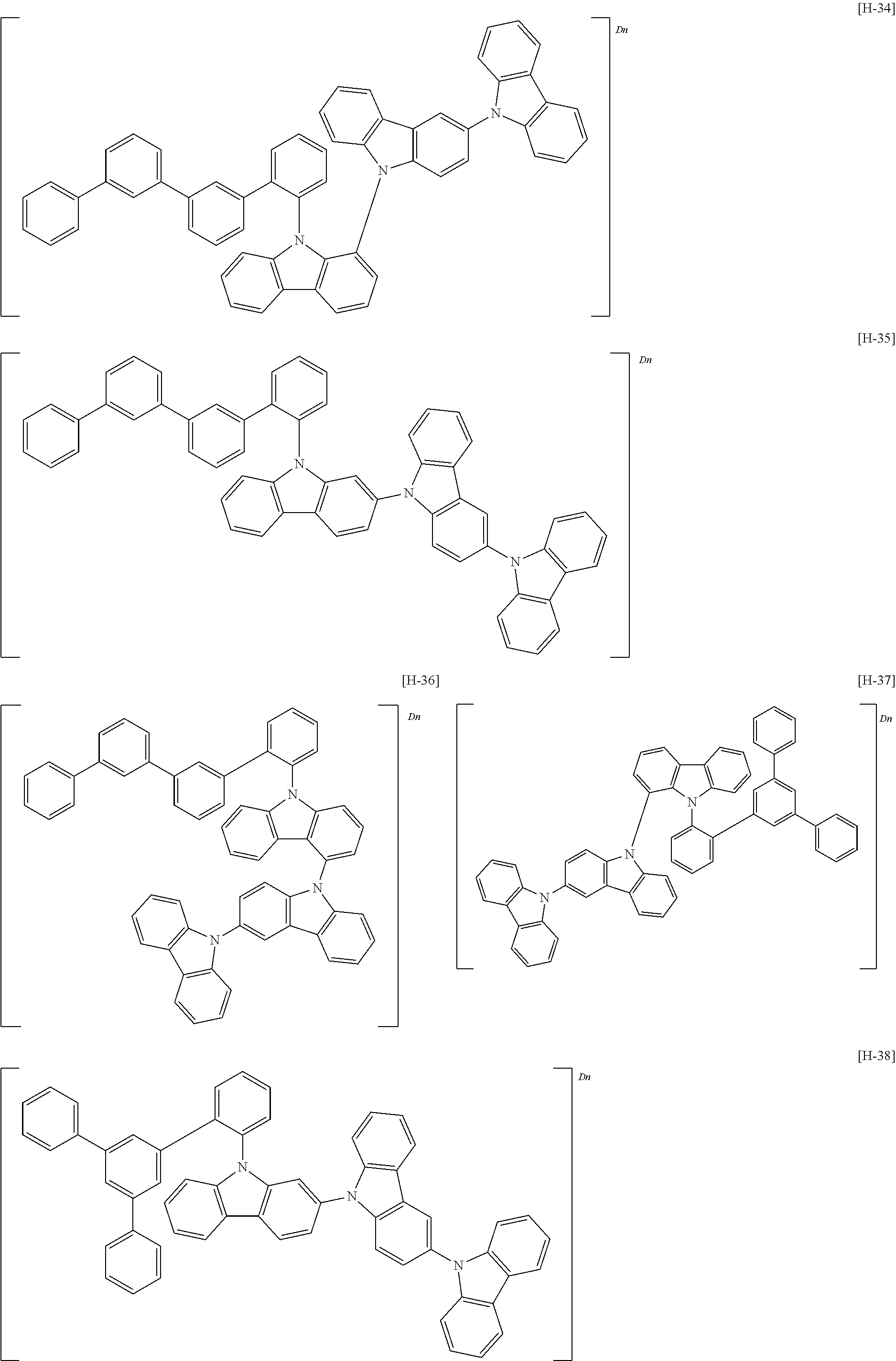 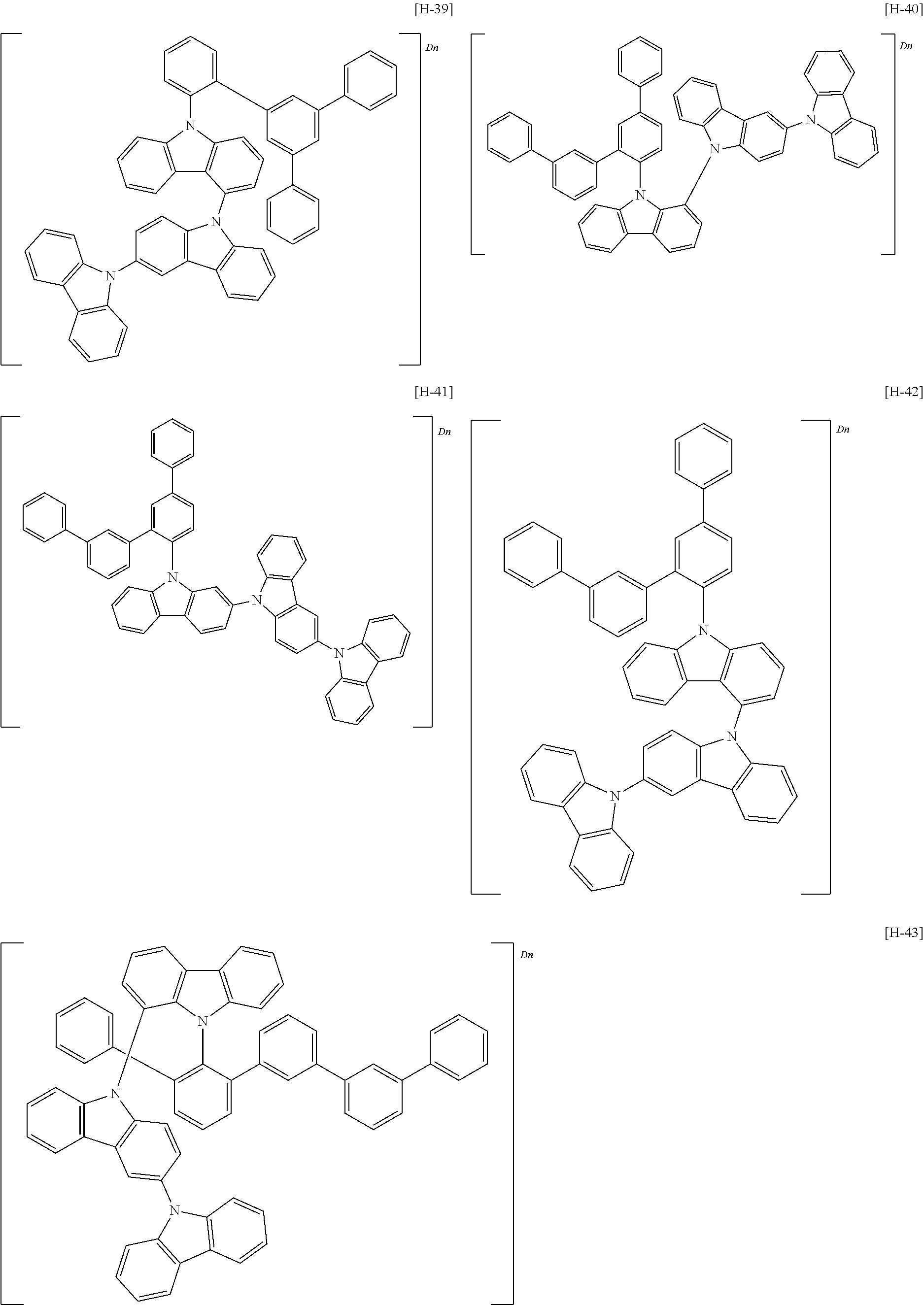 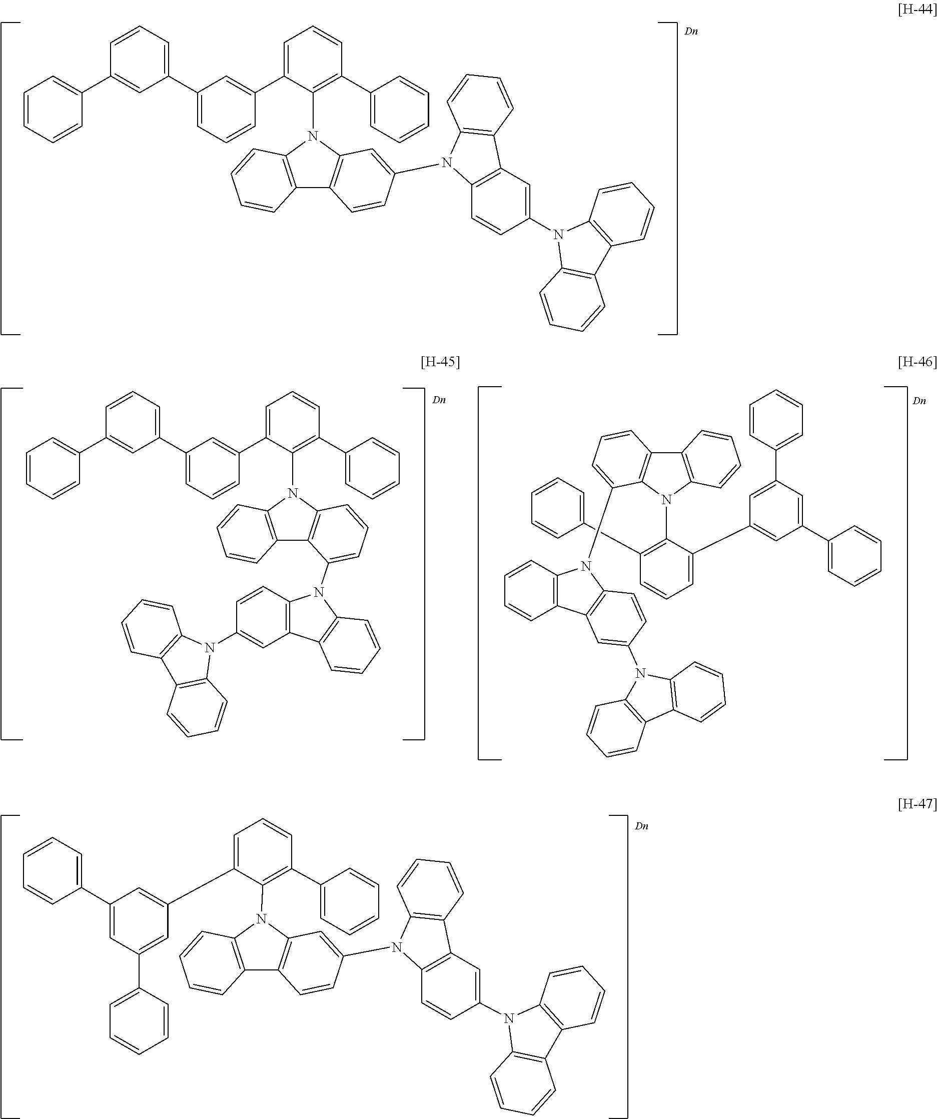 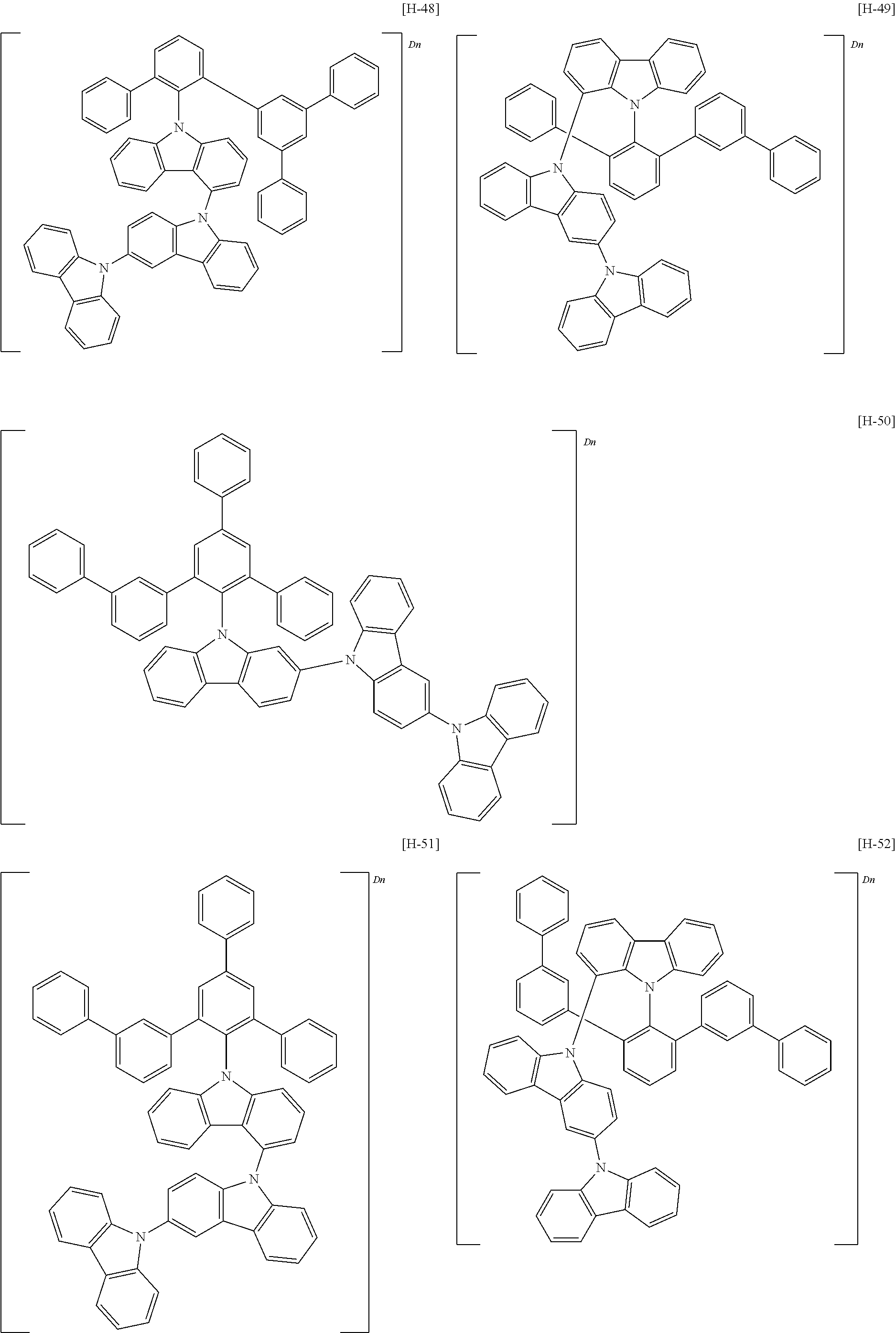 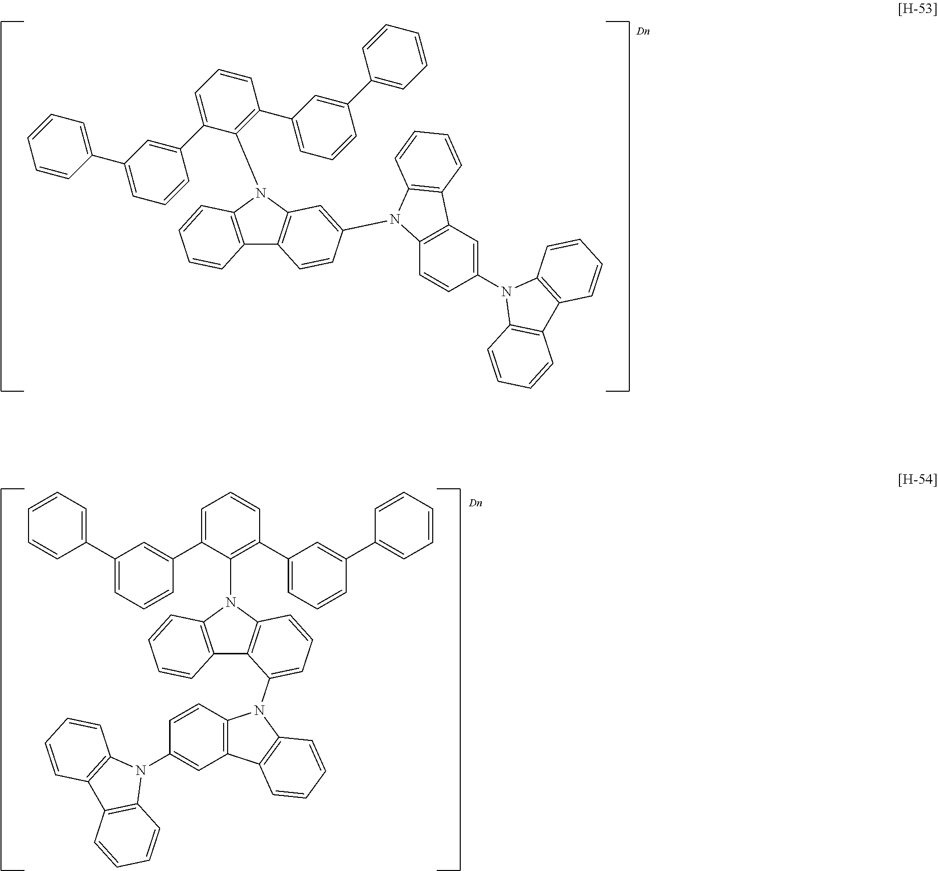
Dn indicates the number of hydrogen atoms replaced by deuterium, and n is an integer between 1 and the maximum number of possible replacements.

7. The compound for an organic optoelectronic device as claimed in claim 6, wherein the compound is substituted with deuterium and is a compound of Group 1-1:

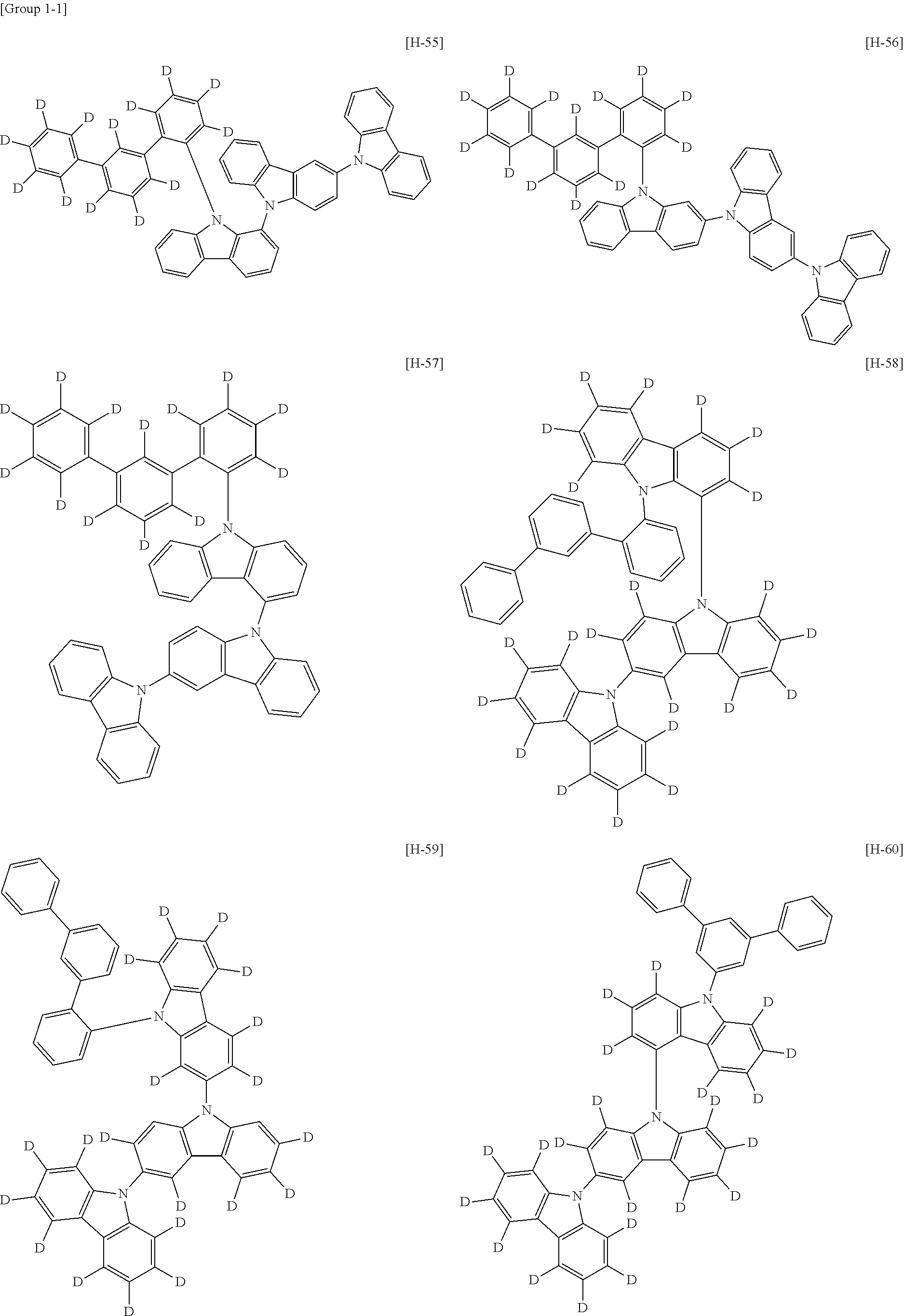

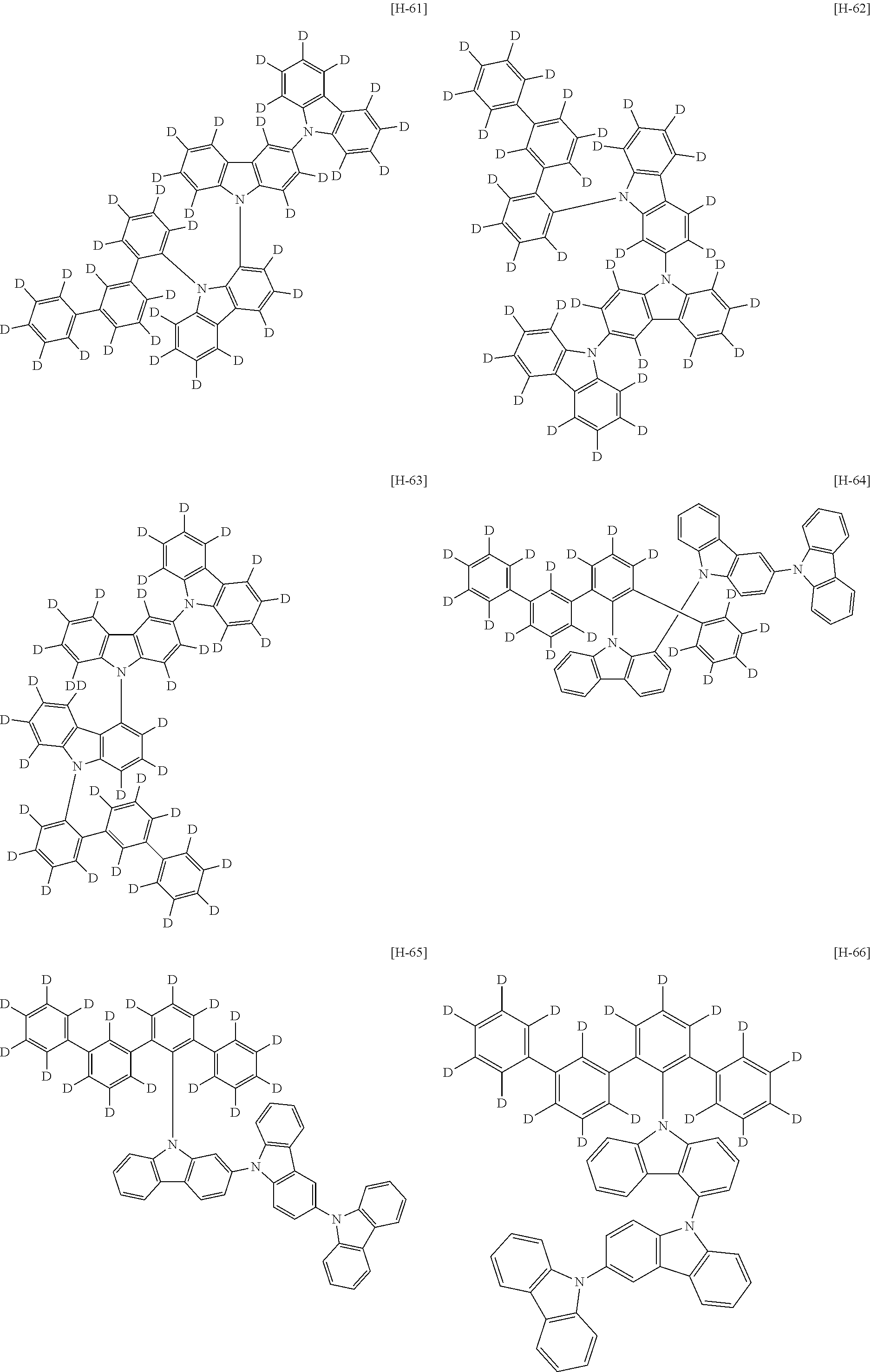

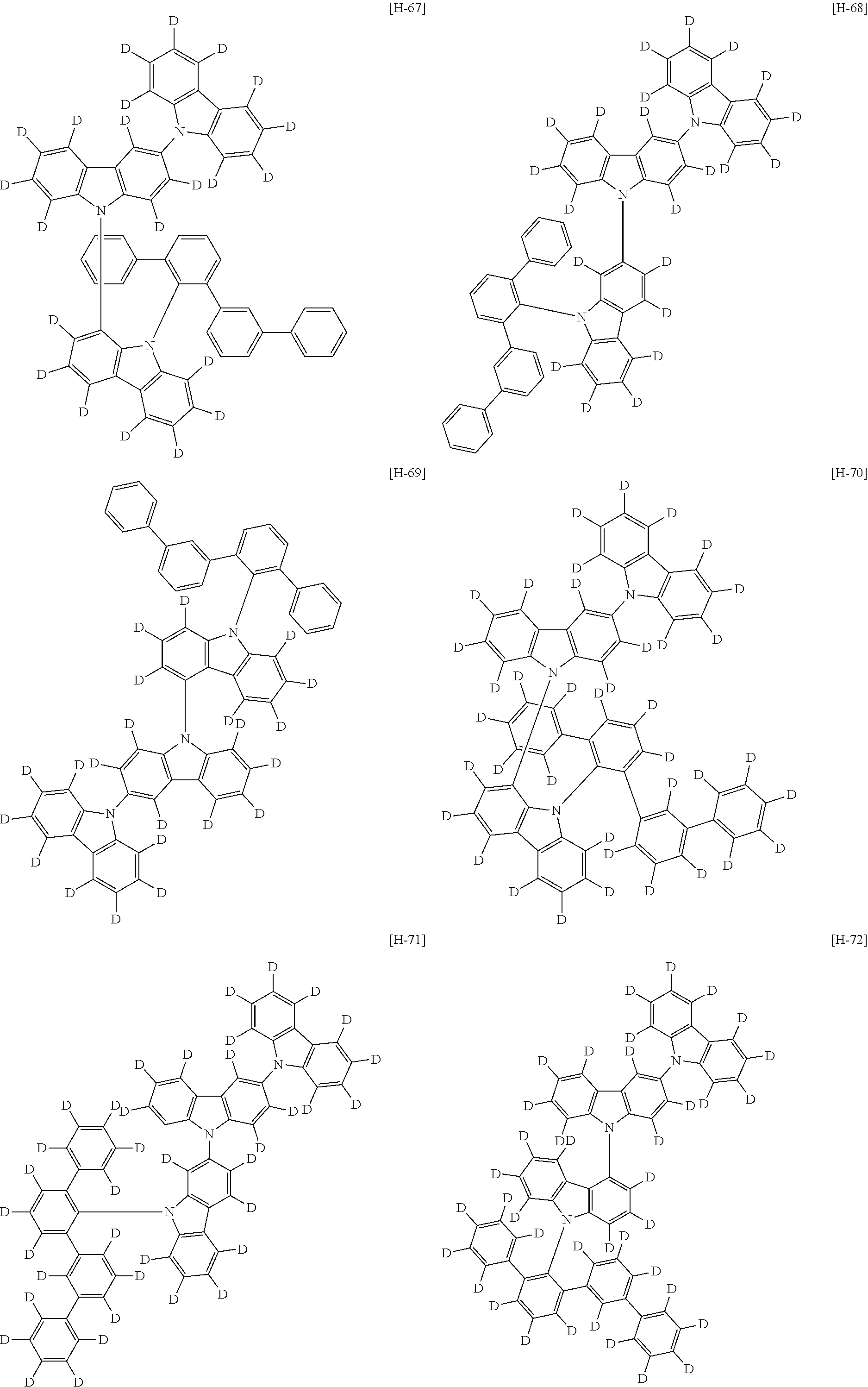

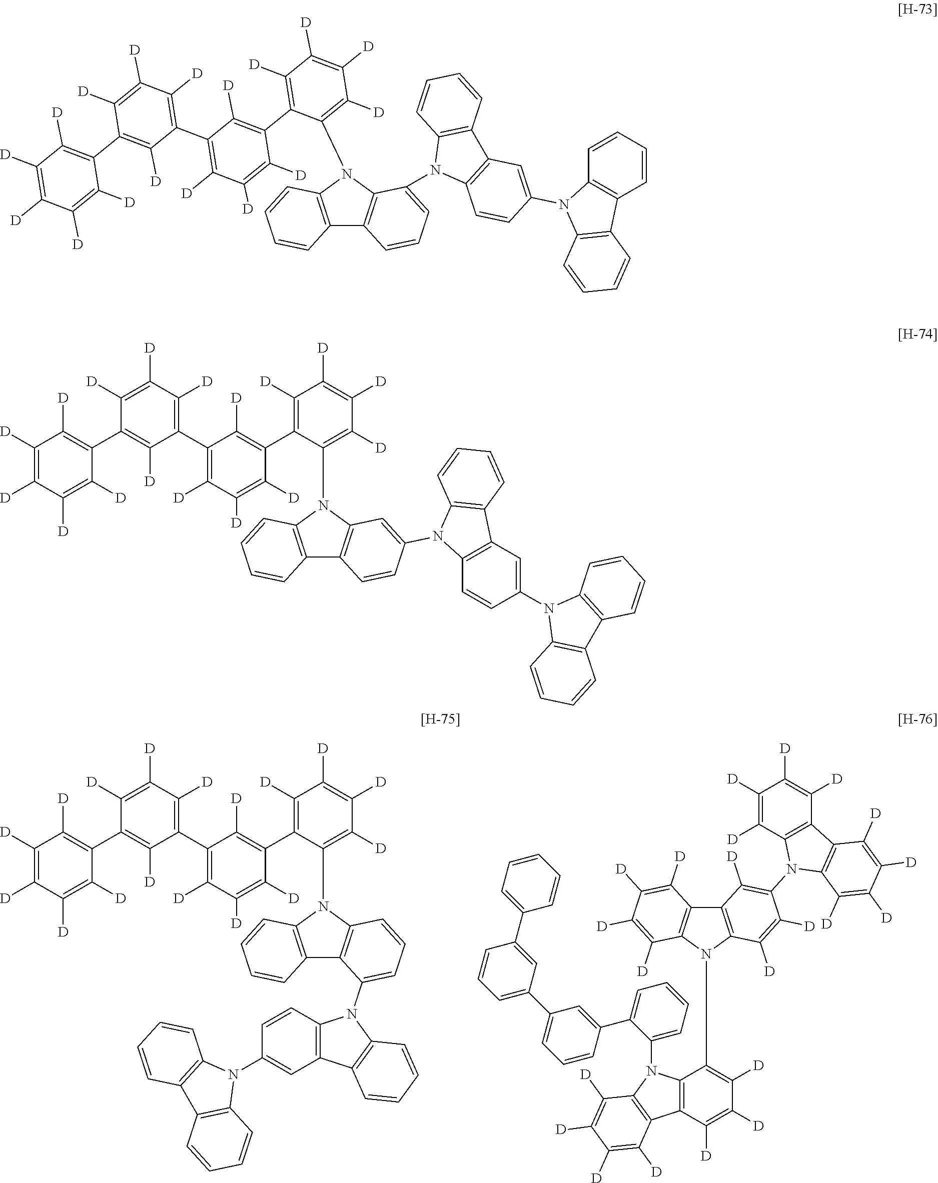

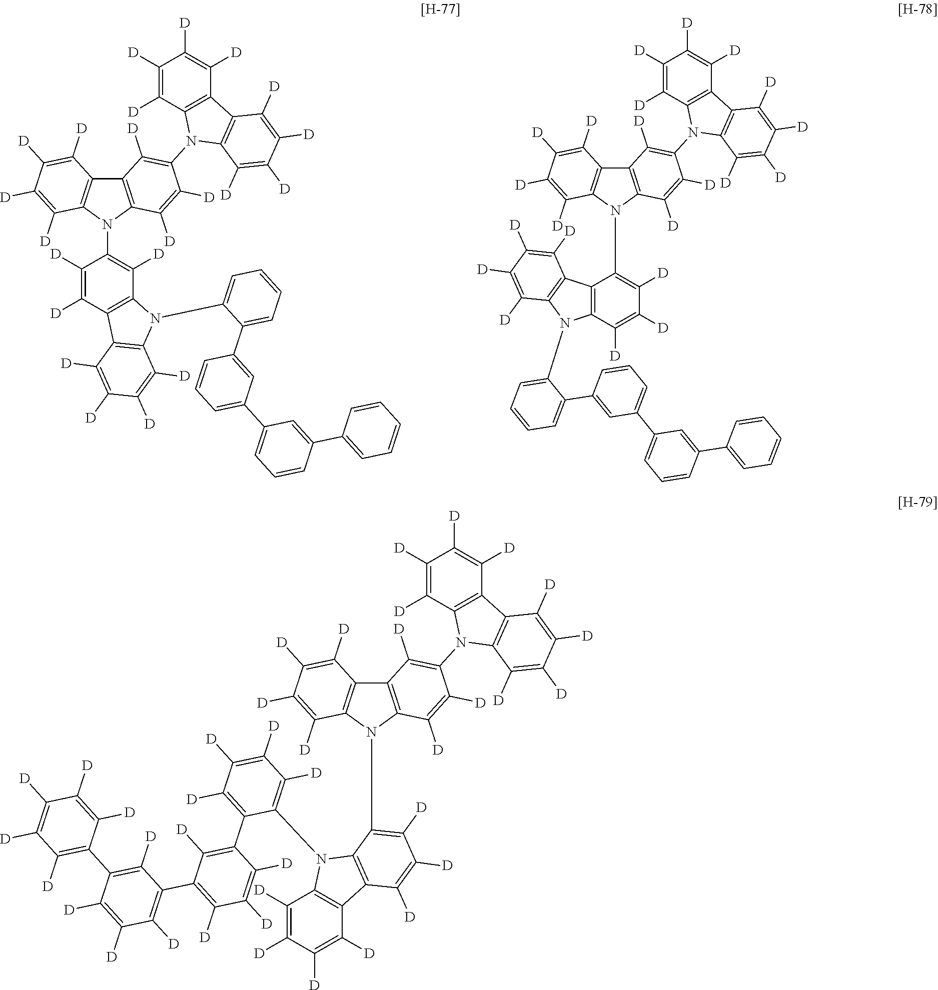

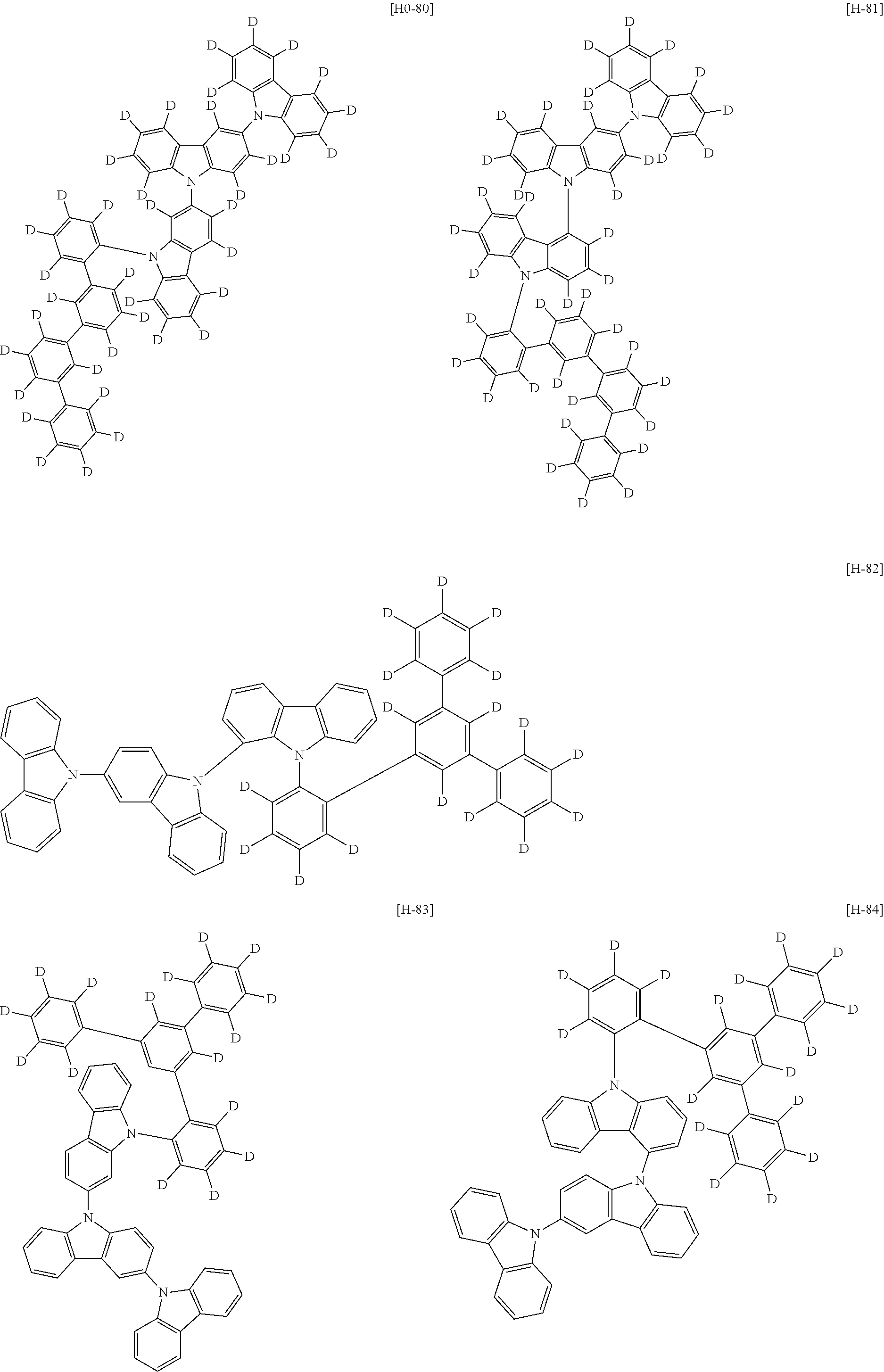

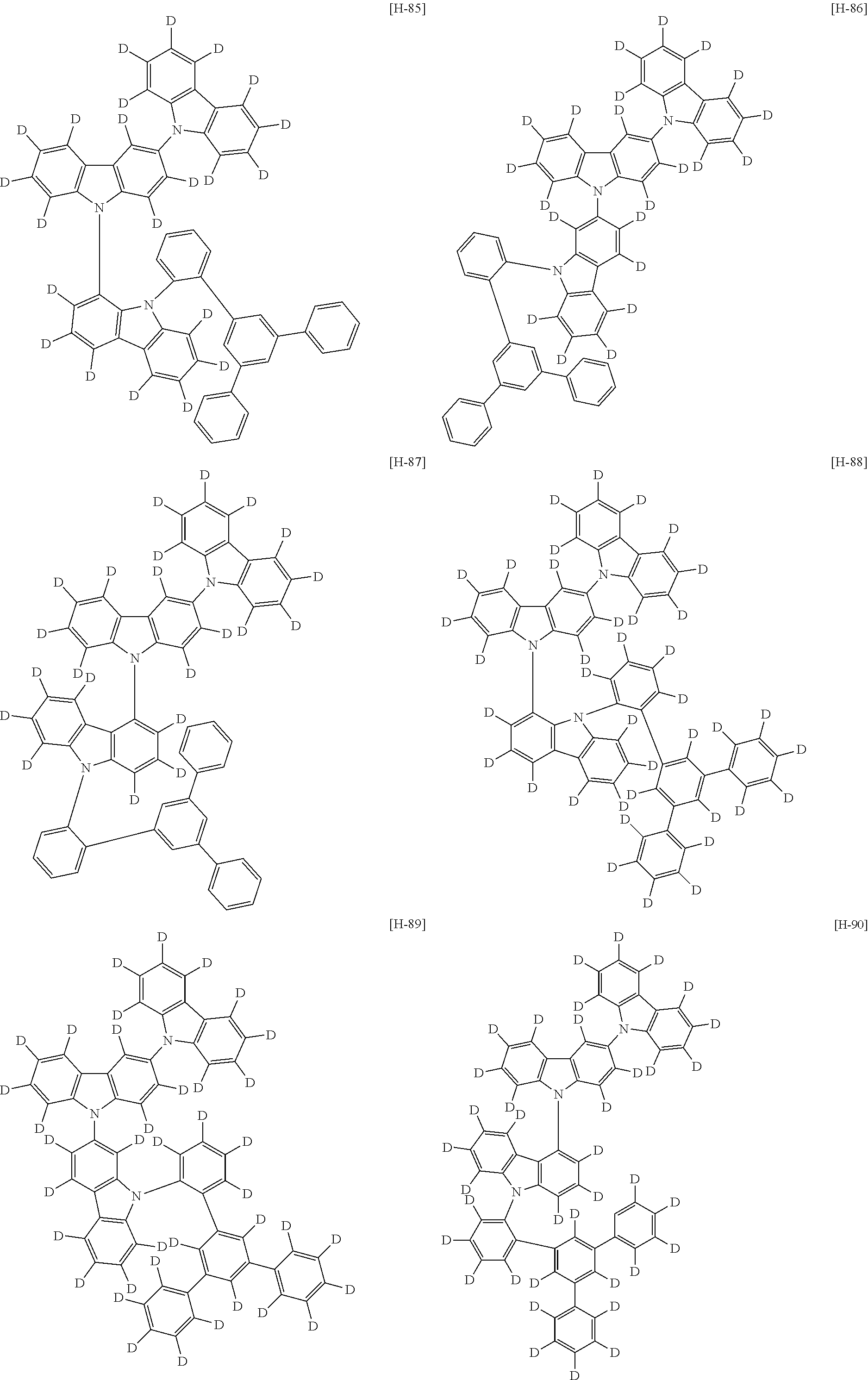

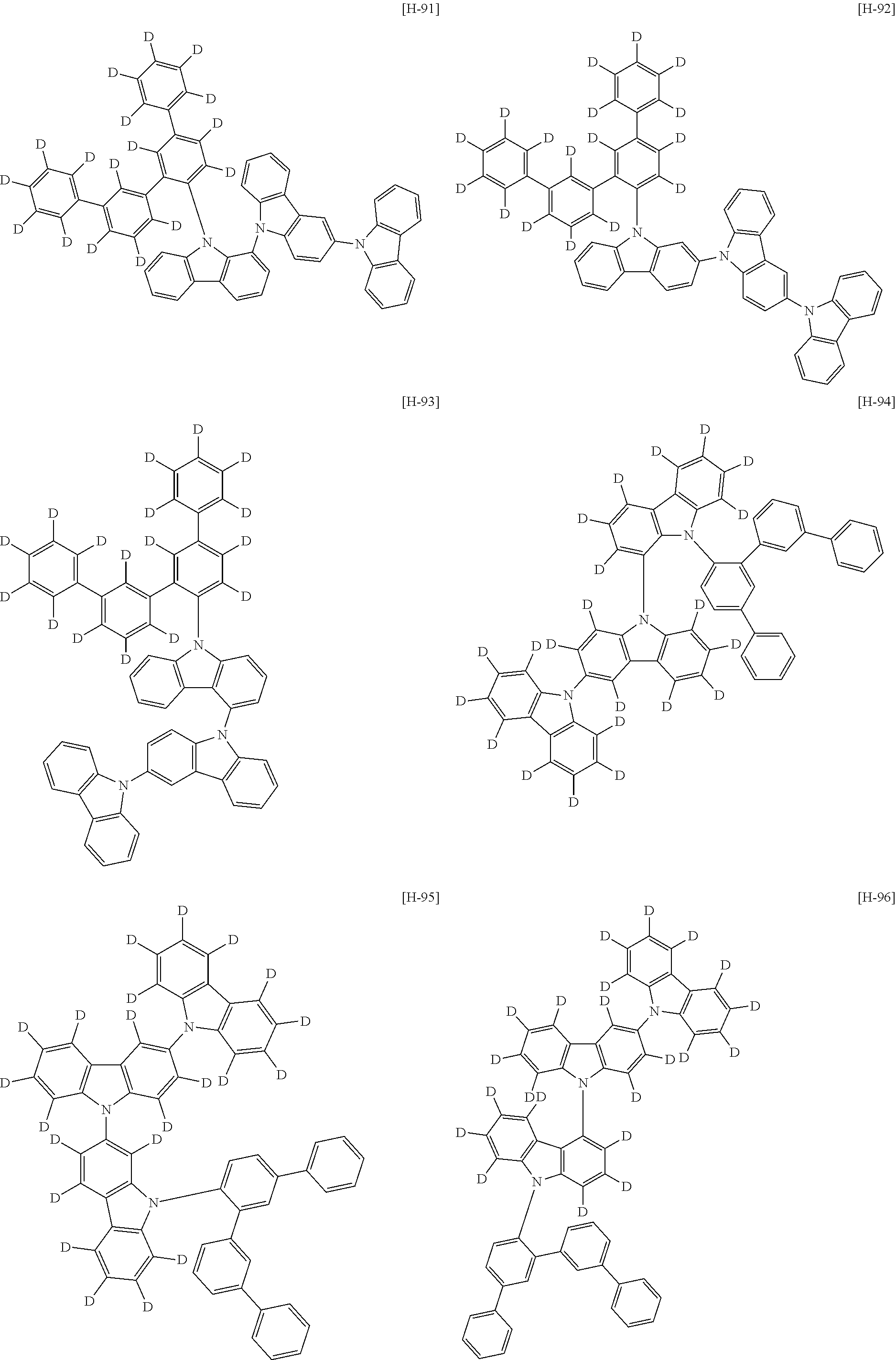

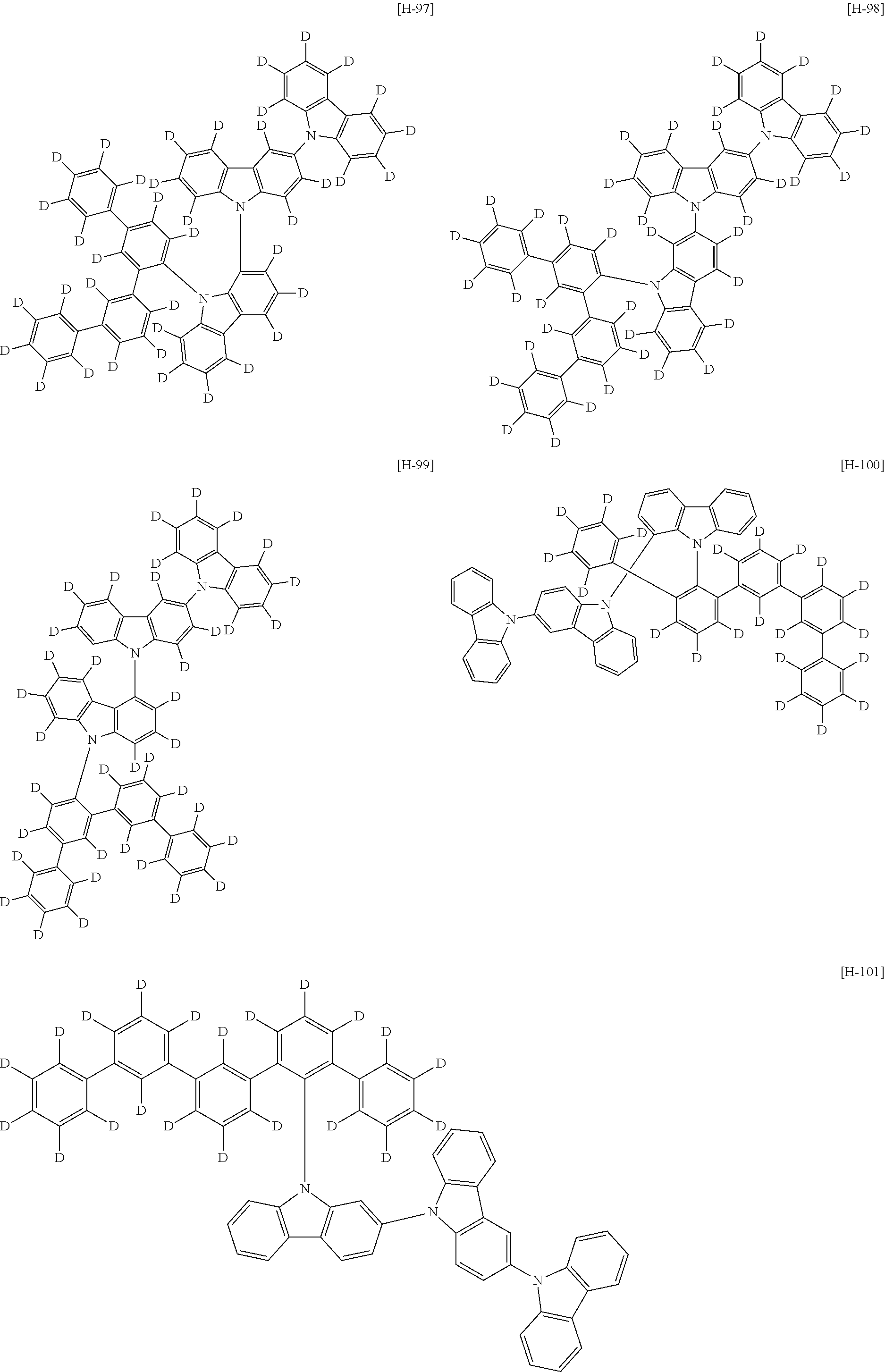

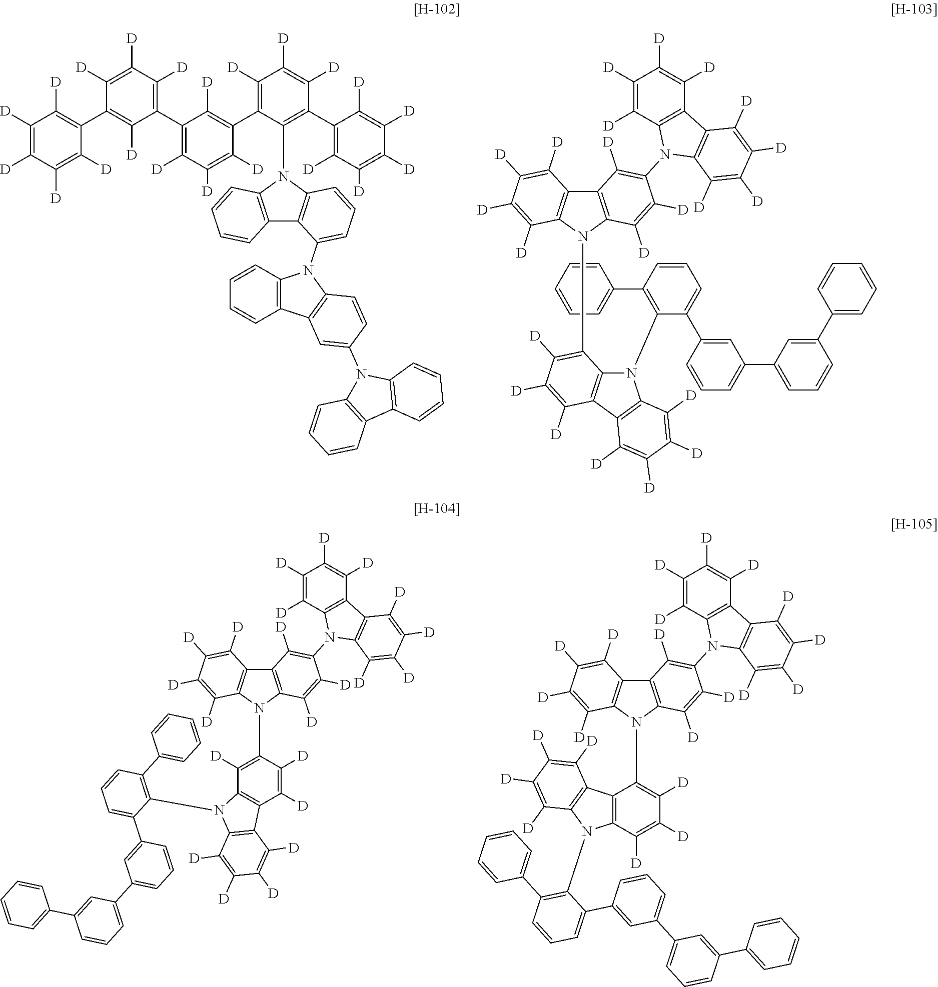

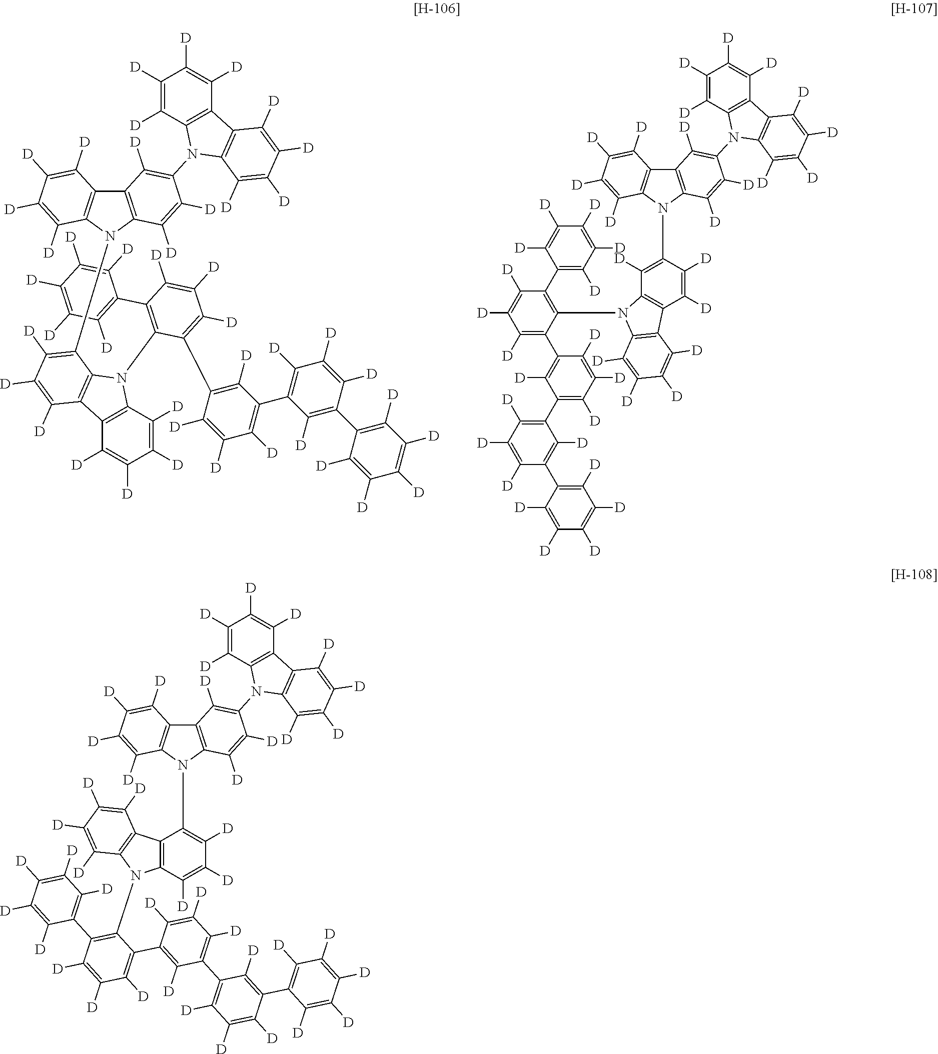

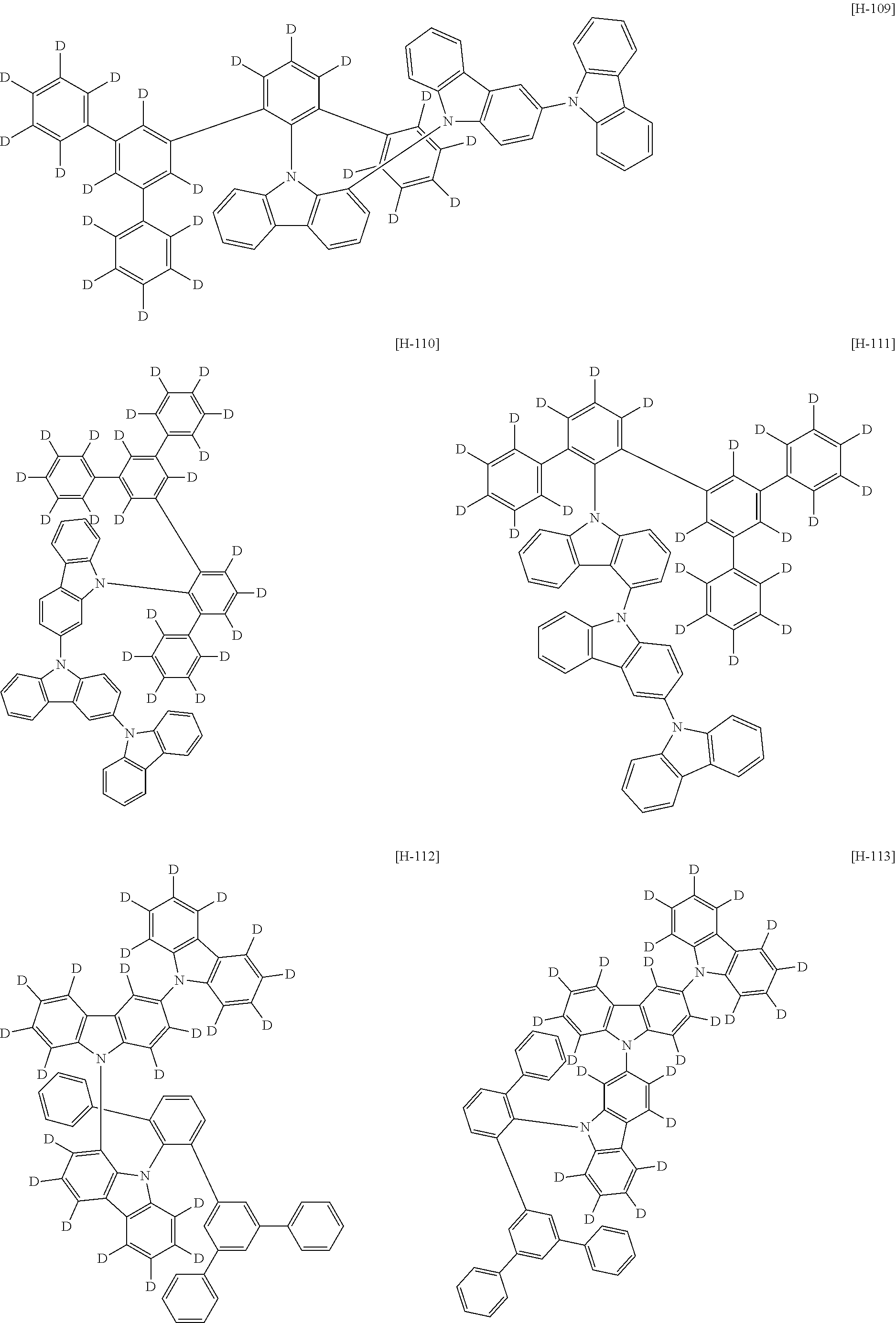

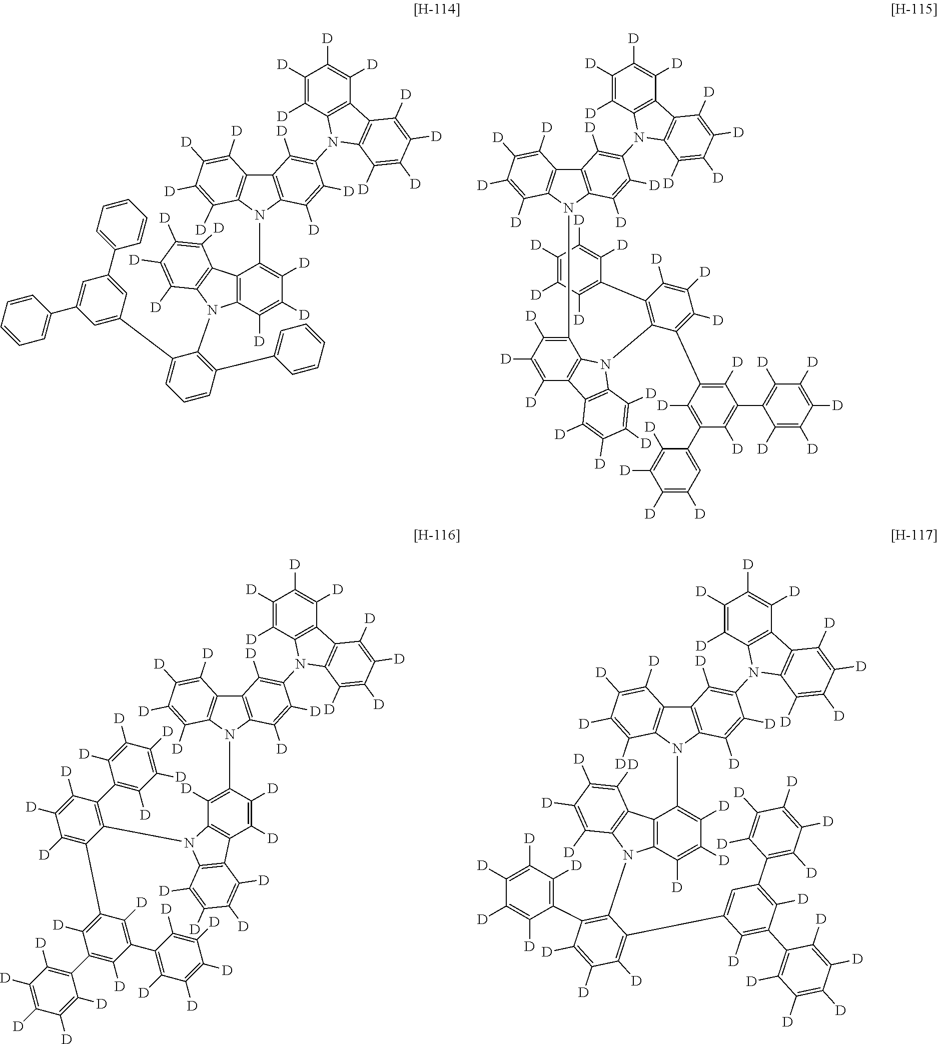

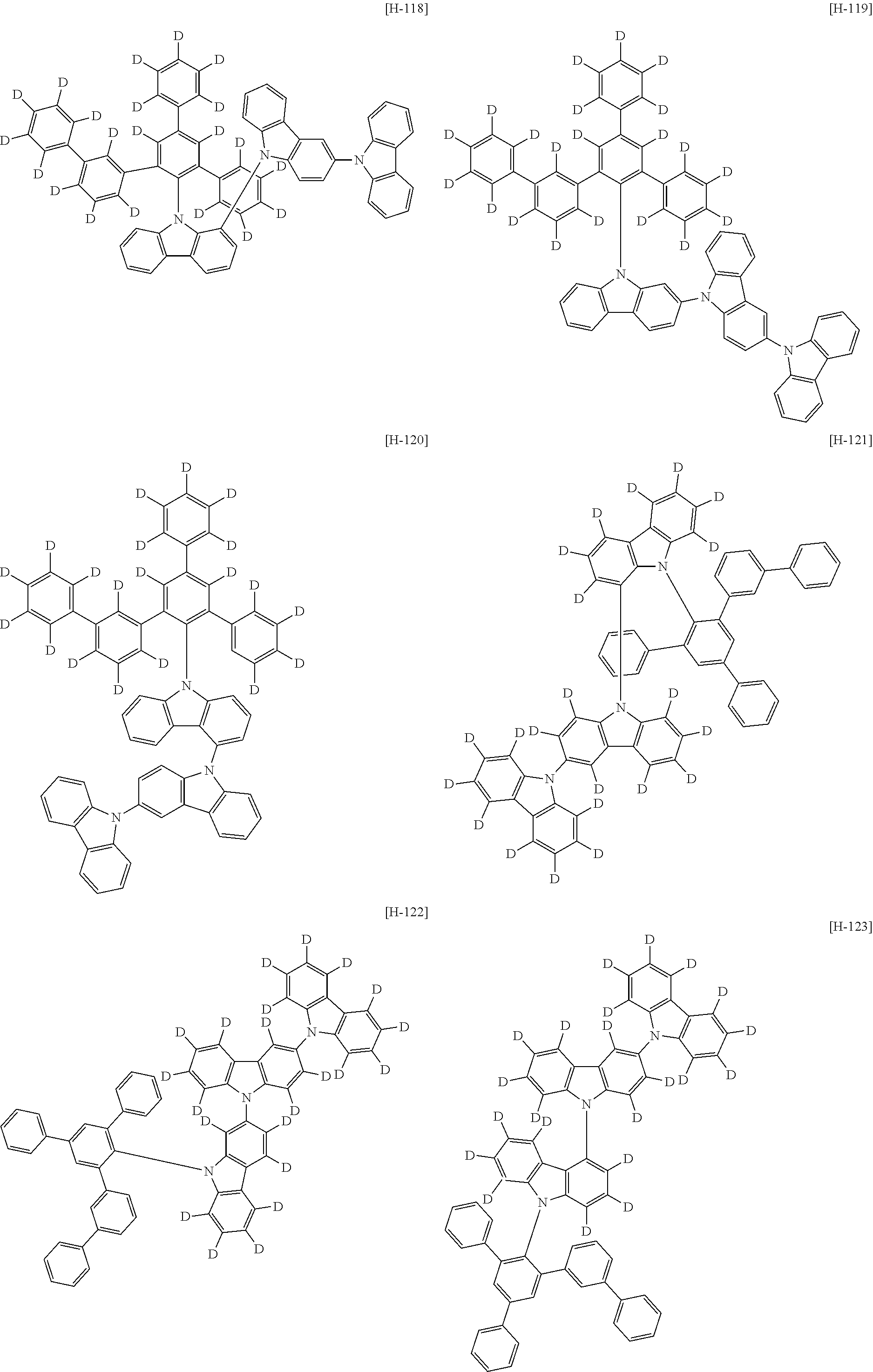

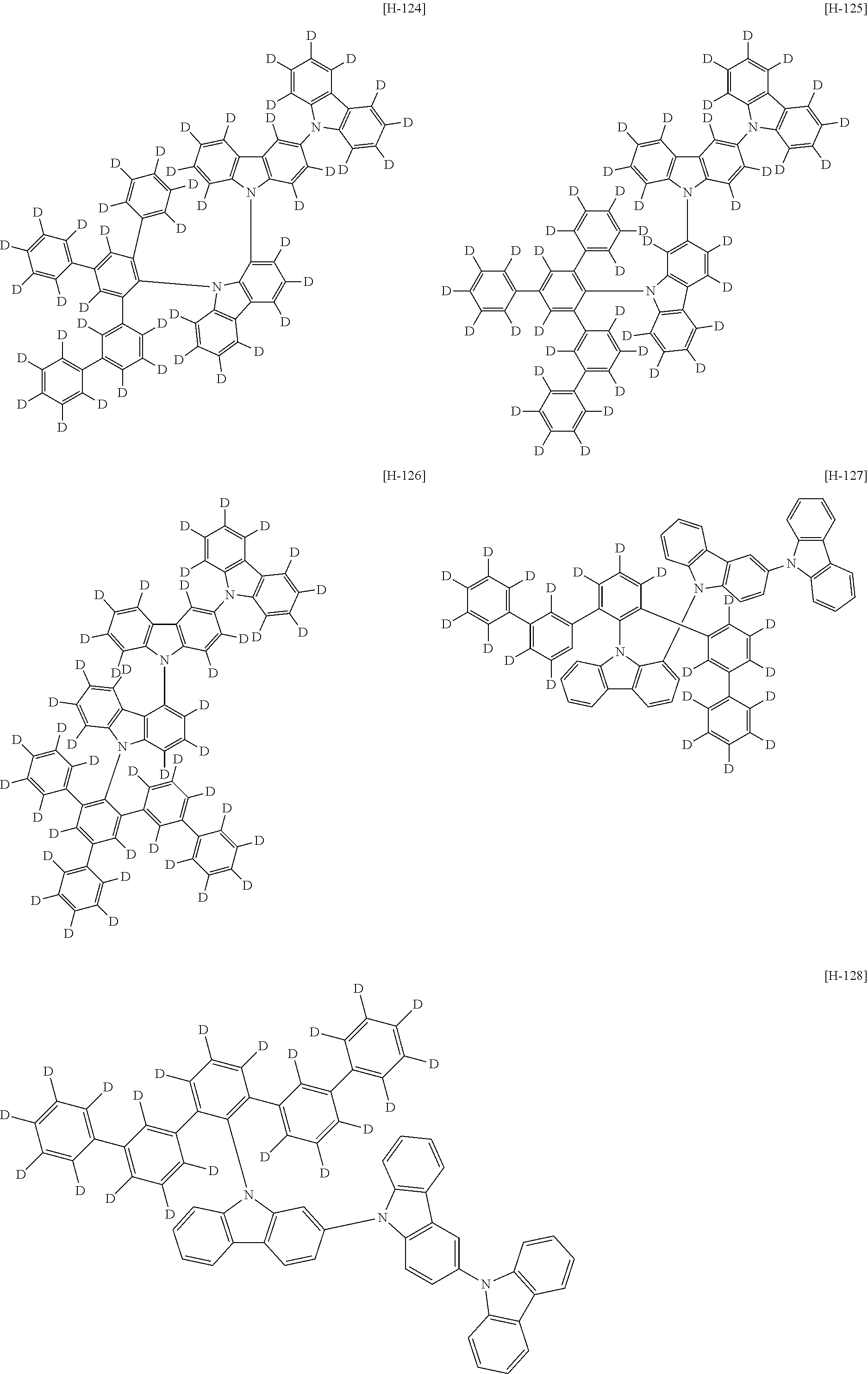

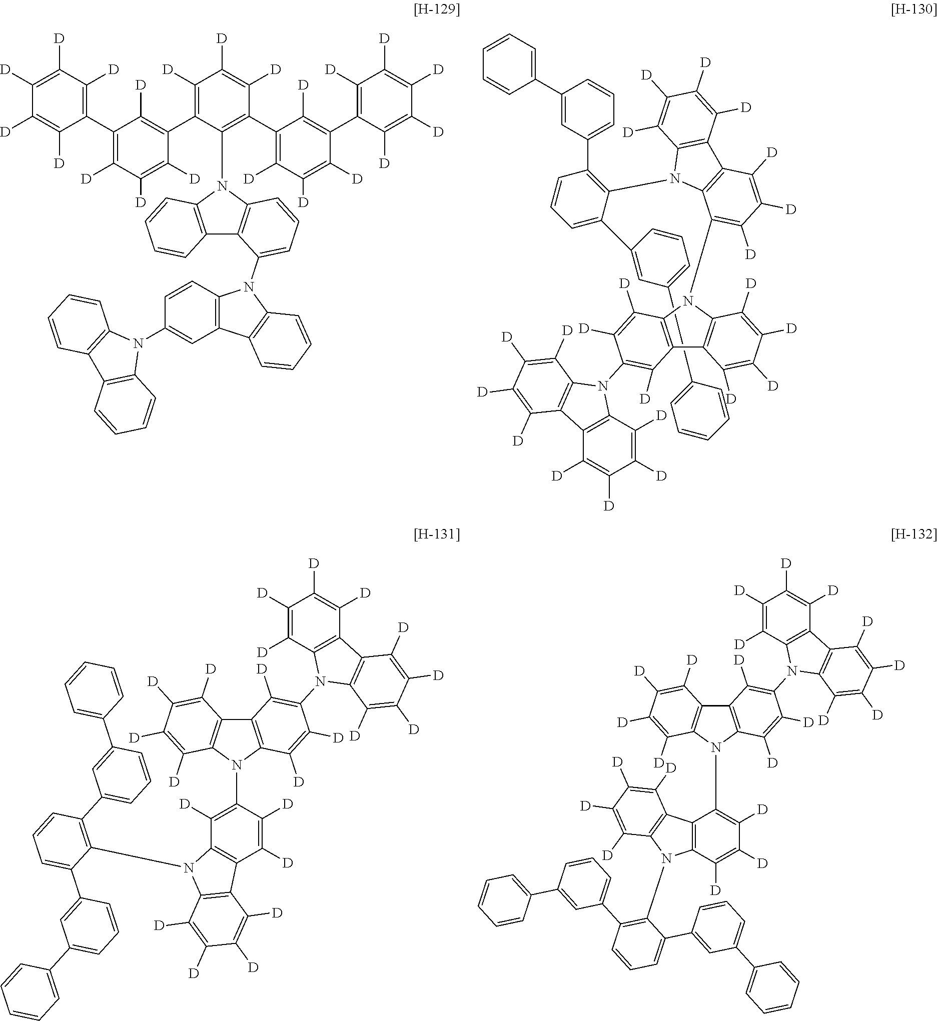

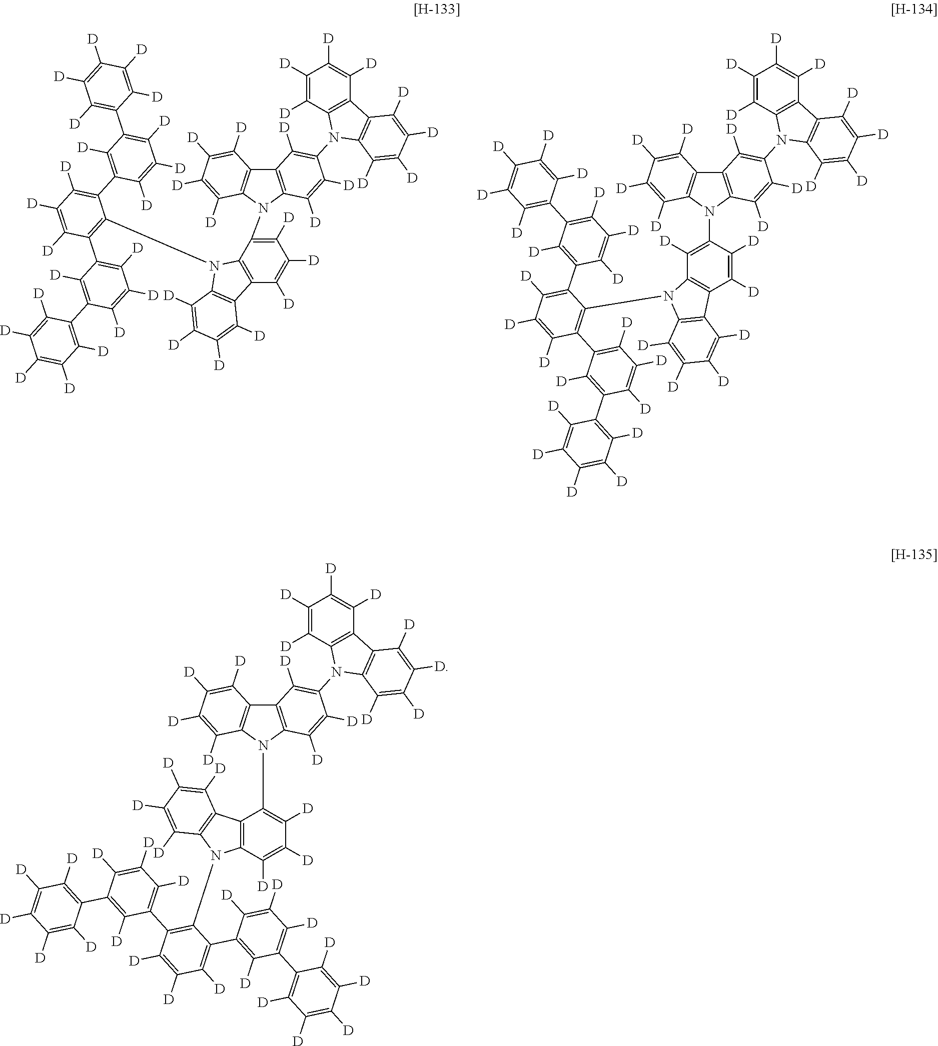

8. A composition for an organic optoelectronic device, comprising:
a first compound; and
a second compound,
wherein:
the first compound is the compound for an organic optoelectronic device as claimed in claim 1, and
the second compound is represented by Chemical Formula 3: 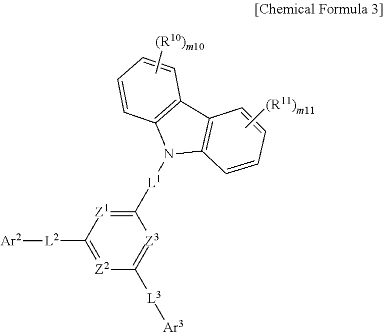
in Chemical Formula 3,
Z¹ to Z³ are each independently N or CR^{b}, provided that at least two of Z¹ to Z³ are N,
L¹ to L³ are each independently a single bond, a substituted or unsubstituted C6 to C20 arylene group, or a substituted or unsubstituted divalent C2 to C20 heterocyclic group,
Ar² and Ar³ are each independently a substituted or unsubstituted C6 to C30 aryl group, a substituted or unsubstituted C2 to C20 heterocyclic group, or a substituted or unsubstituted silyl group,
R^{b}, R¹⁰, and R¹¹ are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen,
m10 and m11 are each independently an integer of 1 to 4, and
each R¹⁰ and R¹¹ is separately present or two adjacent ones are linked to form a ring.

9. The composition for an organic optoelectronic device as claimed in claim 8, wherein in Chemical Formula 3, L¹ is a single bond.

10. The composition for an organic optoelectronic device as claimed in claim 8, wherein:
the second compound is represented by Chemical Formula 3a or Chemical Formula 3b: 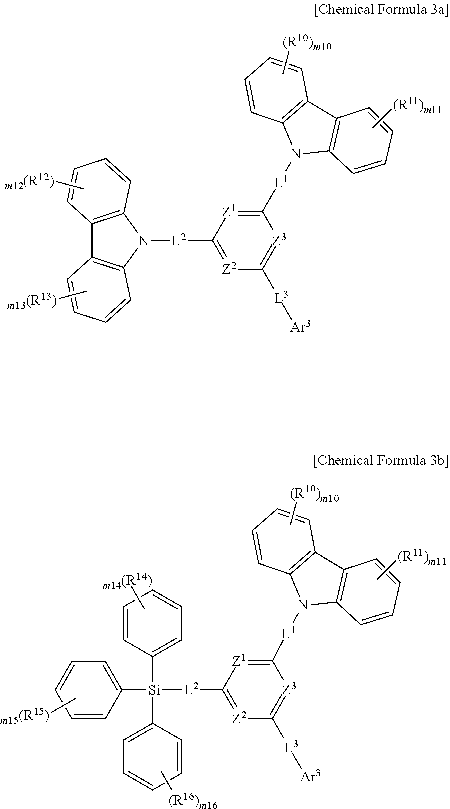
in Chemical Formula 3a and Chemical Formula 3b,
Z¹ to Z³, L¹ to L³, m10, m11, R¹⁰ and R¹¹ are defined the same as those of Chemical Formula 3,
R¹² to R¹⁶ are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen,
m12 and m13 are each independently an integer of 1 to 4,
m14 to m16 are each independently an integer of 1 to 5, and
each R¹² to R¹⁶ is separately present or two adjacent ones are linked to form a ring.

11. The composition for an organic optoelectronic device as claimed in claim 10, wherein:
L¹ is a single bond,
L² is a single bond, a substituted or unsubstituted phenylene group, a substituted or unsubstituted biphenylene group, a substituted or unsubstituted terphenylene group, a substituted or unsubstituted naphthylene group, a substituted or unsubstituted triphenylenylene group, a substituted or unsubstituted dibenzofuranylene group, a substituted or unsubstituted dibenzothiophenylene group, or a substituted or unsubstituted carbazolylene group,
L³ is a single bond or a substituted or unsubstituted phenylene group, and
Ar³ is a substituted or unsubstituted phenyl group, a substituted or unsubstituted biphenyl group, a substituted or unsubstituted terphenyl group, a substituted or unsubstituted dibenzofuranyl group, a substituted or unsubstituted dibenzothiophenyl group, a substituted or unsubstituted carbazolyl group, or a substituted or unsubstituted triphenylsilyl group.

12. The composition for an organic optoelectronic device as claimed in claim 8, wherein at least one of the first compound and the second compound is substituted with one or more deuterium atoms.

13. An organic optoelectronic device, comprising:
an anode and a cathode facing each other, and
an organic layer between the anode and the cathode,
wherein the organic layer includes the compound for an organic optoelectronic device as claimed in claim 1.

14. The organic optoelectronic device as claimed in claim 13, wherein:
the organic layer comprises a light emitting layer, and
the light emitting layer includes the compound for an organic optoelectronic device.

15. The organic optoelectronic device as claimed in claim 14, wherein the light emitting layer further includes a fluorescent dopant, a phosphorescent sensitizer, or a combination thereof.

16. A display device comprising the organic optoelectronic device as claimed in claim 13.

17. An organic optoelectronic device, comprising:
an anode and a cathode facing each other, and
an organic layer between the anode and the cathode,
wherein the organic layer includes the composition for an organic optoelectronic device as claimed in claim 8.

18. The organic optoelectronic device as claimed in claim 17, wherein:
the organic layer comprises a light emitting layer, and
the light emitting layer includes the composition for an organic optoelectronic device.

19. The organic optoelectronic device as claimed in claim 18, wherein the light emitting layer further includes a fluorescent dopant, a phosphorescent sensitizer, or a combination thereof.

20. A display device comprising the organic optoelectronic device as claimed in claim 17.
